(19) United States
(12) Patent Application Publication
(10) Pub. No.: US 2024/0252617 A1
(43) Pub. Date: Aug. 1, 2024

(54) CORONAVIRUS SPIKE PROTEIN DESIGNS, COMPOSITIONS AND METHODS FOR THEIR USE

(71) Applicant: Duke University, Durham, NC (US)

(72) Inventors: Kevin Saunders, Durham, NC (US); Barton F. Haynes, Durham, NC (US)

(21) Appl. No.: 18/276,798

(22) PCT Filed: Feb. 10, 2022
(86) PCT No.: PCT/US2022/015969

§ 371 Date: Aug. 10, 2023

Related U.S. Application Data
(60) Provisional application No. 63/147,948, filed on Feb. 10, 2021.
(60) Provisional application No. 63/149,541, filed on Feb. 15, 2021.
(60) Provisional application No. 63/167,390, filed on Mar. 29, 2021.
(60) Provisional application No. 63/289,312, filed on Dec. 14, 2021.
(60) Provisional application No. 63/303,277, filed on Jan. 26, 2022.

(51) Int. Cl. A61K 39/215; A61K 39/00; A61P 31/14; C07K 14/005; C07K 14/435

(52) U.S. Cl. CPC A61K 39/215; A61P 31/14; C07K 14/005; C07K 14/435; A61K 2039/53; A61K 2039/55555; A61K 2039/575; A61K 2039/70; C07K 2319/50; C12N 2770/20022; C12N 2770/20034

(57) ABSTRACT

The invention is directed to coronavirus based immunogens, including immunogens comprising spike protein and or domains thereof, comprised in multimeric complexes. Provided are also methods of using these immunogens to induce immunogenic responses in a subject.

This application is a National Stage Entry of PCT Application No. PCT/US2022/015969, filed on Feb. 10, 2022, which claims the benefit of and priority to U.S. Application Ser. No. 63/303,277, filed Jan. 26, 2022; U.S. Application Ser. No. 63/289,312, filed Dec. 14, 2021; U.S. Application Ser. No. 63/167,390, filed Mar. 29, 2021; U.S. Application Ser. No. 63/149,541, filed Feb. 15, 2021; and U.S. Application Ser. No. 63/147,948, filed Feb. 10, 2021, the content of each application is herein incorporated by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### GOVERNMENT INTERESTS

This invention was made with government support under the Integrated Preclinical and Clinical AIDS Vaccine Development Grant AI 142596 from NIH, NIAID, DAIDS and a grant from the State of North Carolina from the Federal CARES Act. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on Mar. 18, 2022, is named 2933311-000045-WO1_SL.txt and is 921,603 bytes in size.

### TECHNICAL FIELD

The invention encompasses, in general, modified coronavirus protein SARS-CoV-2 proteins, nucleic acids encoding these, methods of making recombinant proteins and nucleic acids, compositions comprising these and their use in vaccination regimens, and diagnostic assays.

### BACKGROUND

The ongoing global pandemic of the new SARS-CoV-2 coronavirus (CoV) presents an urgent need for the development of effective preventative and treatment therapies.

Development of an effective vaccine for prevention of coronavirus (SARS-2) infection is a global priority.

### SUMMARY

In certain aspects the invention provides modified coronavirus spike proteins designs including without limitation designs based on SARS-CoV-2 (SARS-2), SARS-CoV-1 (CoV1), MERS, or any other coronavirus spike protein. In certain aspects these protein designs comprise a spike protein, various spike portions and/or domains. In certain embodiments, these proteins are designed to form a multimeric complex. In non-limiting embodiments, the domain is a spike receptor binding domain (RBD). In non-limiting embodiments, the domain is a spike N-terminal domain (NTD). In non-limiting embodiments, the domain is a spike fusion peptide (FP). In non-limiting embodiments, the domain is S2. In non-limiting embodiments, the multimeric complexes comprise a ferritin sequence. In non-limiting embodiments, the multimeric complexes comprising a ferritin sequence are designed and are assembled as ferritin fusion proteins. In non-limiting embodiments, the multimeric complexes comprising a ferritin sequence are designed and are assembled via sortase reaction. In non-limiting embodiments, the multimeric complexes comprise encapsulin. These multimeric complexes are nanoparticles.

In certain aspects the invention provides a recombinant fusion protein sequence comprising in order a full-length coronavirus spike protein or a portion thereof, a peptide linker and a self-assembling protein, wherein the fusion protein is comprised in a multimeric protein complex, wherein non-limiting embodiments of sequences are shown in FIG. 18, FIG. 20, FIG. 21, FIG. 22, FIG. 23, and FIG. 45 and Table 1, Table 2, Table 3 and Table 5. In certain aspects the invention provides a recombinant fusion protein comprising a full-length coronavirus spike protein or a portion thereof connected via a linker to a self-assembling protein, wherein the fusion protein is comprised in a multimeric protein complex, wherein non-limiting embodiments of sequences are shown in FIG. 18, FIG. 20, FIG. 21, FIG. 22, FIG. 23, and FIG. 45 and Table 1, Table 2, Table 3 and Table 5.

In certain embodiments, the coronavirus spike proteins or portions thereof is RBD—amino acids 319-541; RBDcore—amino acids 333-524 see FIG. 21B, RBDcoreExt—amino acids 333-527 or RBDcoreExtKK—amino acids 333-529 see FIG. 22. In certain embodiments, the coronavirus spike proteins or portions thereof is N-terminal domain (NTD)—amino acids 27-292, wherein amino acid positions are with respect to CoV2 sequence. In certain embodiments, the linker is described in Table 4. In certain embodiments, the linker is LPXTGGGGGGSG (SEQ ID NO: 1) or GSGLPXTGGGGG (SEQ ID NO: 2), wherein in some embodiments X is E. In certain embodiments, the linker is GGS(n) (SEQ ID NO: 3), wherein n=1, 2, or 3. In certain embodiments, the linker is GGS(n)EKAAKAEEAAR(PP) (SEQ ID NO: 4) the linker is EKAAKAEEAAR(PP)GGS(n) wherein n=1, 2, or 3 (SEQ ID NO: 5).

In certain embodiments, the recombinant fusion protein comprises, consists essentially of or consists of amino acid sequences shown in FIG. 18C-1 through FIG. 18C-15, FIG. 23B.

In certain embodiments, the self-assembling protein is ferritin and the multimeric protein complex is referred to as ferritin nanoparticle.

In certain aspects the invention provides a nucleic acid encoding the coronavirus spike protein or fragment thereof in the recombinant fusion protein of the invention.

In certain aspects the invention provides a multimeric protein complex comprising a recombinant fusion protein sequence comprising in order a full-length coronavirus spike protein or a portion thereof, a peptide linker and a self-assembling protein that forms the multimeric protein complex.

In certain embodiments of the multimeric protein complex the coronavirus spike proteins or portions thereof is RBD—amino acids 319-541; RBDcore—amino acids 333-524 see FIG. 21B, RBDcoreExt—amino acids 333-527 or RBDcoreExtKK—amino acids 333-529 see FIG. 22. In certain embodiments, the coronavirus spike proteins or portions thereof is N-terminal domain (NTD)—amino acids 27-292, wherein amino acid positions are with respect to CoV2 sequence. In certain embodiments of the multimeric protein complex the linker is described in Table 4. In certain embodiments of the multimeric protein complex the linker is LPXTGGGGGGSG (SEQ ID NO: 1) or GSGLPXTGGGGG (SEQ ID NO: 2), wherein in some embodiments X is E. In certain embodiments of the multimeric protein complex the linker is or comprises [GGS](n) (SEQ ID NO: 6), wherein n=1, 2, 3 or 4. In certain embodiments of the multimeric protein complex the linker is [GGS](n)EKAAKAEEAAR(PP) (SEQ ID NO: 7) the linker is EKAAKAEEAAR(PP)[GGS](n) (SEQ ID NO: 8) wherein n=1, 2, 3 or 4. In certain embodiments of the multimeric protein complex comprises, consists essentially of or consists of amino acid sequences shown in FIG. 18C-1 through FIG. 18C-15, FIG. 23B. In certain embodiments of the multimeric protein complex the self-assembling protein is ferritin and the multimeric protein complex is ferritin nanoparticle.

In certain embodiments of the multimeric protein complex, the multimeric protein complex is multispecific comprising two, three, four or more different recombinant fusion protein sequences comprising in order a full-length coronavirus spike protein or a portion thereof, wherein the two, three, four or more full-length coronavirus spike protein or a portion thereof have different sequences, a peptide linker and a self-assembling protein.

In certain embodiments of the multimeric protein complex, the multimeric protein complex is multispecific comprising two or three different recombinant fusion protein sequence comprising in order a full-length coronavirus spike protein or a portion thereof, a peptide linker and a self-assembling protein, wherein the recombinant fusion protein sequences represent different coronaviruses from subgroups 2a, 2b, 2c, 2d or combinations thereof.

In certain embodiments of the multimeric protein complex, the multimeric protein complex is multispecific comprising a combination of two or three different recombinant fusion protein sequence each one comprising in order a full-length coronavirus spike protein or a portion thereof, a peptide linker and a self-assembling protein. In some embodiments, wherein the recombinant fusion protein sequences comprise alpha, beta, gamma, delta coronavirus sequences or combination thereof.

In certain embodiments, the multimeric protein complex comprises one or more recombinant fusion protein sequence group 2b and one or more recombinant fusion protein sequence group 2c betacoronavirus sequence.

In some embodiments of coronavirus combinations, the recombinant fusion protein sequences comprise one SARS-related BatCoV, e.g without limitation SHCO14 or WIV-1, one group 2C MERs-related CoV, e.g. without limitation MERS-CoV or HKU4, and/or one human SARS-related CoV, e.g. without limitation SARS-CoV1 or SARS-CoV-2 sequence or a variant thereof. In some embodiments, these combinations include only two of the three viruses. Non-limiting embodiments of combination of viruses are: MERS-CoV, SARS-CoV-2, SHC014, or any combination thereof; MERS-CoV, SARS-CoV-1, SHC014 or any combination thereof; MERS-CoV, SARS-CoV-2, WIV-1 or any combination thereof; MERS-CoV, SARS-CoV-1, WIV-1 or any combination thereof; HKU4, SARS-CoV-2, SHC014 or any combination thereof; HKU4, SARS-CoV-1, SHC014 or any combination thereof; HKU4, SARS-CoV-2, WIV-1 or any combination thereof; HKU4, SARS-CoV-1, WIV-1 or any combination thereof. In some embodiment, the combination includes sequences from SARS-CoV1 and MERS-CoV1 coronaviruses. In some embodiments, the combination includes viruses for example as represented in FIG. 18, 20, 21 22, 23, 45 and FIG. 18C-1 through FIG. 18C-15. In some embodiments, HuPn and PDCoV-Haiti RBD are combined with a SARS-related betacoronavirus and/or MERS-related CoV to protect humans from severe infection by any of these or similar viruses. In certain embodiments of the multimeric protein complex, the self-assembling protein is ferritin, and wherein the multimeric protein complex is a ferritin nanoparticle.

In certain aspects the invention provides a composition comprising a recombinantly produced fusion protein or a portion thereof and a pharmaceutically acceptable carrier.

In some aspects the invention provides a composition comprising a plurality of multimeric protein complexes comprising a recombinant fusion protein sequence comprising in order a full-length coronavirus spike protein or a portion thereof, a peptide linker and a self-assembling protein that forms the multimeric protein complex. In certain embodiments, the plurality of multimeric protein complexes comprises complexes each having identical recombinant fusion protein sequence comprising in order a full-length coronavirus spike protein or a portion thereof, a peptide linker and a self-assembling protein. In certain embodiments, the plurality of multimeric protein complexes comprises a combination of two, three, or four or more types of multimeric complexes each having different recombinant fusion protein sequence comprising in order a full-length coronavirus spike protein or a portion thereof, a peptide linker and a self-assembling protein. Coronavirus combinations are described throughout.

In certain aspects, the invention provides a composition comprising a nucleic acid encoding a recombinant fusion protein of the invention or a combination thereof and a pharmaceutically acceptable carrier. In certain embodiments, the nucleic acid is an mRNA. In certain embodiments, the mRNA is modified mRNA and the composition further comprises LNPs. Coronavirus combinations are described throughout. In some embodiments, the combination includes sequences from SARS-CoV1 and MERS-CoV1 coronaviruses. In some embodiments, the combination includes viruses for example as represented in FIG. 18, 20, 21 22, 23, 45 and FIG. 18C-1 through FIG. 18C-15.

In certain aspects the invention provides a virus-like particle comprising any one of the recombinant fusion protein of the invention.

In certain aspects the invention provides a host cell comprising a nucleic acid molecule encoding a recombinant fusion protein of the invention.

In certain aspects the invention provides a method of producing a multimeric protein complex of the invention the method comprising: expressing a nucleic acid molecule or vector encoding a recombinant fusion protein in a host cell under conditions suitable to produce the recombinant fusion protein; purifying the recombinant fusion protein from the host cell by any suitable purification methods; contacting/reacting the recombinant fusion protein with a self-assembling ferritin protein in the presence of a sortase enzyme in a conjugation reaction, under suitable conjugation conditions to form by sortase conjugation a multimeric ferritin protein complex which comprises the recombinant fusion protein; and isolating the multimeric ferritin protein complex conjugated to the recombinant fusion protein from the rest of the conjugation reaction, which includes unreacted recombinant fusion protein, self-assembling ferritin protein, multimeric ferritin protein complex unconjugated to the recombinant fusion protein and the sortase enzyme. In some embodiments, the self-assembling ferritin protein is formed in a multimeric protein complex prior to contacting with the recombinant fusion protein.

In certain aspects, the invention provides an immunogenic composition comprising any of the recombinant fusion proteins, nucleic acids encoding these recombinant fusion proteins, multimeric protein complex or VLP comprising recombinant fusion proteins of the invention and a pharmaceutically acceptable carrier and an adjuvant.

In certain aspects the invention provides a method of inducing an immune response to a coronavirus in a subject in need thereof, comprising administering to the subject in an effective amount of any of the recombinant fusion proteins of the invention, nucleic acids encoding these recombinant fusion proteins, multimeric protein complexes or VLPs comprising recombinant fusion proteins of the invention and/or the immunogenic composition of the invention in an amount sufficient to induce an immune response. In non-limiting embodiments the immune response includes a response to more than one sarbecovirus. The methods of inducing an immune response comprising administering compositions as prime, boost, or multiple boosts. In some embodiments, the compositions of the invention are administered as a boost after immunization with SARS-CoV2 vaccine. The optimal immunization scheme can be readily determined by the skilled artisan.

In certain aspects, the invention provides modified coronavirus spike proteins designs including without limitation designs based on SARS-CoV-2 (SARS-2), SARS-CoV-1 (CoV1), MERS, or any other corona virus spike protein. In certain embodiments, the protein design provides a stabilized protein conformation(s) of the SARS-2 trimer. In certain embodiments, the inventive designs are recombinant proteins. In certain embodiments, the inventive designs are nucleic acids. Nucleic acids include without limitation modified mRNAs.

In certain embodiments, a multimeric complex of the invention comprises coronavirus spike proteins or portions thereof, e.g. but not limited to RBD, NTD, FP, S2 or any other portion, from the same human or animal coronaviruses. In certain embodiments, a multimeric complex of the invention comprises coronavirus spike proteins or portions thereof, e.g. but not limited to RBD, NTD, FP, S2 or any other portion, from different human or animal coronaviruses and with different sequences. A multimeric complex comprising coronavirus spike proteins or portions thereof, e.g. but not limited to RBD, NTD, FP, S2 or any other portion, from different human or animal coronaviruses and with different sequences is a multispecific complex.

In certain aspects, the invention also provides compositions comprising a combination of different multimeric complexes comprising different coronavirus spike proteins or portions, wherein each multimeric complex comprises coronavirus spike proteins or portions thereof, e.g. but not limited to RBD, NTD, FP, S2 or any other portion, from the same human or animal coronaviruses. In certain aspects, the invention provides compositions comprising a combination of different multimeric complexes comprising different coronavirus spike proteins or portions, wherein each a multimeric complex of the invention comprises coronavirus spike proteins or portions thereof, e.g. but not limited to RBD, NTD, FP, S2 or any other portion, from different human or animal coronaviruses and with different sequences.

Table 1, Table 3 and Table 3 list non-limiting examples of immunogens designs from different human or animal coronaviruses with different sequences. These sequence designs are non-limiting examples of spike proteins or portions thereof that can be used in multispecific multimeric complexes and/or in multimeric complexes in multispecific compositions. In certain embodiments, the sequences are variants of the same coronavirus, e.g. CoV2. In certain embodiments, the sequences are from different betacoronavirus groups, e.g. 2a, 2b, 2c, 2d. In certain embodiments, the sequences are from CoV1 and CoV2. In certain embodiments, the sequences are from CoV1 and MERS, or MERS related CoV. In certain embodiments, sequences can be from alpha, beta, gamma or delta coronaviruses, or any combinations thereof. In certain embodiments, the multimeric complex comprises spike protein sequence from MERS, Pangolin and bat, e.g. but not limited to RBDs, NTDs, FPs, S2 domains, or any other portion or combinations linked to ferritin. In certain embodiments, the multimeric complex comprises spike protein sequences or portions thereof from any circulating coronavirus strain, e.g. but not limited to California, UK and RSA Cov2 variants linked to ferritin. Multimeric complexes comprising different coronavirus spike proteins or portions thereof could be combined. Certain non-limiting embodiments of multimeric complexes comprise spike protein, various spike portions and/or domains, or combinations thereof from designs listed in Table 1, Table 2 and Table 3. In non-limiting embodiments, to include coverage of group 2B and 2C betacoronaviruses, multimeric complexes comprise one SARS-related BatCoV, e.g without limitation SHC014 or WIV-1, one group 2C MERs-related CoV, e.g. without limitation MERS-CoV or HKU4, and/or one human SARS-related CoV, e.g. without limitation SARS-CoV1 or SARS-CoV-2 sequence or a variant thereof. These combinations can also be paired down to include only two of the three viruses. Non-limiting embodiments of combination of viruses are: MERS-CoV, SARS-CoV-2, SHC014, or any combination thereof; MERS-CoV, SARS-CoV-1, SHC014 or any combination thereof; MERS-CoV, SARS-CoV-2, WIV-1 or any combination thereof; MERS-CoV, SARS-CoV-1, WIV-1 or any combination thereof; HKU4, SARS-CoV-2, SHC014 or any combination thereof; HKU4, SARS-CoV-1, SHC014 or any combination thereof; HKU4, SARS-CoV-2, WIV-1 or any combination thereof; HKU4, SARS-CoV-1, WIV-1 or any combination thereof. Non-limiting embodiments of a pan coronavirus vaccine we will include RBDs or any other spike portion from novel alphacoronaviruses and deltacoronaviruses that can infect humans. In some embodiments, HuPn and PDCoV-Haiti RBD will be combined with a SARS-related betacoronavirus and/or MERS-related CoV to protect humans from severe infection by any of these or similar viruses.

In certain aspects, the invention provides a recombinant fusion protein comprising a full-length coronavirus spike protein or a portion/domain thereof connected via a linker to a self-assembling protein/a multimerization domain, wherein the fusion protein forms a multimeric complex. In certain aspects, the invention provides a recombinant fusion protein comprising a full-length coronavirus spike protein or a portion/domain thereof connected via a linker to a self-assembling protein/a multimerization domain, wherein the fusion protein is comprised in a multimeric complex.

In non-limiting embodiments the portion of the coronavirus spike protein is RBD. In non-limiting embodiments the portion of the coronavirus spike protein is fusion peptide (FP). In certain embodiments, the portion of the coronavirus spike protein is N-terminal domain (NTD). Non-limiting embodiments of sequences are shown in FIG. 18, FIG. 20, FIG. 21, FIG. 22, and FIG. 23 and Table 1, Table 2 and Table 3. In non-limiting embodiments, the recombinant fusion protein is immunogenic.

In non-limiting embodiments the self-assembling protein is ferritin, encapsulin or any other suitable self-assembling protein allowing multimerization.

In certain aspects, the invention provides a composition comprising a recombinantly-produced modified coronavirus spike proteins of the invention or a portion thereof. In certain embodiments, the compositions comprise a plurality of different multimeric complexes each complex comprising a single sequence/specificity recombinantly-produced modified coronavirus spike proteins of the invention or a portion thereof and wherein the composition as whole is multispecific. In certain embodiments, at least one or a few of the plurality of the multimeric complexes comprises a multispecific multimeric complex(es) where each complex comprises two, three, four or more recombinantly-produced modified coronavirus spike proteins of the invention or a portion thereof of different sequences/specificity and wherein each multimeric complex is multispecific. In certain embodiments, the recombinant proteins or fragments are comprised in multimeric complexes. In certain embodiments, the multimeric complex comprises coronavirus spike proteins or portions thereof, e.g. but not limited to RBD, NTD, FP, S2 or any other portion, from different human or animal coronaviruses and with different sequences. Non-limiting examples of different human or animal coronaviruses and with different sequences are listed in Table 1, Table 2 or Table 3. In certain embodiments the sequences are variants of the same coronavirus, e.g. CoV2. In certain embodiments, the sequences are from different betacoronavirus groups, e.g. 2a, 2b, 2c, 2d. In certain embodiments, the sequences are from CoV1 and CoV2. In certain embodiments, the sequences are from CoV1 and MERS. Sequences can be from alpha, beta, gamma or delta coronaviruses. In certain embodiments, the multimeric complex comprises spike protein sequence from SARS1, MERS, Pangolin and/or bat. In certain embodiments, the multimeric complex comprises spike protein sequences from any circulating coronavirus strain, e.g. but not limited to California, UK, delta, omicron and RSA Cov2 variants linked to ferritin. Multimeric complexes comprising different coronavirus spike proteins or portions thereof could be combined.

Compositions with nucleic acid for multispecific NPs.

In non-limiting embodiments, to include coverage of group 2B and 2C betacoronaviruses, combinations in multimeric multispecific complexes or multispecific composition comprise multimeric complexes including one SARS-related BatCoV, e.g without limitation SHCO14 or WIV-1, one group 2C MERs-related CoV, e.g. without limitation MERS-CoV or HKU4, and/or one human SARS-related CoV, e.g. without limitation SARS-CoV1 or SARS-CoV-2 sequence or a variant thereof. These combinations can also be paired down to include only two of the three viruses. Non-limiting embodiments of combination of viruses are: MERS-CoV, SARS-CoV-2, SHC014, or any combination thereof; MERS-CoV, SARS-CoV-1, SHCO14 or any combination thereof; MERS-CoV, SARS-CoV-2, WIV-1 or any combination thereof; MERS-CoV, SARS-CoV-1, WIV-1 or any combination thereof; HKU4, SARS-CoV-2, SHCO14 or any combination thereof; HKU4, SARS-CoV-1, SHCO14 or any combination thereof; HKU4, SARS-CoV-2, WIV-1 or any combination thereof; HKU4, SARS-CoV-1, WIV-1 or any combination thereof. These combinations can also be paired down to include only two of the three viruses. Non-limiting embodiments of a pan coronavirus vaccine we will include RBDs or any other spike portion from novel alphacoronaviruses and deltacoronaviruses that can infect humans. In some embodiments, HuPn and PDCoV-Haiti RBD will be combined with a SARS-related betacoronavirus and/or MERS-related CoV to protect humans from severe infection by any of these or similar viruses.

In certain embodiments the coronavirus spike proteins or portions thereof is RBD—amino acids 319-541; RBDcore—amino acids 333-524 see FIG. 21B, RBDcoreExt—amino acids 333-527 or RBDcoreExtKK—amino acids 333-529 see FIG. 22. In certain embodiments, the coronavirus spike proteins or portions thereof is N-terminal domain (NTD)—amino acids 27-292). Amino acid positions with respect to CoV2 sequence XX.

In non-limiting embodiments the linker is listed in Table 4. In non-limiting embodiments the linker is LPXTGGGGGGSG (SEQ ID NO: 1) or GSGLPXTGGGGG (SEQ ID NO: 2) comprising linker. In some embodiments X is E. In non-limiting embodiments the linker is GSG(n) where n=1, 2, or 3 (SEQ ID NO: 29). In non-limiting embodiments the linker is EKAAKAEEAAR (SEQ ID NO: 9), EKAAKAEEAARP (SEQ ID NO: 10), EKAAKAEEAARPP (SEQ ID NO: 11), GGSEKAAKAEEAAR (SEQ ID NO: 12), GGSEKAAKAEEAARP (SEQ ID NO: 13), or GGSEKAAKAEEAARPP (SEQ ID NO: 14).

In certain embodiments, the invention provides a recombinant fusion protein comprising all the consecutive amino acids after the signal peptide of the polypeptide sequences described herein. For specific non-limiting embodiments of sequences see FIG. 18, FIG. 20, FIG. 21, FIG. 22, and FIG. 23 and Table 1, Table 2 and Table 3. In one embodiment the recombinant fusion protein is shown in FIG. 18C-1 (SEQ ID NO: 97).

In certain aspects the invention provides a nucleic acid encoding the coronavirus spike protein or fragment thereof, and in some embodiment any suitable tag or linker, in the recombinant fusion protein of the invention. In certain aspects the invention provides a nucleic acid encoding the recombinant fusion protein of the invention.

In certain aspects the invention provides a composition comprising a recombinantly produced fusion protein or a portion thereof of and a carrier. In certain embodiments, the compositions are immunogenic. In certain embodiments the compositions comprise an adjuvant. Any suitable adjuvant could be used. In certain embodiments the compositions comprise a plurality of recombinantly produced fusion protein or portion thereof. In certain embodiments each recombinantly produced fusion protein or portion thereof is of the same sequence. In certain embodiments recombinantly produced fusion proteins or portions thereof comprised in a composition have different sequence, e.g. without limitation sequences are from different types, e.g. 2a, 2b, 2c, or 2d, sequences of variants or mutations from the same strain, e.g. different sequences from CoV1 and/or CoV2.

In certain aspects the invention provides composition comprising a nucleic acid encoding a recombinant fusion protein of the invention and a carrier.

In certain aspects the invention provides protein nanoparticle, comprising any one of the recombinant fusion protein of the invention. In certain embodiments, the protein nanoparticle subunit is a ferritin nanoparticle subunit. A composition comprising a plurality of nanoparticles, comprising a multimerized recombinant coronavirus protein of the invention or a portion thereof and a carrier.

In certain aspects the invention provides A virus-like particle comprising any one of the recombinant fusion protein of the invention.

In certain aspects the invention provides a host cell comprising a nucleic acid molecule encoding a recombinant fusion protein of the invention.

In certain aspects the invention provides a method of producing a recombinant fusion protein of the invention, comprising: expressing the nucleic acid molecule or vector comprising a nucleic acid encoding in a host cell to produce the recombinant protein antigen; purifying the recombinant protein antigen; contacting/reacting the recombinant protein antigen with a ferritin protein, wherein in some embodiments the ferritin protein is multimerized in a ferritin multimeric complex, in the presence of a sortase enzyme, under conditions suitable to form a multimeric complex linked to the recombinant protein antigen; and isolating the antigen(s) comprising multimeric complex from unreacted recombinant protein antigen, ferritin and sortase enzyme.

An immunogenic composition comprising any one of the recombinant fusion protein, nucleic acids encoding these recombinant fusion proteins, nanoparticle or VLP of the invention and a pharmaceutically acceptable carrier. In some embodiments, the immunogenic composition further comprises an adjuvant.

A method for inducing an immune response to a coronavirus in a subject, comprising administering to the subject an effective amount of any one of the recombinant fusion proteins of the invention and/or an immunogenic composition of the invention in an amount sufficient to induce an immune response.

In certain embodiments, the method comprising administering a combination of immunogens targeting coronavirus from group 2a, 2b, 2c or 2d.

The recombinant protein of the invention, wherein the recombinant protein is produced by conjugating a spike protein or portion thereof to a multimerizing protein in a sortase mediated conjugation reaction.

In certain aspects, the invention provides modified coronavirus spike proteins, for example but not limited in a stabilized conformation, nucleic acid molecules and vectors encoding these proteins, and methods of their use and production are disclosed. In several embodiments, the coronavirus modified coronavirus spike proteins and/or nucleic acid molecules can be used to generate an immune response to coronavirus in a subject. In additional embodiments, the therapeutically effective amount of the coronavirus modified coronavirus spike proteins and/or nucleic acid molecules can be administered to a subject in a method of treating or preventing coronavirus infection. In certain embodiments, the proteins of the invention could be used in diagnostic assays.

In certain aspects, the invention provides spike ectodomain trimers in a stabilized conformation, nucleic acid molecules and vectors encoding these proteins, and methods of their use and production are disclosed. In several embodiments, the coronavirus spike ectodomain trimers and/or nucleic acid molecules can be used to generate an immune response to coronavirus in a subject. In additional embodiments, the therapeutically effective amount of the coronavirus S ectodomain trimers and/or nucleic acid molecules can be administered to a subject in a method of treating or preventing coronavirus infection. In certain embodiments, the proteins of the invention could be used in diagnostic assays.

In certain embodiments, the modified SARS-2 spike proteins do not include modification as described in US Patent Publication 20200061185.

The invention provides amino acid or nucleic acids sequences encoding such spike protein designs. Provided are also nucleic acids, including modified mRNAs which are stable and could be used as immunogens. Non-limiting embodiments include recombinant proteins, trimers, multimerized proteins, e.g. but not limited to nanoparticles. Provided also are nucleic acids optionally designed as vectors, for example for recombinant expression and/or stable integration, e.g. but not limited, DNA encoding trimer for stable expression, or virus-like particle (VLP) incorporation. In non-limiting embodiments, nucleic acids are mRNA, including but not limited to modified mRNA which are used immunogens. Modified mRNAs could be formulated in any suitable formulation, including but not limited to lipid nanoparticles (LNPs).

In some embodiments a protein design is based on SARS-2 spike protein and is characterized as having 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% similarity or identity to the designs described herein.

In non-limiting embodiments the invention provides modified coronavirus spike proteins or portions thereof designed to form a multimeric complex, nucleic acid molecules and vectors encoding these proteins, and methods of their use and production are disclosed. In several embodiments, the multimeric complexes and/or nucleic acid molecules can be administered in therapeutic amounts to generate an immune response to coronavirus in a subject. In certain embodiments, the therapeutically effective amount can be administered to a subject in a method of treating or preventing coronavirus infection. In certain embodiments, the therapeutically effective amount can be administered to a subject in a method of inhibiting or reducing coronavirus replication in a subject.

In certain aspects, the invention provides a recombinant coronavirus protein comprising all the consecutive amino acids after the signal peptide of the amino acid sequences described herein. For specific non-limiting embodiments of sequences see FIG. 18, FIG. 20, FIG. 21, FIG. 22, FIG. 23, and FIG. 45. Provided are also methods of using these coronavirus proteins to reduce viral replication.

In certain aspects, the invention provides a nucleic acid encoding the modified coronavirus protein of the invention. In non-limiting embodiments, the nucleic acid is a modified mRNA. In certain embodiments, the mRNA is in a composition comprising LNPs.

In certain embodiments, the nucleic acid is comprised in a vector and is operably linked to a promoter.

Any one of the modifications described herein could be engineered in a full length coronavirus spike sequence or in a fragment, e.g. but not limited to the ectodomain, RBD, NTD, or FP.

In certain aspects, the invention provides a composition comprising a recombinantly-produced modified coronavirus spike proteins of the invention or fragments thereof. In certain embodiments, the compositions are multispecific and comprise multimeric complexes each with different specificity. In certain embodiments, the multimeric complexes are multispecific. In certain embodiments, the recombinant proteins or fragments are comprised in multimeric complexes. In certain embodiments, the multimeric complexes comprise ferritin. In certain embodiments, the compositions comprise a carrier.

In certain embodiments, the compositions are immunogenic. In certain embodiments the compositions comprise an adjuvant. Any suitable adjuvant could be used. In non-limited embodiments the adjuvant is TLR7/8 agonist. In certain embodiment the TLR7/8 agonist is a compound represented by Formula (I) or a pharmaceutically acceptable salt thereof. In non-limiting embodiments the adjuvant is 3M-052 or a pharmaceutically acceptable salt thereof. In some embodiments the adjuvant is formulated in alum. In some embodiments, the adjuvant is formulated as stable emulsion (SE). In non-limiting embodiments the adjuvant is 3M-052 formulated in alum. In non-limiting embodiments the adjuvant is 3M-052 formulated in SE. In non-limiting embodiments the adjuvant is alum.

In certain embodiments the invention provides a recombinant fusion protein comprising spike protein domain connected by a linker to a self-assembling protein which multimerizes wherein the fusion protein forms a multimeric complex. In certain embodiments the spike protein domain is RBD, NTD, FP or any other portion. In certain embodiments, the spike protein domain is RBD, wherein the RBD is from a beta coronavirus. In certain embodiments the self-assembling protein is ferritin.

In certain embodiments the invention provides a composition comprising a recombinant fusion protein of the invention and an adjuvant. In certain embodiments the invention provides a composition comprising a recombinant protein of FIG. 18C-1 (SEQ ID NO: 97) and an adjuvant. In certain embodiments the adjuvant is TLR7/8 agonist is a compound represented by Formula (I) or a pharmaceutically acceptable salt thereof. In certain embodiments the adjuvant is formulated as described herein. In certain aspects the invention provides compositions which comprise recombinant fusion proteins comprising spike protein domains with different sequences. In non-limited embodiments, the sequences are from different beta coronavirus groups.

In certain aspects the invention provides methods of using the recombinant fusions proteins of the invention and compositions comprising these to induce immune responses in a subject. Without being bound by a specific theory, administering recombinant fusion proteins of the invention in combination with a TLR7/8 agonist leads to unexpected results, e.g. but not limited to results described in Example 1A and Example 3.

In certain aspects the invention provides a composition comprising a nucleic acid encoding any of the modified coronavirus spike proteins and a carrier. Also disclosed herein are modified mRNA, for example comprising suitable modifications for expression as immunogens. Non-limiting examples include modified nucleosides, capping, polyA tail, and the like. In certain embodiments the compositions comprise an adjuvant.

In certain embodiments the designs produce a soluble protein. In certain embodiments the designs are comprised in a protomer which can form a trimer. In certain embodiments the designs comprise a TM domain.

In certain embodiments the compositions comprise a coronavirus spike ectodomain trimer comprising protomers comprising sequence modification as described herein.

In non-limiting embodiments, the designs comprise additional modifications to allow multimerization. In non-limiting embodiments, wherein the design comprises a soluble ectodomain, additional modifications could be included to allow multimerization. In a non-limiting embodiment, a C-terminal residue of the protomers in the ectodomain is linked to a trimerization domain by a peptide linker, or is directly linked to the trimerization domain. In some embodiments, the trimerization domain is a T4 fibritin trimerization domain. In one example, a T4 Fibritin trimerization domain comprises the amino acid sequence set forth as GYIPEAPRDGQAYVRKDGEWVLLSTF (SEQ ID NO: 15). In some embodiments, a protease cleavage site (such as a thrombin cleavage site) can be included between the C-terminus of the recombinant coronavirus ectodomain and the T4 Fibritin trimerization domain to facilitate removal of the trimerization domain as needed, for example, following expression and purification of the recombinant SARS-2 S ectodomain.

In certain embodiments, the modified coronavirus spike designs further comprise furin protease cleavage site and/or a cathepsin L cleavage site. In certain embodiments, the modified coronavirus spike protein trimer is soluble.

In certain embodiments, a C-terminal residue of the protomers in the ectodomain of the modified SARS-2 spike protein or the portion thereof is linked to a transmembrane domain by a peptide linker, or is directly linked to the transmembrane domain. In certain embodiments, the modified coronavirus spike protein or the portion thereof is linked to form a protein multimerizing/nanoparticle subunit by a peptide linker in a sortase reaction, or is directly linked to the protein multimerizing/nanoparticle subunit. In certain embodiments, the protein nanoparticle subunit is a ferritin nanoparticle subunit.

In certain aspects the invention provides a protein nanoparticle, comprising any one of the protein immunogens of the invention.

In certain aspects the invention provides a virus-like particle comprising any one of the immunogens of the invention.

In certain aspects the invention provides an isolated nucleic acid molecule encoding a protomer of the modified coronavirus spike protein of the invention. In certain embodiments, the nucleic acid molecule is operably linked to a promoter. In certain embodiments, the nucleic acid molecule is an RNA molecule.

In certain aspects, the invention provides a vector comprising a nucleic acid molecule encoding any one of the inventive proteins. In certain embodiments, the vector is a viral vector.

In certain aspects, the invention provides an immunogenic composition comprising any one of the proteins and/or nucleic acids of the invention, and a pharmaceutically acceptable carrier.

In certain aspects, the invention provides a method of producing a recombinant coronavirus protein of the invention, comprising: expressing the nucleic acid molecule or vector comprising a nucleic acid encoding in a host cell to produce the recombinant protein, which in certain embodiments is a trimer; and purifying the recombinant protein.

In certain aspects, the invention provides a recombinant cell comprising a nucleic acid encoding the modified coronavirus spike protein of the invention.

In certain aspects the invention provides a method for generating an immune response to a coronavirus, including but not limited to an SARS-CoV-1, SARS-CoV-2 or any other coronavirus in a subject, comprising administering to the subject an effective amount of any one of the immunogens, wherein the immunogen is a recombinant protein of the invention, a nucleic acid encoding these, and/or a combination thereof to induce an immune response. In certain embodiments, the recombinant protein is formulated with any suitable adjuvant. In certain embodiments, the nucleic acid is DNA which could be administered by any suitable method. In certain embodiments, the nucleic acid is an mRNA, which could be administered by any suitable methods. In certain embodiments, the mRNA is formulated in an LNP. A skilled artisan can readily determine the dose and number of immunizations needed to induce immune response. Various assays are known and used in the art to measure to level, breadth and durability of the induced immune response.

In certain embodiments, the immune response treats, prevents or inhibits infection with coronavirus, including but not limited to the SARS-CoV-1, SARS-CoV-2 virus or any variant thereof. In certain embodiments, the immune response generated by the immunogens inhibits replication of coronavirus, including but not limited to the SARS-CoV-1, SARS-CoV-2 or any variant in the subject.

### BRIEF DESCRIPTION OF DRAWINGS

FIG. 1a-j. SARS-CoV-2 receptor binding domain (RBD) sortase conjugated nanoparticles (scNPs) elicits extremely high titers of SARS-CoV-2 pseudovirus neutralizing antibodies. FIG. 1a. SARS-CoV-2 RBD nanoparticles were constructed by expressing RBD with a C-terminal sortase A donor sequence (blue and red) and a Helicobacter pylori ferritin nanoparticle with N-terminal sortase A acceptor sequences (gray) on each subunit (top left). The RBD is shown in blue with the ACE2 binding site in red. The RBD was conjugated to nanoparticles by a sortase A (SrtA) enzyme conjugation reaction (top right). The resultant nanoparticle is modeled on the bottom left. Nine amino acid sortase linker is shown in orange. Two dimensional class averages of negative stain electron microscopy images of actual RBD nanoparticles are shown on the bottom right. FIG. 1b. Antigenicity of RBD nanoparticles determined by biolayer interferometry against a panel of SARS-CoV-2 antibodies and the ACE2 receptor. Antibodies are color-coded based on epitope and function. N-terminal domain (NTD), nonAbs IE, infection enhancing non-neutralizing antibody; nAb, neutralizing antibody; nonAb, non-neutralizing antibody. Mean and standard error from 3 independent experiments are shown. FIG. 1c. Cynomolgus macaque challenge study scheme. Blue arrows indicate RBD-NP immunization timepoints. Intranasal/intratracheal SARS-CoV-2 challenge is indicated at week 11. FIG. 1d. Macaque serum IgG binding determined by ELISA to recombinant SARS-CoV-2 stabilized Spike ectodomain (S-2P), RBD, NTD, and Fusion peptide (FP). Binding titer is shown as area-under-the curve of the log₁₀-transformed curve. Arrows indicate immunization timepoints. FIG. 1e. Plasma antibody blocking of SARS-CoV-2 S-2P binding to ACE2-Fc and RBD neutralizing antibody DH1041. Group mean and standard error are shown. FIG. 1f. Dose-dependent serum neutralization of SARS-COV-2 pseudotyped virus infection of ACE2-expressing 293T cells. Serum was examined after two immunizations. The SARS-CoV-2 pseudovirus spike has an aspartic acid to glycine change at position 614 (D614G). Each curve represents a single macaque. SARS-CoV-2 RBD nAb DH1043 spiked into normal human serum was used as a positive control. FIG. 1g. Heat map of serum neutralization ID50 and ID80 titers for SARS-COV-2 D614G pseudotyped virus after two immunizations. SARS-CoV-2 RBD nAb DH1043 spiked into normal human serum was used as a positive control. FIG. 1h. SARS-COV-2 D614G pseudotyped virus serum neutralization kinetics. Each curve represents a single macaque. Pre-immunization serum was used as a negative control. FIG. 1i. Comparison of serum neutralization ID50 titers from cynomolgus macaques immunized with recombinant protein RBD nanoparticles (blue) or nucleoside-modified mRNA-LNP expressing S-2P (burgundy) (**P<0.01, Two-tailed Exact Wilcoxon test n=5). Vaccinated macaque serum was collected after two immunizations. Negative and positive controls are shown as in g and h. FIG. 1j. Comparison of serum neutralization titers obtained from RBD-scNP-vaccinated macaques (blue, N=5) and SARS-CoV-2 infected humans (shades of green). Vaccinated macaque serum was collected after two immunizations. Human samples were stratified based on disease severity as asymptomatic (N=34), symptomatic (n=71), and hospitalized (N=60) (**P<0.01, Two-tailed Wilcoxon test).

FIG. 2 a-i. RBD-scNP immunization elicits higher titers of neutralizing antibodies against more transmissible or neutralization-resistant SARS-CoV-2 variants than stabilized spike mRNA-LNP vaccination. FIG. 2a,b. The position of B.1.351 RBD mutations relative to the DH1041 and DH1047 binding sites. The location of K417, E484, and N501 (spheres) are shown in the cryo-EM structures of a DH1041 (red) and b DH1047 (magenta) bound to the RBD (gray) of S trimers (PDB: 7LAA and 7LD1)²⁴. The contact surface on S for each antibody is shown as atoms outlined with dotted lines. The E484K is present in the DH1041 binding surface. DH1047 binds adjacent to all three mutations using a long HCDR3. FIG. 2c ACE2 receptor, ACE2 binding site-targeting neutralizing antibody DH1041, and cross-neutralizing antibody DH1047 ELISA binding to wildtype and mutant SARS-CoV-2 Spike RBD monomers. RBD variants contain mutations found in circulating B.1.351 and P.1 virus strains. Titers are shown as area under the log-transformed curve (log AUC) in a heatmap. FIG. 2d Comparison of serum neutralization titers for SARS-CoV-2 D614G and SARS-CoV-2 B.1.1.7 pseudoviruses from cynomolgus macaques immunized with recombinant protein RBD sortase conjugated nanoparticles (RBD-scNP; blue) or nucleoside-modified mRNA-LNP expressing S-2P (burgundy). ID50 titers (left) and ID80 titers (right) are shown. Horizontal bars are the group geometric mean (**P<0.01, Two-tailed Exact Wilcoxon test n=5). FIG. 2e Fold decrease in neutralization potency between neutralization of SARS-CoV-2 D614G and SARS-CoV-2 B.1.1.7 pseudoviruses. Fold change is shown for RBD-scNP-immunized and mRNA-LNP-immunized macaques based ID50 (left) and ID80 (right) titers. Horizontal bars are the group mean. FIG. 2f Comparison of vaccinated macaque serum neutralization ID50 (left) and ID80 titers (right) against WA-1 and B.1.351 pseudovirus. Symbols and horizontal bars are shown the same as in d (*P<0.05 and **P<0.01, Two-tailed Exact Wilcoxon test n=5). FIG. 2g Fold decrease in neutralization potency between neutralization of SARS-CoV-2 WA-1 and B.1.351 pseudoviruses. Fold change is shown the same as in e. FIG. 2h Comparison of vaccine induced neutralization of SARS-CoV-2 WA-1 and P.1 pseudovirus infection of ACE2 and TMPRSS2-expressing 293 cells. Symbols and horizontal bars are shown the same as in d (*P<0.05 and **P<0.01, Two-tailed Exact Wilcoxon test n=5). FIG. 2i Fold decrease in neutralization potency as shown in e between neutralization of SARS-CoV-2 WA-1 and P.1 pseudoviruses.

FIG. 3 a-h. RBD-scNP vaccine induction of serum cross-neutralization of SARS-related betacoronavirus infection. FIG. 3a. Sera from macaques immunized twice with RBD-scNP, S-2P mRNA-LNP, or RBD mRNA-LNP neutralize replication-competent SARS-related human (SARS-CoV-1 and SARS-CoV-2) and bat (WIV-1 and SHC014) betaCoVs. Each symbol indicates the ID50 of an individual macaque. Horizontal bars are the group mean (*P<0.05, **P<0.01, Two-tailed Exact Wilcoxon test, n=5 or 8). FIG. 3b. Comparison of serum cross-neutralization before (gray, hexagons), after two (light blue, circles) or after three (blue, diamonds) RBD scNP immunizations. Each symbol indicates the ID50 of an individual macaque. Bars indicate the group geometric mean. FIG. 3c. Plasma IgG from macaques immunized twice with RBD-scNP binds to human, bat, and pangolin SARS-related betacoronavirus S protein in ELISA. ECD, ectodomain. FIG. 3d. Cryo-electron microscopy model comparing the epitopes of SARS-CoV-2-specific neutralizing RBD antibody (DH1041) and cross-neutralizing RBD antibody (DH1047). (Left) Cartoon view of Spike bound to DH1047 (magenta; PDB ID: 7LD1) fitted in the cryo-EM map (transparent gray; EMD-23279). Receptor Binding Domain (RBD) is in gray, Receptor Binding Motif (RBM) is in blue, and the rest of Spike is in forest green. (Top, right) Zoomed-in surface view of RBD (gray) and RBM (blue) binding interface with DH1047 (magenta) compared to the neutralizing RBD antibody DH1041 (red; PDB ID: 7LAA; EMD-23246). The RBDs of the two complexes from their respective cryo-EM structures were overlaid for comparison. (Bottom, right) A 180° rotated view of the top-right panel. FIG. 3e. DH1047 epitope is conserved within group 2b betaCoVs. Receptor binding domain (surface representation) colored by conservation within group 2b betacoronaviruses. DH1047 epitope is shown in magenta outline. FIG. 3f. Sequence similarity of RBD for representative betacoronaviruses. Heatmaps displaying pairwise amino acid sequence similarity for 57 representative betacoronaviruses. Dark blue shading indicates high sequence similarity. FIG. 3g. Macaque plasma antibody blocking of SARS-CoV-2 and batCoV-SHC014 S-2P binding to ACE2-Fc (gray) and RBD cross-neutralizing antibody DH1047 (navy blue). Group mean and standard error are shown (n=5). Blocking above 20% (above the dashed line) is considered positive. FIG. 3h. Human serum or macaque plasma antibody blocking of ACE2 (left) and DH1047 (right) binding to SARS-CoV-2 diproline-stabilized spike ectodomain (S-2P). Macaques were immunized with RBD-scNP or S-2P mRNA-LNP twice. Humans were immunized with Pfizer BNT162b2 twice or naturally infected with SARS-CoV-2. Each symbol represents a vaccinated macaque, vaccinated human, or infected individual. Filled bars indicate group mean.

FIG. 4 a-g. RBD-scNP vaccination alone or as a boost completely prevents virus replication in the upper and lower respiratory tract after intranasal and intratracheal SARS-CoV-2 challenge in nearly all macaques. FIG. 4a. Macaque intranasal/intratracheal SARS-CoV-2 challenge study design. Blue and maroon arrows indicate the time points for RBD-scNP and mRNA-LNP immunizations respectively. Challenge occurred 3 and 9 weeks after the final immunization for the RBD-scNP group and S-2P mRNA-LNP/RBD-scNP group respectively. Biospecimens were collected as indicated by the green and orange arrows and challenge occurred. FIG. 4b Infectious virus in macaque BAL fluid two days after challenge. FIG. 4c-f. Quantification of viral subgenomic RNA (sgRNA) in unimmunized (gray) and RBD-scNP-immunized (blue), and S-2P mRNA-LNP prime/RBD-scNP boosted (maroon) macaques. sgRNA encoding the c, d envelope (E) gene or e, f nucleocapsid (N) gene of SARS-CoV-2 was quantified two (left) and four (right) days after SARS-CoV-2 challenge. E or N gene sgRNA was measured in both c, e nasal swabs and d, f bronchoalveolar lavage (BAL) fluids from each macaque. Limit of detection (LOD) for the assay is 150 copies/mL. Each symbol represents an individual macaque with the group mean shown with a horizontal bar. FIG. 4g Nucleocapsid immunohistochemistry of lung tissue sections seven days post challenge. A representative image from one macaque from each group is shown. Red arrows indicate site of antigen positivity. All images are shown at 10× magnification with 100 micron scale bars shown in the bottom right corner. Quantification of antigen positivity observed is shown in the graph. The group mean score is shown by a horizontal bar.

FIG. 5A-D. Molecular and structural characterization of the SARS-CoV-2 RBD sortase A conjugated nanoparticle. FIG. 5a Size exclusion chromatography of RBD and ferritin sortase conjugation. The first peak shows conjugated protein. The second peak contains unconjugated RBD. FIG. 5b Analytical size exclusion trace shows a homogenous nanoparticle preparation. FIG. 5c Negative stain electron microscopy image of RBD-scNPs on a carbon grid. Inset shows a zoomed image of RBD-scNP. The zoomed image shows RBD molecules arrayed around the outside of the ferritin nanoparticle. FIG. 5d Chemical structure of toll-like receptor 7 and 8 agonist 3M-052. Alum formulation of 3M-052 was used to adjuvant RBD-scNP immunization.

FIG. 6A-C. Macaque immunization and challenge study designs. FIG. 6a RBD-scNP immunization regimen used for vaccination of cynomolgus macaques (N=5). Blue arrows indicate timepoints for intramuscular immunizations with RBD-scNP (100 μg) adjuvanted with 3M-052 (5 μg 3M-052 plus 500 μg Alum). Bronchoalveolar lavage (BAL, orange arrows) and nasal swab (green arrows) fluids were collected 7 days before, 2 days after, and 4 days after intratracheal/intranasal SARS-CoV-2 challenge (black arrow). FIG. 6b Transmembrane, diproline-stabilized spike (S-2P) mRNA-LNP prime, RBD-scNP boost vaccination of cynomolgus macaques (N=5). Maroon arrows indicate timepoints for S-2P mRNA-LNP immunization (50 μg mRNA dose). Blue arrows are the same as in FIG. 6a. Macaques were challenged 9 weeks after RBD-scNP boost (week 17 of the study). BAL and nasal swab fluids were collected as in FIG. 6a. Macaques were challenged at week 17 (black arrow). FIG. 6c Monomeric RBD mRNA-LNP immunization of rhesus macaques (N=8). Tan arrows indicate timepoints for RBD mRNA-LNP immunization (50 μg mRNA dose). Blood was collected throughout each study as shown by red arrows in all panels.

FIG. 7. Blood chemical analysis and blood cell counts in RBD-scNP and S-2P mRNA-LNP-vaccinated macaques. Each graph shows values for individual macaques before vaccination and 4 weeks after the RBD-scNP (week 8) or 6 weeks after the second S-2P mRNA-LNP immunization (week 10). RBD-scNP-immunized macaques are shown as blue symbols, and S-2P mRNA-LNP immunized macaques are shown as red symbols. The reference range for each value is shown as gray shaded area for female macaques and cyan shaded area for male macaques. Creatine kinase does not have a reference range indicated. Males are shown as circles and females are shown as triangles.

FIG. 8A-F. ACE-2, RBD neutralizing antibody, and post-vaccination macaque plasma IgG binding to SARS-CoV-2 Spike variants. FIG. 8a,b Plasma IgG from macaques prior to immunization or after being immunized once with RBD-scNP adjuvanted with 3M-052-Alum (blue), RBD-scNP only (gray), or 3M-052-Alum only (white). Binding titers as log AUC were determined a before or b two weeks after a single immunization. Horizontal bars are the group mean. ECD, ectodomain; S-2P, diproline-stabilized spike. FIG. 8c ACE2 receptor and cross-neutralizing antibody DH1047 ELISA binding to SARS-CoV-2 Spike ectodomain (ECD) based on a Danish mink (H69/V70deI/Y453F/D614G/I692V), B.1.351-like (K417N/E484K/N501Y/D614G), and B.1.1.7 (H69/V70del/Y144del/N501Y/A570D/D614G/P681H/T7161/S982A/D1118H) strains. Titers are shown as area under the log-transformed curve (log AUC). FIG. 8d RBD-scNP and S-2P mRNA-LNP-immunized macaque serum IgG ELISA binding to SARS-CoV-2 Spike variants shown in c. Serum was tested after two immunizations. Horizontal bars are the group mean. FIG. 8e ACE2 receptor (gray), cross-neutralizing antibody DH1047 (navy), and ACE2 binding site-targeting neutralizing antibody DH1041 (green) ELISA binding to SARS-CoV-2 Spike RBD monomers. RBD variants contain a subset of mutations found in circulating B.1.351 and P.1 virus strains. Titers are shown as area under the log-transformed curve (log AUC). FIG. 8f RBD-scNP and S-2P mRNA-LNP-immunized macaque serum IgG ELISA binding to SARS-CoV-2 Spike RBD variants shown in e. Serum was tested after two immunizations. Horizontal bars are the group mean.

FIG. 9. Cross-neutralizing antibodies are elicited by recombinant protein RBD-scNP and mRNA-LNP immunization. Each row shows neutralization titer for an individual macaque immunized with one of the three immunogens. A reciprocal serum dilution titer of 87,480 is the upper limit of detection and 20 is the lower limit of detection for this assay. Titers are derived from a nonlinear regression curve fit to the average of duplicate measurements.

FIG. 10A-D. Cross-reactive plasma antibody responses elicited by RBD-NP immunization in macaques. FIG. 10a Plasma IgG from macaques immunized twice with RBD-scNP binds to Spike from human, bat, and pangolin SARS-related coronavirus Spike (S) in ELISA, but not endemic human coronaviruses or MERS-CoV. ECD, ectodomain. FIG. 10b Determination of DH1047 antigen binding fragment (Fab) binding kinetics to RBD monomer by surface plasmon resonance. Each curve shows a different concentration of DH1047 Fab. Binding kinetics are shown to the right from a 1:1 model fit. FIG. 10c Time course of vaccinated macaque plasma IgG binding to human, bat, and pangolin coronavirus S protein by ELISA. Each curve indicates the binding titer for an individual macaque. Arrows indicate immunization time points. FIG. 10d Unimmunized macaque plasma antibody blocking of SARS-CoV-2 S-2P (left) and batCoV-SHC014 (middle) binding to ACE2-Fc, RBD neutralizing antibody DH1041, and RBD cross-neutralizing antibody DH1047. (RIght) Blocking activity in the serum of humans immunized with Pfizer S-2P mRNA-LNP vaccine (N=4). Each symbol represents an individual human or macaque. Black horizontal bars indicate group mean and standard error.

FIG. 11A-B. Sequence conservation among SARS-related betaCoV, MERS-CoV, and endemic human CoVs. Sequence similarity of RBD in 11A and spike protein in 11B for representative betacoronaviruses. Heatmaps displaying pairwise amino acid sequence similarity for 57 representative betacoronaviruses. Dark blue shading indicates high sequence similarity.

FIG. 12A-C. Phylogenetic tree of representative betacoronavirus RBD sequences. Group 2b betaCoVs of interest are shown highlighted in red: LIST THE NAMES. Branch length units are substitutions per site. FIG. 12A shows the entire tree, FIG. 12B shows enlarged view of the two main branches from the top portion of tree, and FIG. 12C shows enlarged view of the bottom main branch.

FIG. 13. Multiple Sequence Alignment of Spike Protein from a Representative Set of Group 2b Betacoronaviruses. SARS-CoV-2 Wuhan-1 spike protein numbering is shown. NTD=N-terminal domain; RBD=receptor binding domain; S1/S2=SARS2 furin cleavage site; FP=fusion peptide; HR1=heptad repeat 1; HR2=heptad repeat 2; CH=central helix; CD=connecting domain; TM=transmembrane domain. ACE2 contact positions in SARS2 (calculated from PDB coordinates 6MOJ and 6LZG) are highlighted in dark red. Figure discloses SEQ ID NOS 56-67, respectively, in order of appearance.

FIG. 14. Serum neutralization titers elicited by two S-2P mRNA-LNP immunizations were boosted by a subsequent RBD-scNP immunization. FIG. 14a,b Serum neutralization of a SARS-CoV-2 D614G and b SARS-CoV-2 B.1.1.7 pseudovirus infection of ACE2-expressing 293 cells. Neutralization titers are ID50 as reciprocal serum dilution for serum collected two weeks after the second (week 6) and third immunization (week 10). Each symbol connected by a line represents the titer for an individual macaque before and after RBD-scNP immunization. Normal human serum spiked with DH1043 was used as a positive control.

FIG. 15. Histology and immunohistochemistry of lung tissue collected seven days after SARS-CoV-2 WA-1 intratracheal and intranasal challenge. FIG. 15a-c Macaques were immunized a thrice with RBD-scNP, b twice with S-2P mRNA-LNP and once with RBD-scNP, or c unimmunized. Each column shows results from an individual macaque. The macaque identification number is shown herein each column. Hematoxylin and eosin stain of lung sections are shown on the top row, with nucleocapsid immunohistochemistry shown on the bottom row for each macaque. Red arrows indicate site of antigen positivity. All images are shown at 10× magnification with 100 micron scale bars shown in the bottom right corner.

FIG. 16. Mucosal SARS-CoV-2 IgG responses in bronchoalveolar lavage (BAL) and nasal wash fluids before and after SARS-CoV-2 challenge. FIG. 16a ELISA binding titers for SARS-CoV-2-specific IgG in 10×BAL fluid from macaques immunized with (blue symbols, left column) RBD-scNP three times or S-2P mRNA-LNP twice and RBD-scNP once (red symbols, left column). Day −7 BAL fluid was collected at week 10 or 16 for the RBD-scNP alone group or the S-2P mRNA-LNP/RBD-scNP group respectively. Group mean and standard error are shown (N=5). FIG. 16b-d 10×BAL fluid blocking of ACE2, RBD neutralizing antibody DH1041, and cross-neutralizing antibody DH1047 binding to SARS-CoV-2 D614G stabilized spike ectodomain. A black horizontal bar indicates the group mean blocking percentage. Blocking above 20% (above the dashed line) is considered positive. FIG. 16e Neat nasal wash fluid from RBD-scNP-immunized or S-2P mRNA-LNP/RBD-scNP-immunized macaques. Day −7 nasal wash fluid was collected at week 16 and 2 and 4 days post challenge for the S-2P mRNA-LNP/RBD-scNP group. Nasal wash fluid was unavailable for the RBD-scNP before challenge, but was collected 2 and 4 days after the week 11 challenge. Group mean and standard error are shown (N=5).

FIG. 17. Scoring of Hematoxylin and Eosin (H&E) staining and immunohistochemistry (IHC) of macaque lung tissue collected seven days after challenge.

FIG. 18A-C shows non-limiting embodiments of amino acid (18B) and nucleic acid sequences (18A) of the invention. In FIG. 18B, spike and RBD portions are bolded. In this figure: Purple (underlined)=signal peptide; Orange=sortase A donor sequence (LPETGG (SEQ ID NO: 16)); Red=HRV3C cleavage site; 8×His tag (SEQ ID NO: 17), twin-StrepTagII (LEVLFQGPGHHHHHHHHSAWSHPQFEKGGGSGGGGSGGSAWSHPQFEK (SEQ ID NO: 18)); Cyan=8×His tag (SEQ ID NO: 17), twin-StrepTagII, HRV3C cleavage site (SAWSHPQFEKGGGSGGGGSGGSAWSHPQFEKHHHHHHHHLEVLFQGP (SEQ ID NO: 19)); Pink=Glycine-serine spacer (GSGS (SEQ ID NO: 20)). Figure shows sequences of soluble SARS-CoV-2 receptor binding domain (RBD) proteins-bold. In some embodiments, the RBD has been modified to include 417, 484, and 501 mutations found in circulating coronaviruses that may have escaped neutralizing antibodies or have a replication fitness advantage. The RBD sequences include a c-terminal sortase A donor sequence to allow for site specific conjugation to scaffolds expressing the n-terminal sortase A acceptor sequence. The donor sequence is a LPETGG (SEQ ID NO: 16) where the third amino acid can vary. The acceptor sequence is composed of 5 or more glycines (SEQ ID NO: 21) appended to the N-terminus. For protein purification purposes, the RBD subunits contain an HRV-3C cleavable His tag and twin streptagII. FIG. 18B-7 shows sequence of the n-terminal domain of SARS-CoV-2 with a C-terminal sortase A tag. For purification it includes an N-terminal His tag and twin streptagII followed by a HRV-3C cleavage site. HV1302346 and HV1302347 are spikes from South African variants of SARS-CoV-2. The variants include mutations at 417, 484, and 501, which are proximal to RBD antibody neutralizing epitopes. Each strain is designed as a HexaPro Spike ectodomain with a sortase A donor sequence appended to its c-terminus. For purification of the Spike, an HRV-3C cleavable His and twin streptagII sequence was added to the c-terminus. FIG. 18C1-15 shows non-limiting embodiments of amino acid sequences of the invention connected via linker to a self-assembling protein/a multimerization domain, e.g. ferritin protein. Sortase conjugate nanoparticles can be made by displaying human or animal coronavirus receptor binding domain. Receptor binding domains can include MERS-CoV (defined as amino acids 381 to 589 of spike), SARS-CoV-1, Canine CoV-HuPn, Bat CoV-WIV-1, Bat CoV SHC014, Pangolin CoV GXP4L, or Porcine CoV from Haiti. In FIG. 18C-1 the sequence shows one embodiment of a spike protein sequence—an RBD domain (bolded and underlined) after sortase conjugation. K417N, E484K, and N501Y are three mutations observed in the RBD of SARS-CoV-2 strain B1.135, which was recently sequenced in South Africa. These three mutations alone or in combination render the virus less sensitive to RBD neutralizing antibodies. The sequence shows one embodiment of ferritin sequence (italicized), and one embodiment of a linker (double underlined). FIGS. 18C2-15 show additional embodiments of ferritin multimeric fusion protein complexes/nanoparticles. The underlined sequence is a signal peptide. In some embodiments, the ferritin sequence comprises N19Q amino acid change. FIG. 18A discloses SEQ ID NOS 68-83, FIG. 18B discloses SEQ ID NOS 84-86, 84, and 87-97, FIG. 18C discloses SEQ ID NOS 97-107, 106, and 108-110, respectively, in order of appearance.

FIG. 19A-B show non-limiting embodiments of sortase designs and a schematic of a sortase reaction conjugating a protein to a lipid (cholesterol) molecule. The protein could be any spike protein design or portion thereof, e.g. but not limited to the ectodomain, RBD, NTD, or FP. The linker between the protein sequence and the ferritin protein sequence could be any suitable linker. In non-limiting embodiments, the lipid molecule could be substituted with a protein which multimerizes, e.g. but not limited to ferritin. Figure discloses SEQ ID NOS 111-116, respectively, in order of appearance.

FIG. 20A-B show non-limiting embodiments of nucleic acid (A) and amino acid sequences of the invention (B). In FIG. 20B, signal peptide underlined, in some sequences a sortase A tag/linker is italicized-GSGLPETGG (SEQ ID NO: 22). FIG. 20A discloses SEQ ID NOS 117-134, 75-78, and 135-146, FIG. 20B discloses SEQ ID NOS 147-164, 90-93, 165-170, 169, and 171-175, and FIG. 20C discloses SEQ ID NOS 176-177, all respectively, in order of appearance.

FIG. 21A-D. FIG. 21A shows truncation of the SARS-CoV-2 spike protein down to the RBD core (“RBDcore_ext”). Ribbon structure of the SARS-CoV-2 ectodomain with successive trimming of the protein down to the RBD core (moving from left to right). The red sphere indicates the N-terminus of the RBD core and the green indicates the C-terminus of the RBD core. The two spheres correspond to amino acid positions: Green=amino acid 333 and Red=527. FIG. 21B shows the positions of amino acid 333 and 527 in the RBDscNP sequence of SEQ ID NO: 97. The core RBD design is HV1302728v2—does not include linker/tag or ferritin. FIG. 21C shows one embodiment of a nucleic acid sequence and amino acid sequence of RBD core design of the SARS CoV2 Wuhan sequence. Underlined is the signal peptide. FIG. 21D shows one embodiment of a nucleic acid sequence of RBD core design of the SARS CoV2 Wuhan sequence. FIG. 21B discloses SEQ ID NO: 97, FIG. 21C discloses SEQ ID NOS 178-179, and FIG. 21D discloses SEQ ID NO: 180.

FIGS. 22A and 22B show non-limiting embodiments of nucleic acid and amino acid sequences of the invention-RBD and NTD designs. In this figure amino acid sequences comprise: a signal peptide, underlined in some of the amino acid sequences: MGWSCIILFLVATATGVHA (SEQ ID NO: 23) or MDSKGSSQKGSRLLLLLVVSNLLLPQGVLA (SEQ ID NO: 24) or MPMGSLQPLATLYLLGMLVASVLA (SEQ ID NO: 25) or MFVFLVLLPLVSS (SEQ ID NO: 26); C terminal sortase A tag, italicized and bolded in sequence HV1302967, BA.2RBDcoreExtKK_mVHss_3CST2: LPETGG (SEQ ID NO: 16) or LPSTGG (SEQ ID NO: 27); HRV-3C protease site, italicized and underlined: LEVLFQGP (SEQ ID NO: 28); His Tag: HHHHHHHH (SEQ ID NO: 17); C tag: EPEA (SEQ ID NO: 30); Twin streptag II: SAWSHPQFEKGGGSGGGGSGGSAWSHPQFEK (SEQ ID NO: 31), or strep tagII. Spike protein sequence or portion thereof (RBD or NTD) are shown as bolded amino acid sequences in representative sequences. The Spike ectodomain (HV1302119; 1-1146) and N-terminal domain (HV1302120 and HV1302125; amino acids 14-307) of Wuhan-1 strain of SARS-CoV-2 are shown. Two isolates of the Spike ectodomain of SARS-CoV-2 Beta variant (HV1302346 and HV1302347) are shown. The Spike proteins are purified to homogeneity to ensure high quality proteins. Following initial purification each of these constructs can be ligated to nanoparticle scaffolds via the C-terminal sortase tag (LPxTGG (SEQ ID NO: 32)). In the presence of the enzyme soratase A, C-terminal sortase A tag is covalently bonded to an acceptor sortase A sequence on a protein of interest that can multimerize as a nanoparticle, e.g. without limitation ferritin protein. The sortase reaction A sortase conjugated immunogen does not comprise the signal peptide and any of the amino acids after the end of the sortase tag. FIG. 22A discloses SEQ ID NOS 181-187, 82-83, and 188-191, FIG. 22B discloses SEQ ID NOS 192-204, respectively, in order of appearance.

FIG. 23A-C show non-limiting embodiments of nucleic acid and amino acid sequences of the invention fusion designs. FIG. 23A shows non-limiting embodiments of nucleic acid sequences (SEQ ID NOS 205-216). FIG. 23B shows non-limiting embodiments of amino acid sequences (SEQ ID NO: 217-228). Mouse heavy chain variable region signal peptide:

(SEQ ID NO: 23)
MGWSCIILFLVATATGVHA (underlined).

Bovine prolactin signal peptide:

(SEQ ID NO: 24)
MDSKGSSQKGSRLLLLLVVSNLLLPQGVLA (Underlined).

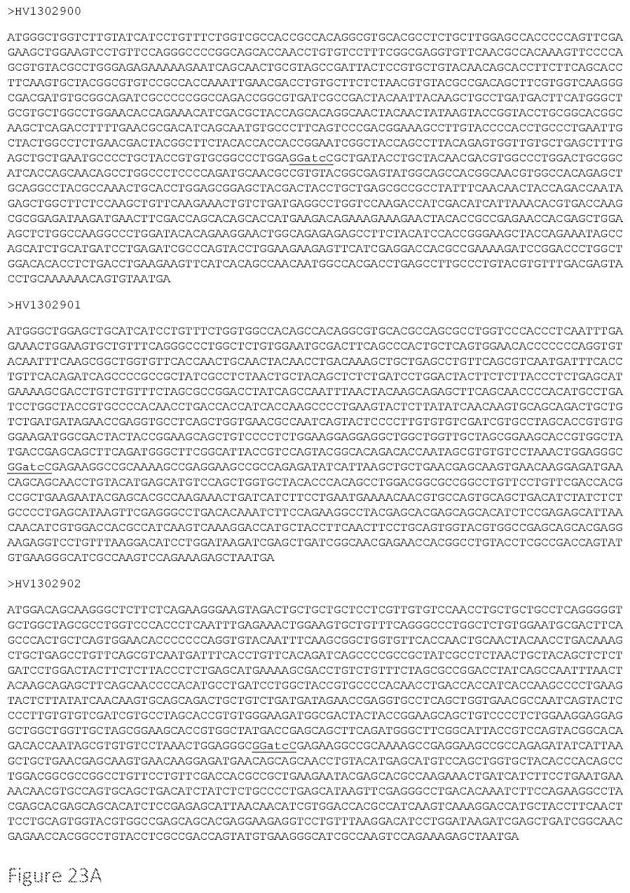

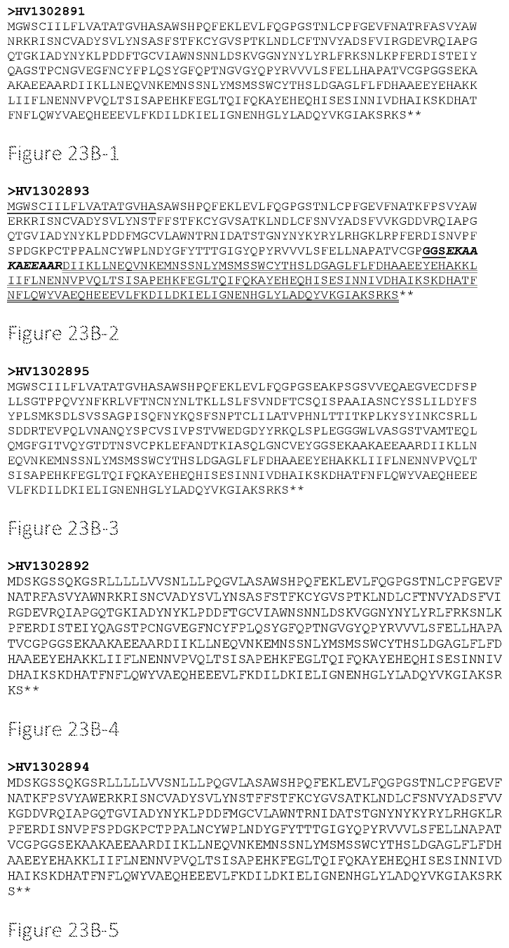

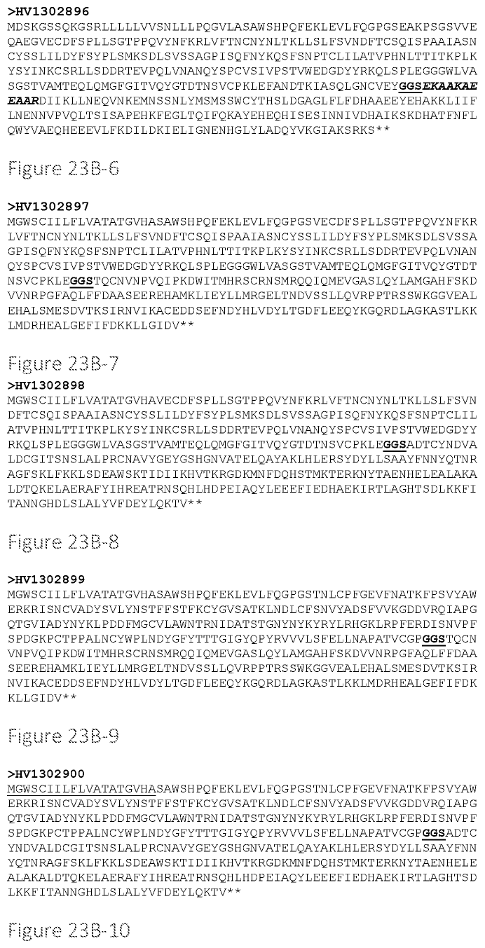

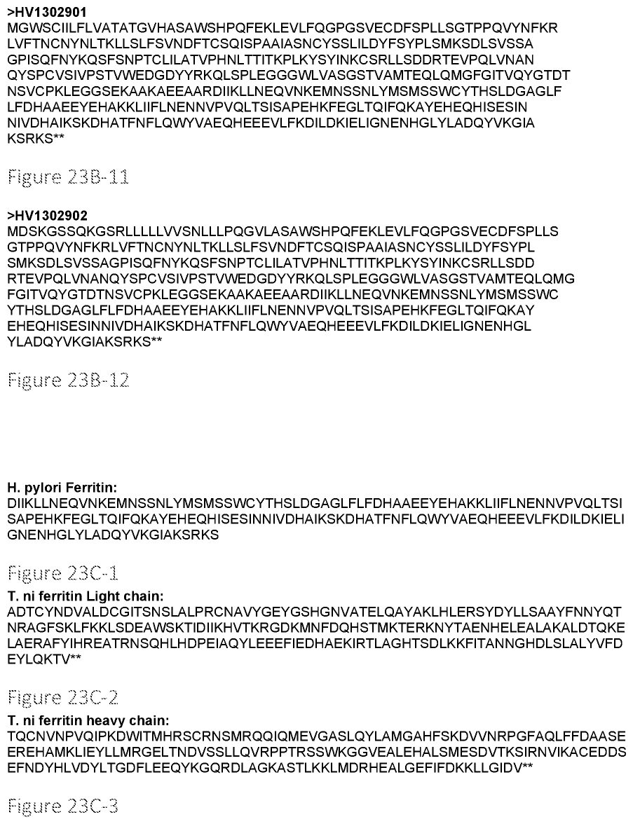

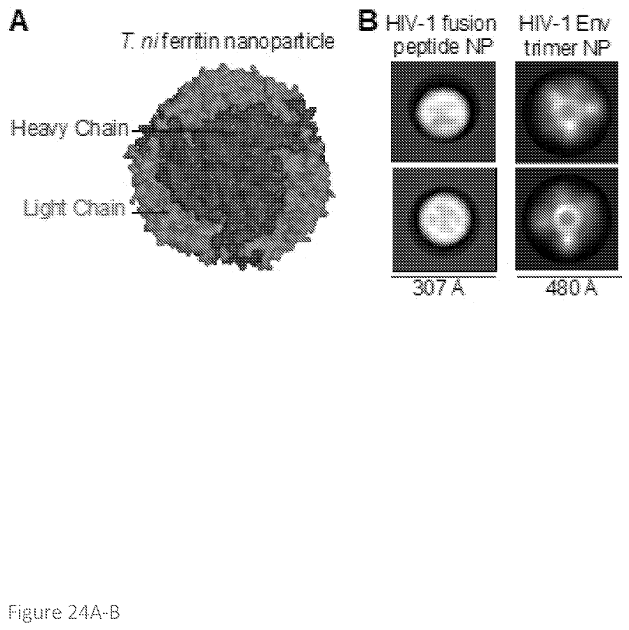

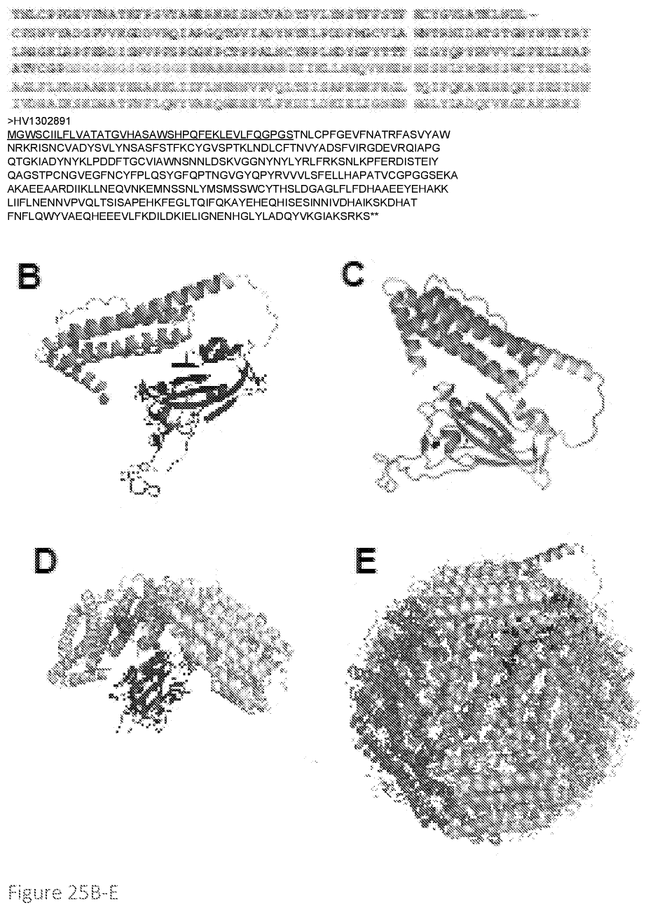

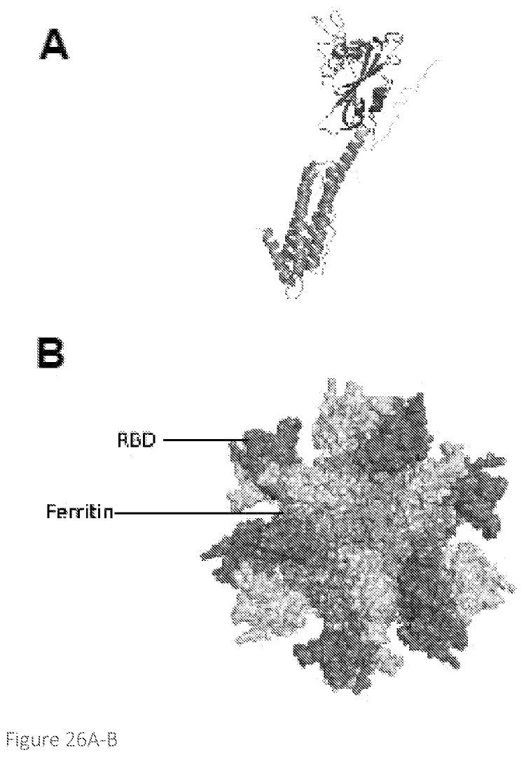

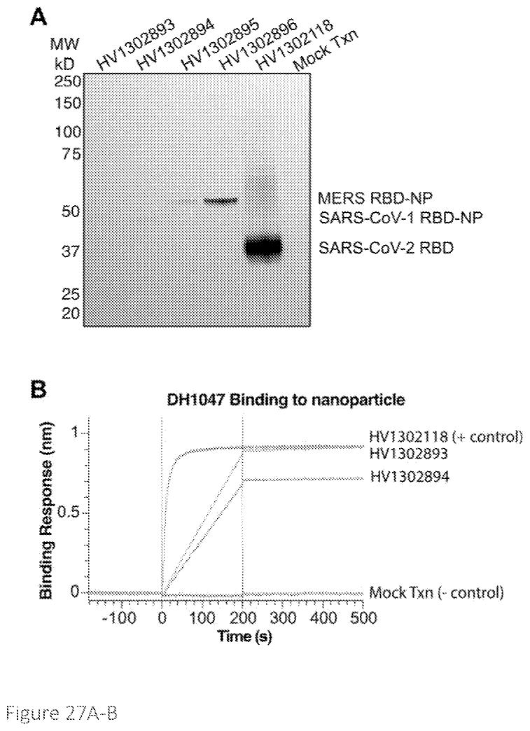

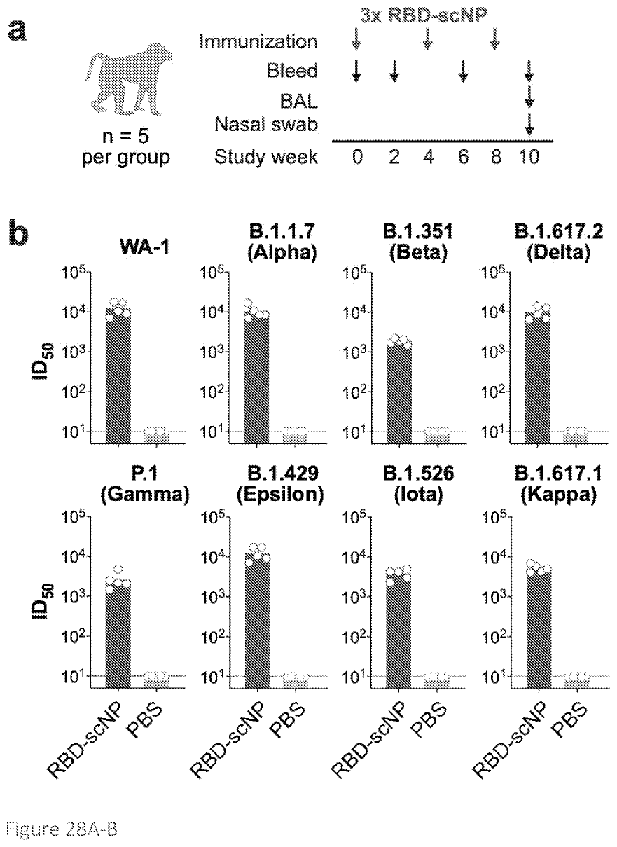

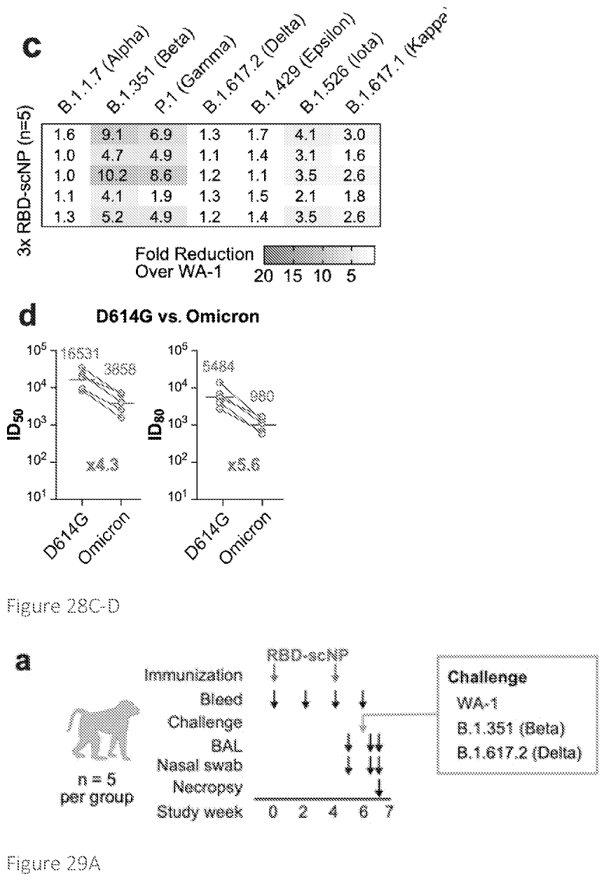

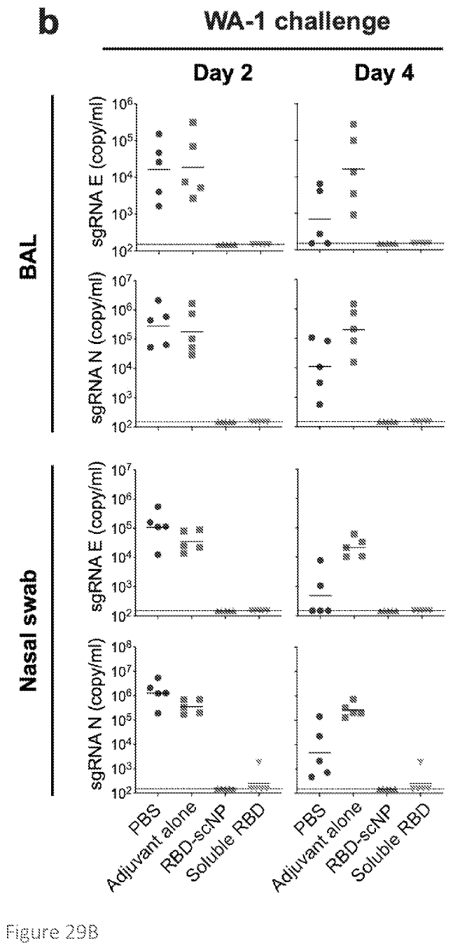

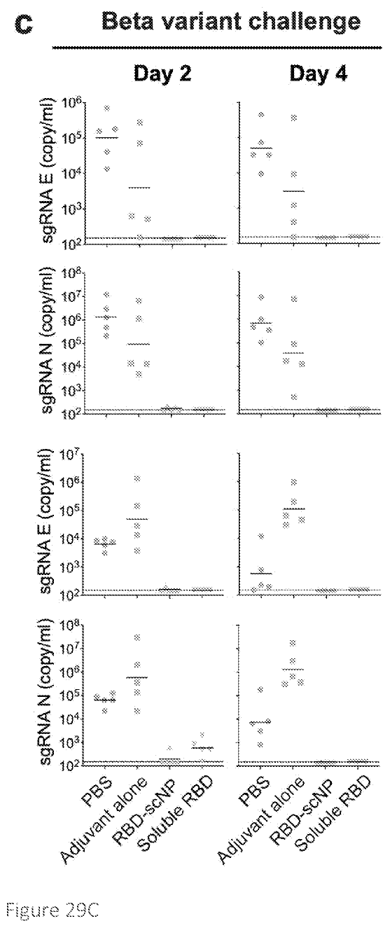

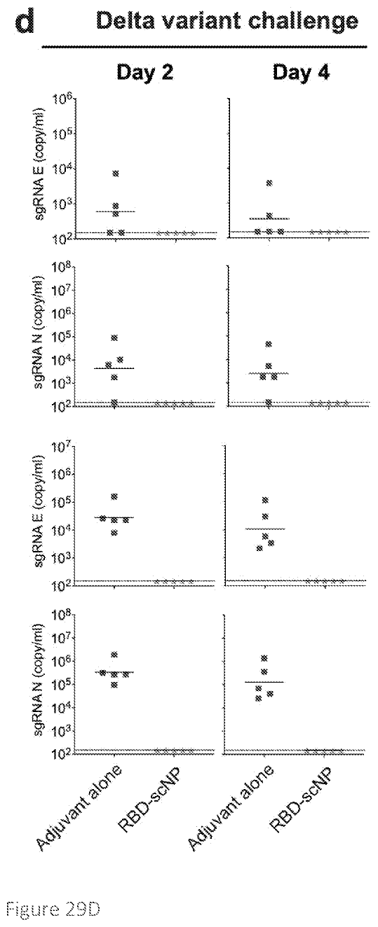

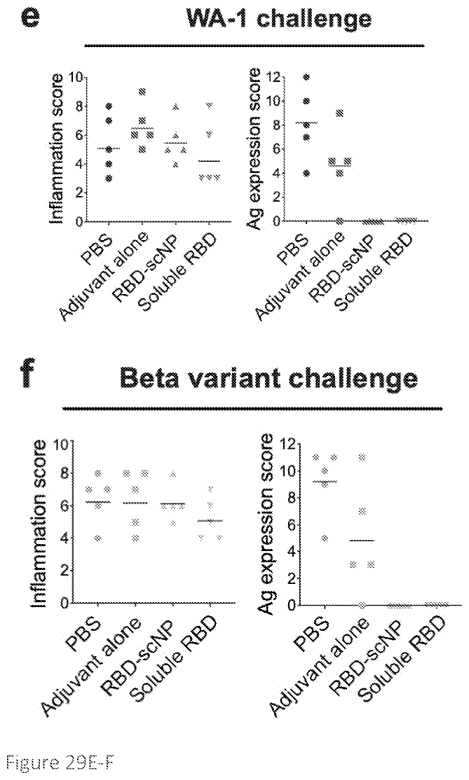

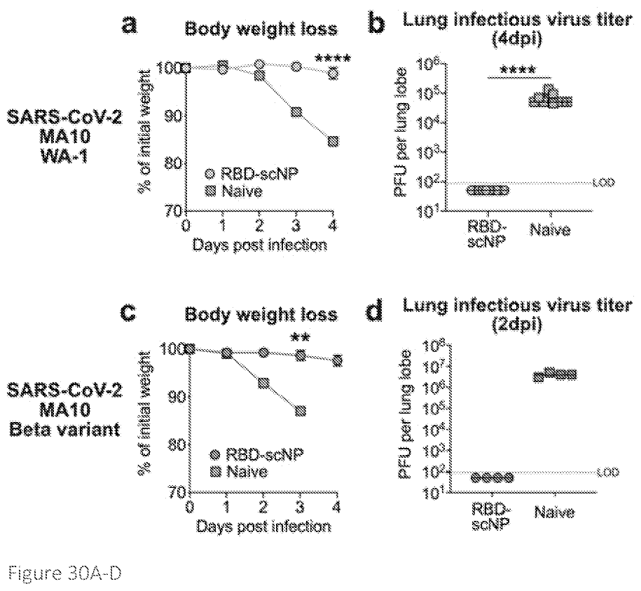

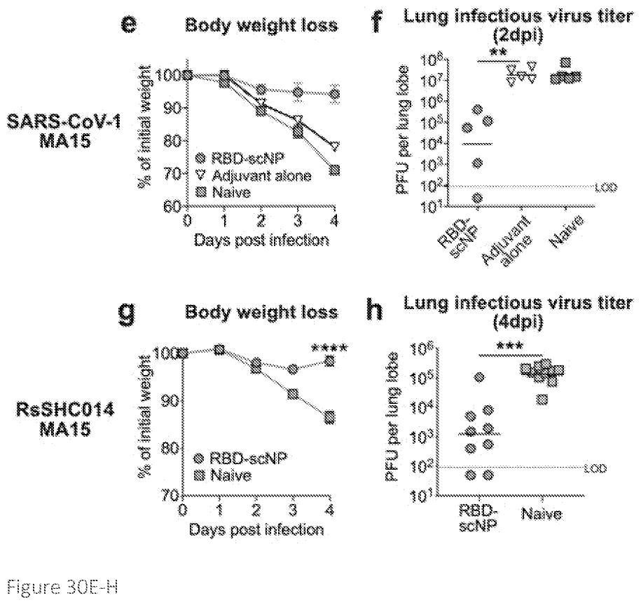

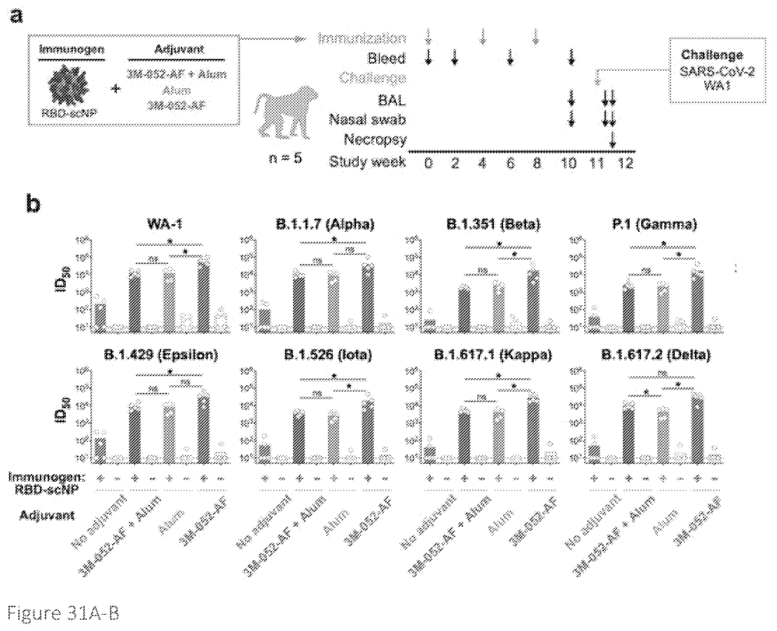

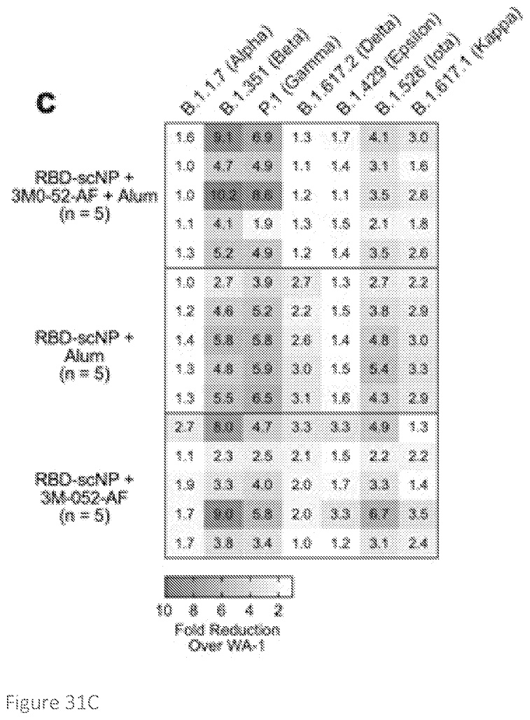

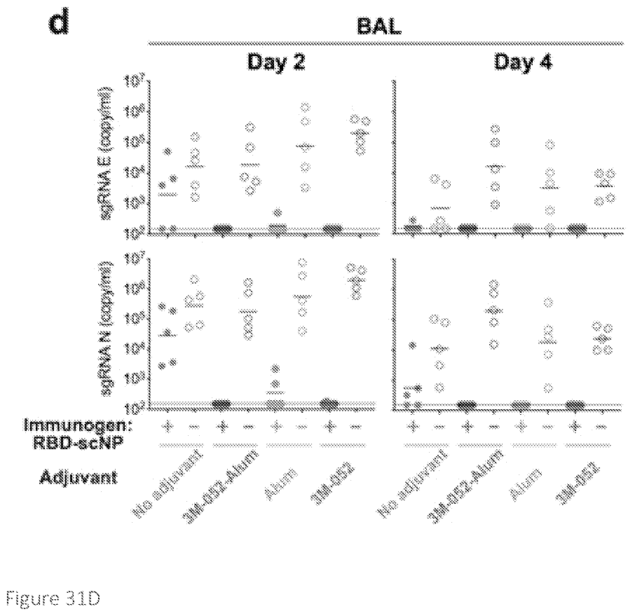

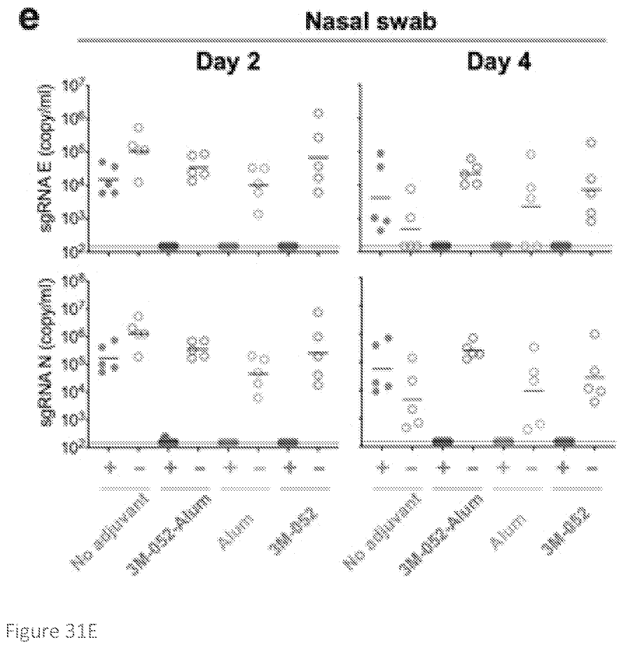

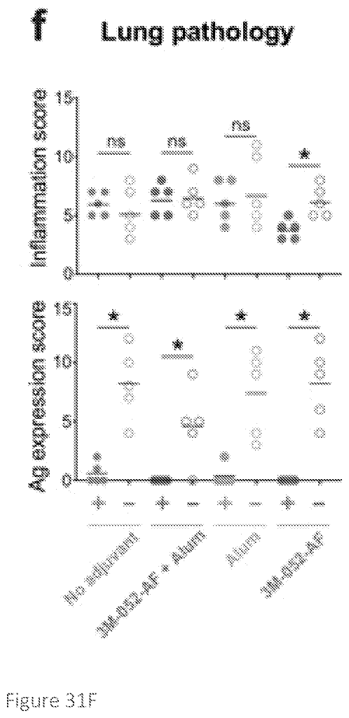

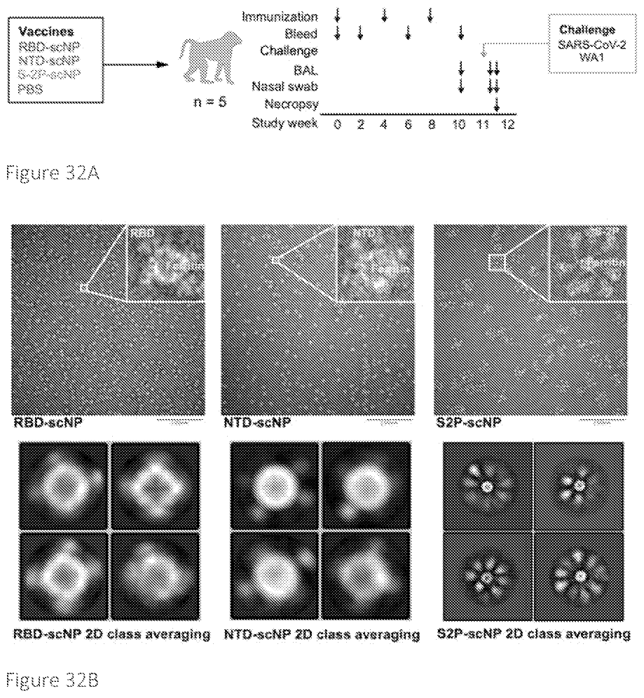

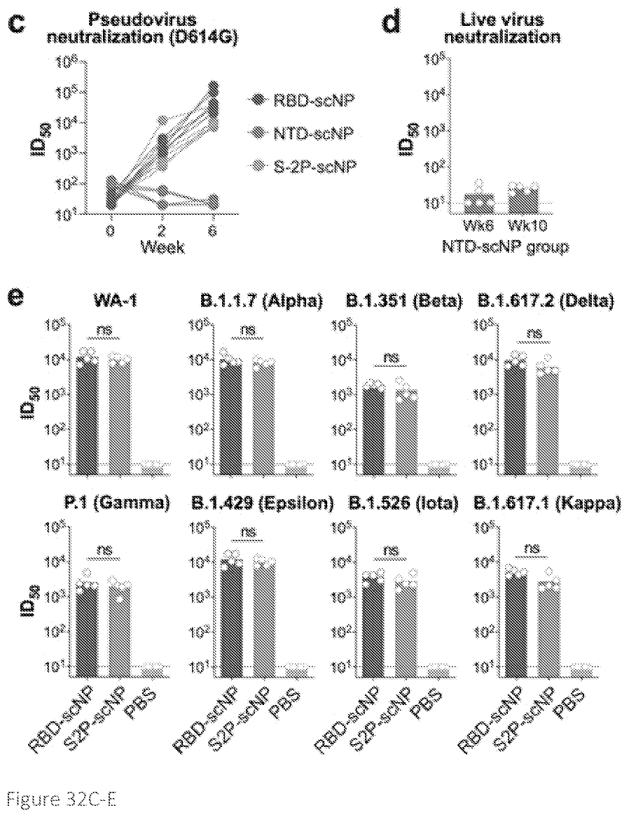

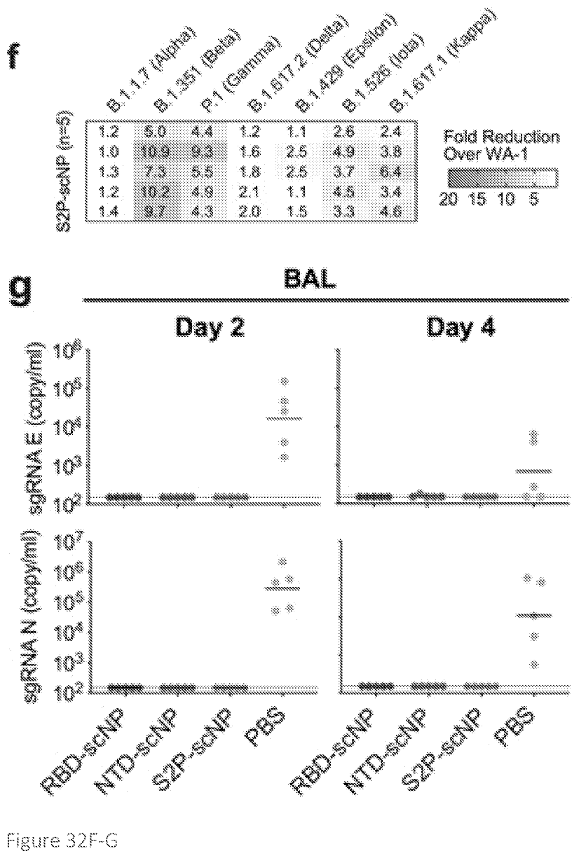

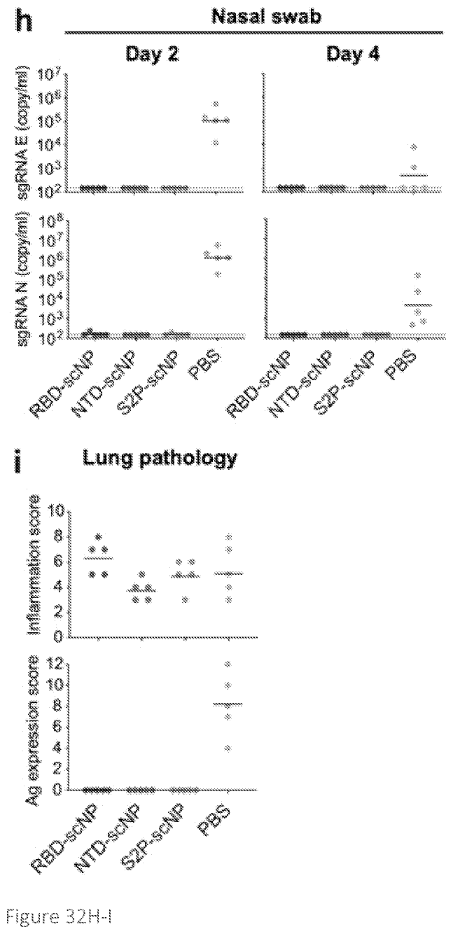

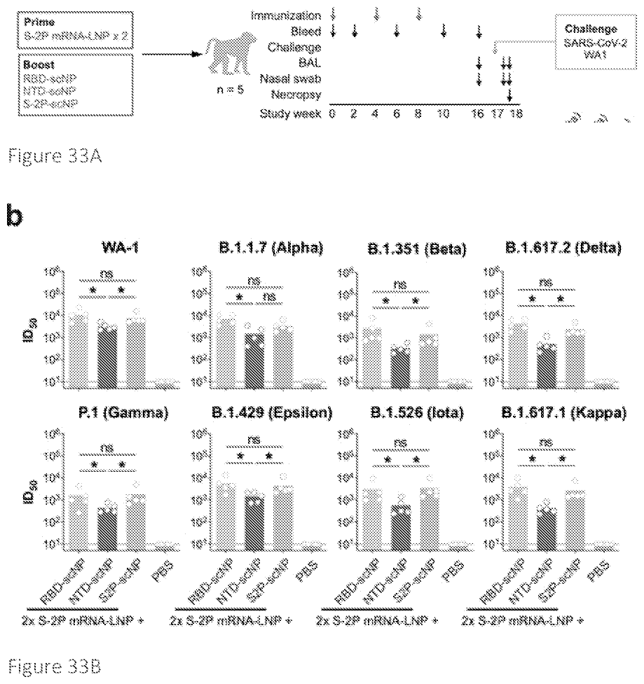

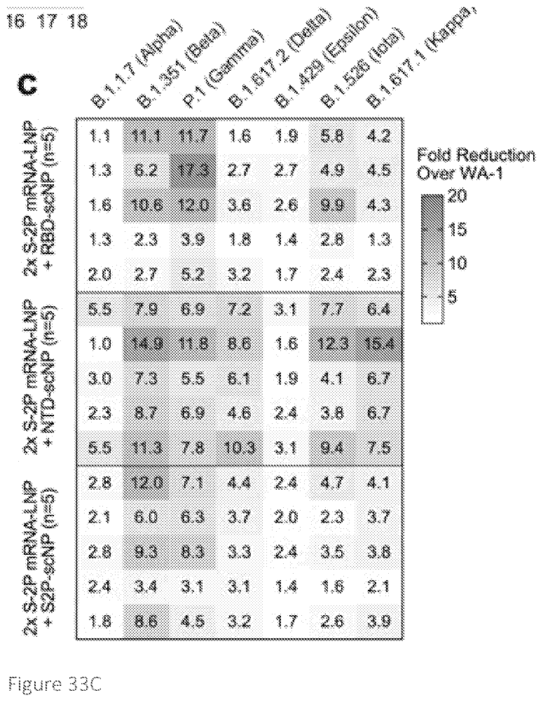

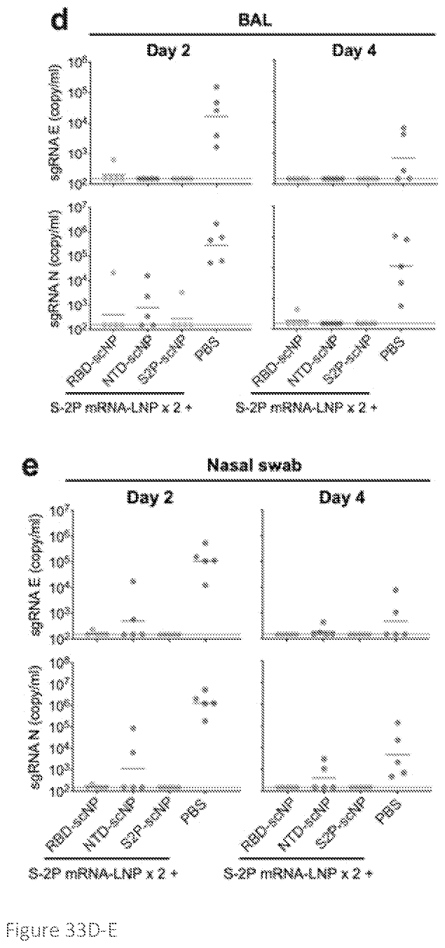

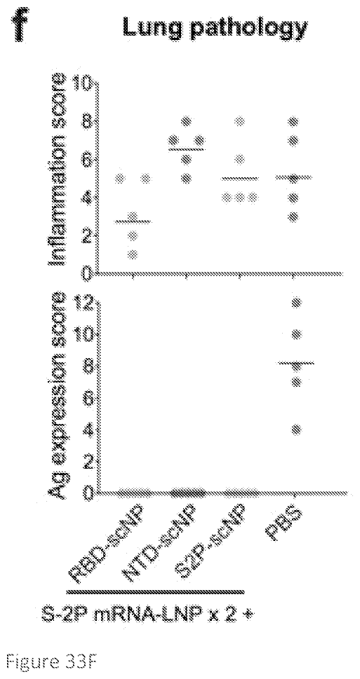

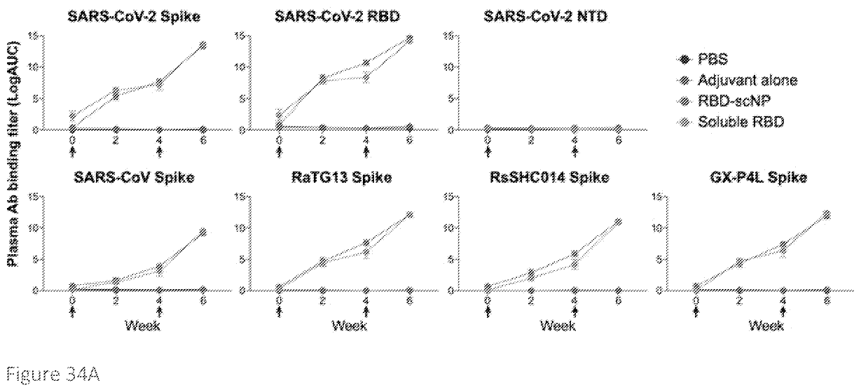

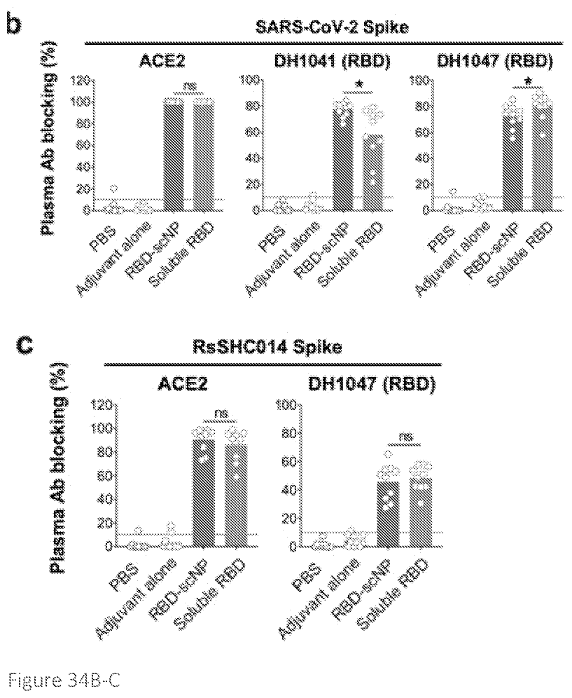

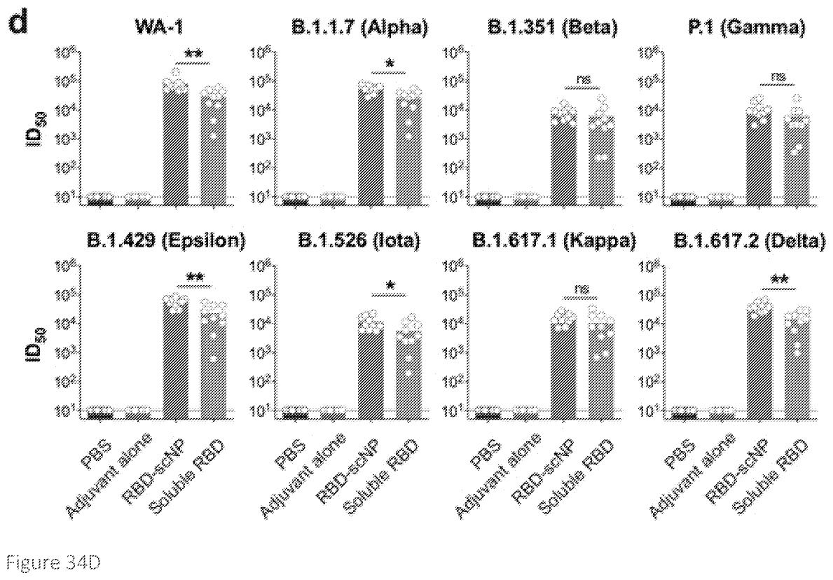

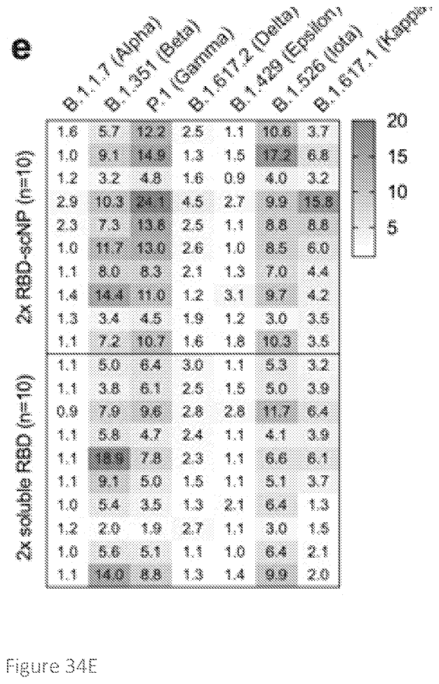

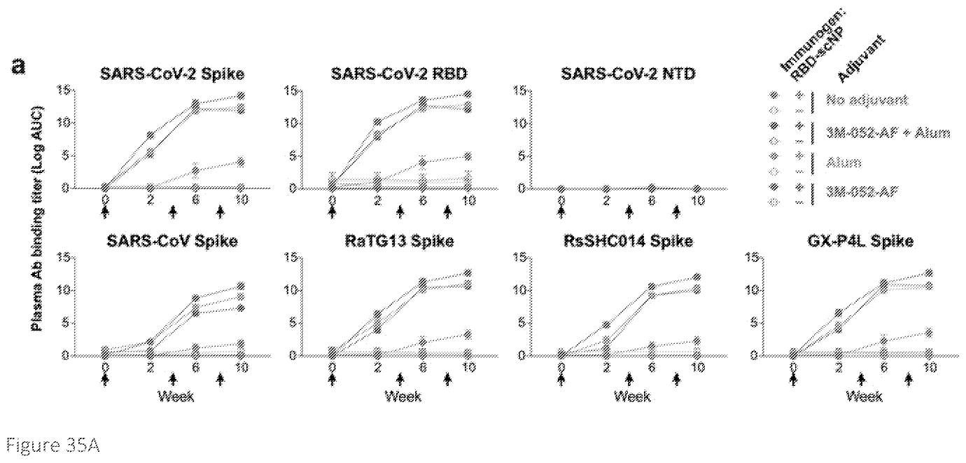

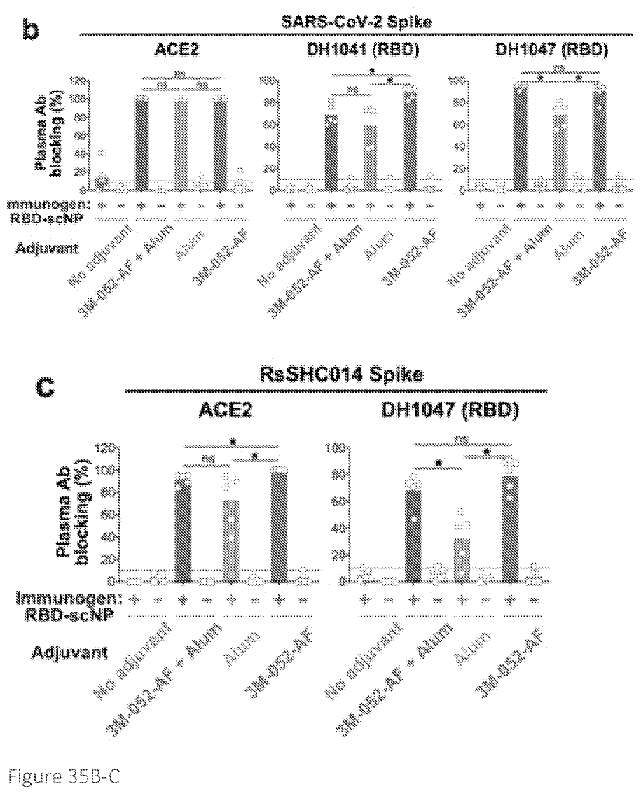

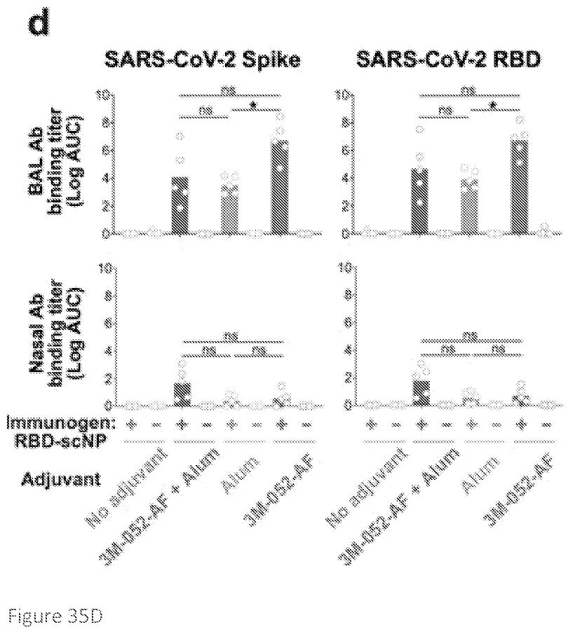

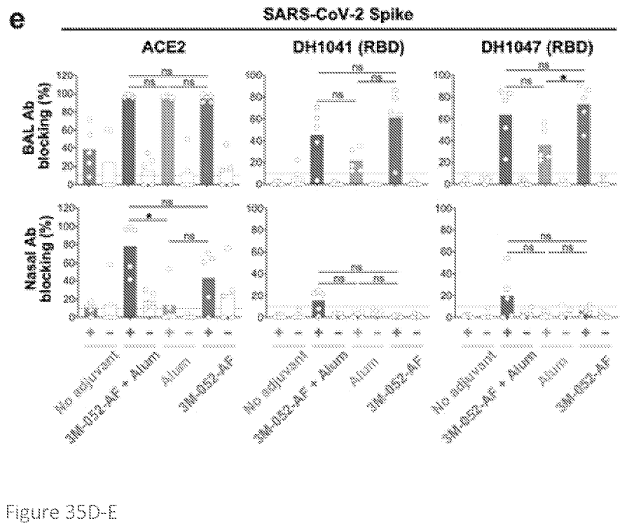

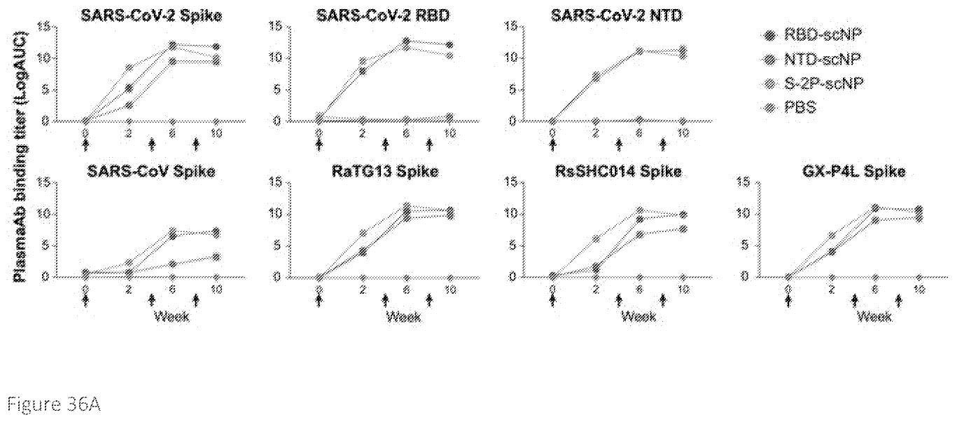

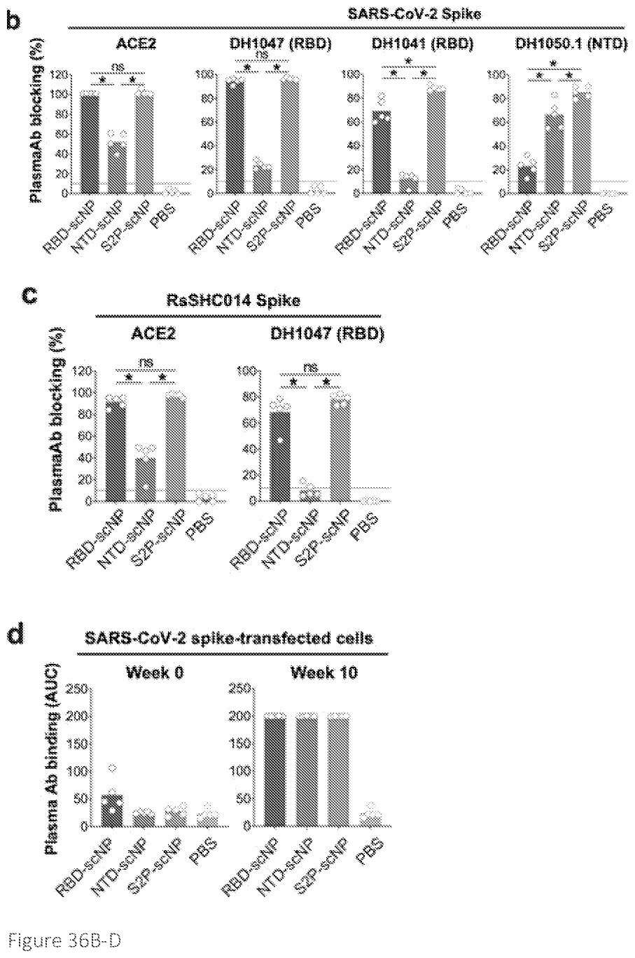

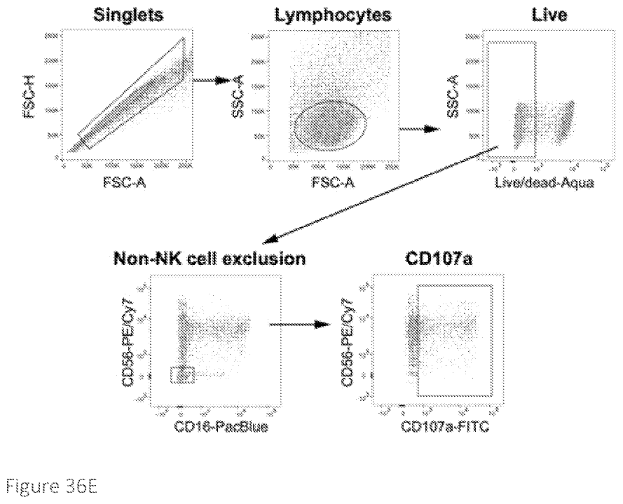

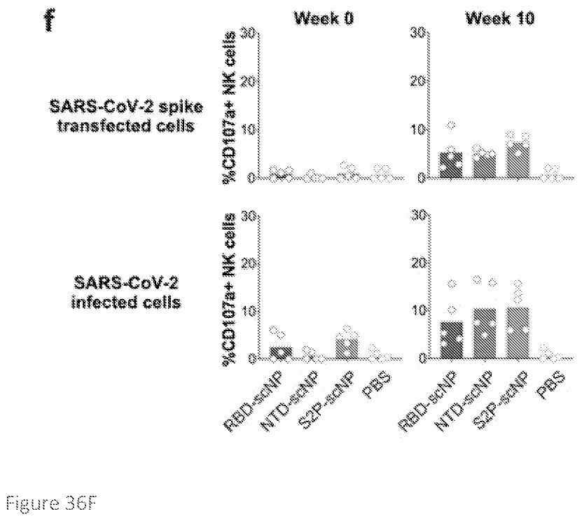

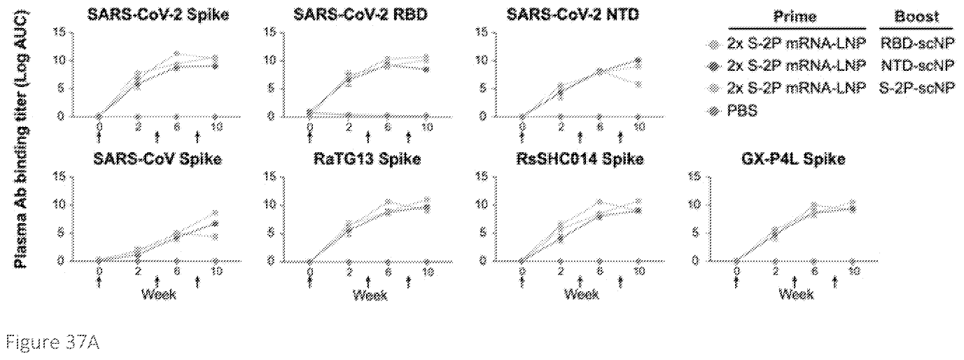

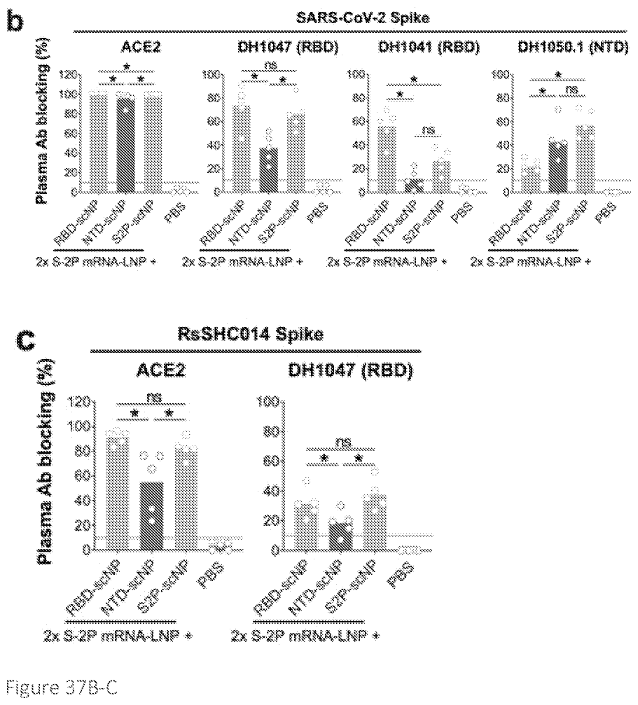

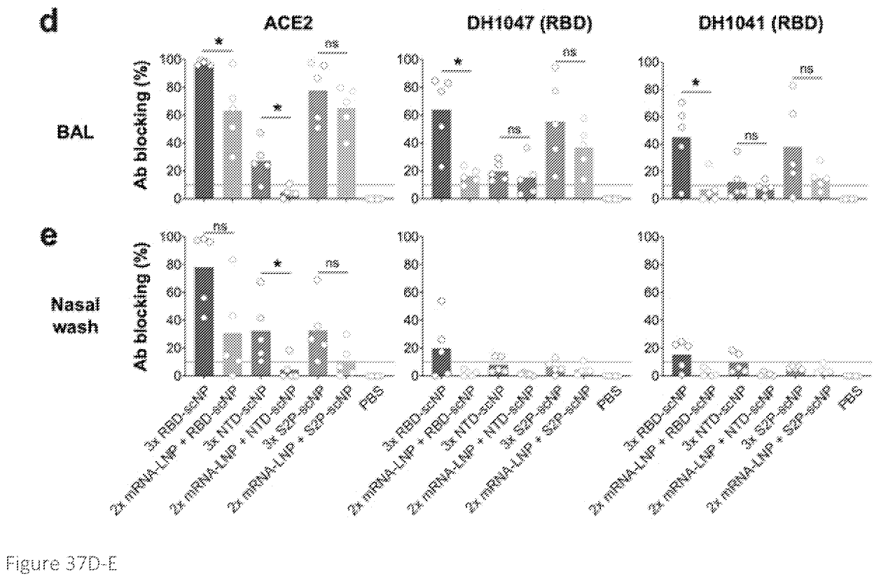

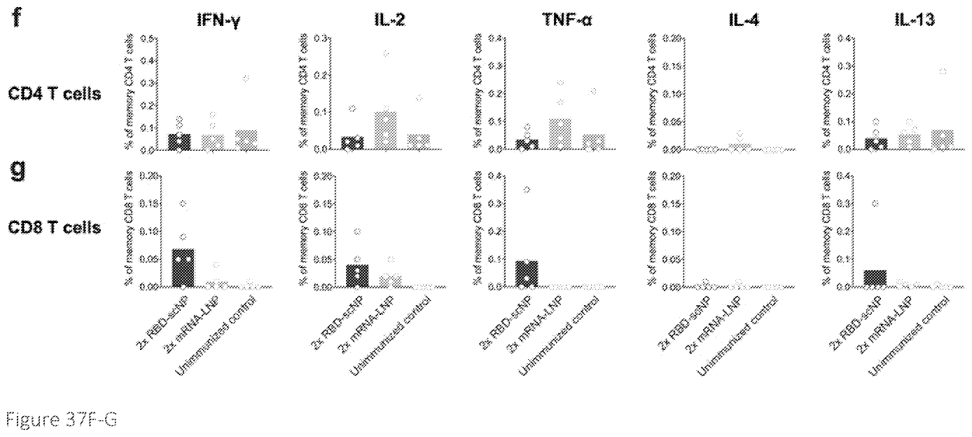

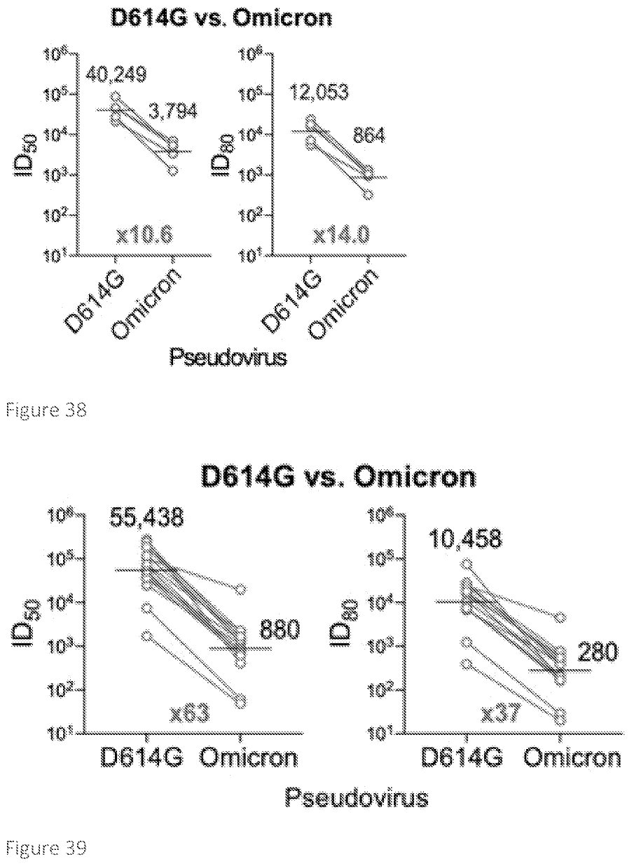

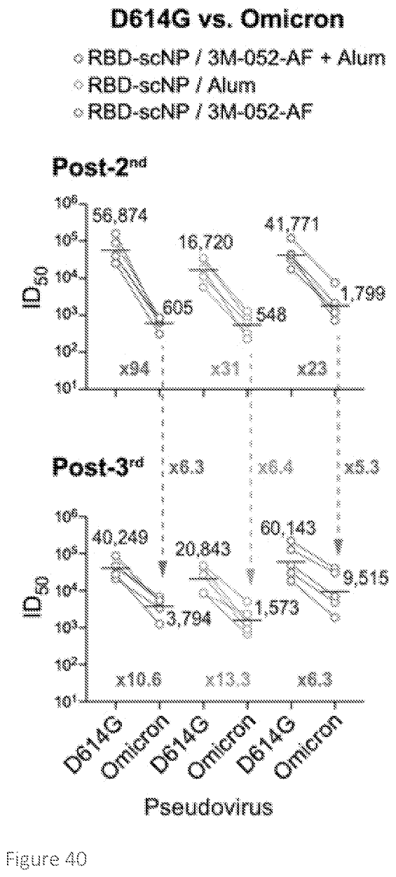

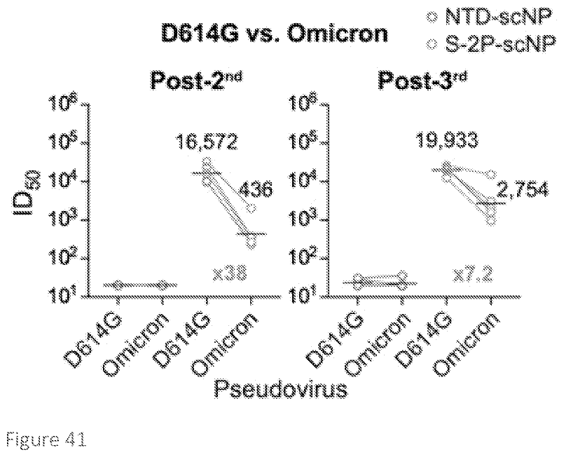

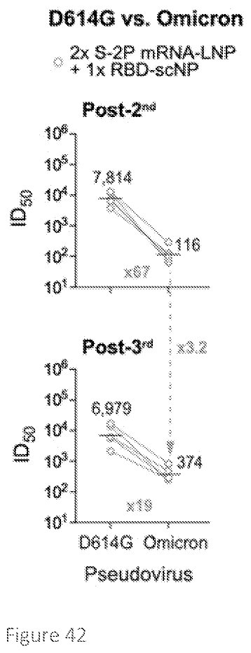

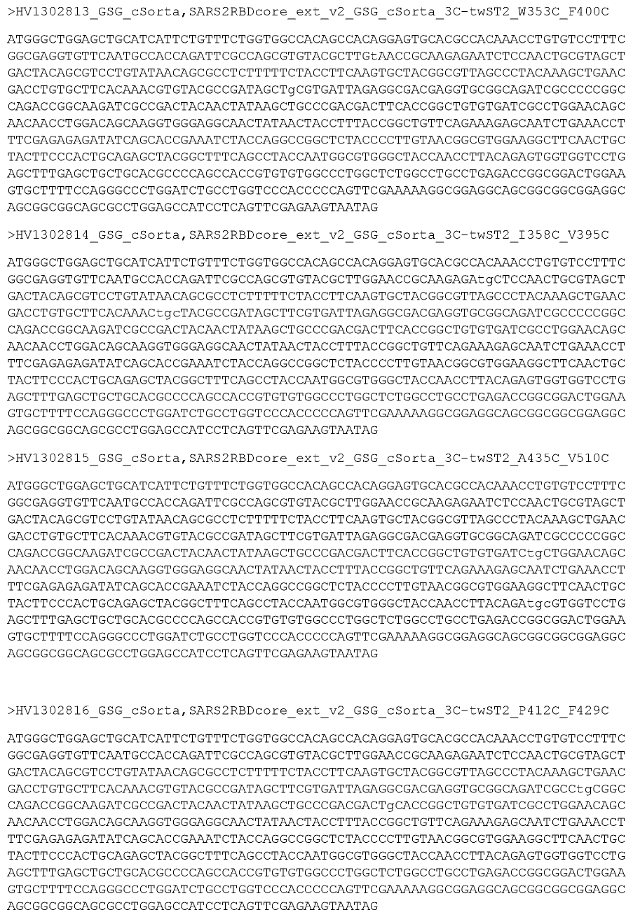

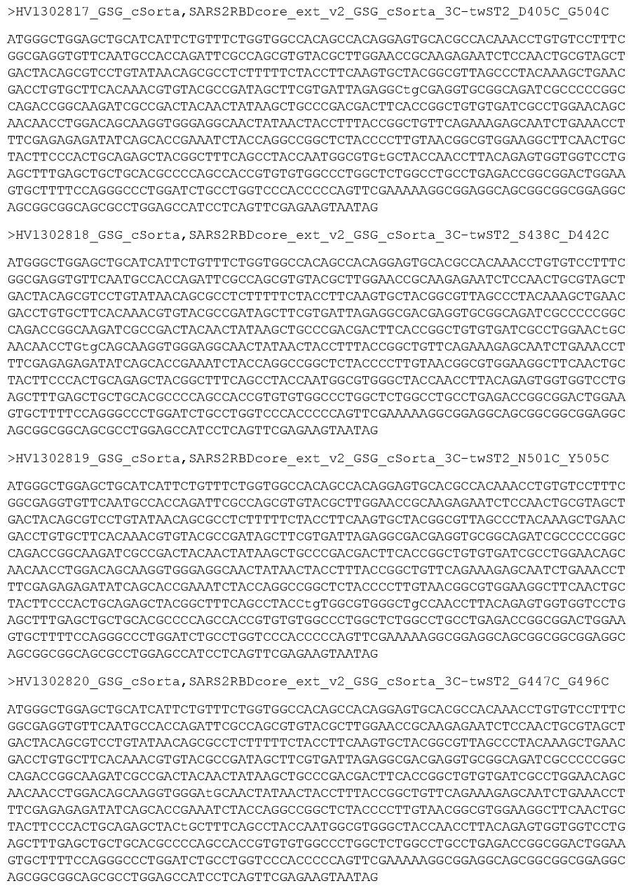

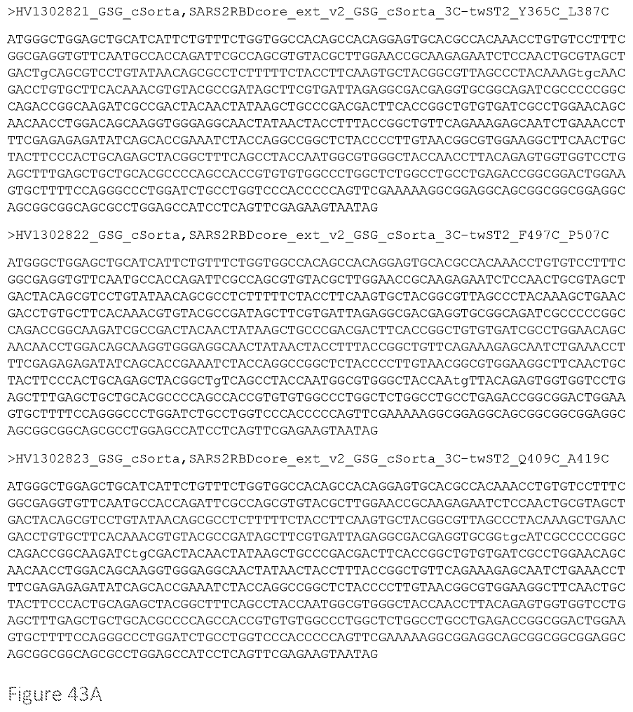

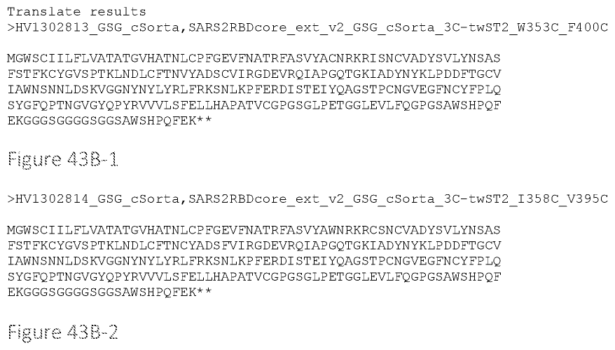

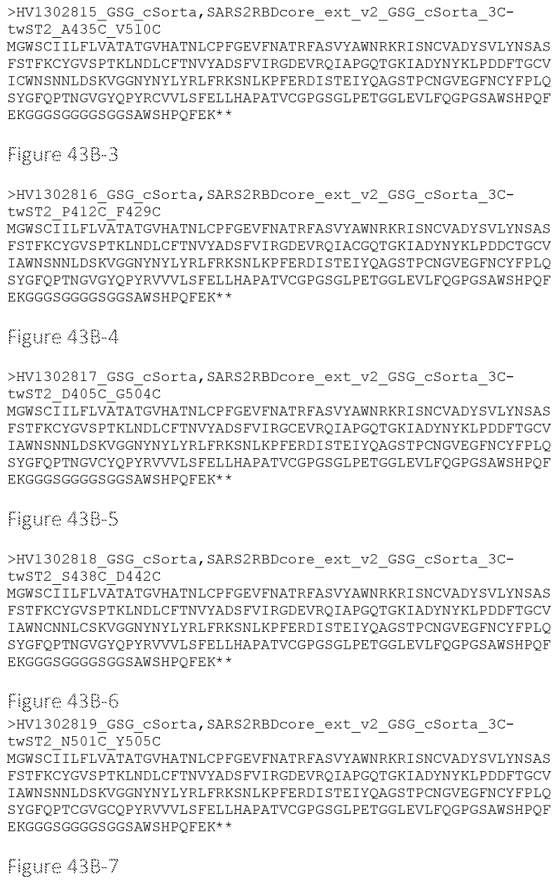

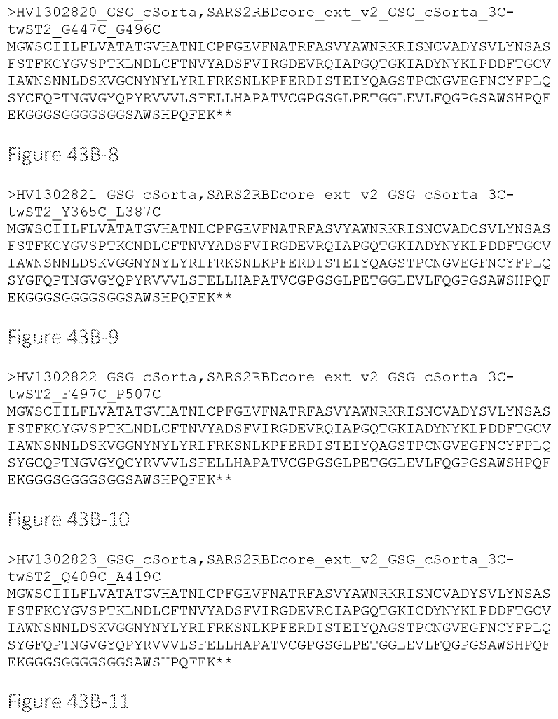

Glycine-serine linker (underlined and bolded): GGS. One embodiment of a rigid linker (italicized and bolded): EKAAKAEEAAR (SEQ ID NO: 9). Other embodiments of a rigid linker: EKAAKAEEAARPP (SEQ ID NO: 11) or EKAAKAEEAARP (SEQ ID NO: 10). HRV-3C protease site: LEVLFQGP (SEQ ID NO: 28). StrepTagII purification tag: SAWSHPQFEK (SEQ ID NO: 33). In some sequences the ferritin sequence is double underlined. In some embodiments, T. ni ferritin designs comprise a glycine serine linker followed immediately by a rigid linker. FIG. 23C shows non-limiting embodiments of ferritin sequences (SEQ ID NOS 229-231).

FIG. 24A-B. Nanoparticle (NP) platform for bivalent betacoronavirus vaccine. FIG. 24A. Surface representation of the structure of the two-component T. ni ferritin. the heavy chain component is colored blue and the light chain component is colored red (PDB:1Z60). FIG. 24B. negative stain electron microscopy images of T. ni ferritin NPs displaying HIV-1 fusion peptide or HIV-1 Env gp140 trimers.

FIG. 25A-E. RBD ferritin nanoparticles with long linkers are predicted not to display RBD on their surface. FIG. 25A. Amino acid sequence of RBD-ferritin fusion protein with a 25 amino acid linker without signal peptide. Ferritin (gray), SARS-CoV-2 RBD (blue), rigid helical 11 aa linker (magenta) and 14 aa flexible glycine-serine linker (green). Sequence in FIG. 25A is >HV1302891 (signal peptide underlined). FIG. 25B. Model of the predicted structure of a single H. pylori ferritin subunit fused with rigid helical 11 aa linker and 14 aa flexible glycine-serine linker to SARS-CoV-2 RBD. Colors are shown as in A. Model was predicted with AlphaFold. B. Heatmap of the certainty of the position of each amino acid in the model. Orange and red means high certainty and blue and green means low certainty. FIG. 25C,D. Alignment of predicted model of RBD-ferritin fusion with the structure of FIG. 25C. three ferritin subunits (yellow) or FIG. 25D. an intact 24-subunit nanoparticle shows the RBD (blue) and the ferritin C-terminus (red dot) are predicted to be inside the nanoparticle. FIG. 25A discloses SEQ ID NOS 232 and 217, respectively, in order of appearance.

FIG. 26A-B. RBD ferritin nanoparticle redesigned with a 11aa rigid linker and a 3 aa flexible linker displays RBD on the surface. FIG. 26A. A single subunit of the RBD (blue) fused to ferritin (gray) by a flexible 3 aa Glycine serine linker (green) and a rigid helical linker (magenta). Purification tag is shown in white. FIG. 26B. Surface representation of a model of the predicted structure of RBD fused to H. pylori ferritin and assembled as a 24-subunit nanoparticle. Burgundy and gold indicate RBDs and gray shows ferritin.

FIG. 27A-B. High-throughput expression screening of SARS-CoV-1 and MERS-CoV ( ) receptor binding domain nanoparticles. FIG. 27A,B. Each nanoparticle was expressed in a 6 mL cell culture for three days. Secreted protein present in the cell culture media was harvested concentrated and 20 microliters was used for a (27A) denaturing, reduced Western blot or (27B.) biolayer interferometry binding assay. Western blot primary antibody detected a N-terminal StrepTagII sequence on each protein. A mock was performed and cell culture media from a transfection (Mock Txn) was used as a negative control for the experiments. SARS-CoV-2 receptor binding domain monomers were used as a positive control.

FIG. 28A-D. RBD-scNP vaccination elicits broad neutralizing antibodies against SARS-CoV-2 variants. FIG. 28A, Schematic of the vaccination study in macaques. Cynomolgus macaques (n=5 per group) were immunized intramuscularly 3 times with 100 μg of RBD-scNP adjuvanted with 3M-052-AF+Alum. The control group was immunized with PBS. Blood and mucosal samples, including Bronchoalveolar lavage (BAL) and nasal swab samples, were collected in the indicated time points for antibody assays. FIG. 28B-C. Plasma antibody (post-3rd immunization) neutralization of SARS-CoV-2 variants pseudovirus infection of 293T-ACE2-TMPRSS2 cells. (b) Neutralization titers were shown as 50% inhibitory dilution (ID50). (c) Reduction of neutralization titers against variants were shown as fold reduction compared to the titers against WA-1. FIG. 28D. Plasma antibody (post-3rd immunization) neutralization titers against pseudoviruses of the SARS-CoV-2 Omicron variants in 293T-ACE2 cells. The mean ID50 and ID80 titers and the fold reduction compared to D614G are shown.

FIG. 29A-F. Two doses of RBD-scNP vaccination elicited in vivo protection from SARS-CoV-2 WA-1, B.1.351 (Beta) or B.1.617.2 (Delta) variant infection. FIG. 29A. Schematic of the vaccination and viral challenge study. Cynomolgus macaques were immunized with 2 doses of RBD-scNP adjuvanted with 3M-052-AF+Alum or the indicated negative controls. Monkeys were intranasally and intratracheally challenged with SARS-CoV-2 WA-1 strain (n=5) or B.1.351 (n=5) or B.1.617.2 (n=5) at week 6. Lower and upper respiratory tract samples were collected on day 0, 2 and 4 post challenge for subgenomic (sgRNA) viral replication test. Animals were necropsied on day 4 post-challenge and organs were collected for pathologic analysis. FIG. 29B-D. SARS-CoV-2 subgenomic RNA (sgRNA) levels for Envelop (E) gene and Nucleocapsid (N) gene in BAL and nasal swab samples collected on Day 2 and 4 after SARS-CoV-2 WA-1 virus challenge, SARS-CoV-2 WA-1 (b), Beta variant challenge (c) or Delta variant challenge (d). Dashed line indicates limit of the detection (150 copies/ml). FIG. 29E-F. Lung inflammation and SARS-CoV-2 viral antigen expression of the SARS-CoV-2 WA-1 virus challenged animals (e) and the Beta variant challenged animals (FIG. 29f). Lung sections from each animal were scored for inflammation by hematoxylin and eosin (H&E) staining, and for the presence of SARS-CoV-2 nucleocapsid by immunohistochemistry (IHC) staining.

FIG. 30A-H. RBD-scNP vaccine protected aged mice from SARS-CoV-2 Beta variant, SARS-CoV-1, and bat CoV RsSHC014 challenge. Aged female BALB/c mice (n=10 per group) were immunized intramuscularly with RBD-scNP formulated with 3M-052-AF+Alum (for the SARS-CoV-2 WA-1, SARS-CoV-2 Beta variant and RsSHC014 study) or with GLA-SE (for the SARS-CoV-1 study). For the SARS-CoV-2 WA-1 and RsSHC014 study, mice were immunized on week 0 and 2, and challenged on week 7. For the SARS-CoV-2 B.1.351 and SARS-CoV-1 study, mice were immunized on week 0 and 4, and challenged on week 6. Body weights were monitored daily and lungs infectious virus titer were measured in one lung lobe collected 2 days post-infection (2 dpi) or 4 days post-infection (4 dpi). FIG. 30A-B. SARS-CoV-2 mouse-adapted 10 (MA10) WA-1 challenge. (a) Weight loss and (b) lung infectious virus titers at 4 dpi. FIG. 30C-D. SARS-CoV-2 MA10 Beta variant challenge. (c) Weight loss and (d) lung infectious virus titers at 2 dpi. FIG. 30E-F. SARS-CoV-1 mouse-adapted 15 (MA15) challenge. (e) Weight loss and (f) lung infectious virus titers at 2 dpi. FIG. 30G-H. Bat CoV RsSHC014 mouse-adapted 15 (MA15) challenge. (g) Weight loss and (h) lung infectious virus titers at 4 dpi. Statistical significance in all the panels were determined using Wilcoxon rank sum exact test. Asterisks show the statistical significance: ns, not significant, *P<0.05, **P<0.01, ***P<0.001, ****P<0.0001. LOD: Limit of detection.

FIG. 31A-F. Neutralizing antibodies and in vivo protection elicited by RBD-scNP vaccine formulated with three different adjuvants. FIG. 31A. Schematic of the vaccination and viral challenge study. Cynomolgus macaques (n=5 per group) were immunized intramuscularly for 3 doses with 100 μg of RBD-scNP adjuvanted with 3M052-AF+Alum, Alum, 3M052-AF, or PBS control. Animals injected with adjuvant alone or PBS were set as control groups. Monkeys were intranasally and intratracheally challenged with SARS-CoV-2 WA-1 at week 11. Blood samples were collected in week 0, 2, 6, 10. Lower and upper respiratory tract samples were collected on day 0, 2 and 4 post challenge for subgenomic RNA (sgRNA) viral replication test. Animals were necropsied on day 4 post-challenge, and organs were collected for pathologic analysis. FIG. 31B-C. Vaccine-induced neutralization titers against SARS-CoV-2 WA-1 and variants. Plasma samples collected after the third vaccination were tested in neutralization assays against pseudovirus viruses in 293T-ACE2-TMPRSS2. (b) Neutralization titers were shown as 50% inhibitory dilution (ID50). (c) Reduction of neutralization titers against variants were shown as fold changes over the titers against WA-1. Animal groups were indicated on the left. FIG. 31D-E. SARS-CoV-2 sgRNA viral replication assays. SARS-CoV-2 E gene or N gene sgRNA were quantified in BAL (d) and nasal swab (e) samples collected before challenge and on day 2 and 4 post-challenge. Dashed line indicates limit of the detection (150 copies/ml). FIG. 31F. Lung inflammation and SARS-CoV-2 viral antigen expression of the challenged animals. Lung sections from each animal were scored for inflammation by H&E staining, and for the presence of SARS-CoV-2 nucleocapsid by IHC staining. Statistical significance in all the panels were determined using Wilcoxon rank sum exact test. Asterisks show the statistical significance: ns, not significant, *P<0.05.

FIG. 32A-I. NTD-scNP and S2P-scNP protected macaques from SARS-CoV-2 WA-1 infection in both lower and upper respiratory tracts. FIG. 32A. Schematic of the vaccination and viral challenge study in macaques. Cynomolgus macaques (n=5 per group) were immunized intramuscularly for 3 times with 100 μg of RBD-scNP, NTD-scNP, or S2P-scNP adjuvanted with 3M-052-AF+Alum. The control group was immunized with PBS. Blood samples were collected at weeks 0, 2, 6, 10 and monkeys were intranasally and intratracheally challenged with SARS-CoV-2 WA-1 strain at week 11. Lower and upper respiratory tract samples were collected on days 0, 2 and 4 post-challenge for sub-genomic (sgRNA) RNA viral replication assays. Animals were necropsied on day 4 post-challenge, and organs were collected for pathologic analysis. FIG. 32B. Negative-stain electron microscopy of RBD-scNP, NTD-scNP, and S2P-scNP. The insets show zoomed-in image of a representative scNPs. The 2D class averaging of 14,300 RBD-scNP particles, 10,800 NTD-scNP particles or 1,034 S2P-scNP particles were generated using RELION. The size of each box is 257 Å, 257 Å, and 1,029 Å for RBD-scNP, NTD-scNP and S2P-scNP, respectively. FIG. 32C. Pseudovirus neutralization. Plasma samples collected in week 0, 2, 6 were tested in neutralization assays against pseudotyped SARS-CoV-2 D614G strain in 293T/ACE2.MF cells. FIG. 32D. Live virus neutralization of the NTD-scNP-induced antibodies. Plasma samples from the NTD-scNP group collected in week 6 and 10 were tested in neutralization assays against live SARS-CoV-2 virus in Vero-E6 cells. FIG. 32E-F. Plasma antibody neutralization titers against pseudoviruses of the SARS-CoV-2 variants in 293T-ACE2-TMPRSS2 cells. (e) Neutralization titers were shown as 50% inhibitory dilution (ID50). (f) Reduction of neutralization titers of the S2P-scNP group against variants were shown as fold changes over the titers against WA-1. Statistical significance in all the panels were determined using Wilcoxon rank sum exact test. Asterisks show the statistical significance: ns, not significant. FIG. 32G-H. SARS-CoV-2 sgRNA levels for the E gene and N gene in bronchoalveolar lavage (BAL) samples (g) and nasal swab samples (FIG. 32H) collected on Day 2 and 4 post-challenge. FIG. 32I. Lung inflammation and SARS-CoV-2 viral antigen expression of the challenged animals. Lung sections from each animal were scored for inflammation by H&E staining, and for the presence of SARS-CoV-2 nucleocapsid by IHC staining.

FIG. 33A-F. Neutralizing antibodies and in vivo protection induced by the mRNA-LNP prime-scNP boost vaccination regimen against SARS-CoV-2 WA-1 infection. FIG. 33A. Schematic of the prime-boost vaccination and viral challenge study. Cynomolgus macaques (n=5 per group) were immunized intramuscularly with 2 doses of S-2P mRNA-LNP vaccine, then boosted with 100 μg of RBD-scNP, NTD-scNP, or S2P-scNP adjuvanted with 3M-052-AF+Alum. Animals in the control group were immunized with PBS. Blood samples were collected in week 0, 2, 6, 10, 16, and monkeys were intranasally and intratracheally challenged with SARS-CoV-2 WA-1 strain at week 17. Lower and upper respiratory tract samples were collected on day 0, 2 and 4 post challenge for sgRNA viral replication test. Animals were necropsied on day 4 post-challenge for pathologic analysis. FIG. 33B-C. Plasma antibody neutralization titers against pseudoviruses of the SARS-CoV-2 variants in 293T-ACE2-TMPRSS2 cells. (b) Neutralization titers were shown as 50% inhibitory dilution (ID50). (c) Reduction of neutralization titers against variants were shown as fold changes over the titers against WA-1. Animal groups and animal IDs were indicated on the left. Statistical significance in all the panels were determined using Wilcoxon rank sum exact test. Asterisks show the statistical significance: ns, not significant, *P<0.05. FIG. 33d-e. SARS-CoV-2 sgRNA levels for E gene and N gene in BAL (d) and nasal swab (e) samples collected before challenge and on Day 2 and 4 post-challenge. FIG. 33F. Lung inflammation and SARS-CoV-2 viral antigen expression of the challenged animals. Lung sections from each animal were scored for inflammation by H&E staining, and for the presence of SARS-CoV-2 nucleocapsid by IHC staining.

FIG. 34A-E. RBD-scNP elicited higher titers of neutralizing antibodies than soluble RBD. FIG. 34A. Plasma antibody binding titers to SARS-CoV-2 spike, RBD and NTD, as well as spike proteins of SARS-CoV, bat coronaviruses RaTG13, RsSHC014, and pangolin coronavirus GX-P4L. ELISA binding titers were shown by log area-under-curve (AUC). Dot and error bars indicate mean±SEM of all animals in each group. FIG. 34B-C. Plasma antibody blocking activity after the 2nd immunization. ELISA was performed to test plasma antibodies blocking ACE2, human RBD neutralizing antibodies DH1041 and DH1047 binding to SARS-CoV-2 Spike protein (b), or plasma samples blocking ACE2, DH1041 and DH1047 binding to RsSHC014 Spike protein (c). Data are expressed as percentages of blocking of ACE or the indicated antibody by 1:50 diluted plasma samples. FIG. 34D-E. Plasma antibody neutralization titers against pseudoviruses of the SARS-CoV-2 variants in 293T-ACE2-TMPRSS2 cells. (d) Neutralization titers were shown as 50% inhibitory dilution (ID50). (e) Reduction of neutralization titers against variants were shown as fold changes over the titers against WA-1.

FIG. 35A-E. Serum and mucosal antibody responses elicited by RBD-scNP formulated with three different adjuvants. Related to FIG. 31. FIG. 35A. Plasma antibody binding titers. ELISA was performed to test plasma samples binding to SARS-CoV-2 spike, RBD and NTD, as well as spike proteins of SARS-CoV, bat coronaviruses RaTG13, RsSHC014, and pangolin coronavirus GX-P4L. ELISA binding titers were shown by mean SEM of log area-under-curve (AUC). FIG. 35B-C Plasma antibody blocking activities after the 3rd immunization. ELISA was performed to test plasma samples blocking ACE2, human RBD neutralizing antibodies DH1041 and DH1047 binding to SARS-CoV-2 spike protein (b), or plasma samples blocking ACE2 and DH1047 binding to RsSHC014 spike protein (c). Data are expressed as percentages of blocking of ACE or the indicated antibody by 1:50 diluted plasma samples. FIG. 35D. Mucosal antibody binding titers in the lower and upper respiratory tracts after the 3rd immunization. ELISA was performed to test 10× concentrated bronchoalveolar lavage (BAL) or unconcentrated nasal wash samples binding to SARS-CoV-2 spike and RBD. ELISA binding titers are expressed as log AUC. FIG. 35E. Mucosal antibody blocking activities in the lower respiratory tracts after the 3rd immunization. ELISA for 10× concentrated BAL or unconcentrated nasal wash samples blocking ACE2, DH1041 and DH1047 binding to SARS-CoV-2 spike protein were performed, and percentages of blocking of ACE or the indicated antibody were shown. Statistical significance in all the panels were determined using Wilcoxon rank sum exact test. Asterisks show the statistical significance: ns, not significant, *P<0.05.

FIG. 36A-F. RBD-scNP, NTD-scNP and S2P-scNP induced spike-specific binding antibodies and mediated ADCC. Related to FIG. 32. FIG. 36A. Plasma antibody binding titers. ELISA was performed to test plasma samples binding to SARS-CoV-2 spike, RBD or NTD recombinant proteins, as well as to spike proteins of SARS-CoV, bat coronaviruses RaTG13, RsSHC014, and pangolin coronavirus GX-P4L. ELISA binding titers were are expressed as log area-under-curve (AUC). Dot and error bars indicate mean±SEM of all animals in each group. FIG. 36B-C. Plasma antibody blocking activity after the 3rd immunization. ELISA was performed to test plasma antibodies blocking ACE2, human RBD neutralizing antibodies DH1041 and DH1047, and human NTD neutralizing antibody DH1050.1 binding to SARS-CoV-2 spike protein (b), or plasma samples blocking ACE2, DH1041 and DH1047 binding to RsSHC014 spike protein (c). Data are expressed as percentages of blocking of ACE or the indicated antibody by 1:50 diluted plasma samples. FIG. 36D. Plasma antibodies staining on SARS-CoV-2 spike-transfected 293T cells. Pre-immunization (week 0) and pre-challenge (week 10) samples were tested. FIG. 36E. The gating strategy for the NK cell degranulation ADCC assay. Purified human NK cells were mixed with SARS-CoV-2 spike-transfected cells or SARS-CoV-2 infected cells in the presence of 1:500 diluted plasma samples. NK cell degranulation was detected based on CD107a expression. f. RBD-scNP-, NTD-scNP- and S2P-scNP-induced antibodies mediated ADCC. The percentages of CD107a+NK cells were shown when NK cells were assayed with plasma antibodies (left) or purified IgG (right) in SARS-CoV-2 spike transfected 293T cells (top row) or SARS-CoV-2 infected Vero E6 cells (bottom row). Fab2 fragments were set as a negative control for purified, whole IgG.

FIG. 37A-G. Antibody responses and T cell responses elicited by scNP vaccines as a booster vaccination in macaques that received two doses of S-2P mRNA-LNP vaccine. FIG. 37A. Plasma antibody binding titers. ELISA was performed to test plasma antibodies binding to SARS-CoV-2 spike, RBD and NTD, as well as spike proteins of SARS-CoV, bat coronaviruses RaTG13, RsSHC014, and pangolin coronavirus GX-P4L. ELISA binding titers were shown by log area-under-curve (AUC). Dot and error bars indicate mean±SEM of all animals in each group. FIG. 37B-C. Plasma antibody blocking activity after the 3rd immunization. ELISA was performed to test plasma antibodies blocking ACE2, human RBD neutralizing antibodies DH1041 and DH1047, and human NTD antibody DH1050.1 binding to SARS-CoV-2 spike protein (b), or plasma samples blocking ACE2, DH1041 and DH1047 binding to RsSHC014 spike protein (c). Data are expressed as percentages of blocking of ACE or the indicated antibody by 1:50 diluted plasma samples. FIG. 37D-E. Comparison of mucosal antibody blocking activities induced by 3 doses of scNP vaccination or 2 doses of S2P mRNA-LNP+1 dose of scNP vaccination. ELISA for 10× concentrated bronchoalveolar lavage (BAL) samples (d) and neat nasal wash samples (e) blocking the binding of ACE2 or neutralizing antibody (DH1041 or DH1047) on SARS-CoV-2 spike were performed. Data are expressed as percentages of blocking of ACE or the indicated antibody by mucosal samples. Statistical significance in all the panels were determined using Wilcoxon rank sum exact test. Asterisks show the statistical significance: ns, not significant, *P<0.05. f-g. T cell responses induced by RBD-scNP vaccine or the S2P mRNA-LNP vaccine. FIG. 37F-G. Percentages of cytokine-secreting CD4+(f) or CD8+(g) memory T cells were quantified in monkeys received 2 doses of RBD-scNP immunization, or 2 doses of S-2P mRNA-LNP, or unimmunized controls. The intracellular staining for IFN-γ, IL-2, TNF-α, IL-4 or IL-13 was performed following stimulation with SARS-CoV-2 spike protein peptide pool. Percentages of cytokine-secreting cells were shown for individual monkeys as dots, and for mean values of each vaccine group as bars.

FIG. 38. Three doses of RBD-scNP immunization induced neutralizing antibodies against SARS-CoV-2 Omicron variant. Plasma antibody (n=5, post-3rd immunization) neutralization titers against pseudoviruses of the SARS-CoV-2 Omicron variants in 293T-ACE2 cells. The geometric mean ID50 and ID80 titers and the fold reduction compared to SARS-CoV-2D614G are shown. Adjuvant is 3M052-5 ug/Alum 500 ug.

FIG. 39. Two doses of RBD-scNP immunization induced neutralizing antibodies against SARS-CoV-2 Omicron variant. Plasma antibody (n=15, post-2nd immunization) neutralization titers against pseudoviruses of the SARS-CoV-2 Omicron variants in 293T-ACE2 cells. The geometric mean ID50 and ID80 titers and the fold reduction compared to D614G are shown. Adjuvant is 3M052-5 ug/Alum 500 ug.

FIG. 40. Neutralizing antibodies against SARS-CoV-2 Omicron variant elicited by RBD-scNP vaccine formulated with three different adjuvants. Plasma antibody (post-2nd and post-3rd immunization) neutralization titers against pseudoviruses of the SARS-CoV-2 Omicron variants in 293T-ACE2 cells. The geometric mean ID50 titers and the fold reduction compared to D614G are shown. The dashed arrows indicate fold increase of ID50 titer induced by the 3rd boost. Adjuvants as above the graphs. Antibodies that cross-reacted with SARS-CoV-2 Omicon and neutralized Omicron were more potent and less fold-reduction compared to 614G SARS-CoV-2 were higher when the adjuvant used was 3M052-AF 5 ug. Dose of Alum alone was 500 ug. Dose of 3M052/Alum together was 5 ug and 500 ug respectively.

FIG. 41. Neutralizing antibodies against SARS-CoV-2 Omicron variant induced by NTD-scNP and S2P-scNP vaccines. NTD-scNP- or S2P-scNP-induced plasma antibody (post-2nd and post-3rd immunization) neutralization titers against pseudoviruses of the SARS-CoV-2 Omicron variants in 293T-ACE2 cells. The geometric mean ID50 titers and the fold reduction compared to D614G are shown. Adjuvant used was 3M052/Alum 5 ug/500 ug respectively.

FIG. 42. Neutralizing antibodies against SARS-CoV-2 Omicron variant induced by RBD-scNP as a heterologous boost for S2P mRNA-LNP vaccine. Plasma antibody (post-2nd and post-3rd immunization) neutralization titers against pseudoviruses of the SARS-CoV-2 Omicron variants in 293T-ACE2 cells. The geometric mean ID50 titers and the fold reduction compared to D614G are shown. The dashed arrow indicates fold increase of ID50 titer induced by the 3rd boost. Adjuvant used for RBD-scNP was 3M052/Alum 5 ug/500 ug respectively.

FIG. 43A-B show non-limiting embodiments of nucleic acid (SEQ ID NOS 233-243) (FIG. 43A) and amino acid sequences (SEQ ID NOS 244-253, and 36) (FIG. 43B) of the invention. Amino acid sequences in FIG. 43B include a signal peptide, e.g.

(SEQ ID NO: 23)
MGWSCIILFLVATATGVHA.

### DETAILED DESCRIPTION

On Mar. 11, 2020, the World Health Organization (WHO) characterized the ongoing spread of COVID-19, a highly contagious respiratory disease caused by the new betacoronavirus SARS-CoV-2 (SARS-2), a pandemic. As the virus continues to spread, there is an urgent need to understand as much as possible, as rapidly as possible, about this new virus.

The ongoing global pandemic of the new SARS-CoV-2 coronavirus (CoV) presents an urgent need for the development of effective preventative and treatment therapies. The viral-host cell fusion (S) protein spike is a prime target for such therapies owing to its critical role in the virus lifecycle. The spike protein is divided into two regions: the N-terminal S1 domain that caps the C-terminal S2 fusion domain. Binding to host receptor via the Receptor Binding Domain (RBD) in S1 is followed by proteolytic cleavage of the spike by host proteases. Large conformational changes in the S-protein result in S1 shedding and exposure of the fusion machinery in S2. Class I fusion proteins such as the CoV spike protein that undergo large conformational changes during the fusion process must, by necessity, be highly flexible and dynamic. Indeed, cryo-EM structures of the SARS-CoV-2 (SARS-2) spike reveal considerable flexibility and dynamics in the S1 domain, especially around the RBD that exhibits two discrete conformational states—a “down” state that is shielded from receptor binding, and an “up” state that is receptor-accessible. Walls, A. C. et al. Structure, Function, and Antigenicity of the SARS-CoV-2 Spike Glycoprotein. Cell, doi:https://doi.org/10.1016/j.cell.2020.02.058 (2020); Wrapp, D. et al. Cryo-EM structure of the 2019-nCoV spike in the prefusion conformation. Science 367, 1260-1263, doi:10.1126/science.abb2507 (2020).

The transmembrane SARS-2 spike protein spike trimer mediates attachment and fusion of the viral membrane with the host cell membrane and is therefore critical for the viral life cycle. Displayed on the surface of the virus, the spike protein is a prime target for vaccine and therapeutics design.

The SARS-2 spike protein displays striking structural similarities with the S proteins of the previously identified SARS-CoV, MERS-CoV, and other human and murine CoV viruses. However, most S-targeting antibodies to SARS and MERS do not cross-react with SARS-2. Conformational evasion is among the many host immune evasion tools available to viruses. Dramatic shifts in the conformational ensemble of states for CoVs have in fact been demonstrated.

In certain aspects the invention provides modified coronavirus spike proteins designs including but not limited to protein designs comprising spike protein and/or various spike portions/domains from SARS-CoV-2 (SARS-2), SARS-CoV-1 (CoV1), MERS, or any other coronavirus spike protein, wherein in certain embodiments these proteins are designed to form multimeric complexes. The invention provides amino acid and nucleic acid sequences of recombinant coronavirus spike proteins or portions thereof, wherein in certain embodiments these spike proteins or portions/domains are multimerized, and could be used as an antigen to induce an immunogenic response. In some embodiments the antigen comprises any suitable portion from a spike protein. In non-limiting embodiments are portions of the spike protein which comprise epitopes conserved between different coronaviruses. In some embodiments the antigen comprises RBD domain from a spike protein. In some embodiments the antigen comprises NTD domain from a spike protein. In some embodiments the antigen comprises FP domain from a spike protein. The sequence of the spike protein is any suitable sequence coronavirus sequence including without limitation SARS-CoV1, SARS-CoV2, MERS, bat coronavirus, pangolin or other animal coronaviruses. In non-limiting embodiments the spike protein sequences comprise any variation in amino acid sequences, including without limitation Wuhan SARS-CoV2 sequence, UK SARS-CoV2 variant B.1.1.7, South African variant 1.351, US SARS-CoV-2 variants with L452R mutations and Brazilian variant P.1, delta, omicron and omicron variants. Additional SARS-2 spike protein sequences from circulating viruses are found in the GISAID EpiFlu™ Database. These sequences could also be designed with any of the modifications described herein.

Provided are also nucleic acids, including modified mRNAs which are stable and could be used as antigens to induce an immune response. Provided also are nucleic acids optionally designed as vectors, for example for recombinant expression and/or stable integration, e.g. but not limited, gp160 DNA encoding trimer for stable expression, or VLP incorporation.

The SARS-2 Spike (S) protein includes the receptor binding domain and is a target for neutralizing antibodies. We have designed recombinant protein and DNA constructs that express SARS-2 coronavirus spike protein (GenBank Accession number: YP_009724390.1), for example, as the full-length, transmembrane spike protein, a truncated version of the spike protein that lacks the c-terminal transmembrane domain and cytoplasmic tail, or various spike domains. The truncated spike protein is secreted from expressing cells, whereas the full-length version of the plasmid is expressed on the cell surface. Additional SARS-2 spike protein sequences from circulating viruses are found in the GISAID EpiFlu™ Database. These sequences could also be modified with any of the modifications described herein.

In non-limiting embodiments, the spike protein designs have several modifications from the wildtype reference sequence from GenBank. First, the SARS-2 protein sequence encodes furin cleavage sites and a cathepsin L cleavage site. The recombinant protein will be made with and without these protease cleavage sites to see if they affect protein quality, yield, and immunogenicity. Second, the natural signal peptide that directs intracellular trafficking of the spike protein will be exchanged for the bovine prolactin signal peptide. The bovine prolactin signal peptide is a strong signal peptide that directs proteins into the secretory pathway. This signal peptide is predicted by the SignalP 5.0 program to be cleaved off of the mature spike protein more efficiently than the natural virus signal peptide sequence. Third, the secreted spike protein should trimerize in order to resemble the native, membrane-bound spike protein on coronavirus virions. However, the truncated, secreted spike protein lacks the transmembrane domain and thus may not form a stable trimeric protein. To facilitate trimerization, we added a trimerization domain to the c-terminus of some truncated S proteins. The trimerization domain can be a 29 amino acid sequence called foldon for T4 bacteriophage fibritin protein (Strelkov S V et al. Biochemistry. 1999; Frank S et al. J Mol Biol. 2001). Fourth, we have encoded de novo cysteines to the protein sequence to create new intramolecular and intermolecular disulfide bonds. The bonds prevent conformational changes within the S protein. Non-limiting examples are represented by Clusters modifications 1-11. Fifth, we have encoded two new prolines in between HR1 and the central helix in the spike protein to stabilize the polypeptide turns in the S2 protein (Pallesen et al. PNAS. 2017). Sixth, we have added an AviTag to the truncated S proteins to facilitate functionalization by streptavidin binding.

For development as a vaccine immunogen, we have also created multimeric nanoparticles that display coronavirus spike protein or fragments on their surface. The rationale for creating such immunogens is that presenting multiple copies of the immunogen allows for a more avid interaction between the immunogen and naïve B cell receptors during the immune response. Thus, weak affinity interactions between the B cell receptor and immunogen are enhanced due to the multiple interactions that work in concert. Without wishing to be bound by theory, this improved interaction with B cells underlies the improved uptake of multimeric immunogens by B cells. The internalized immunogen is then presented to T cells in the context of MHC molecules. The T cells in turn provide the required costimulatory signals to the B cells to promote B cell maturation. Additionally, the SARS-CoV-2 spike protein has 22 glycosylation sites, which could interact with lectins to facilitate trafficking to secondary lymphoid organs. Multimerization of viral spike glycoproteins improves their interaction with mannose binding lectin, thereby increasing antigen trafficking to sites with abundant immune cells.

The nanoparticle immunogens are composed of various portions of SARS-CoV-2 spike protein and self-assembling ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the spike protein on its surface. The spike protein is displayed as a soluble Spike trimer that has the transmembrane domain and cytoplasmic tail removed and a foldon trimerization domain added. To focus antibodies to neutralizing targets, the spike protein will be truncated down to only the receptor binding domain (RBD), which is a known target for neutralizing antibodies. Without wishing to be bound by theory, this construct can generate neutralizing antibodies, while not eliciting binding antibodies to other sites that mediate antibody-dependent enhancement of virus infectivity (PMID: 25073113, PMID: 21775467).

Multimeric complexes presenting of antigens—nanoparticles. Presenting multiple copies of antigens to B cells has been a longstanding approach to improving B cell receptor recognition and antigen uptake (Batista and Neuberger, 2000). The improved recognition of antigen is due to the avid interaction of multiple antigens with multiple B cell receptors on a single B cells, which results in clustering of B cells and stronger cell signaling. Furthermore, multimeric presentation improves antigen binding to mannose binding lectin which promotes antigen trafficking to B cell follicles (Tokatlian et al., 2019). Self-assembling complexes comprising multiple copies of an antigen are one strategy of immunogen design approach for arraying multiple copies of an antigen for recognition by the B cell receptors on B cells (Kanekiyo et al., 2013; Ueda et al., 2020).

In some instances, the gene of an antigen can be fused via a linker/spacer to a gene of a protein which could self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen.

Multivalent/multimeric presentation using ferritin. Ferritin is a well-known protein that self-assembles into a hollow particle composed of repeating subunits. In some species ferritin nanoparticles are composed of 24 copies of a single subunit, whereas in other species it is composed of 12 copies each of two subunits. We chose here the two subunit ferritin nanoparticle from Trichoplusia ni since the viral antigen of choice can be fused to, for example, one or both chains of the ferritin nanoparticle. Thus, by fusing the antigen to only one chain the valency can be 12 individual antigens or by fusing the antigen to both chains the valency can be increased to 24 copies of the antigen. While 24 copies of the antigen may increase the interaction with lectins that enhance antigen trafficking, it can also make the nanoparticle unstable or preclude access to epitopes on the sides of the exogenous antigen because the antigens are too densely clustered on the nanoparticle. In such instances having the ability to form nanoparticles with lower antigen valency because the antigen is fused to only one ferritin chain would be beneficial.

Structural considerations and ferritin selection in design of nanoparticle. Previous studies have attempted to use ferritin to display complex antigens on their surface. However, the fusion of heterologous proteins to ferritin and the formation of nanoparticles is inconsistent. Often the nanoparticle fails to form or does so inefficiently resulting in low yields. This necessitates many rounds of iterative protein design to find the optimal fusion protein construct. We inspected the structure of Helicobacter pylori ferritin to determine reasons why fusing the gene of a protein of interest to the 5′ end of the ferritin gene might disrupt nanoparticle formation upon translation. Without being bound by theory, several considerations are discussed. First, the N-terminus of Helicobacter pylori ferritin subunits adopt an alpha helix structure. The fusion of the exogenous protein must occur at the point in the sequence where the helix has turned away from the center of the nanoparticle. Therefore, the N-terminus can be truncated to the Asp residue at position 5 since it is the last apical amino acid. Second, the sequence of the heterologous fused protein will have to be compatible with the alpha helix secondary structure. The heterologous fused protein cannot break the alpha helix secondary structure or the ferritin subunit will not properly fold. At the same time, the sequence of heterologous fused protein cannot continue the alpha helix structure or the extended helix will clash with adjacent ferritin chains. To disrupt the alpha helix we have designed long linkers, which could be flanked by prolines (e.g. PGS19 and PGS34) to break the secondary structure of the peptide to which the heterologous protein is attached. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine. Third, the tertiary structure of each H. pylori ferritin subunit causes the N-terminal helix to be distal to the three-fold symmetrical axis in the center of the trimeric subunits. Thus, the interaction or clustering of the heterologous antigens requires crossing over top of two or more ferritin subunit alpha helices. The heterologous protein has to attach to the ferritin subunit in such a way that it can turn the peptide chain to the left to cross over top of the helices. Such a redirection of the peptide chain can only happen if the heterologous protein does not continue the alpha helical secondary structure of the N-terminus of H. pylori ferritin.

Trichoplusia ni (T. ni) ferritin is an alternative sequence to the H. pylori ferritin. T. ni has two ferritin chains whose structures are different from Helicobacter pylori ferritin. In contrast to H. pylori ferritin chains, the two chains of T. ni ferritin have long flexible N-terminal regions. The limited intramolecular interactions of these N-terminus with other ferritin chains allows it to be easily fused to a heterologous protein without disrupting the overall ferritin chain structure or assembly of the particle. Without being bound by theory fusion proteins where an antigen sequence is fused to T. ni ferritin sequence overcomes some of the technical issues when H. pylori ferritin is used.

Increasing the valency of self-assembling nanoparticles. Encapsulin nanoparticles have 60 or more subunits to which heterologous proteins can attach. This provides an increase of 36 copies of a heterologous antigen of choice compared to ferritin nanoparticles. Encapsulins also have the advantage of being able to encapsulate other proteins inside the nanoparticle (Sutter et al., 2008). Structures of Thermotoga maritima, Myxococcus xanthus, and Pyrococcus furiosus encapsulins have been solved, which allows for determination of the sites of attachment (Gabashvili et al., 2020). The structure of Thermotoga maritima encapsulin showed that it had 60 repeat subunits that comprised one nanoparticle (Sutter et al., 2008). Some encapsulin nanoparticles such as Pdu nanoparticles are composed of three or more subunits making it more complicated to express and form as an intact nanoparticle (Crowley et al., 2010; Havemann et al., 2002). The N-terminus of T. maritima encapsulin points into the lumen of the particle whereas the C-terminus points out away from the nanoparticle. Thus, the C-terminus of Thermotoga maritima encapsulin is the optimal attachment site for a heterologous protein so that the folding of the encapsulin subunit or the assembly of the nanoparticle in not disrupted. The C-terminus is also an optimal site for attachment since it lacks secondary structure and is just a flexible loop pointing away from the nanoparticle. Encapsulin nanoparticles displaying Epstein-Bar virus glycoproteins have been successfully achieved validating their use as a nanoparticle scaffold (Kanekiyo et al., 2015).

Selection of SARS-CoV-2 antigens. The Spike (S) protein of coronaviruses (CoV) is the main target for neutralizing, protective antibodies (Piccoli et al., 2020). Within the ectodomain of the S protein there are three domains of the SARS-CoV-2 S protein that are known to contain neutralizing epitopes (Piccoli et al., 2020. These three domains are the receptor binding domain (RBD; amino acids 319-541), N-terminal domain (NTD; amino acids 27-292), and S2 domain (begins at amino acid 819 goes until the end of the protein). Thus, these spike domains are suitable immunogens to include coronavirus vaccine eliciting antibody responses. However, there are differences in neutralizing activity for antibodies binding to each of these three sites. The RBD (amino acids 319-541) is the target of most potent neutralizing antibodies identified thus far (Liu et al., 2020; Piccoli et al., 2020). Cross-neutralizing RBD antibodies—antibodies that can neutralize multiple coronaviruses—have been isolated from SARS-CoV-1-infected individuals and shown to protect against multiple coronaviruses (Wec et al., 2020). Thus, RBD subunit vaccines are the primary focus of vaccines aiming to prevent infection against multiple different coronaviruses or potently inhibit a single coronavirus. The second most potent neutralizing antibodies are against the NTD (Li et al., 2021; Liu et al., 2020), and only weakly neutralizing antibodies have been found against the S2 domain (Li et al., 2021). While sequences for SARS-CoV-2 and MERS are provided for these vaccine targets, vaccines using the same design strategy could be made from any coronavirus of interest. In such designs, the RBD, NTD, FP, or spike ectodomain would be fused to a ferritin or encapsulin subunit.

Rational selection of sites of vulnerability for cross-neutralizing CoV antibodies. While the RBD elicits potent neutralizing antibodies against the matching virus, the RBD sequence varies across different groups of coronaviruses. This variation or unusual antibody characteristics may be the reason that we found only four potent cross-neutralizing antibodies among 463 antibodies we characterized (Li et al., 2021). We compared the amino acid sequence of betacoronaviruses and identified the fusion peptide (FP) within the S2 domain (amino acids 819-1240) as highly conserved across betacoronaviruses. The FP is targeted by neutralizing antibodies against HIV-1 (Kong et al., 2016). Without wishing to be bound by theory, the coronavirus FP could be a neutralizing determinant for CoV as well since both HIV-1 and coronaviruses have type I viral fusion proteins.

Vaccines to elicit cross-CoV group neutralizing antibodies. Herein, non-limiting embodiments of vaccine immunogens based on ferritin nanoparticles from H. pylori and T. ni that display RBD subunits and the highly conserved FP are described. The RBD includes amino acids 319 to 541, but could also be truncated to 333 and 529 (RBDTRUNC), depending on which construct expresses more highly. In some embodiments, RBD and fusion peptide are derived from SARS-CoV-2 and Middle East respiratory syndrome coronavirus (MERS-CoV). These two coronaviruses were used as representative strains of CoV from group 2b and 2c respectively. However, the vaccine immunogen can be altered to include other CoV sequences such as SARS-CoV-1, SHC014, or HKU3-1. Similarly, variants of individuals CoVs such as 501Y.V2, or any other coronavirus. The RBD and FP will be displayed on a ferritin nanoparticle or an encapsulin nanoparticle to increase the valency of the immunogen as described herein. T. maritima encapsulin was used since its 60-mer nanoparticle consists of only one repeating subunit to which a heterologous protein could be attached. H. pylori ferritin was used for nanoparticle designs given its established safety in humans vaccinated with influenza HA ferritin nanoparticle vaccines. T. ni ferritin was also used in designs since it allows for fusion of viral antigens to, for example, one or both ferritin chains and it has a flexible N-terminus on one of its chains as discussed herein. Flexible glycine-serine linkers were used to connect the C-terminus of the RBD or FP to the N-terminus of individual ferritin chains. Similarly, flexible glycine-serine linkers were used to connect the RBD to the C-terminus of encapsulin subunits. The length of these linkers can be varied to provide sufficient distance away from the individual encapsulin or ferritin subunit. Additionally, the glycine linkers are flanked with two prolines to break secondary structure at the site of fusion to RBDs or nanoparticle subunits.

For ease of purification the proteins were designed with secretory signal peptides. The signal peptides were selected from a panel of signal peptide sequences based on computational signal peptidase cleavage predictions. The fusion proteins are designed with or without amino acids that bind to various affinity purification resins appended to each subunit. Purification tags include polyhistidine tags and twin strepTagII, but could be other available affinity resin target sequences. To remove the purification from the final protein, the amino acids of the purification tags are preceded by a protease cleavage site for HRV-3C. Clinical vaccine products will not include the purification tags.

We have fused the FP to the N-terminus of H. pylori ferritin (HV1302136), and expressed nanoparticles displaying 24 copies of the SARS-CoV-2 FP. The nanoparticles were homogenous as determined by analytical Superose6 size exclusion and negative stain electron microscopy. To test the immunogenicity of the FP nanoparticle we immunized BALB/c mice 4 times and assessed binding antibodies to various coronaviruses. Serum IgG bound to human, bat, and pangolin spike proteins. We also examined neutralization against human and bat coronavirus. Three of five serum samples neutralized MERS-CoV.

Translatability to different vaccine platforms. Nucleic vaccines are an attractive platform since they can be rapidly manufactured and vaccination with them can elicit lasting humoral immunity (Pardi et al., 2018; Saunders et al., 2020). Since nucleic acid vaccines express the protein in vivo the quality of the immunogen is controlled by designing a gene that results in properly-folded protein. Without wishing to be bound by theory, the designs put forward here can fold properly making them suitable for nucleic vaccine platforms such as lipid nanoparticle encapsulated mRNA or DNA electroporation.

Presentation of antigens as particulates reduces the B cell receptor affinity necessary for signal transduction and expansion (See Batista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. The stronger B cell receptor interaction leads to stronger B cell activation and proliferation. Provided are spike proteins, including but not limited to receptor binding domains, ectodomains, peptides, N-terminal domains as particulate, high-density array on liposomes or other particles, for example but not limited to nanoparticles. See e.g. He et al. Nature Communications 7, Article number: 12041 (2016), doi:10.1038/ncomms12041; Bamrungsap et al. Nanomedicine, 2012, 7 (8), 1253-1271.

To improve the interaction between the naïve B cell receptor and immunogens, spike designs can be created to wherein the spike is presented on particles, e.g. but not limited to nanoparticle. In some embodiments, a coronavirus spike protein or a portion thereof, e.g. RBD, NTD, or FP, could be fused to ferritin. The spike could originate from any coronavirus for which amino acid sequences are known or can be derived. Ferritin protein self assembles into a small nanoparticle with three fold axis of symmetry. At these axes the spike protein is fused. Therefore, the assembly of the three-fold axis also clusters three spike protomers together to form a spiketrimer. Each ferritin particle has 8 axes which equates to 8 trimers being displayed per particle. See e.g. Sliepen et al. Retrovirology 201512:82, DOI: 10.1186/s12977-015-0210-4.

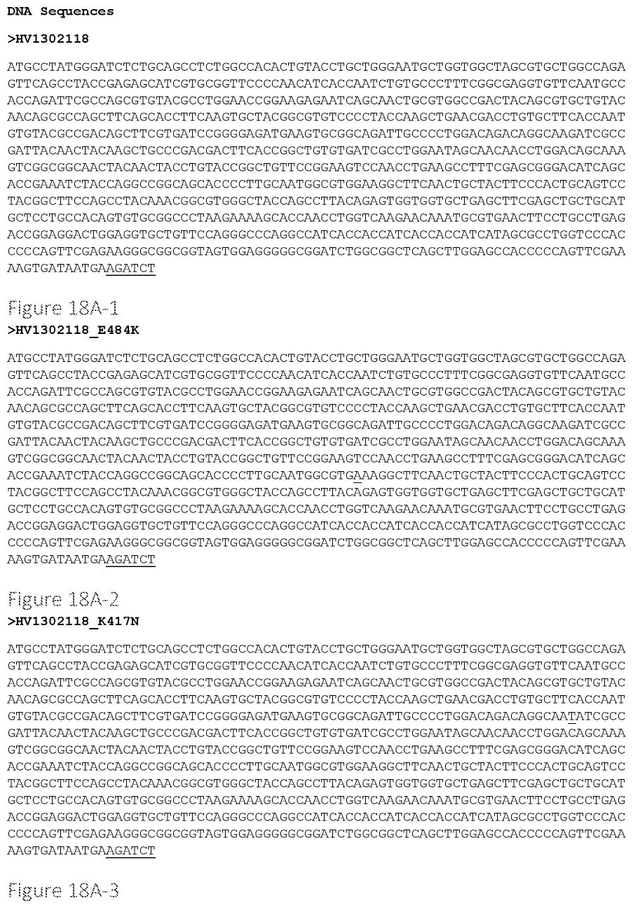

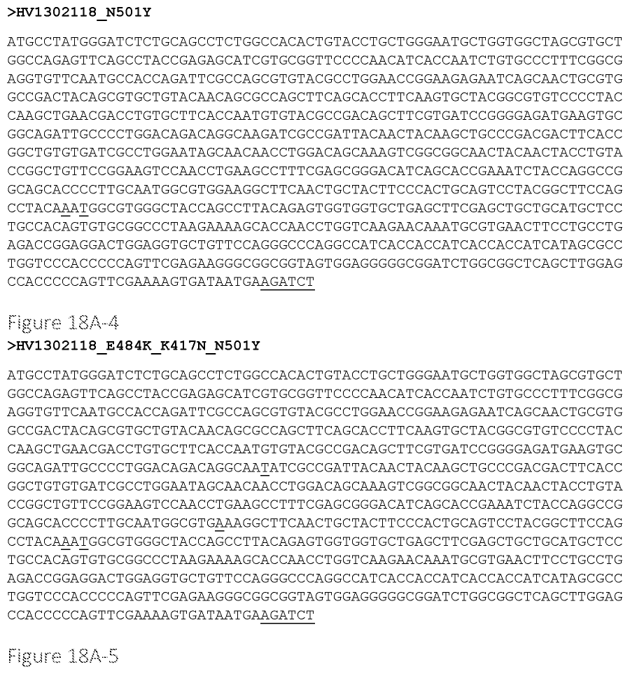

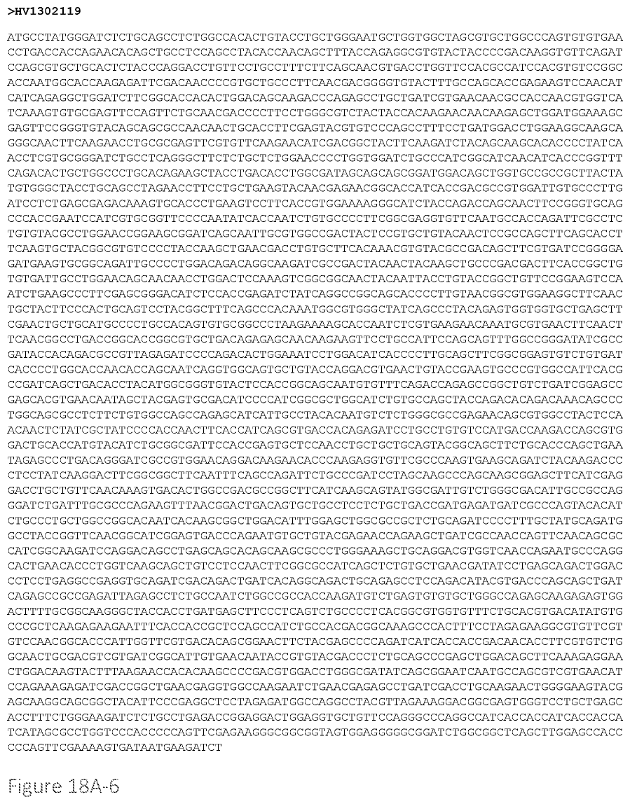

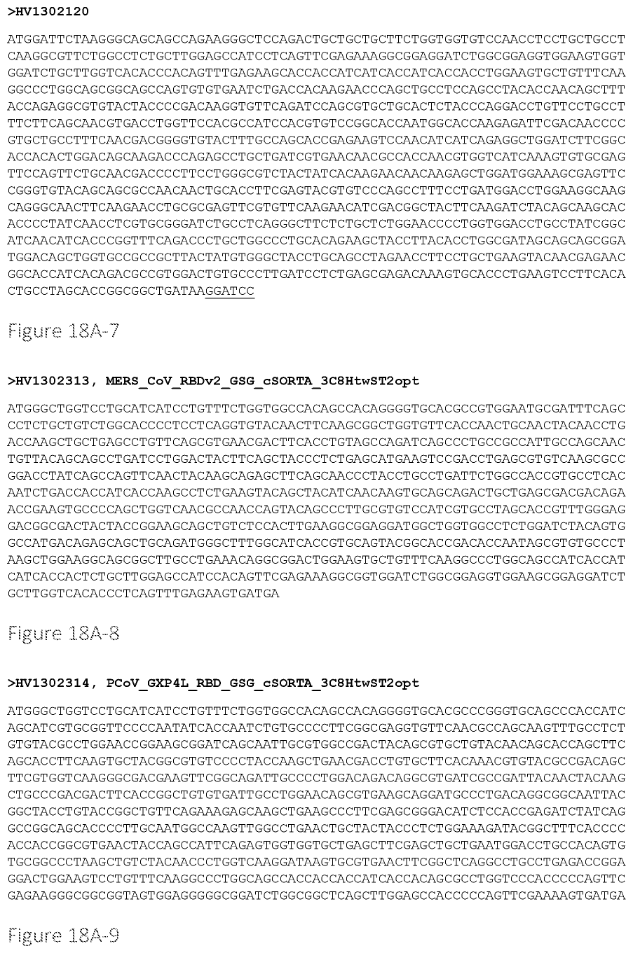

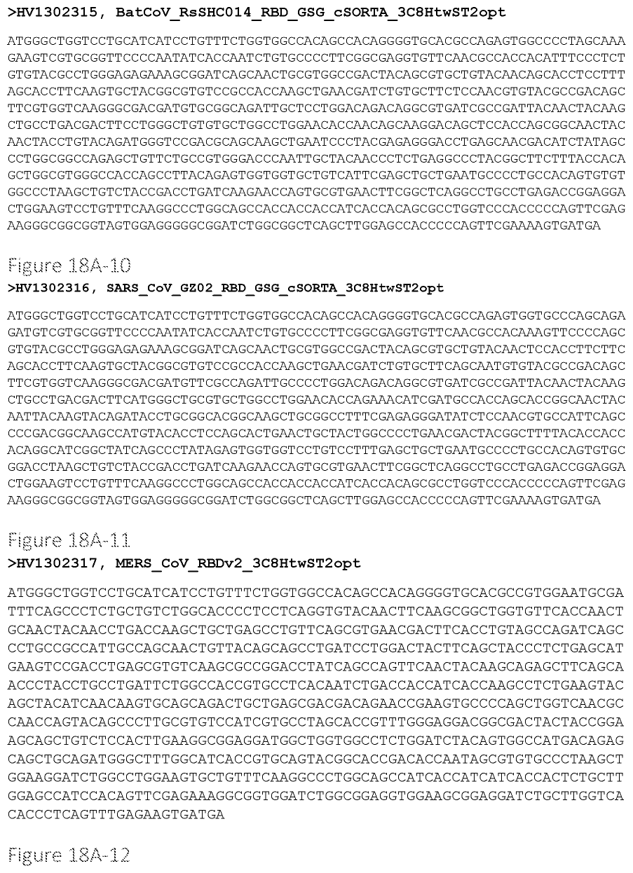

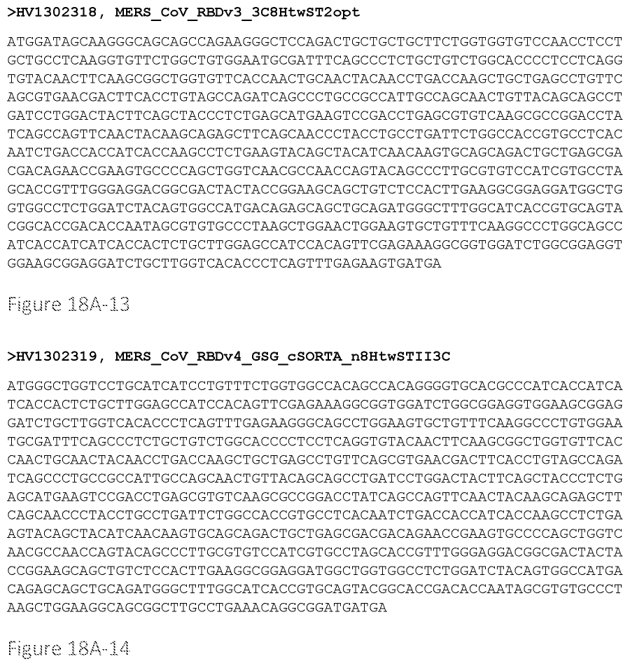

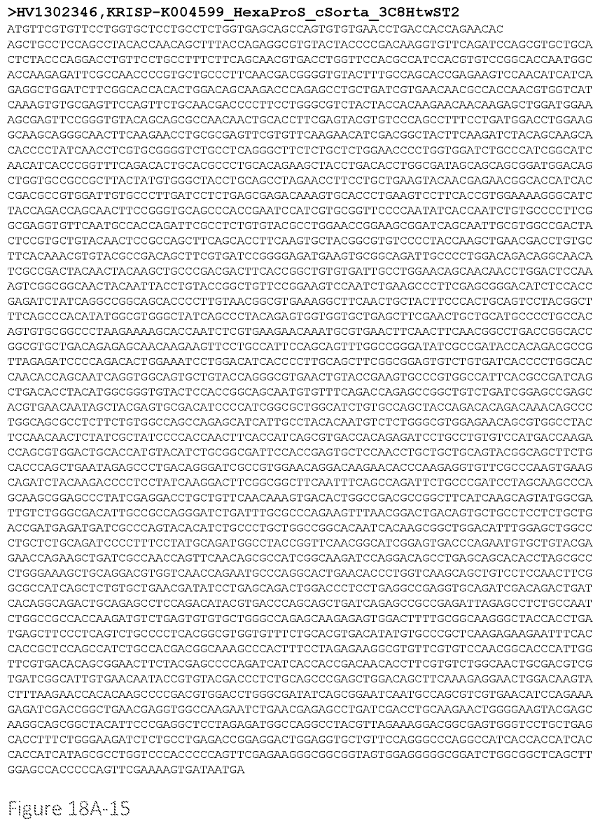

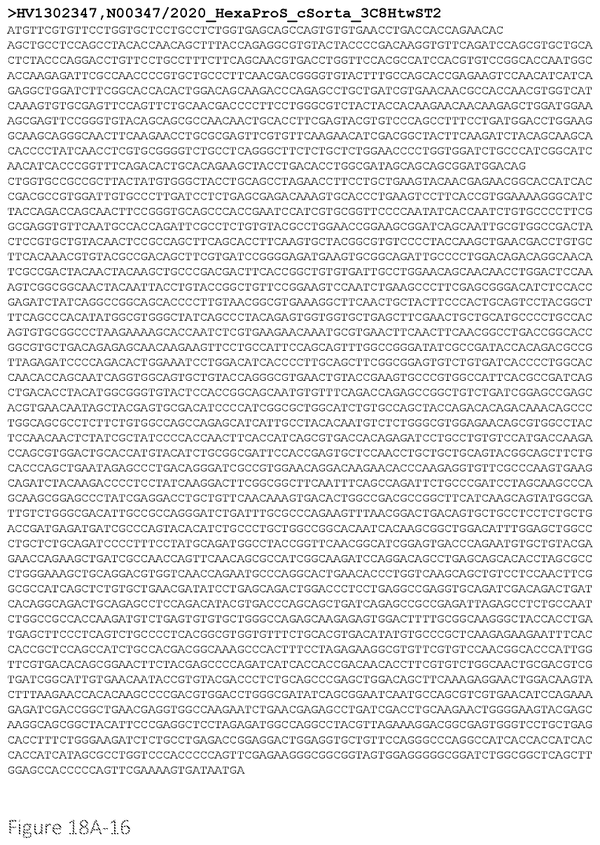

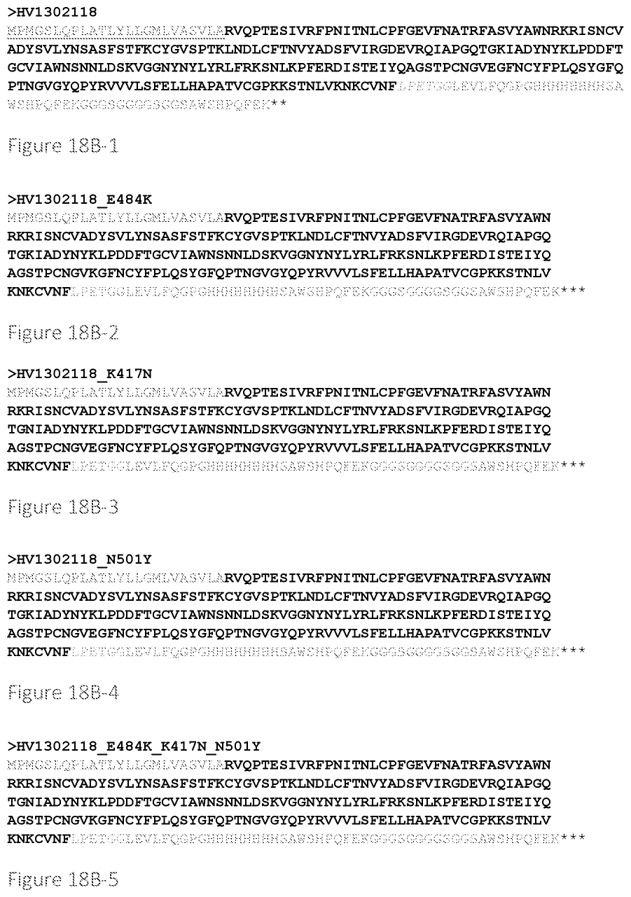

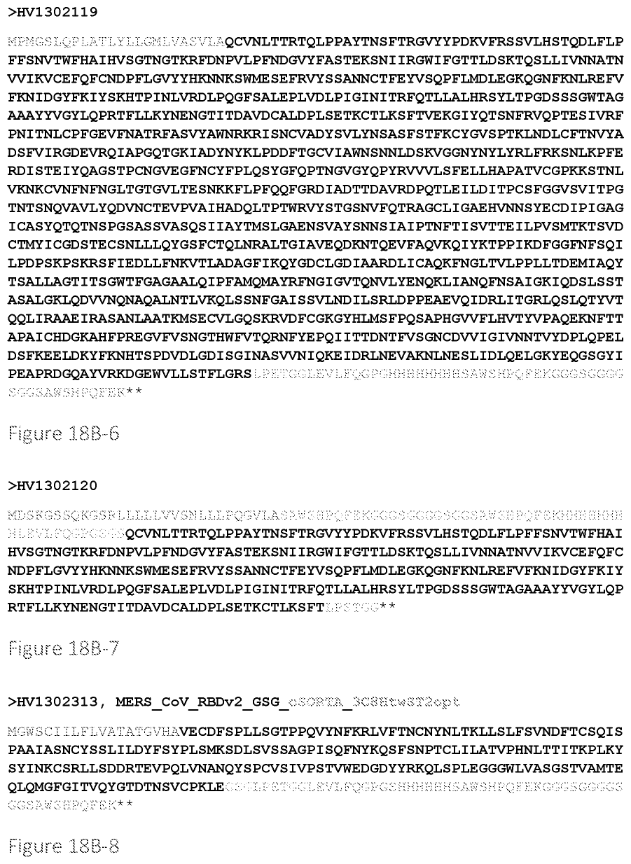

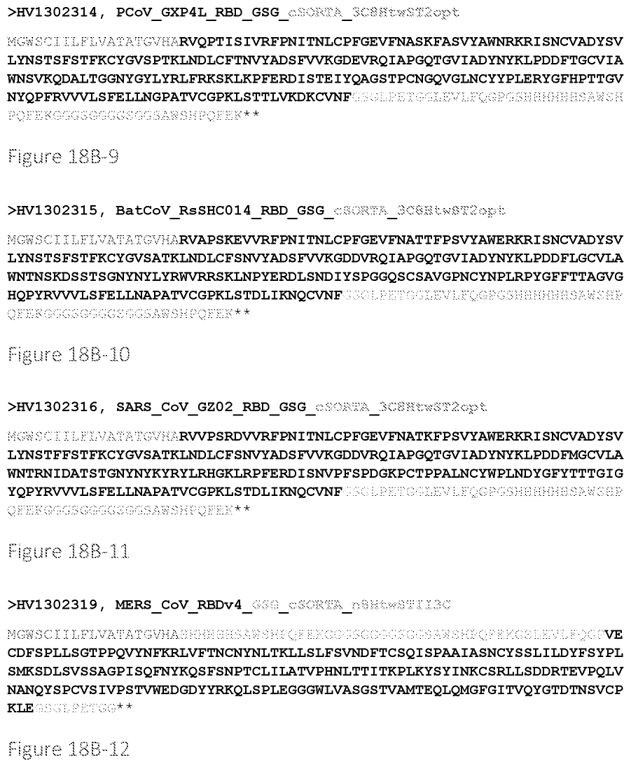

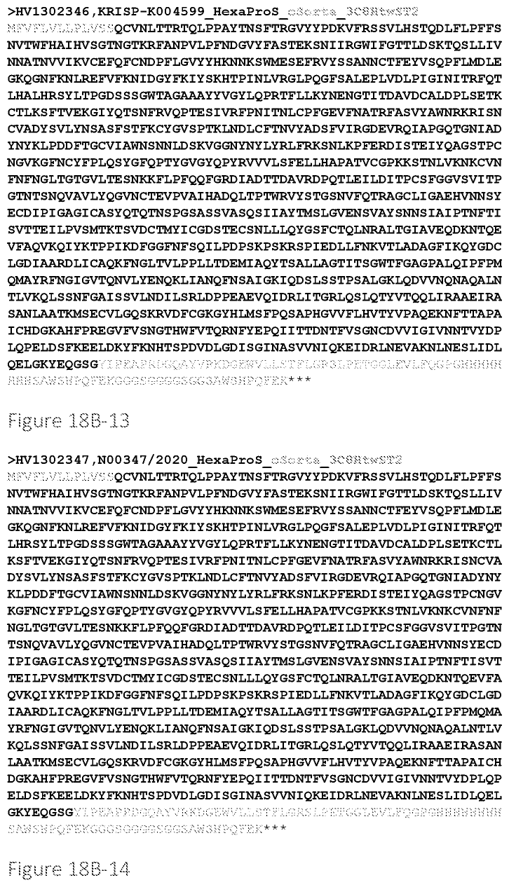

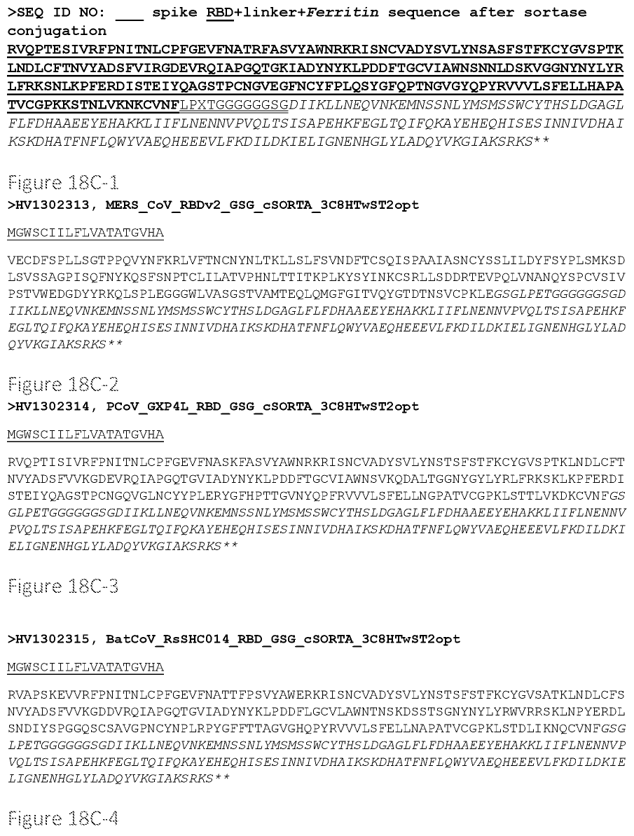

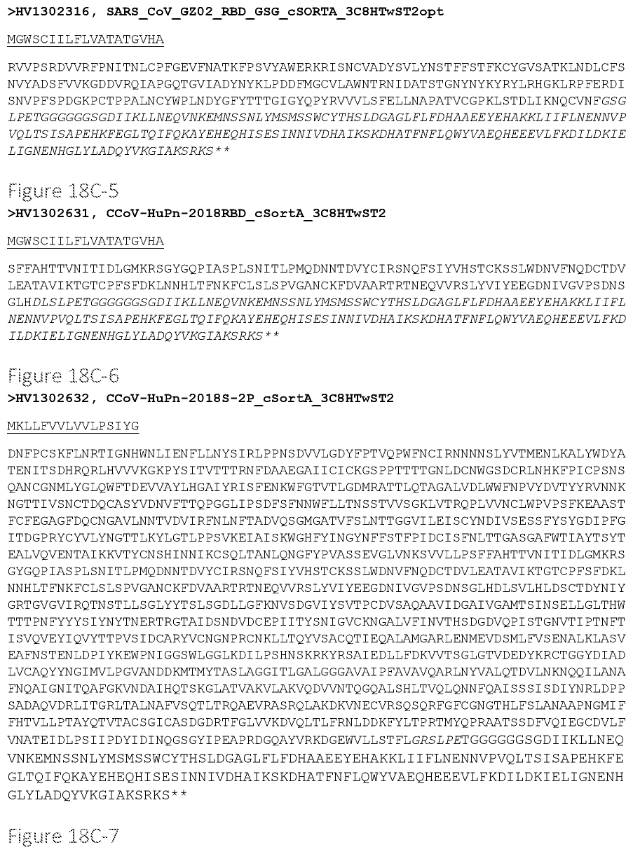

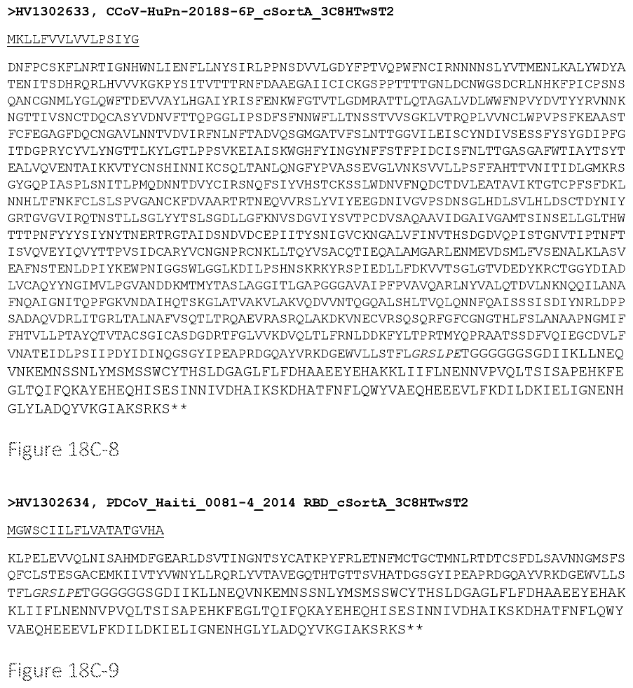

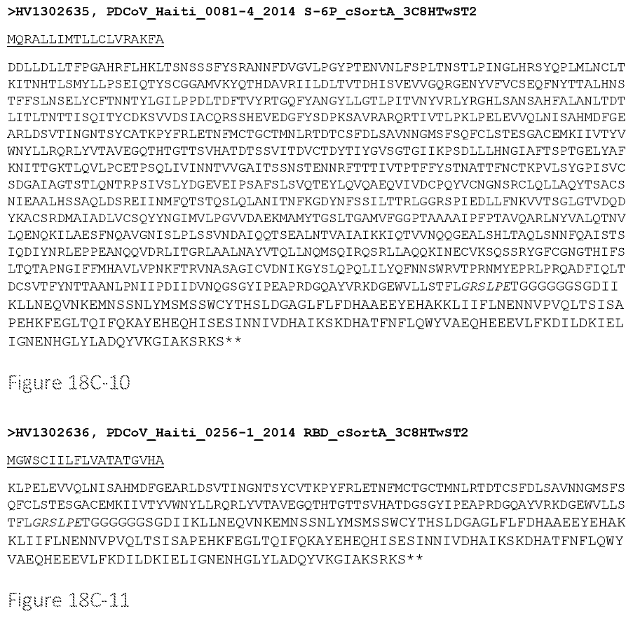

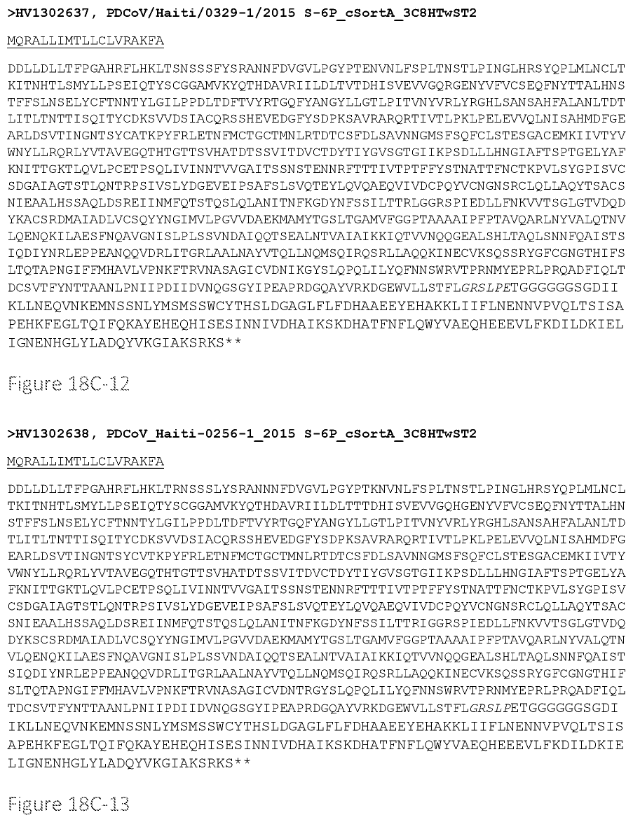

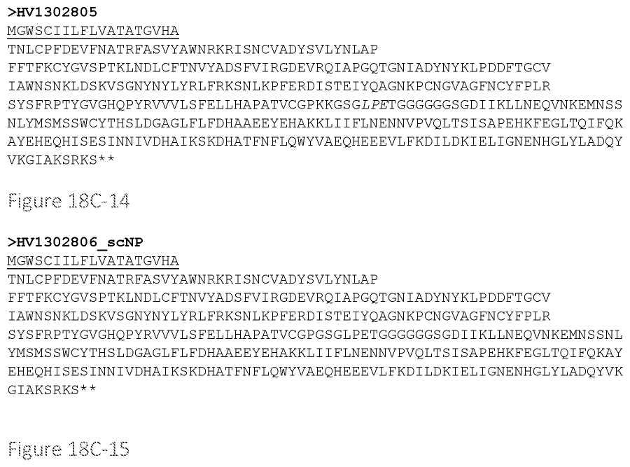

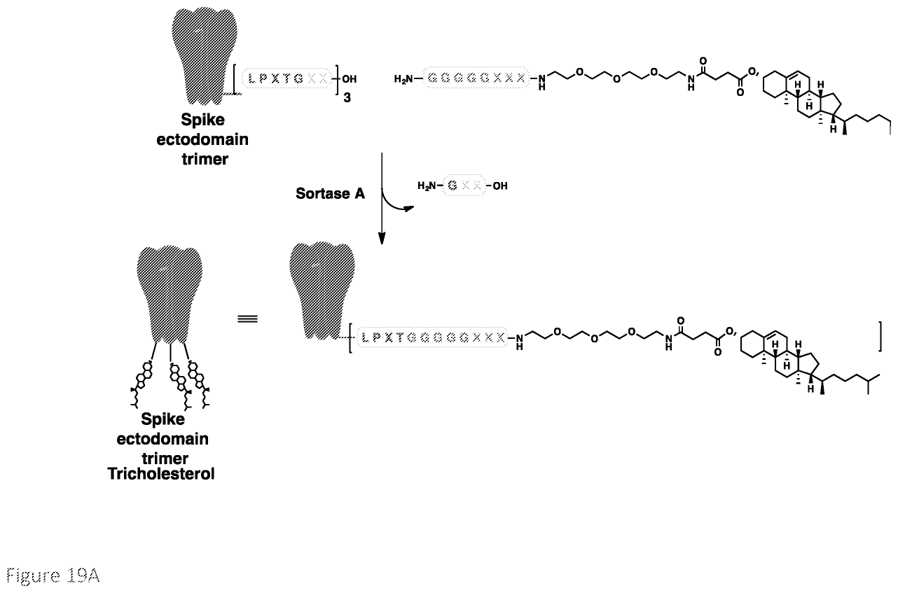

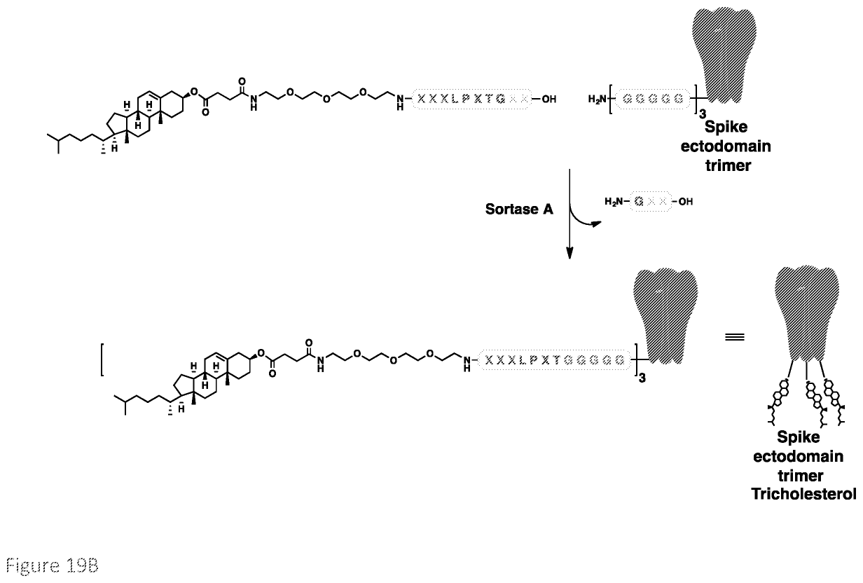

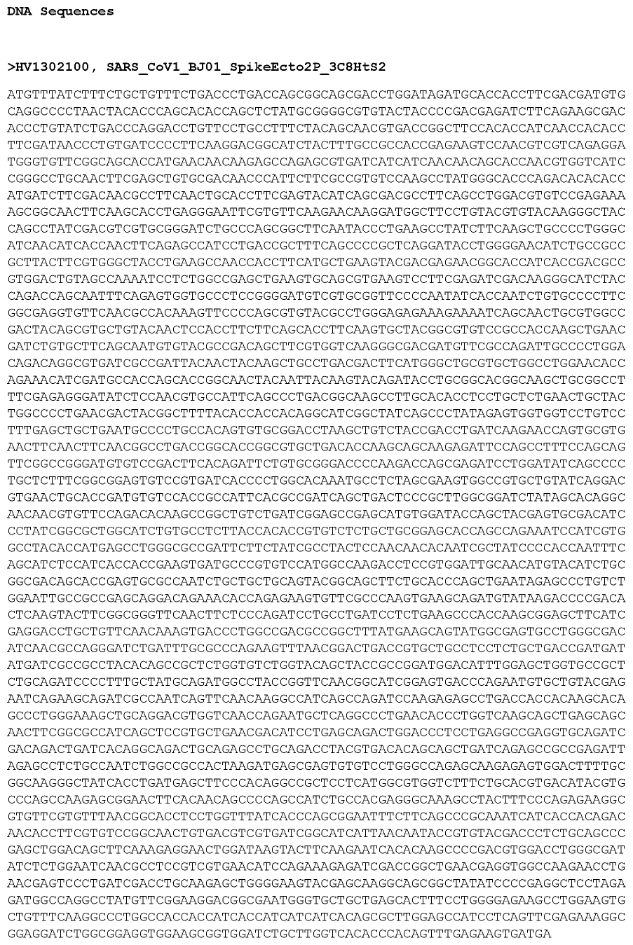

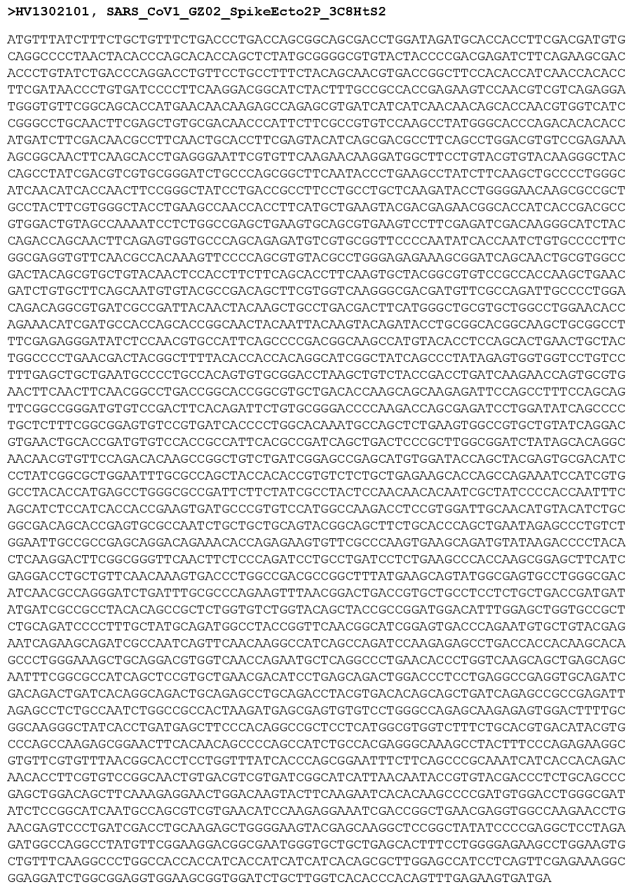

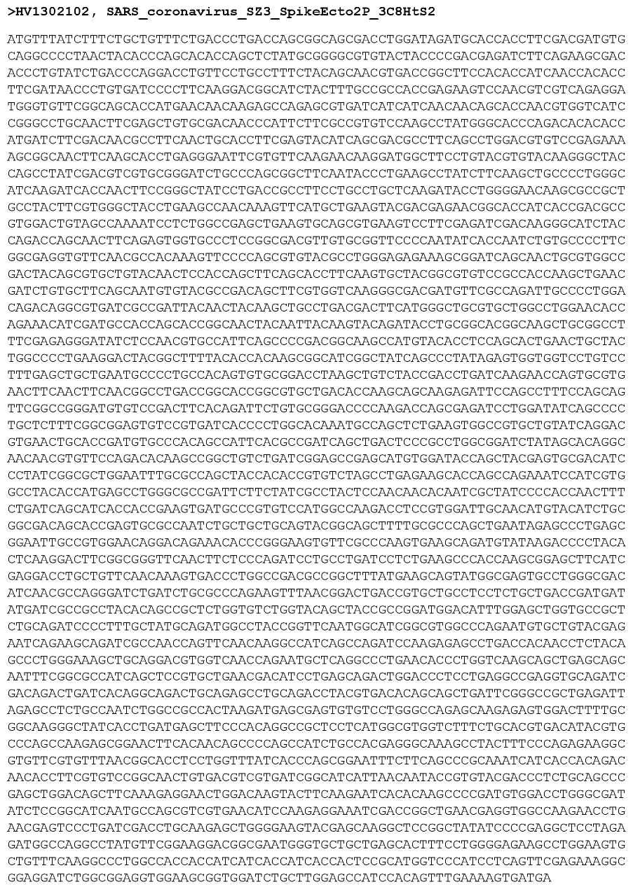

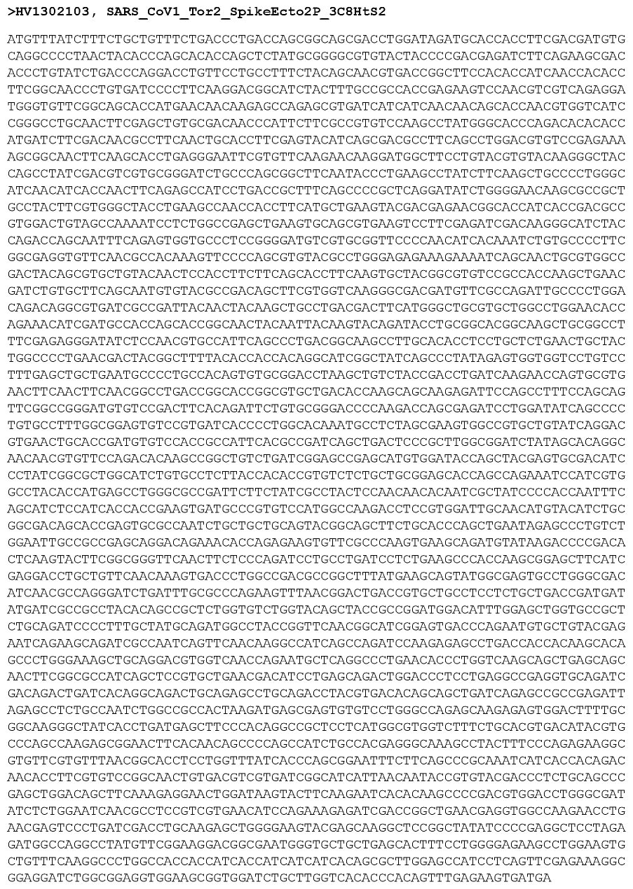

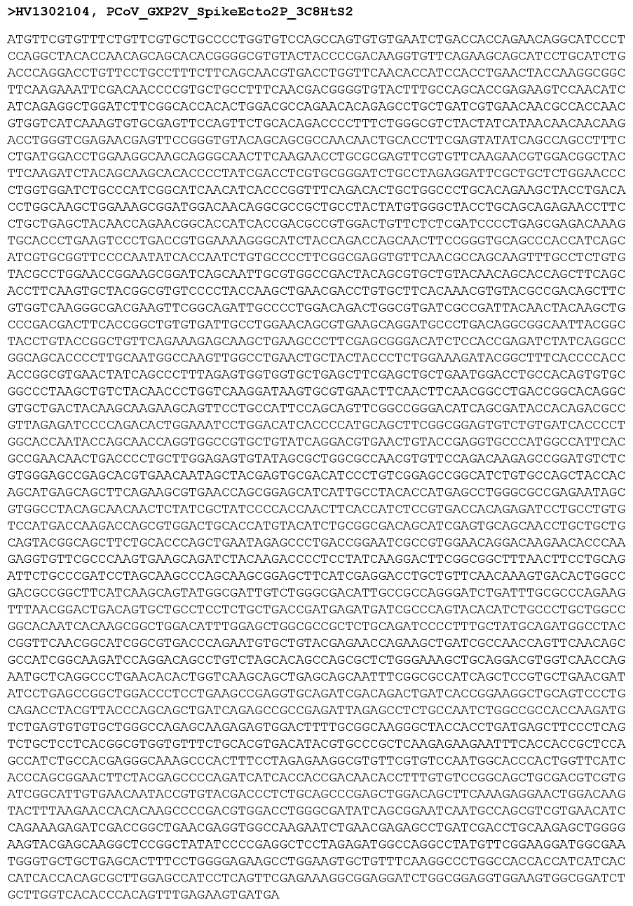

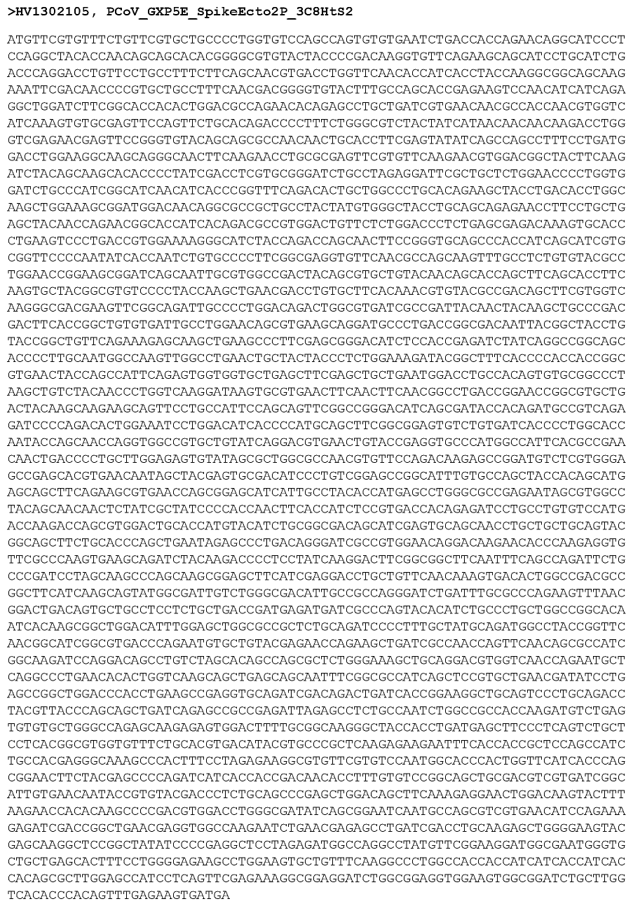

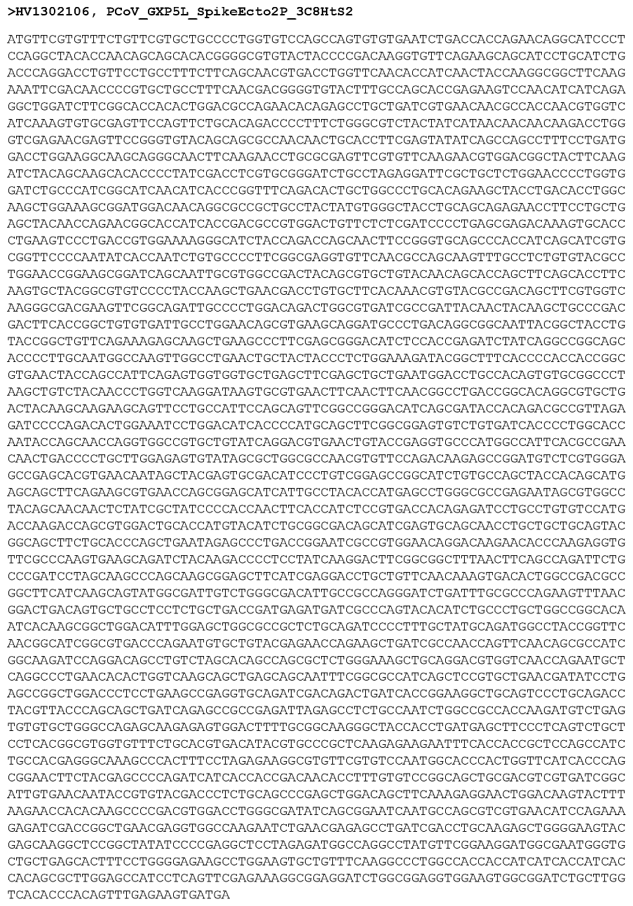

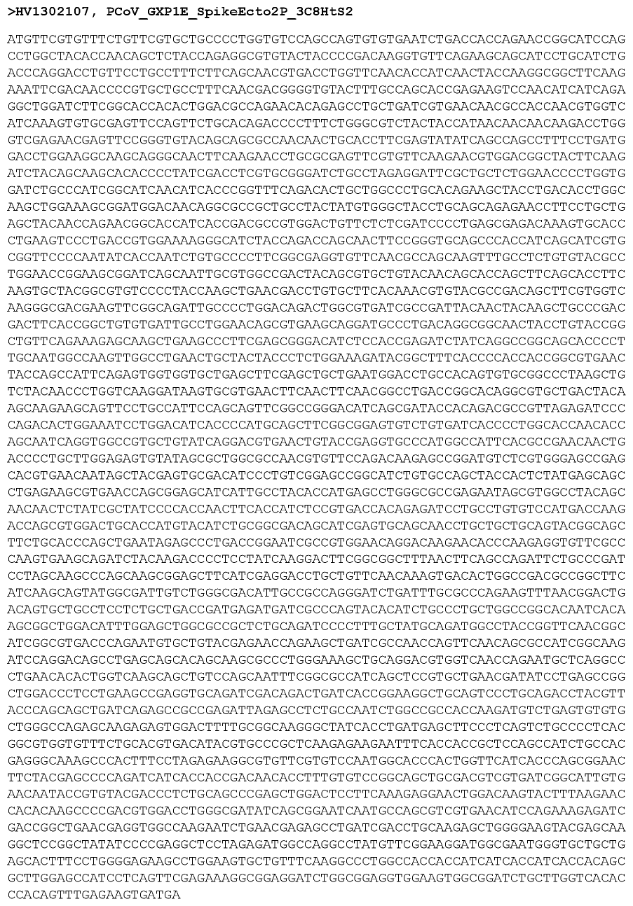

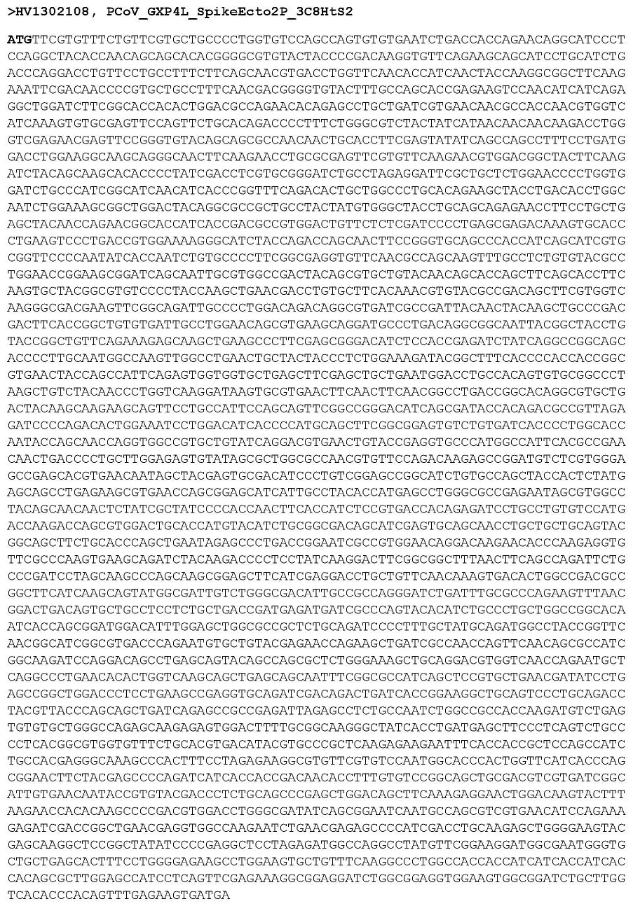

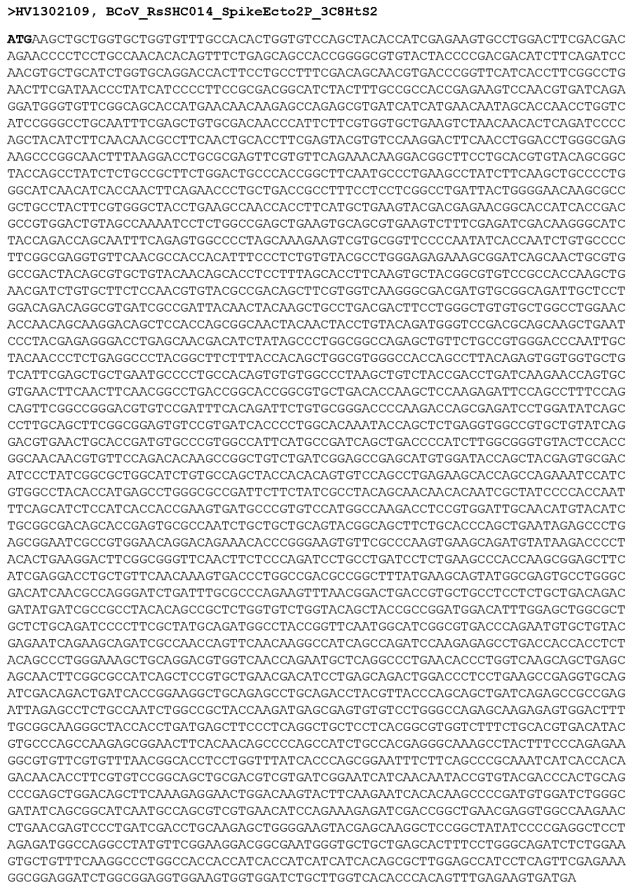

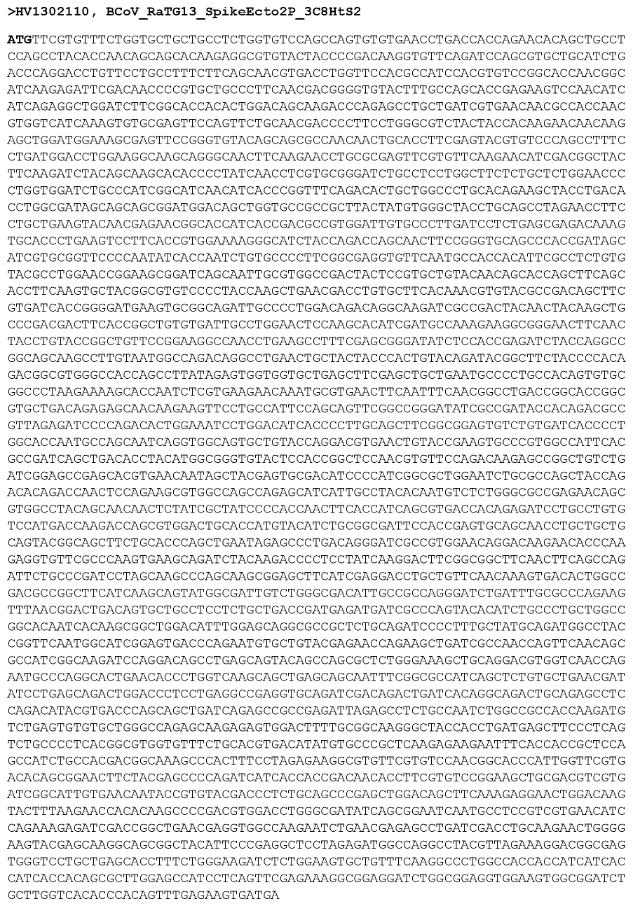

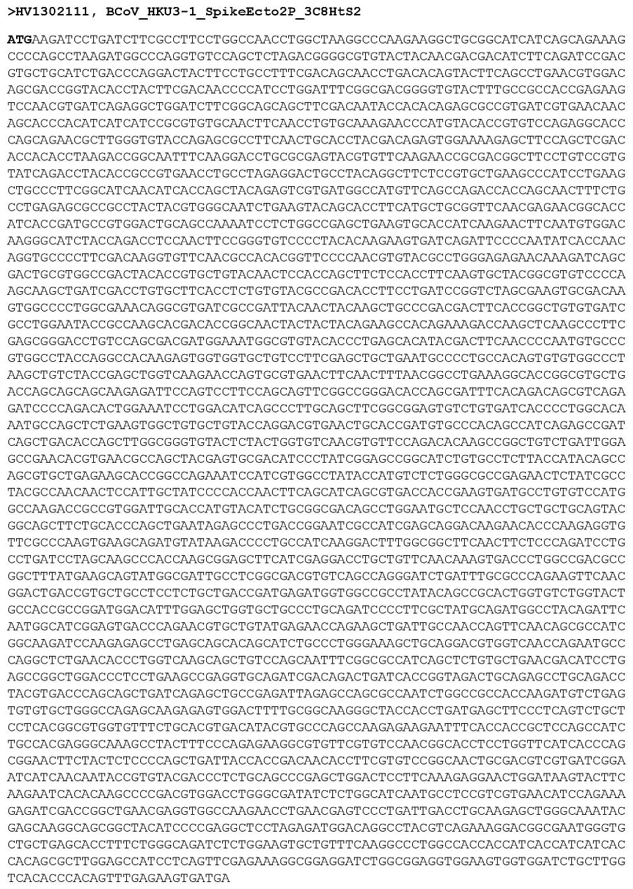

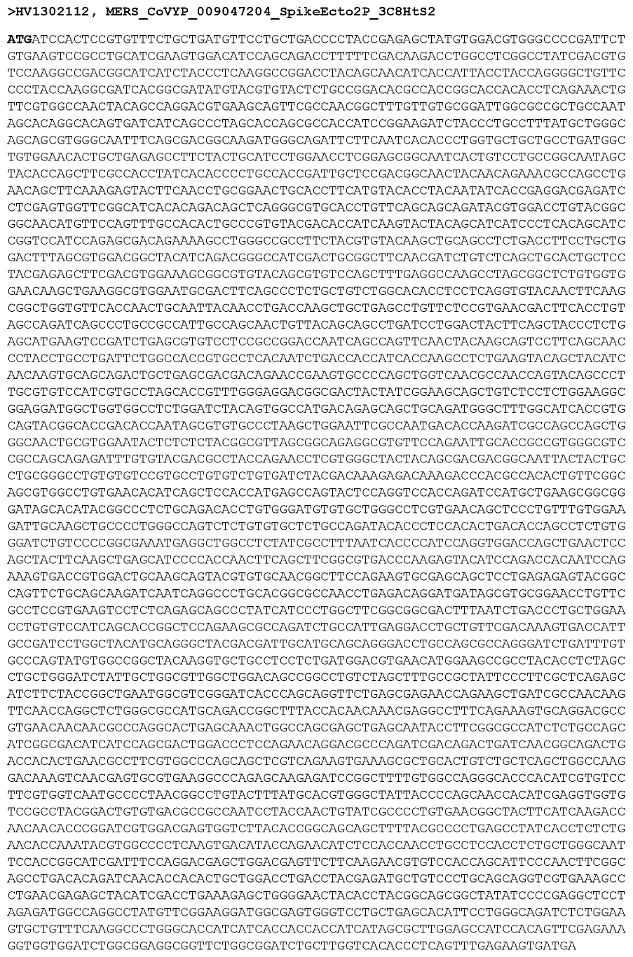

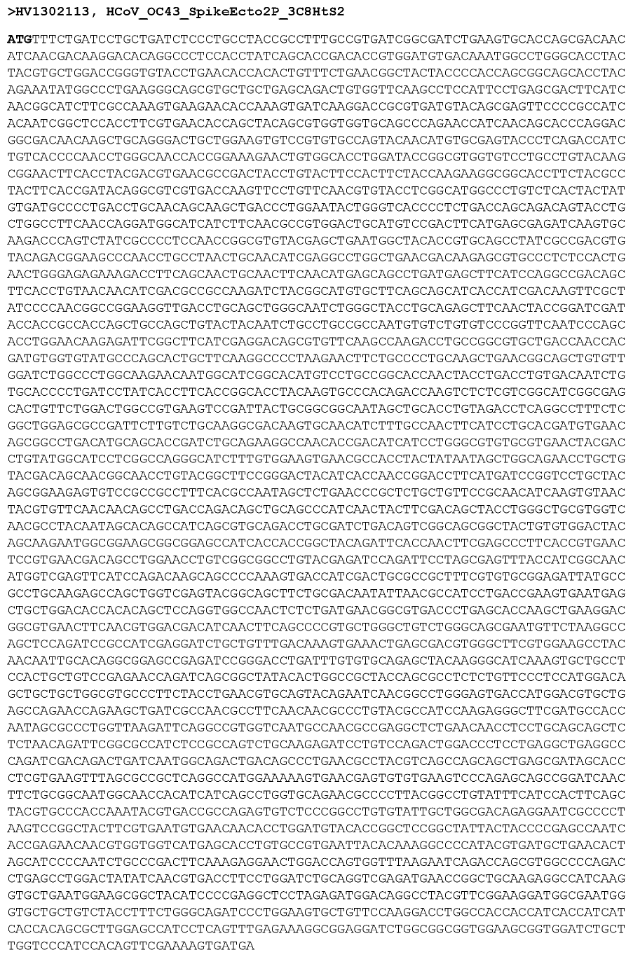

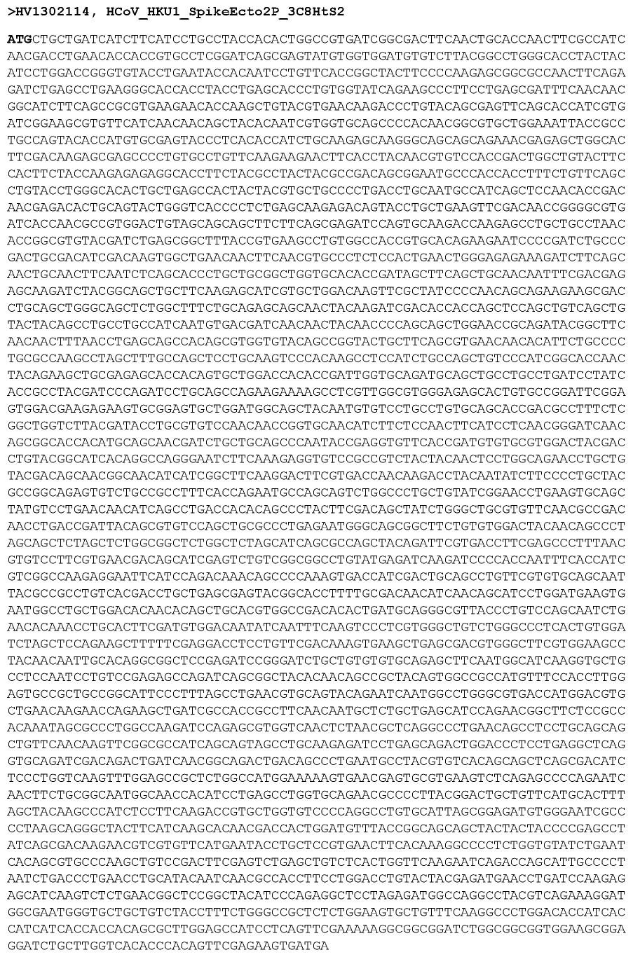

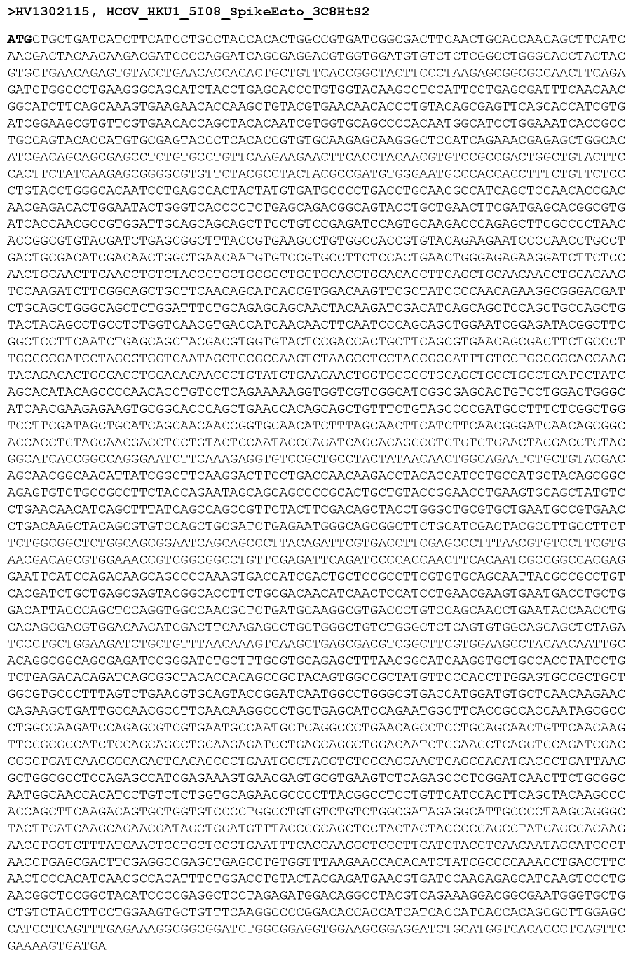

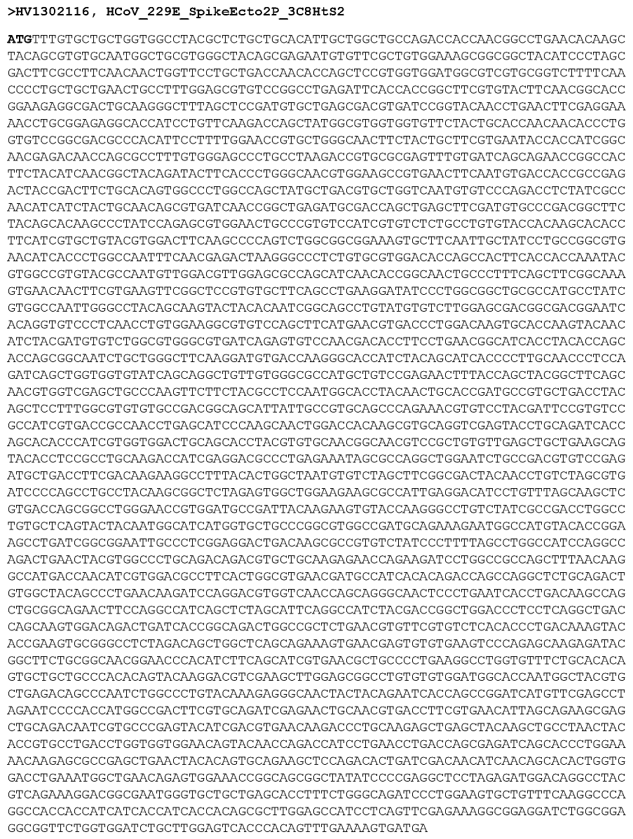

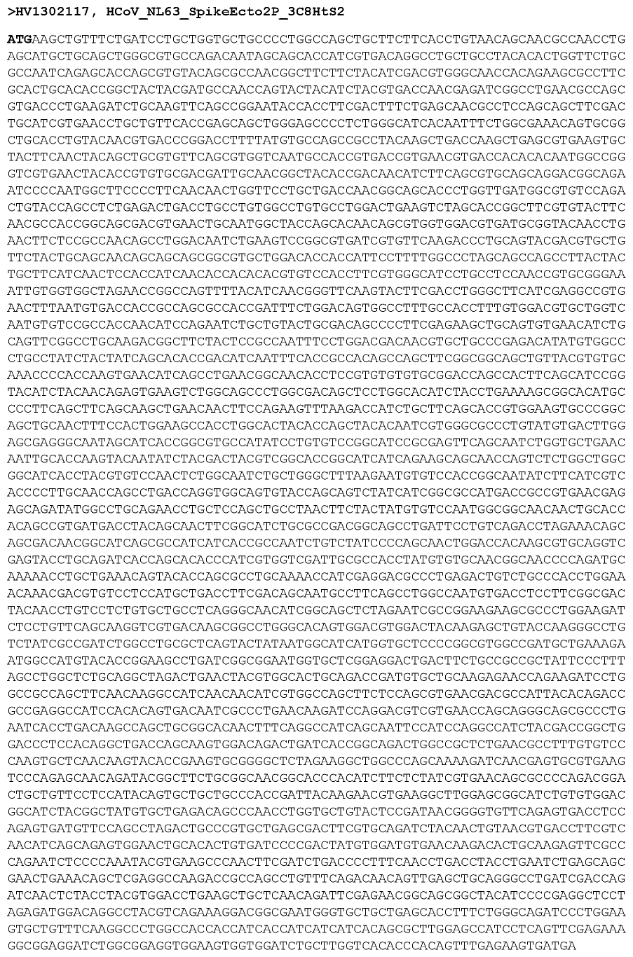

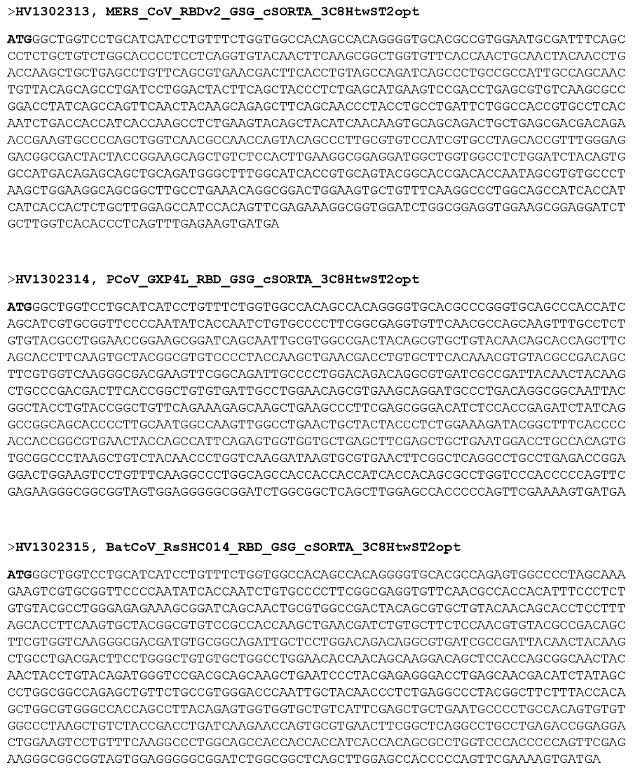

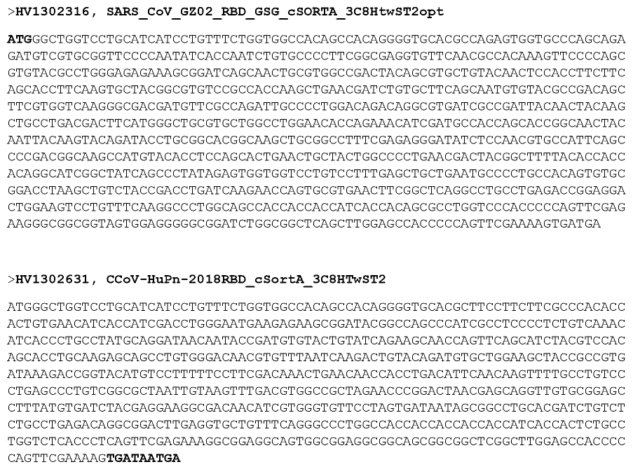

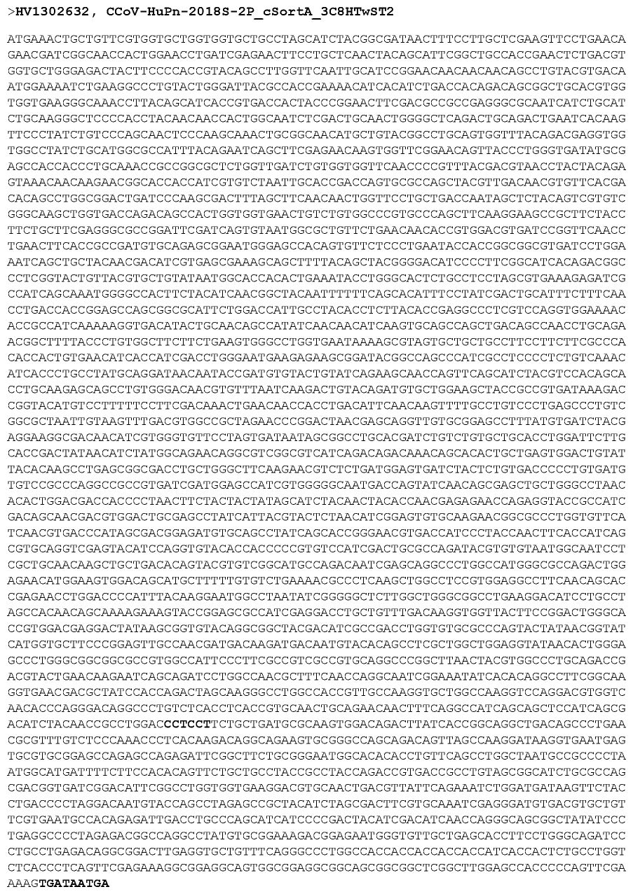

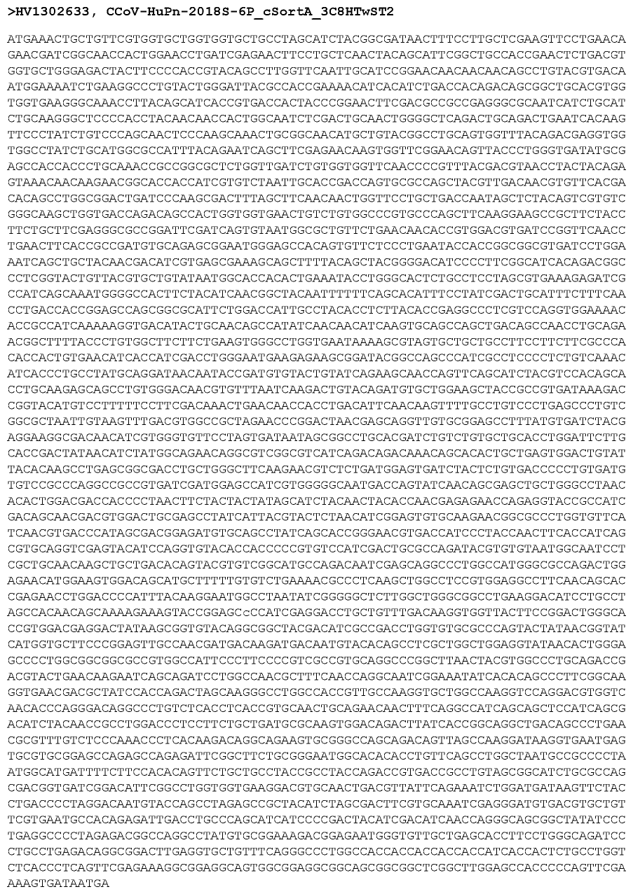

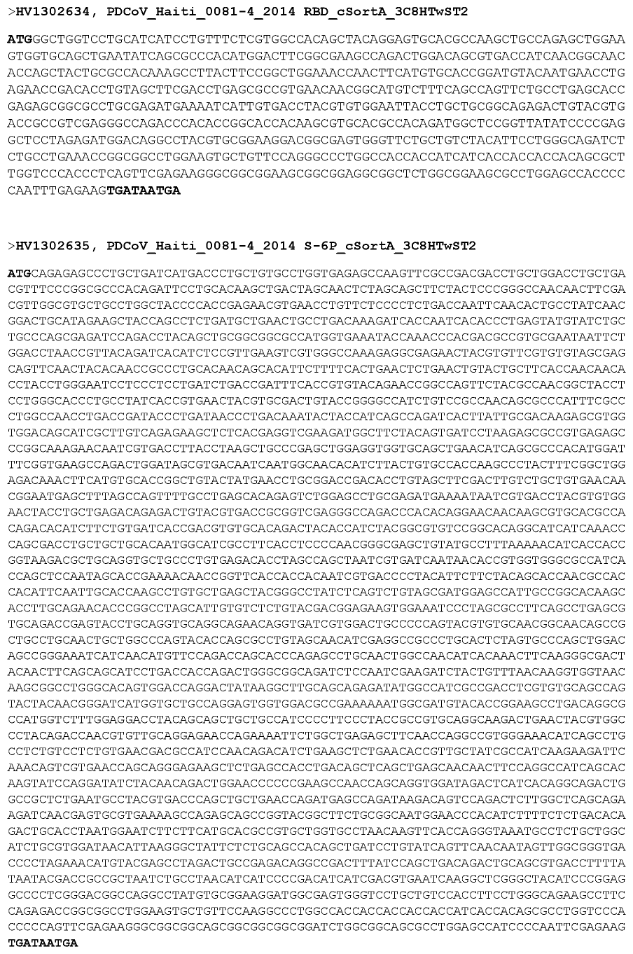

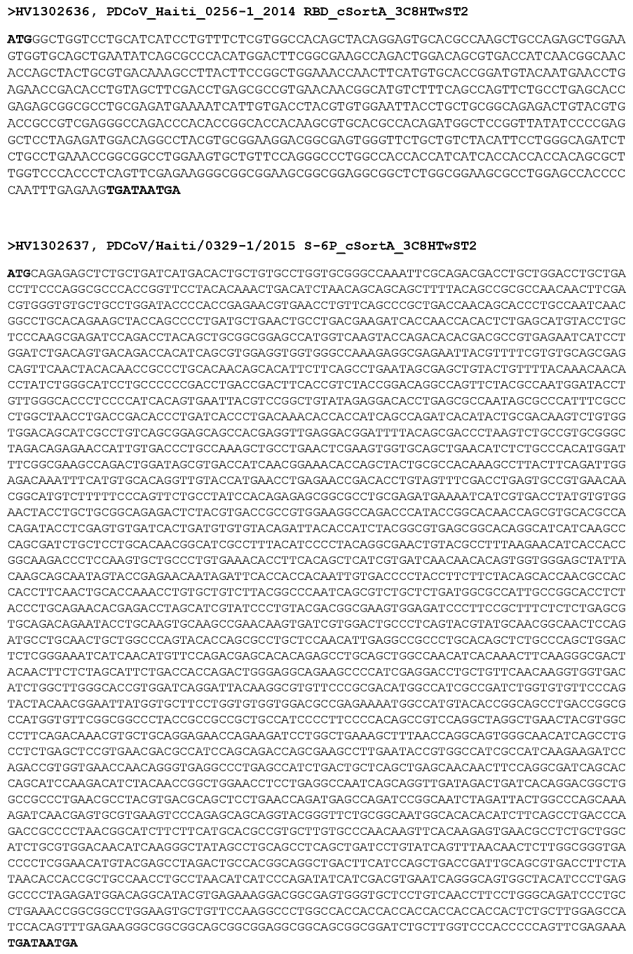

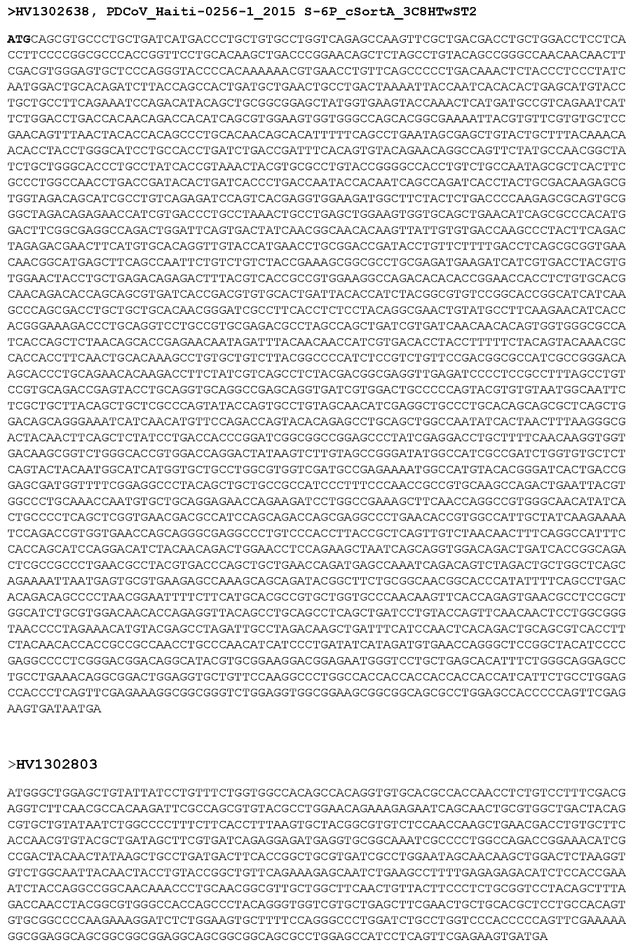

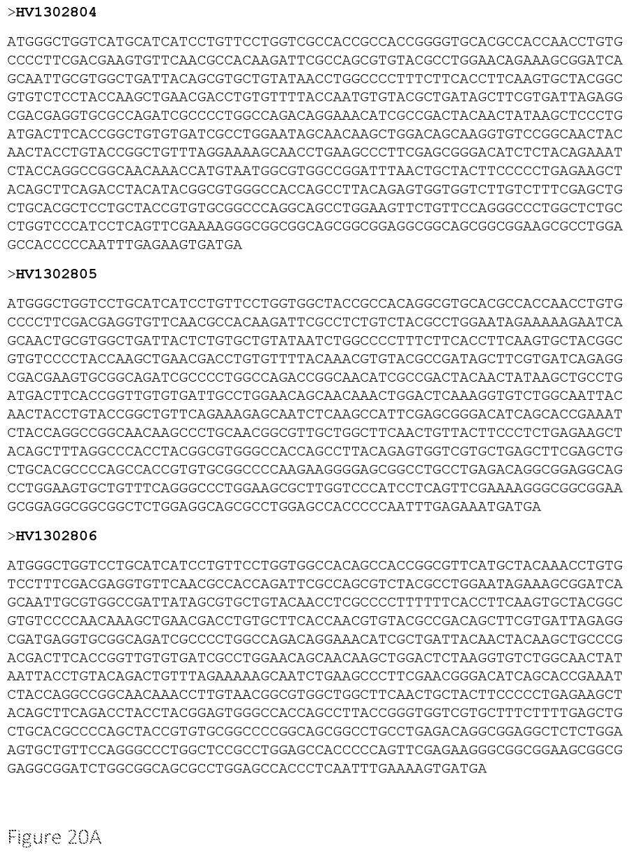

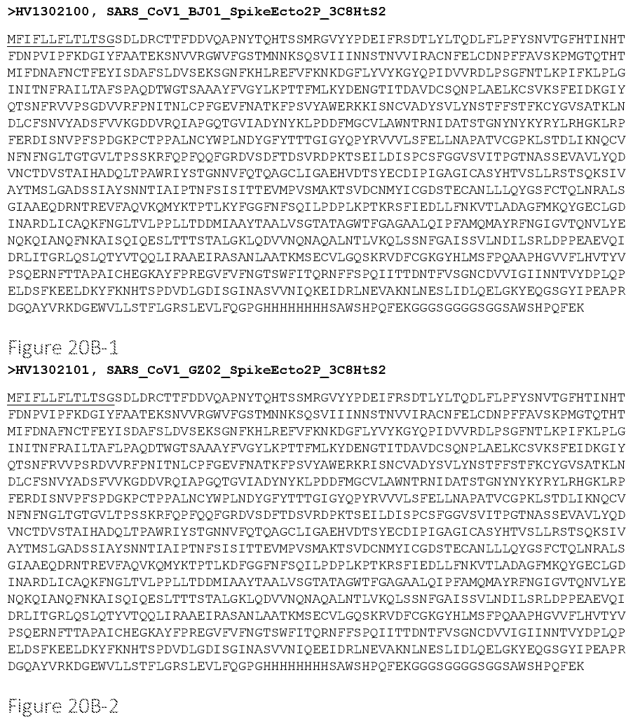

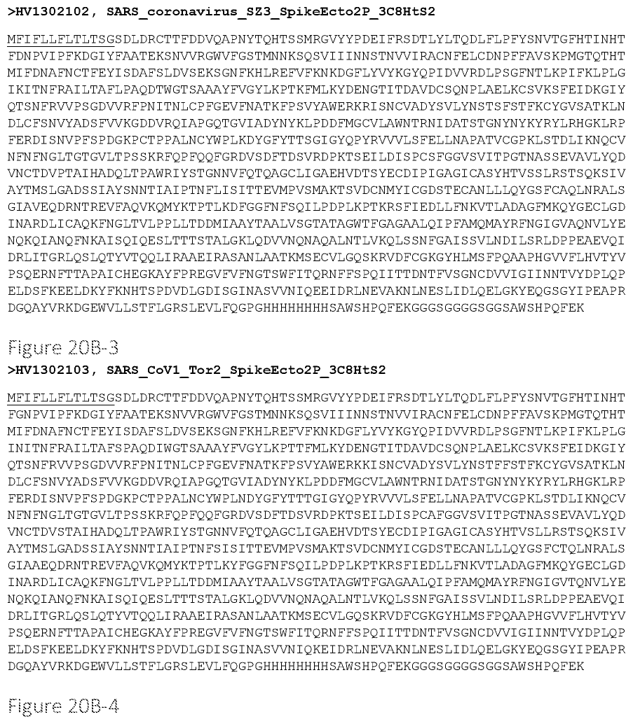

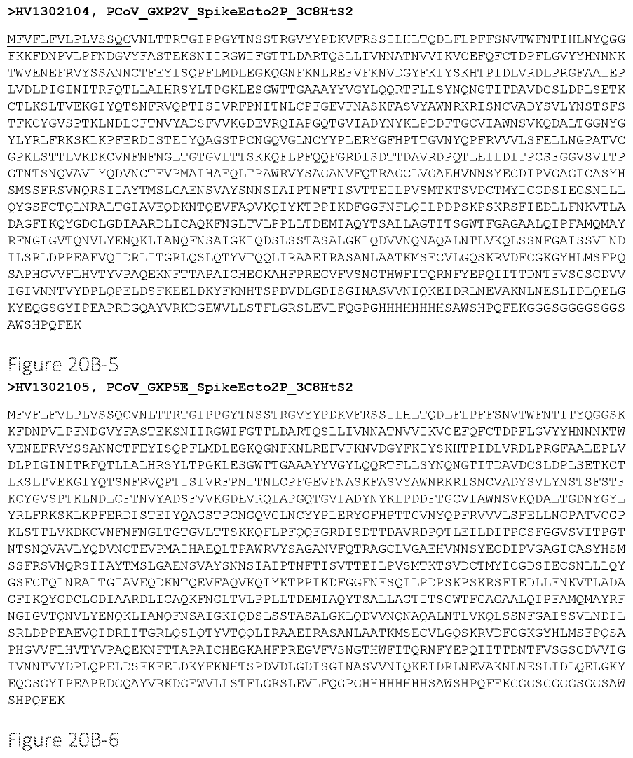

Any suitable ferritin sequence could be used. In non-limiting embodiments, ferritin sequences are disclosed in WO/2018/005558. Non-limiting embodiments of ferritin are disclosed in FIG. 18C1-15, and FIG. 23C1-3. In some embodiments, the ferritin sequence comprises N19Q amino acid change.

Ferritin nanoparticle linkers: The ability to form coronavirus spike ferritin nanoparticles relies on self-assembly of 24 ferritin subunits into a single ferritin nanoparticle. The addition of a ferritin subunit to the C-terminus of coronavirus spike ectodomain, peptides, or other domains may interfere with the ability of the ferritin subunit to fold properly and or associate with other ferritin subunits. When expressed alone ferritin readily forms 24-subunit nanoparticles, however appending it to coronavirus spike has only yielded low titers of nanoparticles if any. Since the ferritin nanoparticle forms in the absence of spike, the spike could be sterically hindering the association of ferritin subunits. Thus, we designed ferritin with elongated glycine-serine linkers to further distance the spike from the ferritin subunit. To make sure that the glycine linker is attached to ferritin at the correct position, we created constructs that attach at second amino acid position or the fifth amino acid position. The first four n-terminal amino acids of natural Helicobacter pylori ferritin are not needed for nanoparticle formation but may be critical for proper folding and oligomerization when appended to spike. Thus, we designed constructs with and without the Leucine, serine, and lysine amino acids following the glycine-serine linker. The goal will be to find a linker length that is suitable for formation of spike nanoparticles when ferritin is appended to most spikes. Any suitable linker between the spike subunit and ferritin could be used, so long as the fusion protein is expressed and the trimer is formed.

Another approach to multimerize expression constructs uses staphylococcus Sortase A transpeptidase ligation to conjugate inventive spike ectodomain trimers or spike subunits, for e.g. but not limited to cholesterol. Non-limiting embodiments of spike designs for use in Sortase A reaction are shown in FIGS. 19A-B. The trimers can then be embedded into liposomes via the conjugated cholesterol.

To conjugate the trimer, a C-terminal LPXTG tag (SEQ ID NO: 34) or a N-terminal pentaglycine repeat tag (SEQ ID NO: 35) is added to the spike trimer gene, where X signifies any amino acid, for example Ala, Ser, Glu. Cholesterol is also synthesized with these two tags. Sortase A is then used to covalently bond the tagged spike subunit to the cholesterol.

The sortase A-tagged spike trimer protein or portion thereof can also be used to conjugate the trimer to other peptides, proteins, or fluorescent labels. In non-limiting embodiments, the sortase A tagged trimers or spike portions are conjugated to ferritin to form nanoparticles.

The invention provides design of various coronavirus spike protein derivatives wherein the spike comprises a linker which permits addition of a lipid, such as but not limited to cholesterol, or multimerizing protein via a Sortase A reaction. See e.g. Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J Org. Chem. 2007, 72, 3909-3912.

### REFERENCES

Batista, F. D., and Neuberger, M. S. (2000). B cells extract and present immobilized antigen: implications for affinity discrimination. EMBO J 19, 513-520.
Crowley, C. S., Cascio, D., Sawaya, M. R., Kopstein, J. S., Bobik, T. A., and Yeates, T. O. (2010). Structural insight into the mechanisms of transport across the Salmonella enterica Pdu microcompartment shell. J Biol Chem 285, 37838-37846.
Gabashvili, A. N., Chmelyuk, N. S., Efremova, M. V., Malinovskaya, J. A., Semkina, A. S., and Abakumov, M. A. (2020). Encapsulins-Bacterial Protein Nanocompartments: Structure, Properties, and Application. Biomolecules 10.
Havemann, G. D., Sampson, E. M., and Bobik, T. A. (2002). PduA is a shell protein of polyhedral organelles involved in coenzyme B(12)-dependent degradation of 1,2-propanediol in Salmonella enterica serovar typhimurium LT2. J Bacteriol 184, 1253-1261.
Kanekiyo, M., Bu, W., Joyce, M. G., Meng, G., Whittle, J. R., Baxa, U., Yamamoto, T., Narpala, S., Todd, J. P., Rao, S. S., et al. (2015). Rational Design of an Epstein-Barr Virus Vaccine Targeting the Receptor-Binding Site. Cell 162, 1090-1100.
Kanekiyo, M., Wei, C. J., Yassine, H. M., McTamney, P. M., Boyington, J. C., Whittle, J. R., Rao, S. S., Kong, W. P., Wang, L., and Nabel, G. J. (2013). Self-assembling influenza nanoparticle vaccines elicit broadly neutralizing H1N1 antibodies. Nature 499, 102-106.
Kong, R., Xu, K., Zhou, T., Acharya, P., Lemmin, T., Liu, K., Ozorowski, G., Soto, C., Taft, J. D., Bailer, R. T., et al. (2016). Fusion peptide of HIV-1 as a site of vulnerability to neutralizing antibody. Science 352, 828-833.
Li, D., Edwards, R. J., Manne, K., Martinez, D. R., Schafer, A., Alam, S. M., Wiehe, K., Lu, X., Parks, R., Sutherland, L. L., et al. (2021). The functions of SARS-CoV-2 neutralizing and infection-enhancing antibodies in vitro and in mice and nonhuman primates. bioRxiv.
Liu, L., Wang, P., Nair, M. S., Yu, J., Rapp, M., Wang, Q., Luo, Y., Chan, J. F., Sahi, V., Figueroa, A., et al. (2020). Potent neutralizing antibodies against multiple epitopes on SARS-CoV-2 spike. Nature 584, 450-456.
Pardi, N., Hogan, M. J., Naradikian, M. S., Parkhouse, K., Cain, D. W., Jones, L., Moody, M. A., Verkerke, H. P., Myles, A., Willis, E., et al. (2018). Nucleoside-modified mRNA vaccines induce potent T follicular helper and germinal center B cell responses. J Exp Med 215, 1571-1588.
Piccoli, L., Park, Y. J., Tortorici, M. A., Czudnochowski, N., Walls, A. C., Beltramello, M., Silacci-Fregni, C., Pinto, D., Rosen, L. E., Bowen, J. E., et al. (2020). Mapping Neutralizing and Immunodominant Sites on the SARS-CoV-2 Spike Receptor-Binding Domain by Structure-Guided High-Resolution Serology. Cell 183, 1024-1042 e1021.
Saunders, K. O., Pardi, N., Parks, R., Santra, S., Mu, Z., Sutherland, L., Scearce, R., Barr, M., Eaton, A., Hernandez, G., et al. (2020). Lipid nanoparticle encapsulated nucleoside-modified mRNA vaccines elicit polyfunctional HIV-1 antibodies comparable to proteins in nonhuman primates. bioRxiv.
Sutter, M., Boehringer, D., Gutmann, S., Gunther, S., Prangishvili, D., Loessner, M. J., Stetter, K. O., Weber-Ban, E., and Ban, N. (2008). Structural basis of enzyme encapsulation into a bacterial nanocompartment. Nat Struct Mol Biol 15, 939-947.
Tokatlian, T., Read, B. J., Jones, C. A., Kulp, D. W., Menis, S., Chang, J. Y. H., Steichen, J. M., Kumari, S., Allen, J. D., Dane, E. L., et al. (2019). Innate immune recognition of glycans targets HIV nanoparticle immunogens to germinal centers. Science 363, 649-654.
Ueda, G., Antanasijevic, A., Fallas, J. A., Sheffler, W., Copps, J., Ellis, D., Hutchinson, G. B., Moyer, A., Yasmeen, A., Tsybovsky, Y., et al. (2020). Tailored design of protein nanoparticle scaffolds for multivalent presentation of viral glycoprotein antigens. Elife 9.
Wec, A. Z., Wrapp, D., Herbert, A. S., Maurer, D. P., Haslwanter, D., Sakharkar, M., Jangra, R. K., Dieterle, M. E., Lilov, A., Huang, D., et al. (2020). Broad neutralization of SARS-related viruses by human monoclonal antibodies. Science 369, 731-736.

Using the protein designs described herein which are based on SARS-CoV-2 spike sequence, a skilled artisan can readily make protein designs from any other coronavirus.

### Nucleic Acid Sequences

In certain aspects, the invention provides nucleic acids comprising sequences encoding proteins of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive protein designs. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

In some embodiments the antibodies are administered as nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, US Pub 20180344838A1 at least at paragraphs [0260]-[0281] for non-limiting embodiments of chemical modifications, wherein each content is incorporated by reference in its entirety. In non-limiting embodiments, a modified mRNA comprises pseudouridine. In some embodiments, the modified mRNA comprises 1-methyl-pseudouridine.

mRNAs delivered in LNP formulations have advantages over non-LNPs formulations. See US Pub 20180028645A1.

In certain embodiments the nucleic acid encoding a protein is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequences described herein, or a variant thereof or a fragment thereof. Accordingly, in one embodiment, the invention provides an RNA molecule encoding one or more of inventive antibodies. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7mG(5′)ppp(5′)NlmpNp). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. An RNA molecule may have a 3′ poly-A tail. It may also include a poly-A polymerase recognition sequence (e.g. AAUAAA) near its 3′ end. In some embodiments, an RNA molecule useful with the invention may be single-stranded. In some embodiments, an RNA molecule useful with the invention may comprise synthetic RNA.

The recombinant nucleic acid sequence can be an optimized nucleic acid sequence. Such optimization can increase or alter the immunogenicity of the protein. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for in vitro transfection of mRNA and detection of protein expression are known in the art.

Methods for expression and immunogenicity determination of nucleic acid encoded proteins are known in the art.

A non-limiting embodiment of a neutralization assay is described in Zhao, G., Du, L., Ma, C. et al. A safe and convenient pseudovirus-based inhibition assay to detect neutralizing antibodies and screen for viral entry inhibitors against the new human coronavirus MERS-CoV. Virol J 10, 266 (2013). https://doi.org/10.1186/1743-422X-10-266, which content is incorporated by reference in its entirety. This assay could be adapted for use for SARS CoV-2.

Non-limiting embodiments of determining antibody responses are described in the following publication: “SARS-CoV-2 specific antibody responses in COVID-19 patients” Okba et al. https://doi.org/10.1101/2020.03.18.20038059. See also US Patent Publication 20200061185 which is incorporated by reference in its entirety.

Non-limiting embodiments of various assays, reagents, and technologies for evaluating the immunogens of the invention are described in Muthumani et al. Science Translational Medicine 19 Aug. 2015: Vol. 7, Issue 301, pp. 301ra132, DOI: 10.1126/scitranslmed.aac7462. The assays, reagents, and techniques could be adapted for use for SARS CoV-2.

Recombinant protein production of coronavirus is known. See e.g. in US Patent Pub 20200061185 which disclosure is incorporated by reference in its entirety.

In some embodiments the SARS-2 S proteins of the invention are in a trimeric configuration. These designs could comprise any suitable trimerization domain.

Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to a recombinant coronavirus S ectodomain described herein (e.g., by linkage to the C-terminus of S2) to promote trimerization of the recombinant coronavirus S ectodomain.

In some examples, the C-terminus of the S2 subunit of the S ectodomain can be linked to a T4 fibritin Foldon domain. In specific examples, the T4 fibritin Foldon domain can include the amino acid sequence GYIPEAPRDGQAYVRKDGEWVLLSTF (SEQ ID NO: 15), which adopts a .beta.-propeller conformation, and can fold and trimerize in an autonomous way (Tao et al. 1997 Structure 5:789-798). Optionally, the heterologous trimerization is connected to the recombinant coronavirus S ectodomain via a peptide linker, such as an amino acid linker. Non-limiting examples of peptide linkers that can be used include glycine, serine, and glycine-serine linkers.

In some embodiments, the SARS-2 spike ectodomain trimer can be membrane anchored, for example, for embodiments where the coronavirus S ectodomain trimer is expressed on an attenuated viral vaccine, or a virus like particle. In such embodiments, the protomers in the trimer can each comprise a C-terminal linkage to a transmembrane domain, such as the transmembrane domain (and optionally the cytosolic tail) of corresponding coronavirus. For example, the protomers of a disclosed SARS-2 ectodomain trimer can be linked to a SARS-2 S transmembrane and cytosolic tail. In some embodiments, one or more peptide linkers (such as a gly-ser linker, for example, a 10 amino acid glycine-serine peptide linker can be used to link the recombinant S ectodomain protomer to the transmembrane domain.

The protomers linked to the transmembrane domain can include any of the modifications provided herein (or combinations thereof) as long as the recombinant coronavirus S ectodomain trimer formed from the protomers linked to the transmembrane domain retains the certain properties (e.g., the coronavirus S prefusion conformation).

The inventive protein or fragments thereof can be produced using recombinant techniques, or chemically or enzymatically synthesized.

In some embodiments a protein nanoparticle is provided that includes one or more of the disclosed recombinant SARS-CoV-2 S proteins, including but not limited to SARS-2 S trimers. Non-limiting example of nanoparticles include ferritin nanoparticles, encapsulin nanoparticles, Sulfur Oxygenase Reductase (SOR) nanoparticles, and lumazine synthase nanoparticles, which are comprised of an assembly of monomeric subunits including ferritin proteins, encapsulin proteins, SOR proteins, and lumazine synthase, respectively. Additional protein nanoparticle structures are described by Heinze et al., J Phys Chem B., 120(26):5945-52, 2016; Hsia et al., Nature, 535(7610):136-9, 2016; and King et al., Nature, 510(7503):103-8, 2014; each of which is incorporated by reference herein. To construct such protein nanoparticles a protomer of the SARS-2 coronavirus S ectodomain trimer can be linked to a subunit of the protein nanoparticle (such as a ferritin protein, an encapsulin protein, a SOR protein, or a lumazine synthase protein) and expressed in cells under appropriate conditions. The fusion protein self-assembles into a nanoparticle any can be purified.

In some embodiments, a protomer of a disclosed recombinant SARS-CoV-2 spike protein ectodomain trimer can be linked to a ferritin subunit to construct a ferritin nanoparticle. Ferritin nanoparticles and their use for immunization purposes (e.g., for immunization against influenza antigens) have been disclosed in the art (see, e.g., Kanekiyo et al., Nature, 499:102-106, 2013, incorporated by reference herein in its entirety). Ferritin is a globular protein that is found in all animals, bacteria, and plants, and which acts primarily to control the rate and location of polynuclear Fe(III).sub.2O.sub.3 formation through the transportation of hydrated iron ions and protons to and from a mineralized core. The globular form of the ferritin nanoparticle is made up of monomeric subunits, which are polypeptides having a molecule weight of approximately 17-20 kDa.

Following production, these monomeric subunit proteins self-assemble into the globular ferritin protein. Thus, the globular form of ferritin comprises 24 monomeric, subunit proteins, and has a capsid-like structure having 432 symmetry. Methods of constructing ferritin nanoparticles are known to the person of ordinary skill in the art and are further described herein (see, e.g., Zhang, Int. J. Mol. Sci., 12:5406-5421, 2011, which is incorporated herein by reference in its entirety).

In non-specific examples, the ferritin polypeptide is E. coli ferritin, Helicobacter pylori ferritin, insect ferritin, human light chain ferritin, bullfrog ferritin or a hybrid thereof, such as E. coli-human hybrid ferritin, E. coli-bullfrog hybrid ferritin, or human-bullfrog hybrid ferritin. Exemplary amino acid sequences of ferritin polypeptides and nucleic acid sequences encoding ferritin polypeptides for use to make a ferritin nanoparticle including a recombinant SARS-2 spike protein can be found in GENBANK, for example at accession numbers ZP 03085328, ZP 06990637, EJB64322.1, AAA35832, NP 000137 AAA49532, AAA49525, AAA49524 and AAA49523, which are specifically incorporated by reference herein in their entirety. In some embodiments, a recombinant protein of the invention can be linked to a ferritin subunit to form a nanoparticle.

Polynucleotides encoding a protomer of any of the disclosed recombinant proteins are also provided. These polynucleotides include DNA, cDNA and RNA sequences which encode the protomer, as well as vectors including the DNA, cDNA and RNA sequences, such as a DNA or RNA vector used for immunization. The genetic code to construct a variety of functionally equivalent nucleic acids, such as nucleic acids which differ in sequence but which encode the same protein sequence, or encode a conjugate or fusion protein including the nucleic acid sequence.

In several embodiments, the nucleic acid molecule encodes a precursor of the protomer, that, when expressed in an appropriate cell, is processed into a recombinant SARS-2 S protomer that can self-assemble into the corresponding recombinant trimer. For example, the nucleic acid molecule can encode a recombinant SARS-2 S ectodomain including a N-terminal signal sequence for entry into the cellular secretory system that is proteolytically cleaved in the during processing of the recombinant protein in the cell. Embodiments further comprise recombinant proteins with different signal peptide sequences.

In some embodiments, the nucleic acid molecule encodes a precursor S polypeptide that, when expressed in an appropriate cell, is processed into a recombinant SARS-2 S protomer including S1 and S2 polypeptides, wherein the recombinant protein includes any of the appropriate modifications described herein, and optionally can be linked to a trimerization domain, such as a T4 Fibritin trimerization domain.

Exemplary nucleic acids can be prepared by molecular and cloning techniques. A wide variety of cloning methods, host cells, and in vitro amplification methodologies are well known to persons of skill, and can be used to make the nucleic acids and proteins of the invention.

The polynucleotides encoding a disclosed recombinant protomer can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of nucleotides. The term includes single and double stranded forms of DNA.

Polynucleotide sequences encoding a disclosed recombinant protomer can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the disclosed recombinant protomer can be expressed in vitro by DNA transfer into a suitable host cell. The cell may be prokaryotic or eukaryotic. The term also includes any progeny of the subject host cell. All progeny may not be identical to the parental cell since there may be mutations that occur during replication. Methods of stable transfer, meaning that the foreign DNA is continuously maintained in the host, are known in the art.

Host systems for recombinant production can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4.sup.th Ed., Humana Press). Examples of mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI.sup.−/− cells, or HEK-293F cells.

In some embodiments, the disclosed recombinant coronavirus S ectodomain protomer can be expressed in cells under conditions where the recombinant coronavirus S ectodomain protomer can self-assemble into trimers which are secreted from the cells into the cell media. In such embodiments, each recombinant coronavirus S ectodomain protomer contains a leader sequence (signal peptide) that causes the protein to enter the secretory system, where the signal peptide is cleaved and the protomers form a trimer, before being secreted in the cell media. The medium can be centrifuged and recombinant coronavirus S ectodomain trimer purified from the supernatant.

A nucleic acid molecule encoding a protomer can be included in a viral vector, for example, for expression of the immunogen in a host cell, or for immunization of a subject as disclosed herein. In some embodiments, the viral vectors are administered to a subject as part of a prime-boost vaccination. In several embodiments, the viral vectors are included in a vaccine, such as a primer vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif; Protein Sciences Corp., Meriden, Conn.; Stratagene, La Jolla, Calif).

In several embodiments, the viral vector can include an adenoviral vector that expresses a protomer of the invention. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/02231 1.

In some embodiments, a virus-like particle (VLP) is provided that comprises a recombinant protomer of the invention. In some embodiments, a virus-like particle (VLP) is provided that includes a recombinant trimer of the invention. Such VLPs can include a recombinant coronavirus S ectodomain trimer that is membrane anchored by a C-terminal transmembrane domain, for example the recombinant coronavirus S ectodomain protomers in the trimer each can be linked to a transmembrane domain and cytosolic tail from the corresponding coronavirus. VLPs lack the viral components that are required for virus replication and thus represent a highly attenuated, replication-incompetent form of a virus. However, the VLP can display a polypeptide (e.g., a recombinant coronavirus S ectodomain trimer) that is analogous to that expressed on infectious virus particles and can eliciting an immune response to the corresponding coronavirus when administered to a subject. Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

The immunogens of the invention could be combined with any suitable adjuvant.

Non-limiting examples of evaluating the immunogenicity and effectiveness of the immunogens of the invention are shown in US Patent Pub 20200061185 which disclosure is incorporated by reference in its entirety.

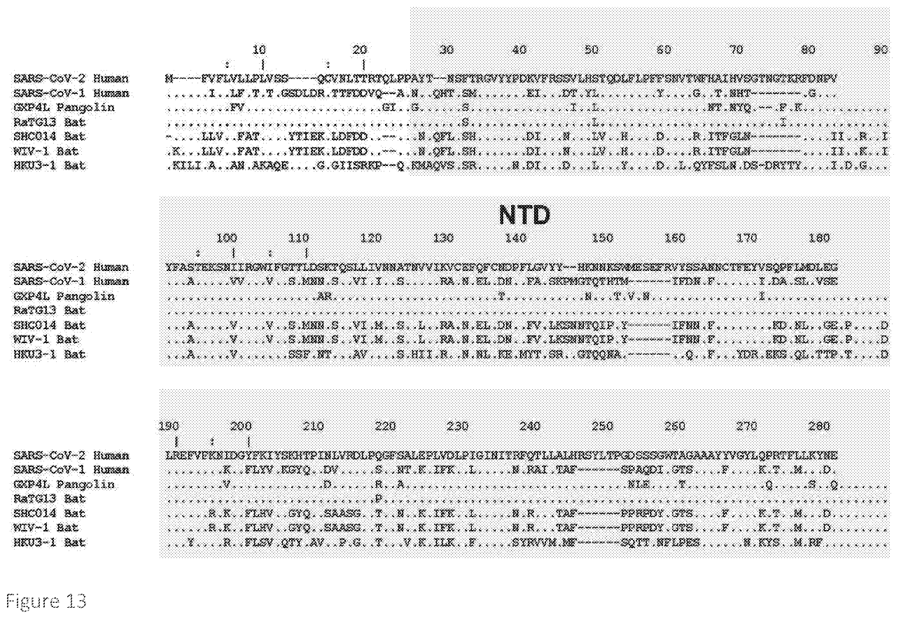

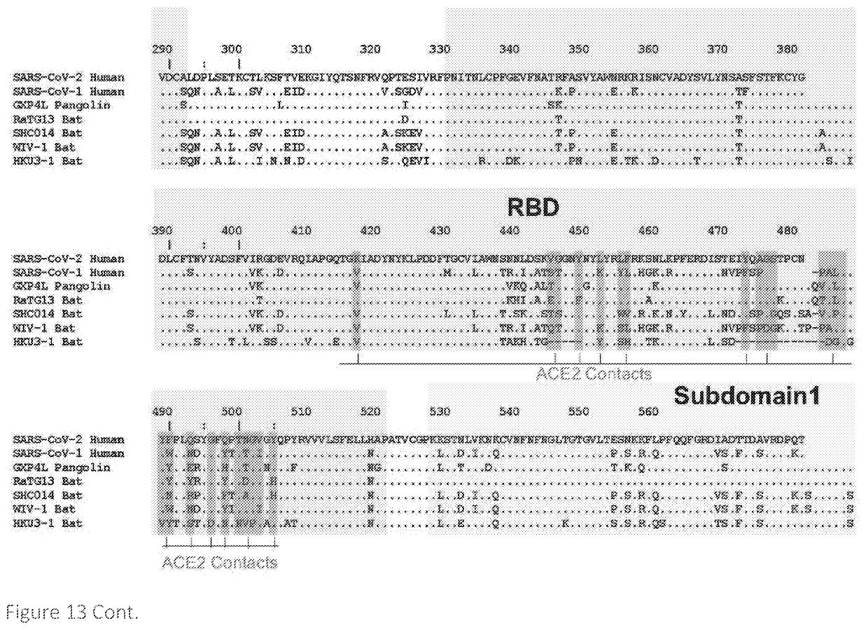

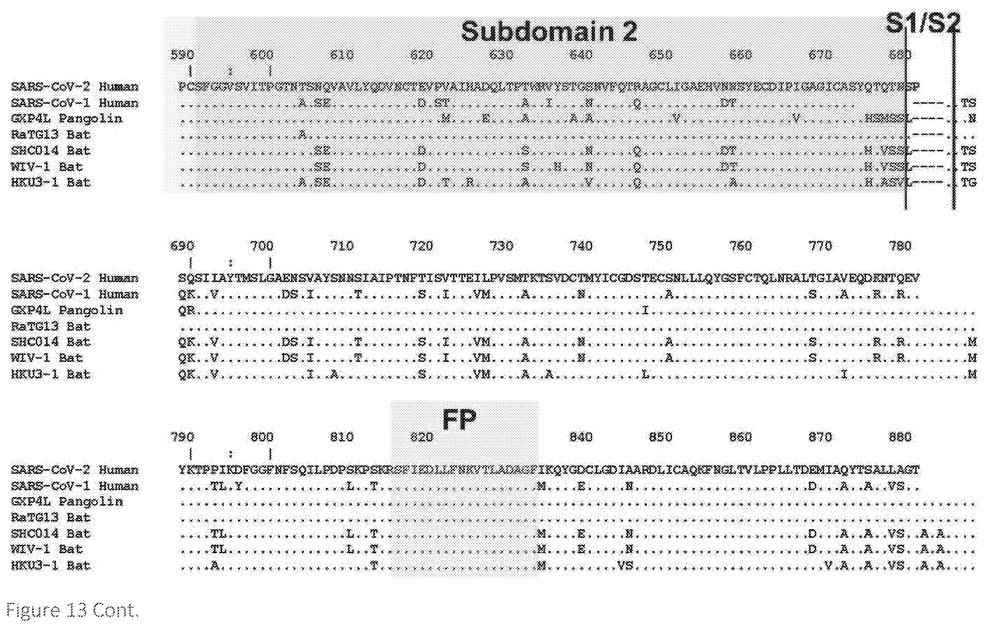

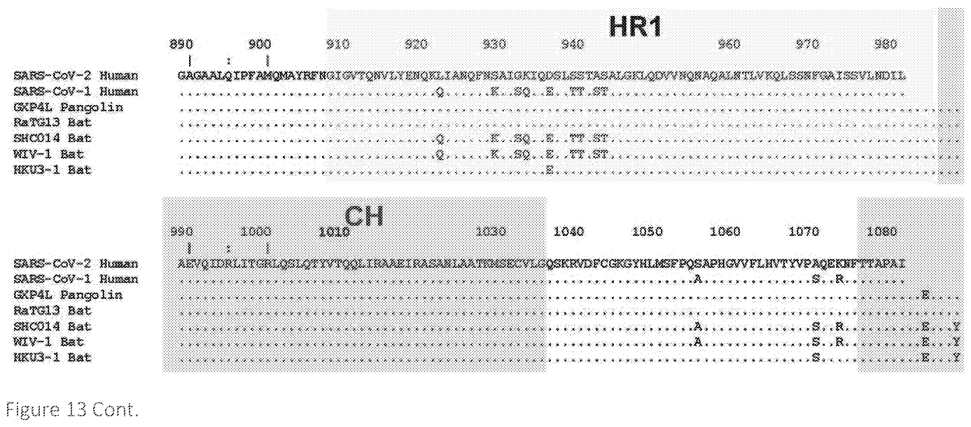

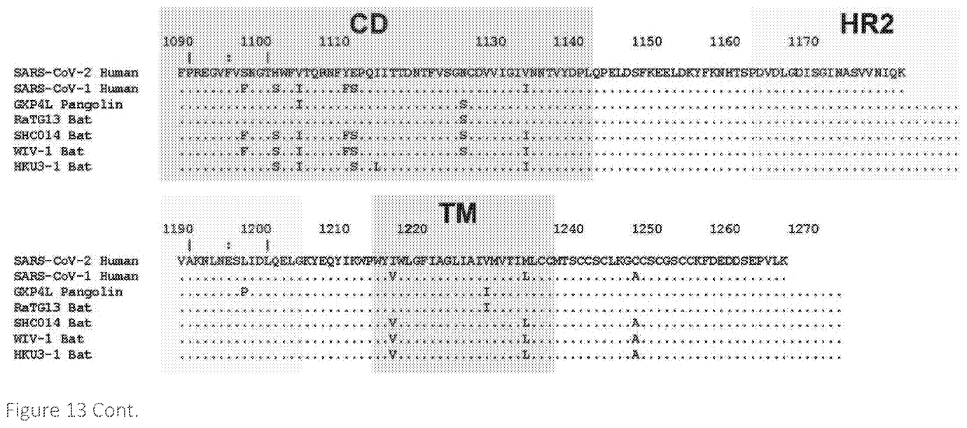

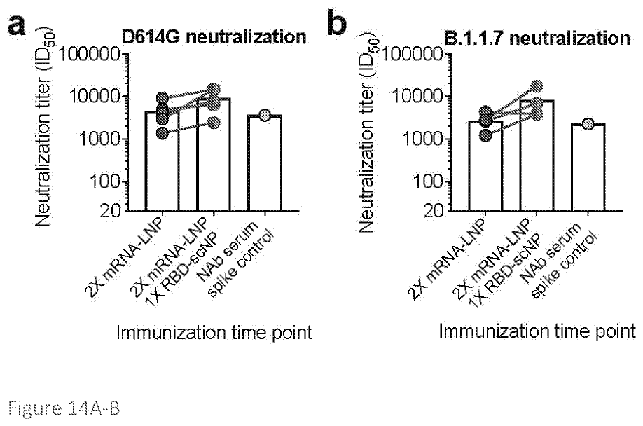

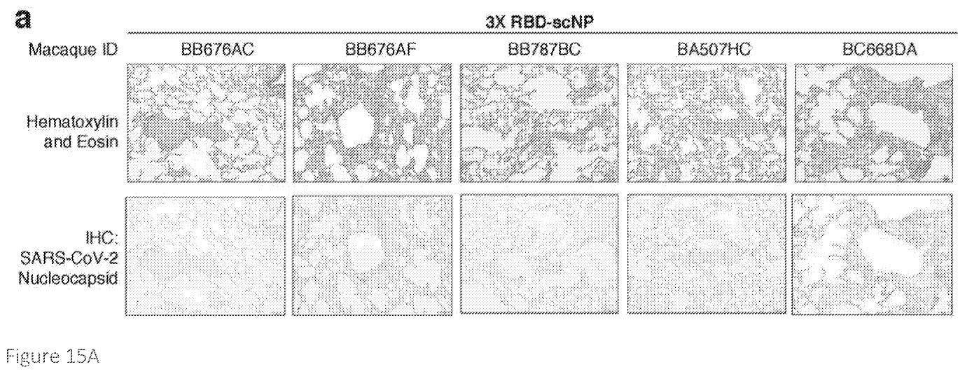

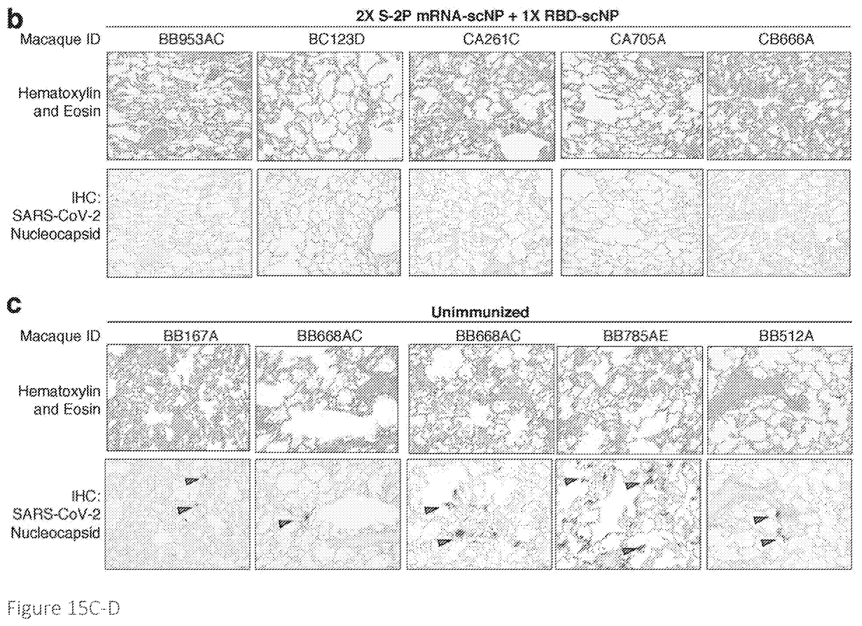

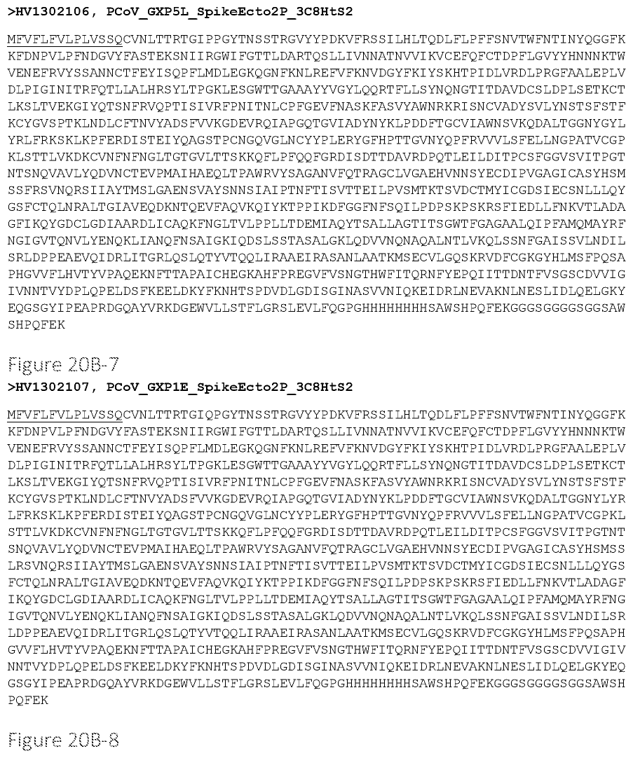

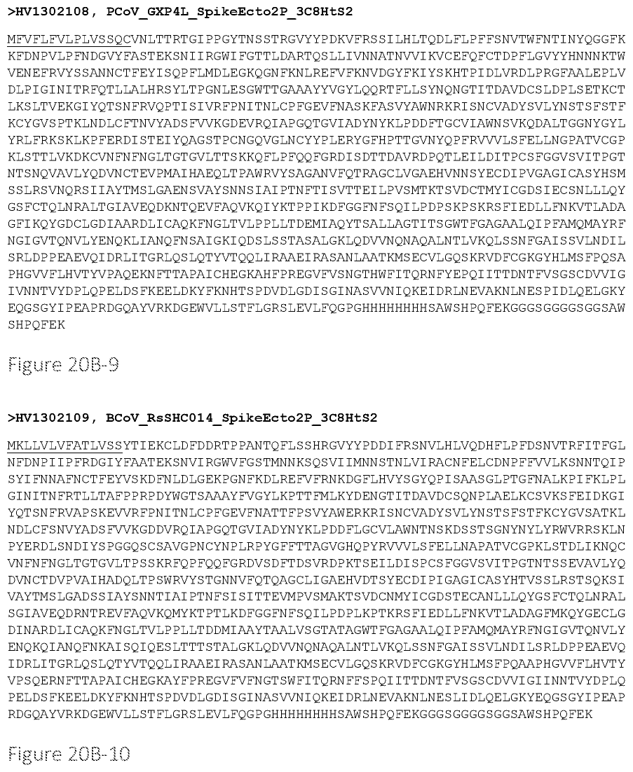

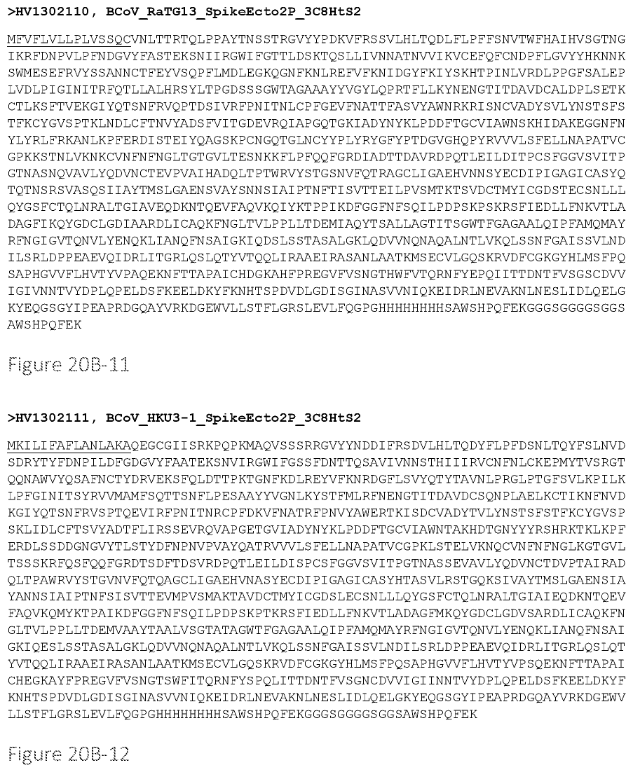

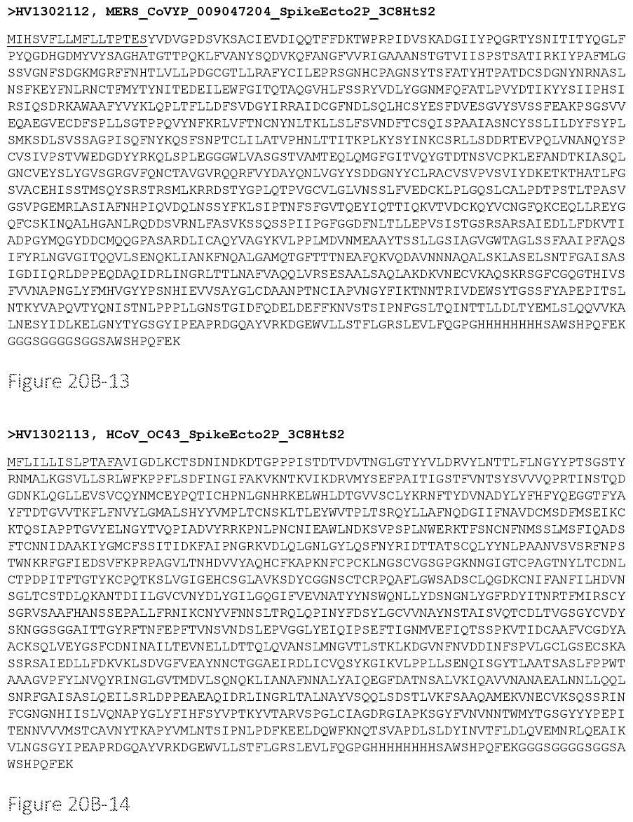

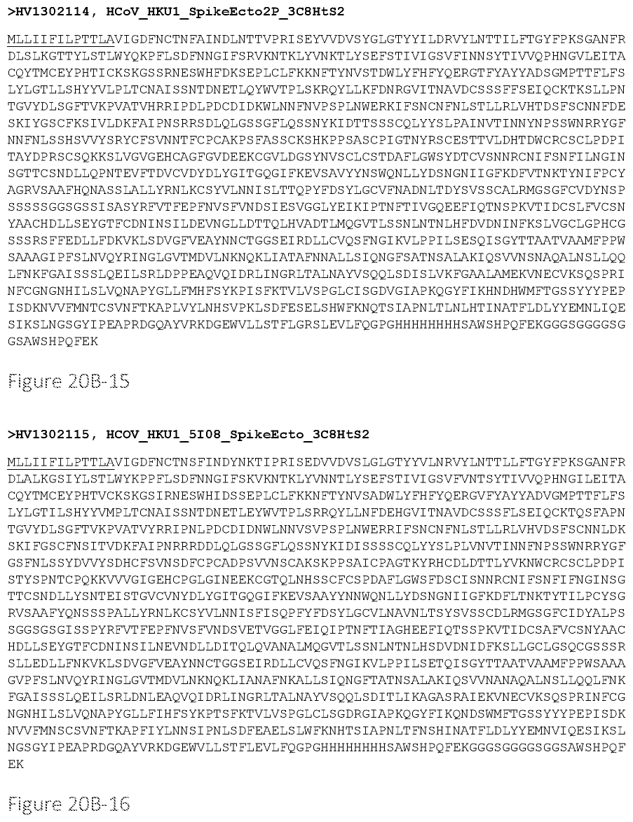

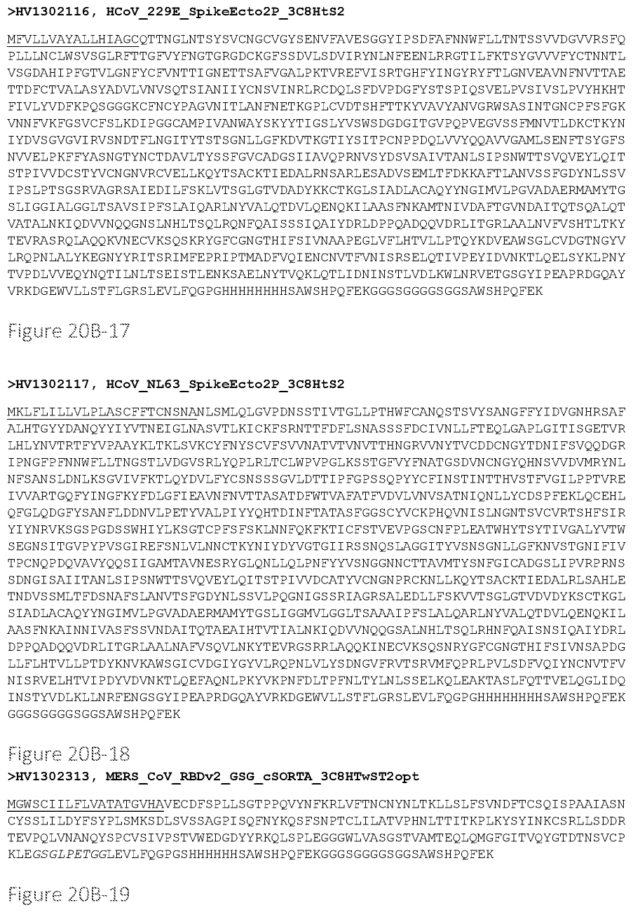

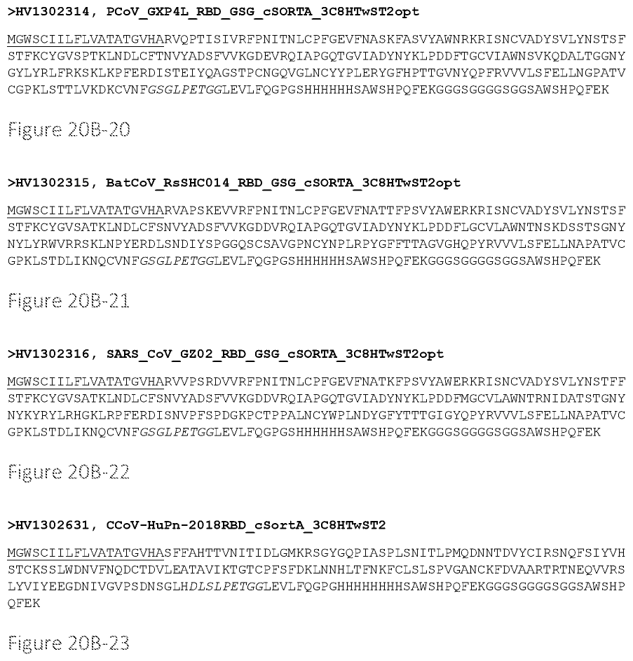

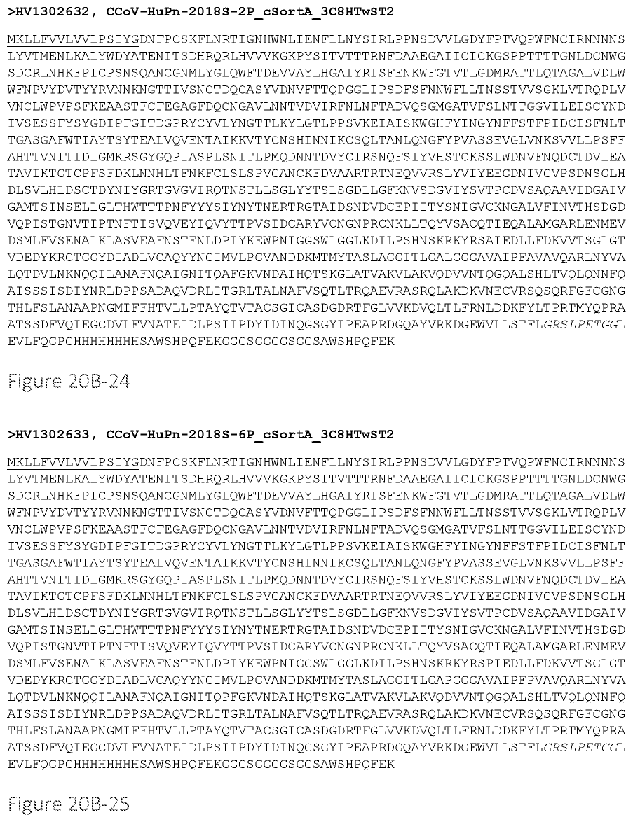

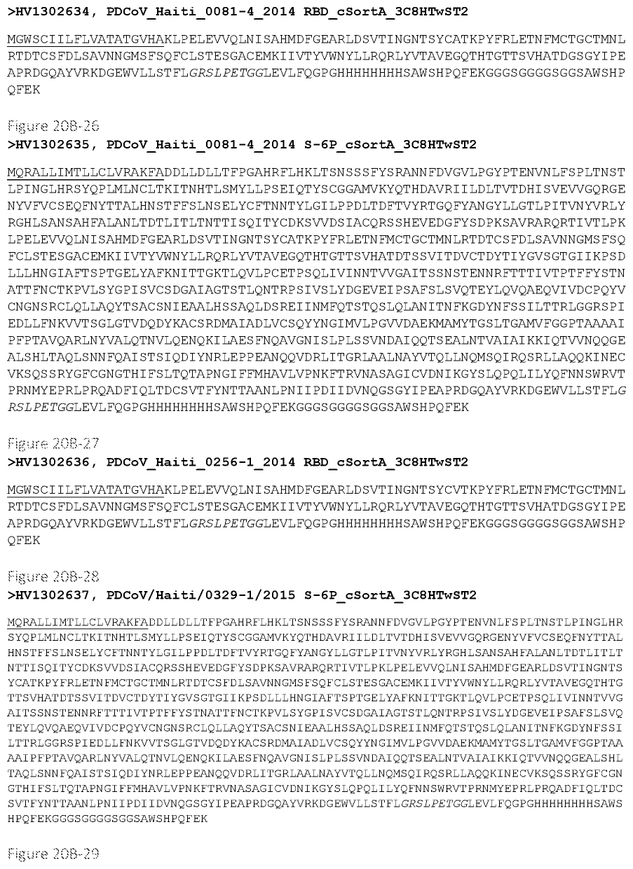

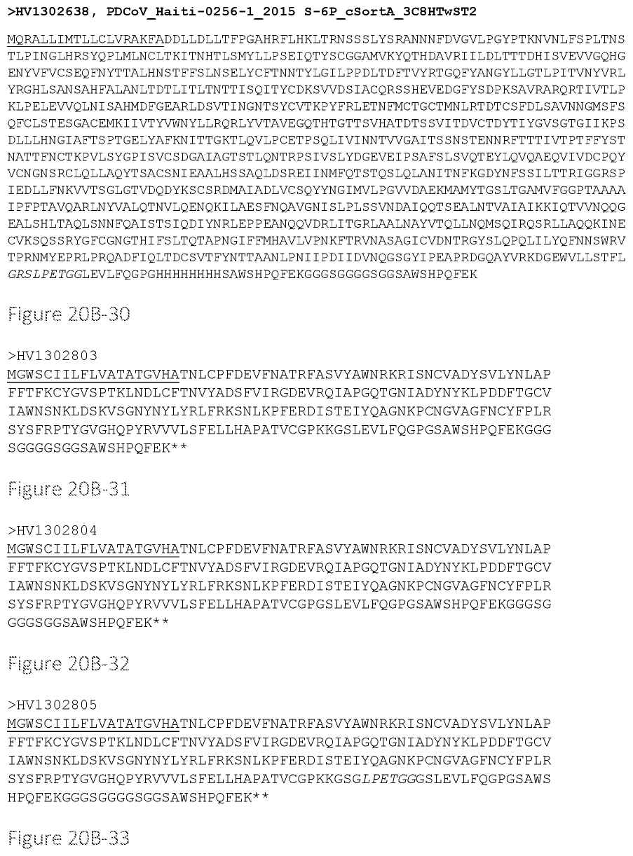

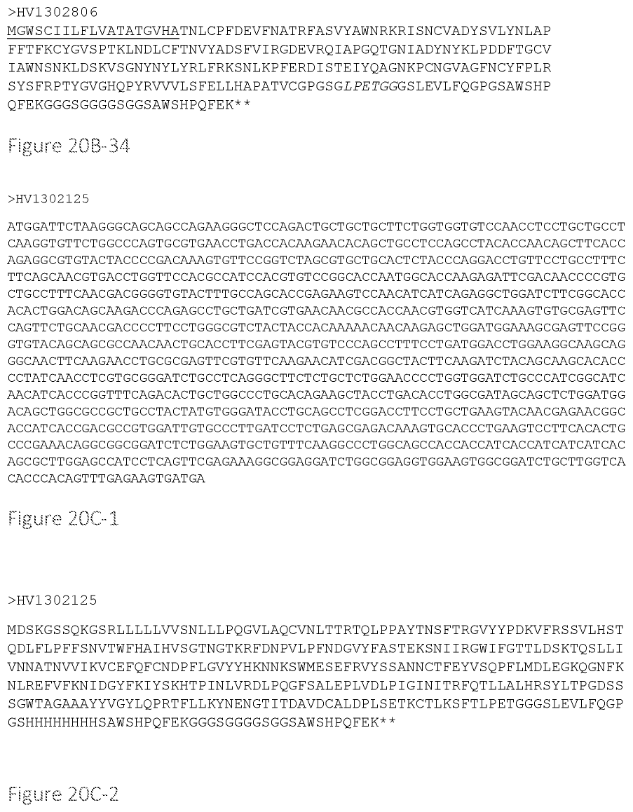

TABLE 1
Non-limiting embodiments of immunogens of the invention. FIG. 18 shows non-limiting embodiments
of amino acid sequences and nucleic acid sequences. Immunogens could be administered as proteins,
nucleic acids, or combination thereof. A skilled artisan can readily determine the bounds of an
RBD, NTD, or FP domain. In some embodiments, the spike sequence is from CoV2 Wuhan variant. In
some embodiments, the spike sequence is from MERS variant. Using the protein designs described
herein which are based on SARS-CoV-2 spike sequence, a skilled artisan can readily make protein
designs from any other coronavirus, including without limitation any variants. A skilled artisan
appreciates that various elements in the sequences depicted in FIG. 18, including without limitation
HRV-3C cleavage site, 8X his tag (SEQ ID NO: 17), and a twin StrepTagII (IBA), are included to
facilitate expression and/or purification and are removed from recombinant proteins used as immunogens
in animal or clinical studies. The sequences herein include c-terminal sortase A donor/tag sequences
to allow for site specific conjugation to multimerzing scaffolds expressing the n-terminal sortase
A acceptor sequence. The donor sequence is a LPXTGG (SEQ ID NO: 32) where the third amino acid
X can vary. In some embodiments the sortase tag comprises additional amino acid so that the sequence
is GSGLPXTGG (SEQ ID NO: 37). In one embodiment X is E. The acceptor sequence is composed of 5
or more glycines (SEQ ID NO: 21) appended to the N-terminus.

| Plasmid ID | Protein Name |
|---|---|
| HV1302313 | MERS_CoV_RBDv2_GSG_cSORTA_3C8HtwST2opt |
| HV1302314 | PCoV_GXP4L_RBD_GSG_cSORTA_3C8HtwST2opt |
| HV1302315 | BatCoV-RsSHC014_RBD_GSG_cSORTA_3C8HtwST2opt |
| HV1302316 | SARS_CoV_GZ02_RBD_GSG_cSORTA_3C8HtwST2opt |
| HV1302317 | MERS_CoV_RBDv2_3C8HtwST2opt (FIG. 18A-12) |
| HV1302318 | MERS_CoV_RBDv3_3C8HtwST2opt (FIG. 18A-13) |
| HV1302319 | MERS_CoV_RBDv4_GSG_cSORTA_n8HtwSTII3C |
| HV1302118 | SARS-CoV-2 RBD SortA_3C8HtS2 |
| HV1302118_E484K | SARS-CoV-2 RBD_E484K SortA_3C8HtS2 |
| HV1302118_K417N | SARS-CoV-2 RBD_K417N SortA_3C8HtS2 |
| HV1302118_N501Y | SARS-CoV-2 RBD_N501Y SortA_3C8HtS2 |
| HV1302118_E484K_K417N_N501Y | SARS-CoV-2 RBD_K417N_E484K_N501Y |
| | SortA_3C8HtS2 |
| HV1302119 | SARS-CoV-2 S-2P-foldon SortA_3C8HtS2 |
| HV1302120 | SARS-COV-2_NTD_c-SORTA_n3C8HTWSTII |
| HV1302346 | KRISP-K004599_HexaProS_cSORTA_3C8HtwST2 |
| HV1302347 | N00347/2020_HexaProS_cSORTA_3C8HtwST2 |
| SEQ ID NO: 97 | spike RBD + linker + Ferritin any |
| | other RBD domain can be inserted |
| | in this fusion protein |
| | any other ferritin sequence can be |
| | used in this fusion protein |
| | an RBD core + linker + Ferritin can |
| | be designed based on SEQ ID NO: 97. |
| HV1302125 | SARS-COV-2_NTD_C-SORTA_c3C8HTWSTII (FIG. 20C) |

Initial designs of the SARS-CoV-2 receptor binding domain (RBD) were based on the sequence of the virus spike and were not structurally-informed designs. These RBD designs resulted in a protein that included a portion of the S1 subdomain which truncated within beta sheet structure. In practice, in some instances these RBDs were unstable making them difficult to develop as a vaccine. With the solving of the structure of SARS-CoV-2 spike, RBD core protein designs with different boundaries were rationally designed and encompassed only the portion of the spike that is RBD subunit. These truncated RBD core proteins lack the fragment of S1 that stopped in the middle of a beta sheet and caused the protein folding to be less stable. The RBD cores retain their binding to neutralizing RBD antibodies, but lack the extraneous C terminal peptide sequence. See FIGS. 14A and 14B. In FIG. 14A, the two spheres correspond to amino acid positions: Green=amino acid 333 and Red=527. FIG. 14B shows the positions of amino acid 333 and 527 in the RBDscNP sequence of SEQ ID NO: 97—This sequence is HV1302118. The core SARS CoV2 RBD design is HV1302728v2—does not include linker/tag or ferritin. RBD core designs for any other RBD domain could be designed using the SARS CoV2 RBD core sequence and amino acid positions.

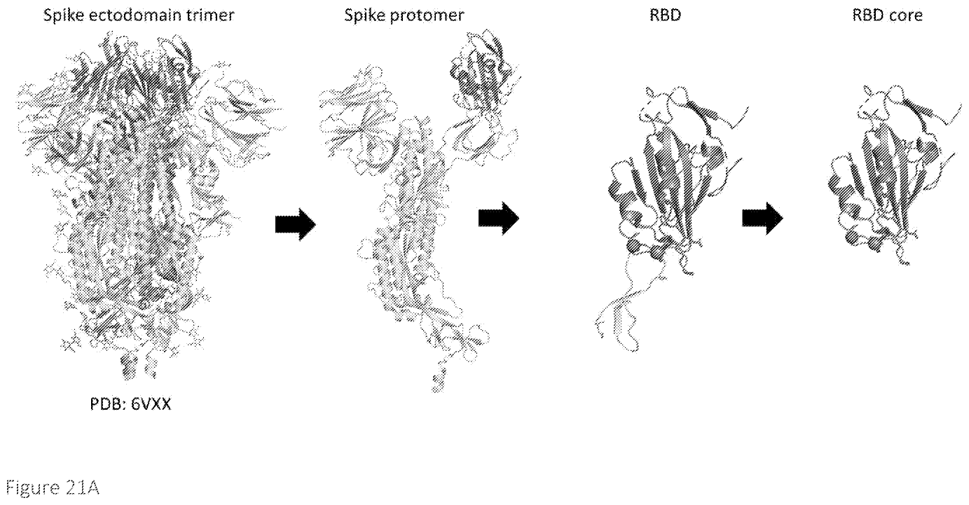

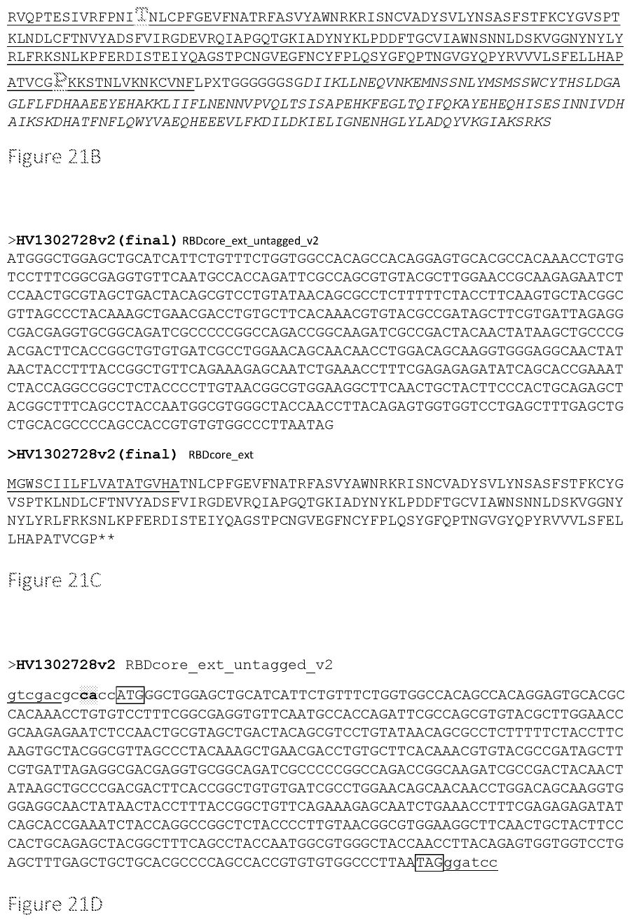

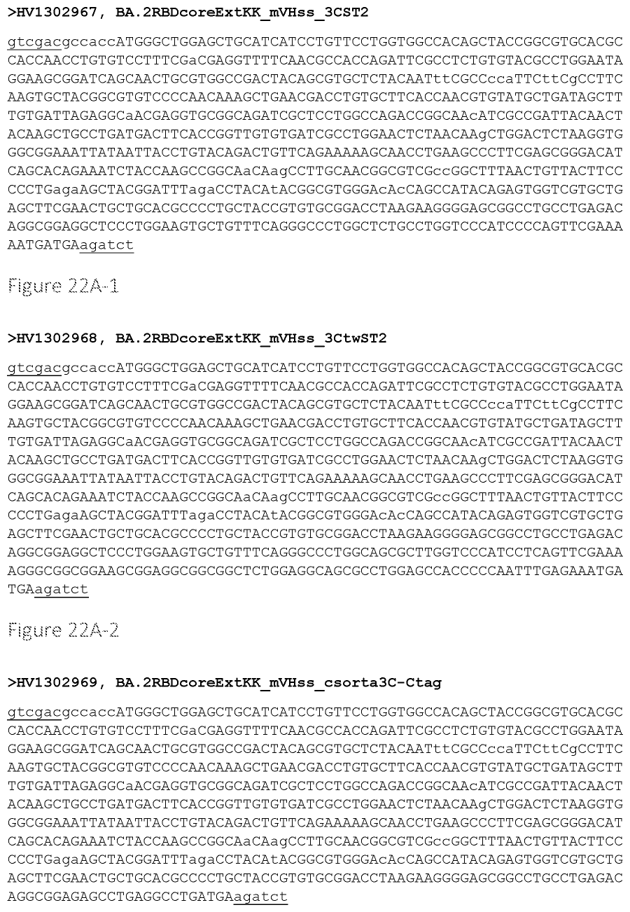

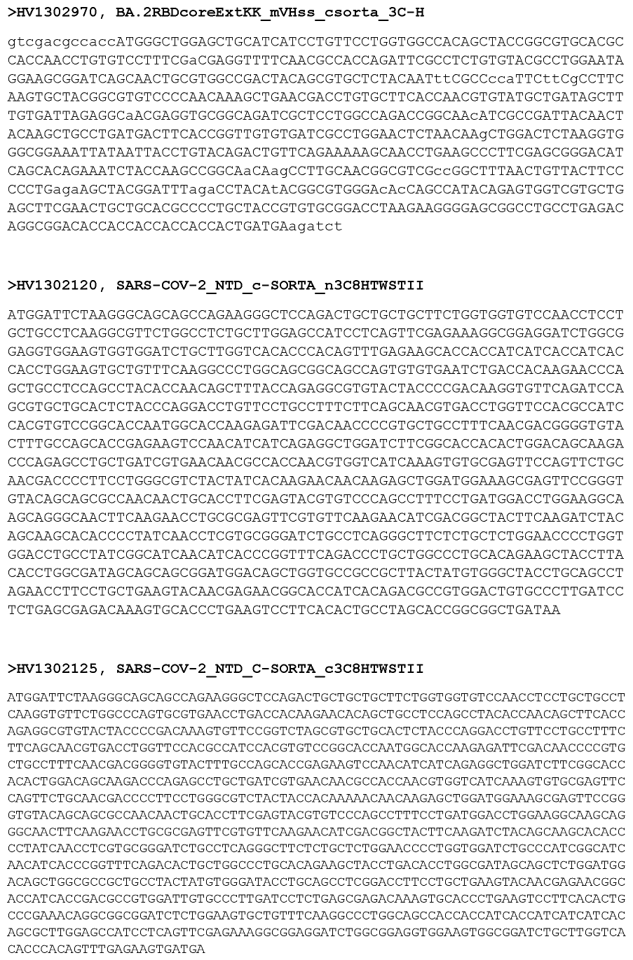

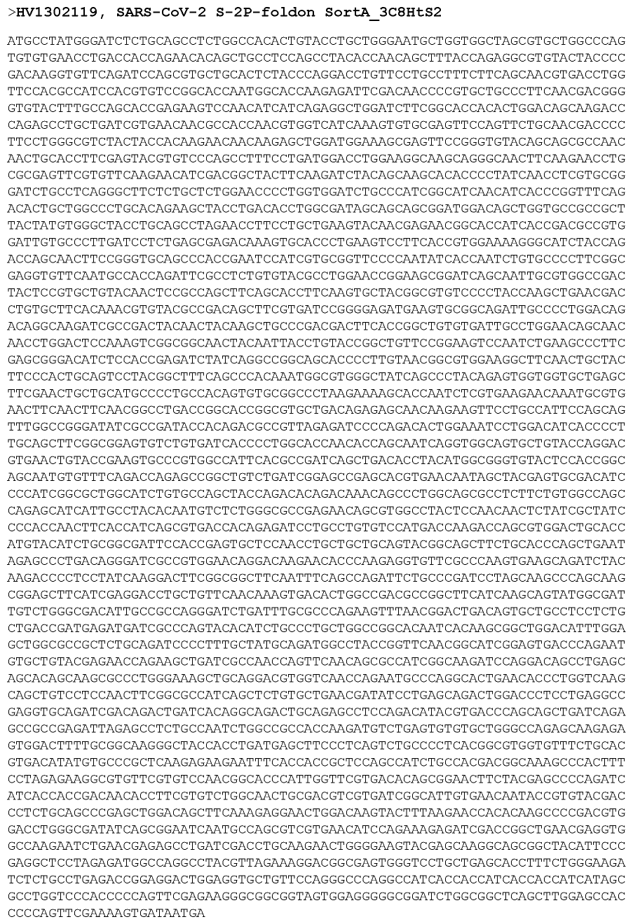

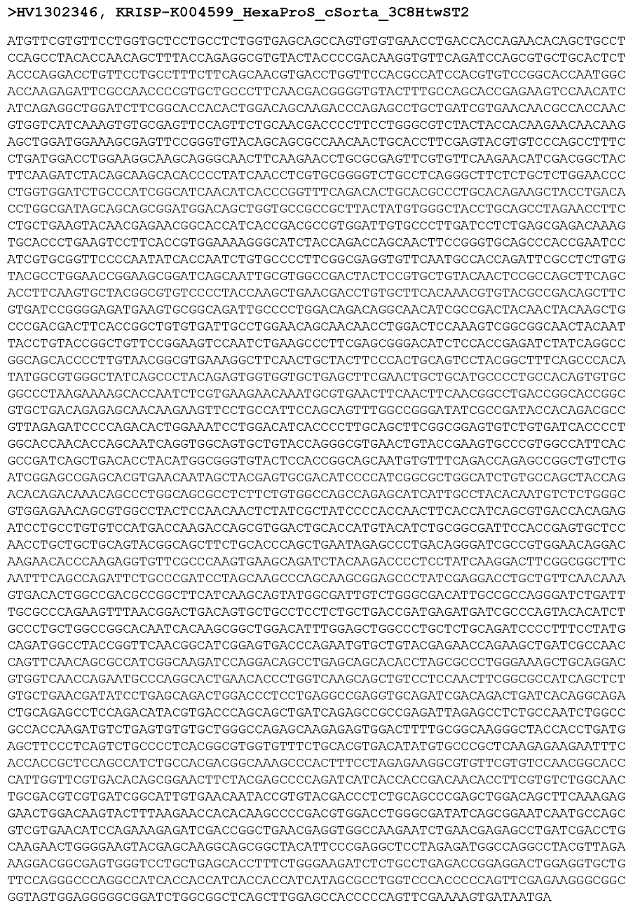

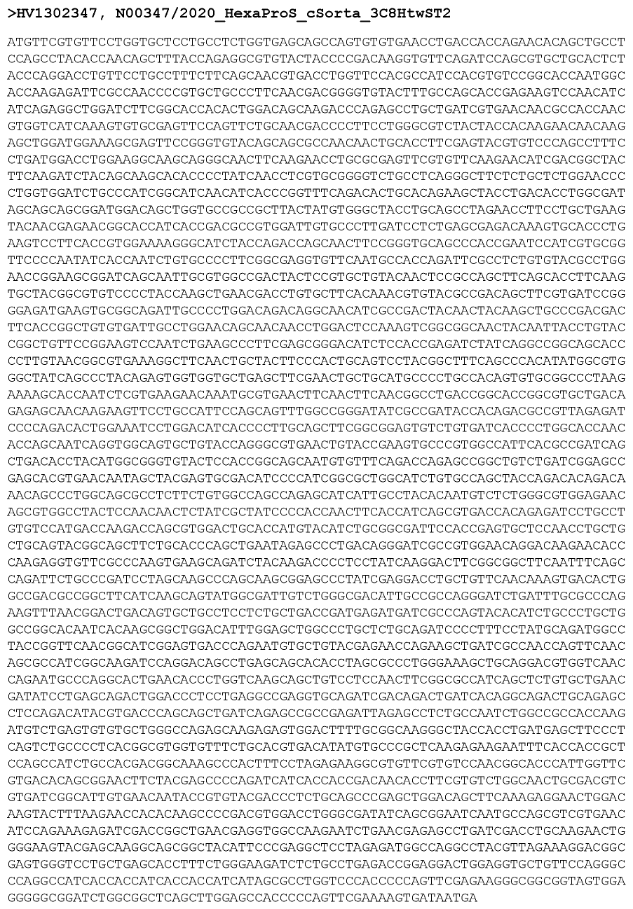

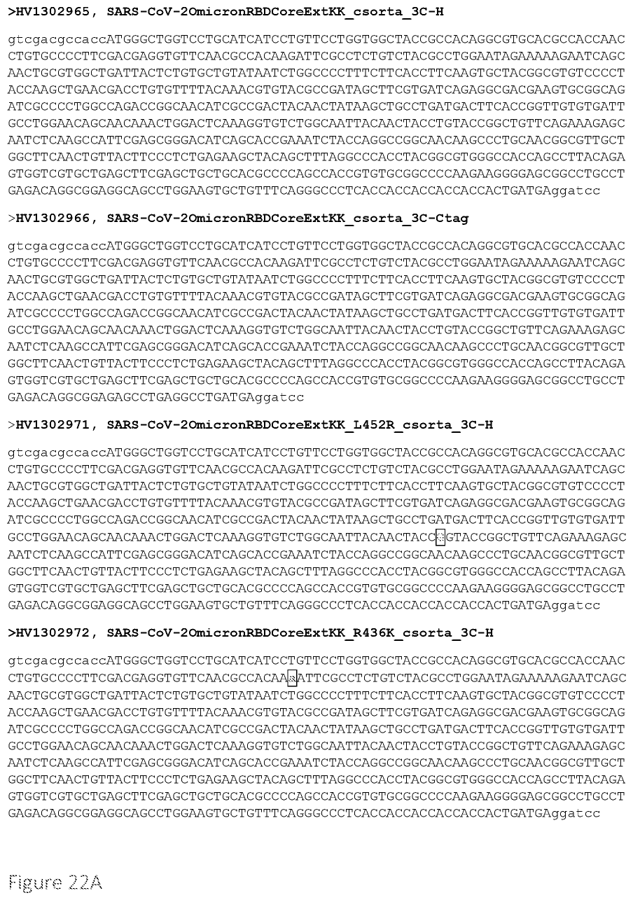

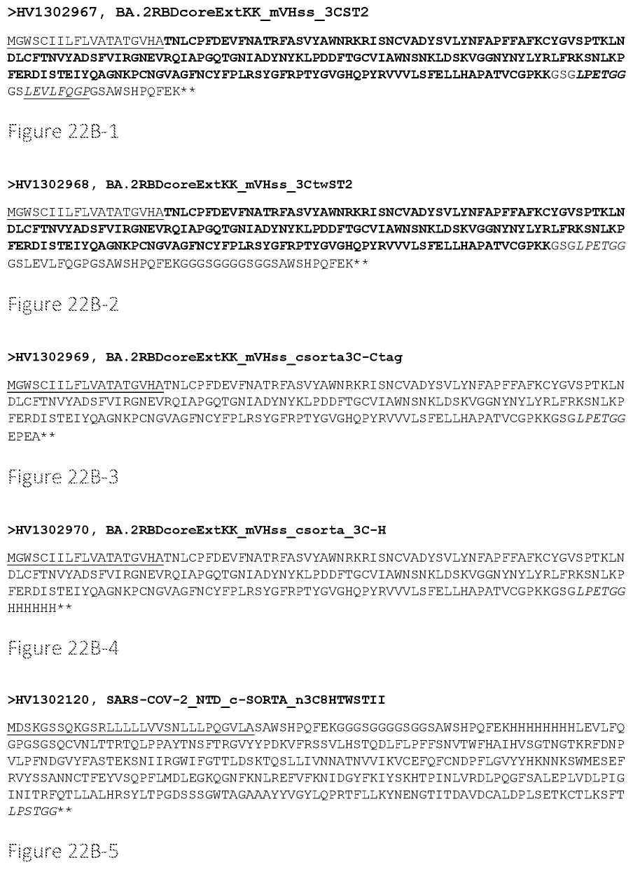

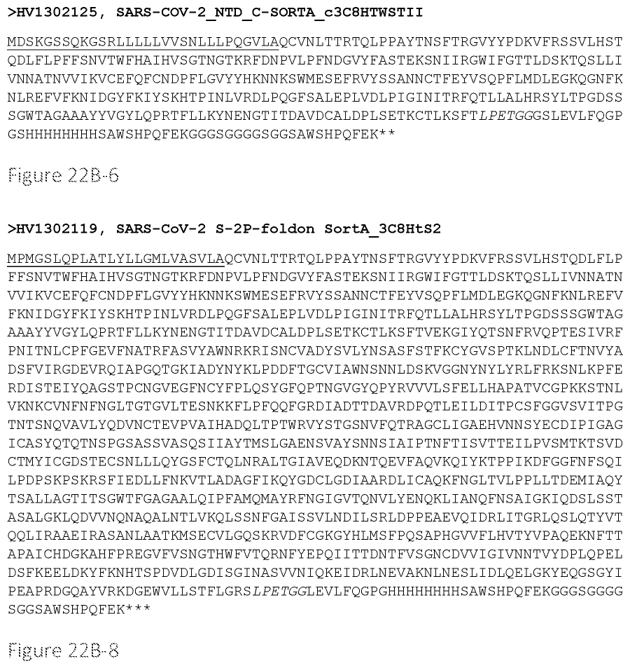

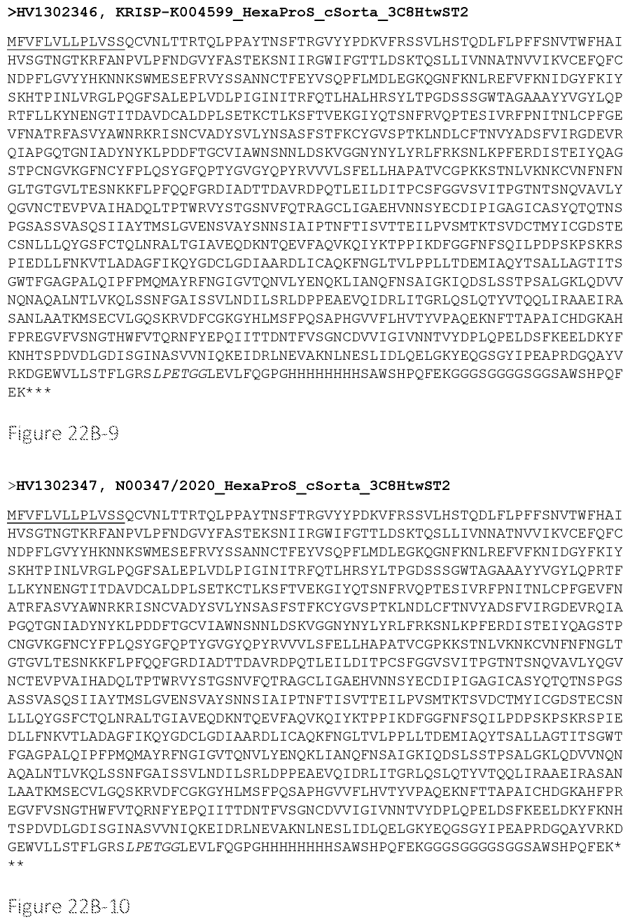

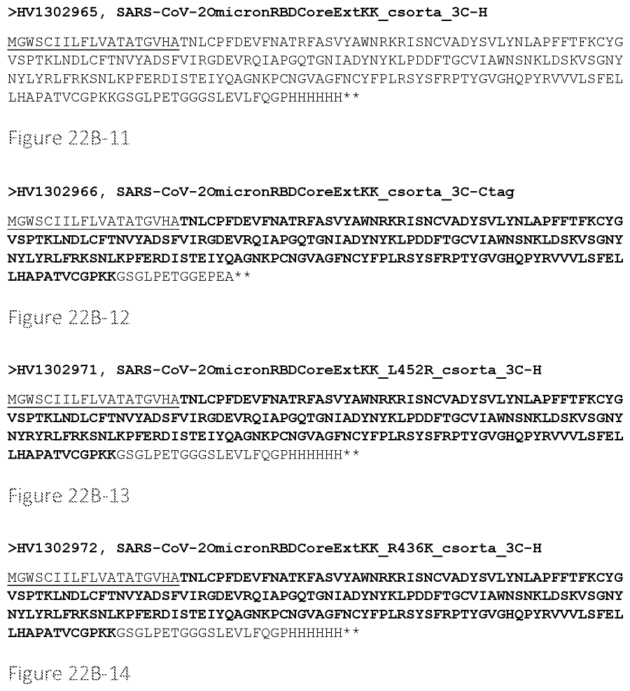

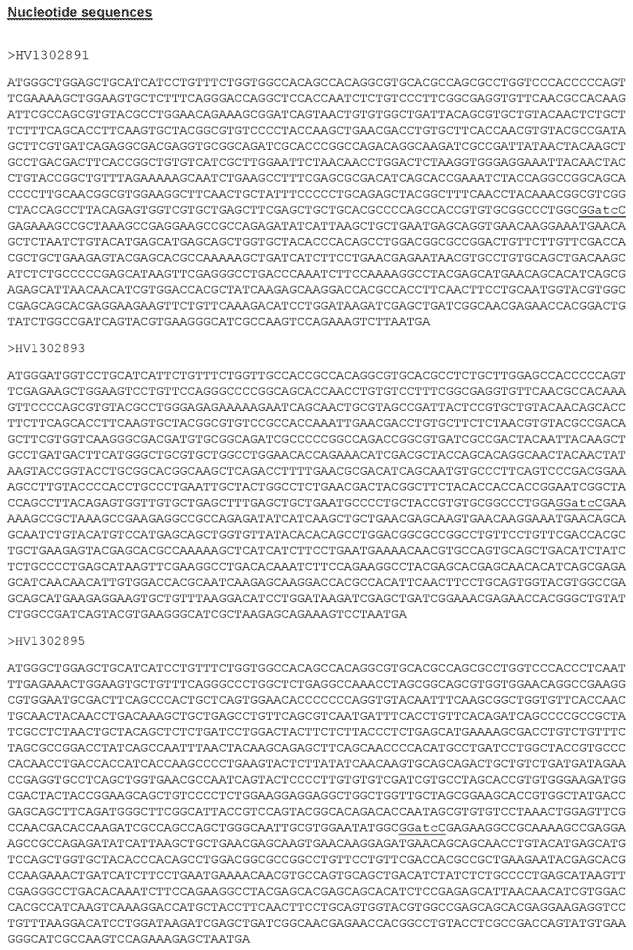

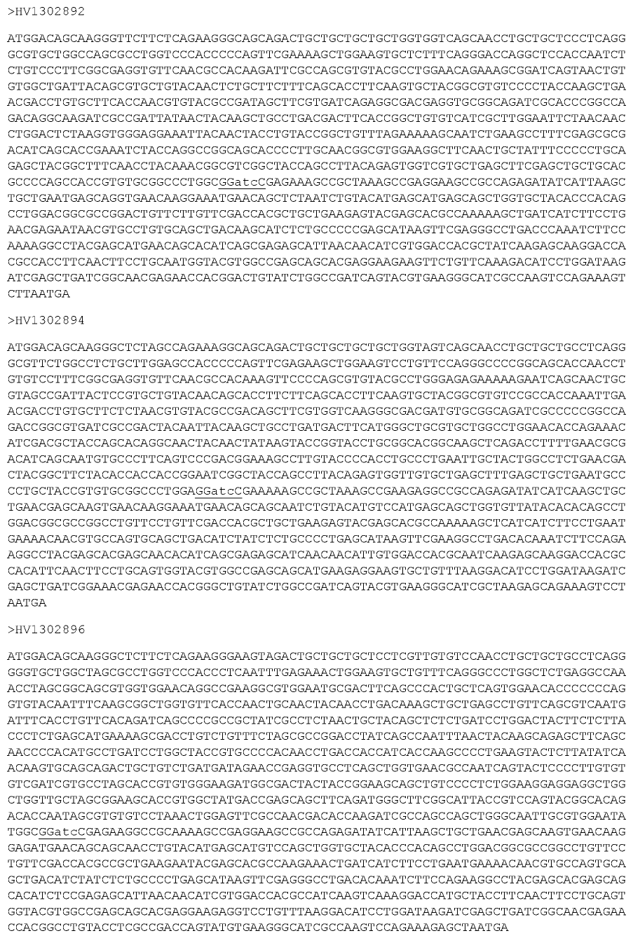

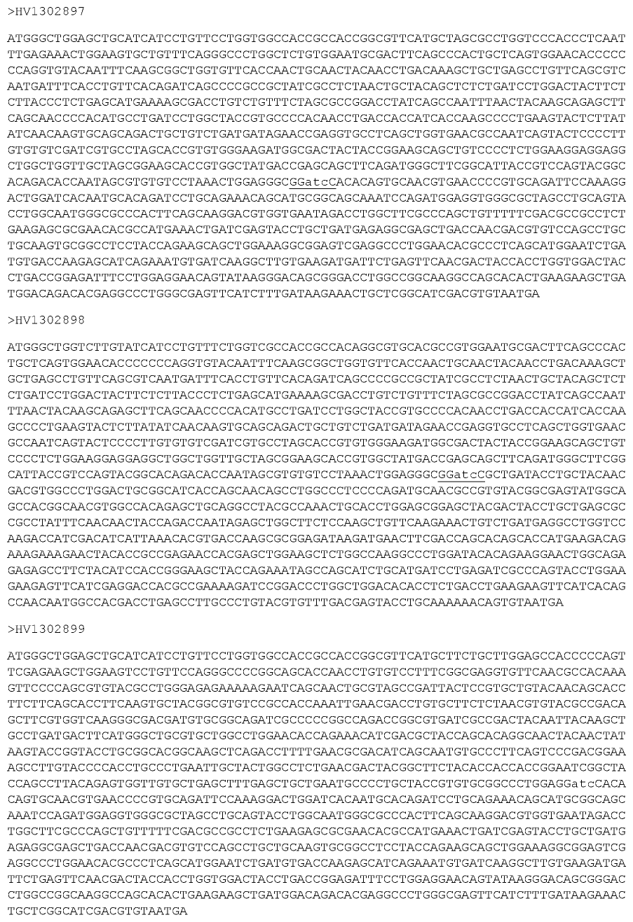

TABLE 2
Non-limiting embodiments of immunogens of the invention. FIGS. 20A, 20B, 21, 22A and 22B show
non-limiting embodiments of amino acid sequences and nucleic acid sequences. Immunogens could
be administered as proteins, nucleic acids, or combination thereof. A skilled artisan can
readily determine the bounds of an RBD, NTD, or FP domain. In some embodiments, the spike
sequence is from CoV2 Wuhan variant. In some embodiments, the spike sequence is from MERS
variant. Using the protein designs described herein which are based on SARS-CoV-2 spike sequence,
a skilled artisan can readily make protein designs from any other coronavirus, including
without limitation any variants. A skilled artisan appreciates that various elements in the
sequences depicted in FIG. 20A and 20B, including without limitation HRV-3C cleavage site,
8X his tag (SEQ ID NO: 17), and a twin StrepTagII (IBA), are included to facilitate expression
and/or purification and are removed from recombinant proteins used as immunogens in animal
or clinical studies. Sequences herein include c-terminal sortase A donor/tag sequences to
allow for site specific conjugation to multimerizing scaffolds expressing the n-terminal
sortase A acceptor sequence. The donor sequence is a LPXTGG (SEQ ID NO: 32)where the third
amino acid X can vary. In some embodiments the sortase tag comprises additional amino acid
so that the sequence is GSGLPXTGG (SEQ ID NO: 37). In one embodiment X is E. The acceptor
sequence is composed of 5 or more glycines (SEQ ID NO: 21) appended to the N-terminus.

| Plasmid ID | Protein ID | Coronavirus |
|---|---|---|
| HV1302100 | SARS_CoV1_BJ01_SpikeEcto2P_3C8HtS2 | SARS-CoV1 |
| HV1302101 | SARS_CoV1_GZ02_SpikeEcto2P_3C8HtS2 | SARS-CoV1 |
| HV1302102 | SARS_coronavirus_SZ3_SpikeEcto2P_3C8HtS2 | SARS-CoV1 |
| HV1302103 | SARS_CoV1_Tor2_SpikeEcto2P_3C8HtS2 | SARS-CoV1 |
| HV1302104 | PCoV_GXP2V_SpikeEcto2P_3C8HtS2 | Pangolin CoV |
| | | GXP2 |
| HV1302105 | PCoV_GXP5E_SpikeEcto2P_3C8HtS2 | Pangolin CoV |
| | | GXP5E |
| HV1302106 | PCoV_GXP5L_SpikeEcto2P_3C8HtS2 | Pangolin CoV |
| | | GXP5L |
| HV1302107 | PCoV_GXP1E_SpikeEcto2P_3C8HtS2 | Pangolin CoV |
| | | GXP1E |
| HV1302108 | PCoV_GXP4L_SpikeEcto2P_3C8HtS2 | Pangolin CoV |
| | | GXP4L |
| HV1302109 | BCOV_RsSHC014_SpikeEcto2P_3C8HtS2 | Bat Coronavirus |
| | | RsSHC014 |
| HV1302110 | BCOV_RaTG13_SpikeEcto2P_3C8HtS2 | Bat Coronavirus |
| | | RaTG13 |
| HV1302111 | BCOV_HKU3-1_SpikeEcto2P_3C8HtS2 | Bat Coronavirus |
| | | HKU3-1 |
| HV1302112 | MERS_CoVYP_009047204_SpikeEcto2P_3C8HtS2 | MERS |
| HV1302113 | HCoV_OC43_SpikeEcto2P_3C8HtS2 | Human CoV OC43 |
| HV1302114 | HCoV_HKU1_SpikeEcto2P_3C8HtS2 | Human CoV HKU1 |
| HV1302115 | HCOV_HKU1_5I08_SpikeEcto_3C8HtS2 | Human CoV HKU1 |
| HV1302116 | HCoV_229E_SpikeEcto2P_3C8HtS2 | Human CoV 229E |
| HV1302117 | HCoV_NL63_SpikeEcto2P_3C8HtS2 | Human CoV NL63 |
| HV1302313 | MERS_CoV_RBDv2_GSG_cSORTA_3C8HtwST2opt | MERS |
| HV1302314 | PCoV_GXP4L_RBD_GSG_cSORTA_3C8HtwST2opt | Pangolin CoV |
| | | GXP4L |
| HV1302315 | BatCoV_RsSHC014_RBD_GSG_cSORTA_3C8HtwST2opt | Bat Coronavirus |
| | | RsSHC014 |
| HV1302316 | SARS_CoV_GZ02_RBD_GSG_cSORTA_3C8HtwST2opt | SARS-CoV1 |
| HV1302631 | CCoV-HuPn-2018RBD_cSortA_3C8HTwST2 | Human |
| | | Pneumonia |
| | | Coronavirus |
| HV1302632 | CCoV-HuPn-2018S-2P_cSortA_3C8HTwST2 | Human |
| | | Pneumonia |
| | | Coronavirus |
| HV1302633 | CCoV-HuPn-2018S-6P_cSortA_3C8HTwST2 | Human |
| | | Pneumonia |
| | | Coronavirus |
| HV1302634 | PDCoV_Haiti_0081-4_2014 RBD_cSortA_3C8HTwST2 | Porcine |
| | | Deltacoronavirus |
| HV1302635 | PDCoV_Haiti_0081-4_2014 S-6P_cSortA_3C8HTwST2 | Porcine |
| | | Deltacoronavirus |
| HV1302636 | PDCoV_Haiti_0256-1_2014 RBD_cSortA_3C8HTwST2 | Porcine |
| | | Deltacoronavirus |
| HV1302637 | PDCoV/Haiti/0329-1/2015 S-6P_cSortA_3C8HTwST2 | Porcine |
| | | Deltacoronavirus |
| HV1302638 | PDCoV_Haiti-0256-1_2015 S-6P_cSortA_3C8HTwST2 | Porcine |
| | | Deltacoronavirus |
| HV1302803 | SARS-CoV-2OmicronRBDExtCore_3C-twST2 | SARS-CoV-2 |
| | | variant |
| HV1302804 | SARS-CoV-2OmicronRBDExtCoreKK_3C-twST2 | SARS-CoV-2 |
| | | variant |
| HV1302805 | SARS-CoV-2OmicronRBDCoreExtKKcsorta_3C-twST2 | SARS-CoV-2 |
| | | variant |
| HV1302806 | SARS-CoV-2OmicronRBDCoreExtcsorta_3C-twST2 | SARS-CoV-2 |
| | | variant |
| HV1302728v2 | RBDcore_ext_untagged_v2 (FIG. 21C and 21D) | SARS-CoV-2 |
| | | variant |
| HV1302125 | | |
| | Sequences of below designs are disclosed in | |
| | FIG. 22A (nucleic acids) and 22B (amino acids) | |
| HV1302967 | BA.2RBDcoreExtKK_mVHss_3CST2 | SARS-CoV-2 |
| | | variant |
| HV1302968 | BA.2RBDcoreExtKK_mVHss_3CtwST2 | SARS-CoV-2 |
| | | variant |
| HV1302969 | BA.2RBDcoreExtKK_mVHss_csorta3C-Ctag | SARS-CoV-2 |
| | | variant |
| HV1302970 | BA.2RBDcoreExtKK_mVHss_csorta_3C-H | SARS-CoV-2 |
| | | variant |
| HV1302120 | SARS-COV-2_NTD_c-SORTA_n3C8HTWSTII | SARS-CoV-2 |
| | | variant |
| HV1302125 | SARS-COV-2_NTD_C-SORTA_c3C8HTWSTII | SARS-CoV-2 |
| | | variant |
| HV1302119 | SARS-CoV-2 S-2P-foldon SortA_3C8HtS2 | SARS-CoV-2 |
| | | variant |
| HV1302346 | KRISP-K004599_HexaProS_cSorta_3C8HtwST2 | |
| HV1302347 | N00347/2020_HexaProS_cSorta_3C8HtwST2 | |
| HV1302965 | SARS-CoV-2OmicronRBDCoreExtKK_csorta_3C-H | SARS-CoV-2 |
| | | variant |
| HV1302966 | SARS-CoV-2OmicronRBDCoreExtKK_csorta_3C-Ctag | SARS-CoV-2 |
| | | variant |
| HV1302971 | SARS-CoV-2OmicronRBDCoreExtKK_L452R_csorta_3C-H | SARS-CoV-2 |
| | | variant |
| HV1302972 | SARS-CoV-2OmicronRBDCoreExtKK_R436K_csorta_3C-H | SARS-CoV-2 |
| | | variant |

TABLE 3
Non-limiting embodiments of immunogens of the invention. FIGS.
23A-B show non-limiting embodiments of amino acid sequences
and nucleic acid sequences. Immunogens could be administered
as proteins, nucleic acids, or combination thereof. A skilled
artisan can readily determine the bounds of an RBD, NTD,
or FP domain. In some embodiments, the spike sequence is
from CoV2 Wuhan variant. In some embodiments, the spike sequence
is from MERS variant. Using the protein designs described
herein which are based on SARS-CoV-2 spike sequence, a skilled
artisan can readily make protein designs from any other coronavirus,
including without limitation any variants. A skilled artisan
appreciates that various elements in the sequences depicted
in FIG. 23A and 23B, including without limitation HRV-3C
protease site: LEVLFQGP (SEQ ID NO: 28), StrepTagII purification
tag: SAWSHPQFEK (SEQ ID NO: 33), are included to facilitate
expression and/or purification and are removed from recombinant
proteins used as immunogens in animal or clinical studies.

| ID | Protein name |
|---|---|
| HV1302891 | SARS2RBDcoreExt.3GS11EKR.HPferritin_nST2_mVHss |
| HV1302892 | SARS2RBDcoreExt.3GS11EKR.HPferritin_nST2_bPrIss |
| HV1302893 | SARS1RBDcoreExt.3GS11EKR.HPferritin_nST2_mVHss |
| HV1302894 | SARS1RBDcoreExt.3GS11EKR.HPferritin_nST2_bPrlss |
| HV1302895 | MERSRBD.3GS11EKR.HPferritin_nST2_mVHss |
| HV1302896 | MERSRBD.3GS11EKR.HPferritin_nST2_bPrIss |
| HV1302897 | MERSRBDcore.3GS.TniFerritinHC_nST2_mVHss |
| HV1302898 | MERSRBDcore.3GS.TniFerritinLC_nST2_mVHss |
| HV1302899 | SARS1RBDcoreExt.3GS.TniFerritinHC_nST2_mVHss |
| HV1302900 | SARS1RBDcoreExt.3GS.TniFerritinLC_nST2_mVHss |
| HV1302901 | MERSRBDcore.3GS11EKR.HPferritin_nST2_mVHss |
| HV1302902 | MERSRBDcore.3GS11EKR.HPferritin_nST2_bPrIss |

TABLE 4
Non-limiting embodiments of various linkers

| | |
|---|---|
| C-terminal Linker/sortase tag fused | Linker between spike or |
| to spike or portion thereof before | portion thereof and |
| sortase mediated conjugation | multimerizing protein |
| These are short linkers to allow | (e.g. ferritin) after |
| better access to the sortase A | sortase conjugation |
| acceptor peptide. The design | |
| goal is to extend the sortase A | |
| tag away from the RBD so that it | |
| can connect to the nanoparticle | |
| with sterically hitting the RBD | |
| beside it. | |
| LPXTG(GG) (SEQ ID NO: 38) | LPXTGGGGGGSG (X could be E or S) |
| LPXTGG(G) (SEQ ID NO: 39) | (SEQ ID NO: 41) |
| LPXTGGG (SEQ ID NO: 40) | Or |
| (G) and (GG) are optional | LPXTGGGGG (X could be E or S) |
| | (SEQ ID NO: 42) |
| GSGLPXTG(GG) (SEQ ID NO: 43) | GSGLPXTGGGGG (X could be E or S) |
| GSGLPXTGG(G) (SEQ ID NO: 44) | (SEQ ID NO: 46) |
| GSGLPXTGGG (SEQ ID NO: 45) | |
| (G) and (GG) are optional | |
| Linkers in direct fusions | |
| between spike or portion | |
| thereof and multimerizing | |
| protein (e.g. ferritin) | |
| [GGS](n) (SEQ ID NO: 47) | Embodiment of a flexible linker |
| | Where n = 1, 2, 3, 4, 5, 6-10. |
| EKAAKAEEAAR (SEQ ID NO: 9) | Embodiments of rigid linkers |
| EKAAKAEEAARP(SEQ ID NO: 10) | |
| EKAAKAEEAARPP (SEQ ID NO: 11) | |
| GGSEKAAKAEEAAR(SEQ ID NO: 12) | Embodiments of flexible and |
| GGSEKAAKAEEAARP(SEQ ID NO: 13) | rigid linkers |
| GGSEKAAKAEEAARPP (SEQ ID NO: 14) | |

Tables 1, 2 and 3 list spike proteins sequences or portions thereof, e.g. RBD or NTD, that can be conjugated or otherwise arrayed on nanoparticles to create multimeric/multivalent immunogens. The sequences encompass spike proteins from animal coronaviruses and human coronaviruses. Together, these spike proteins or portions thereof can be used to elicit broad immune responses against coronaviruses. These coronaviruses include those with pandemic potential and those that are endemic to the human population.

Table 1, Table 2 and Table 3 list non-limiting examples of immunogens designs from different human or animal coronaviruses with different sequences. These sequence designs are non-limiting examples of spike proteins or portions thereof that can be used in multispecific multimeric complexes and/or in multimeric complexes in multispecific compositions. Multispecific multimeric complexes are also referred to as mosaic nanoparticles. Mosaic sortase conjugate nanoparticles can be made by displaying multiple human or animal coronavirus receptor binding domains on the surface of nanoparticles. Receptor binding domains can include MERS-CoV (HV1302313), SARS-CoV-1 (HV1302316), Canine CoV-HuPn (HV1302631), Bat CoV SHC014 (HV1302315), Pangolin CoV (PCoV) GXP4L (HV1302314), or Porcine CoV Haiti_0081-4_2014 RBD (HV1302634). These RBD scNPs would span alpha, beta, gamma, and delta coronaviruses representing a means to elicit pan-coronavirus neutralizing antibodies.

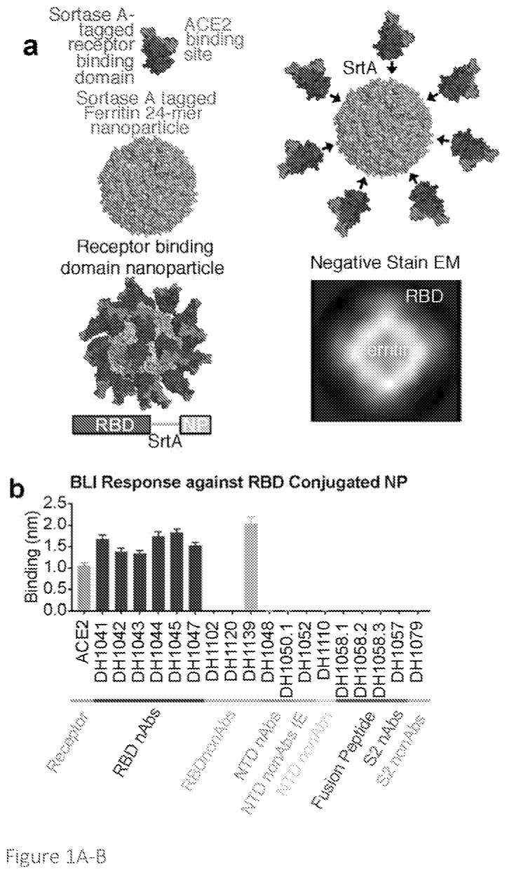

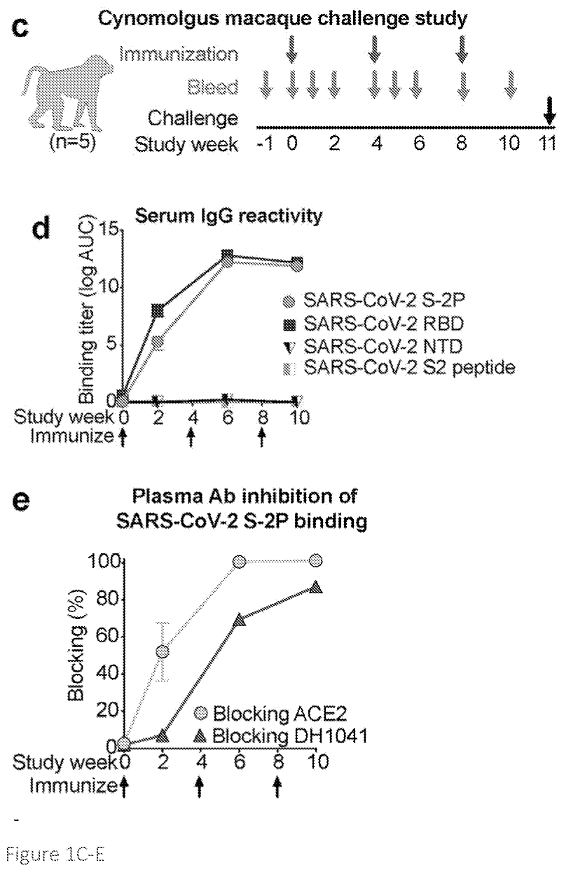

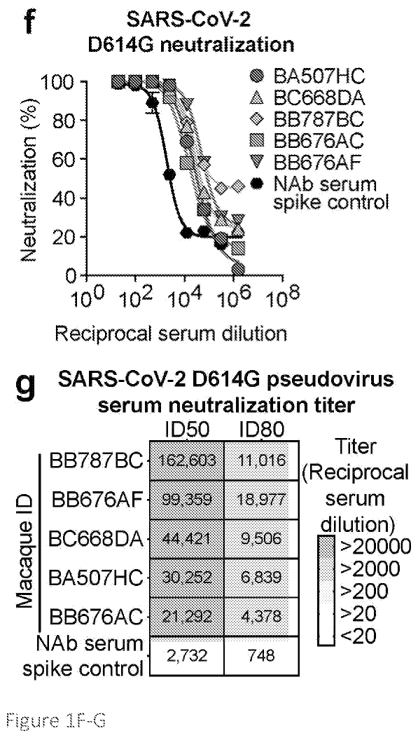

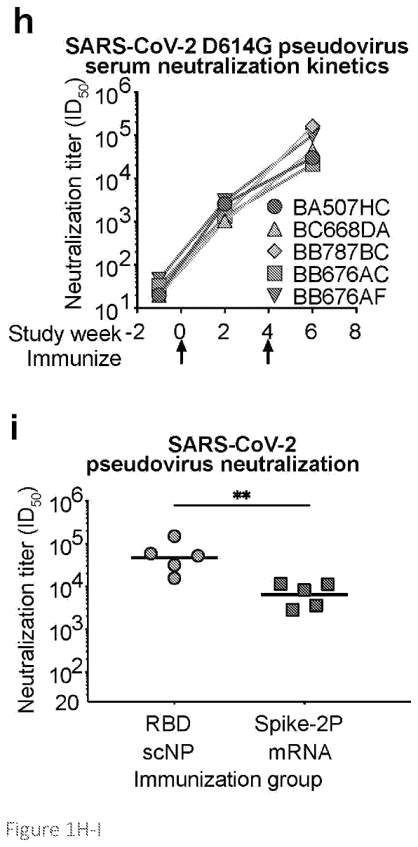

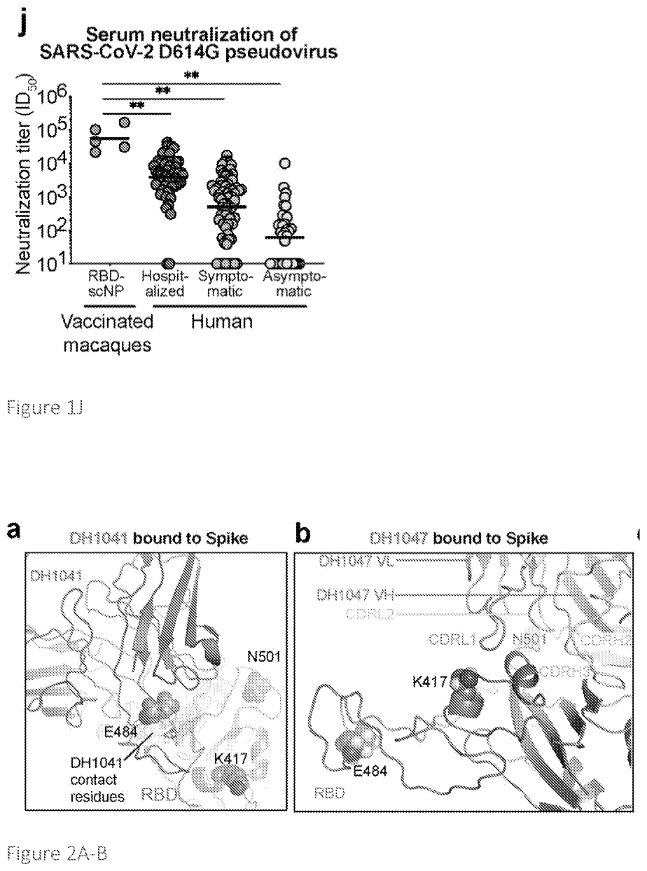

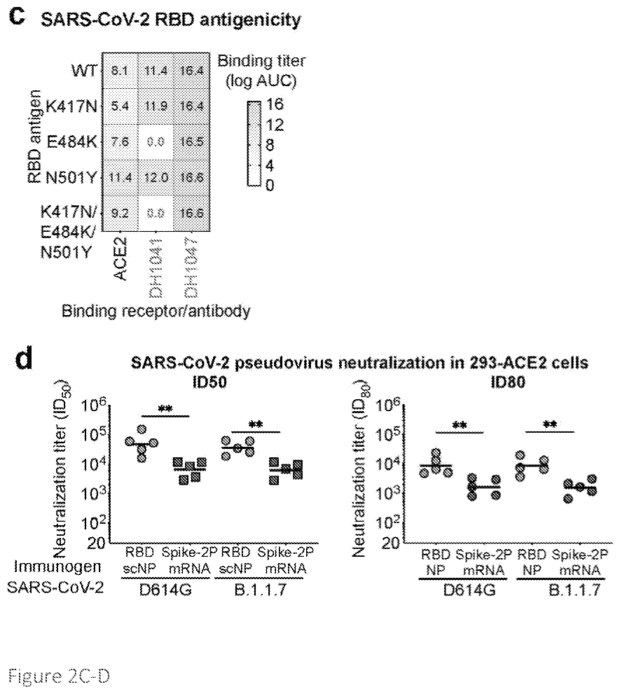

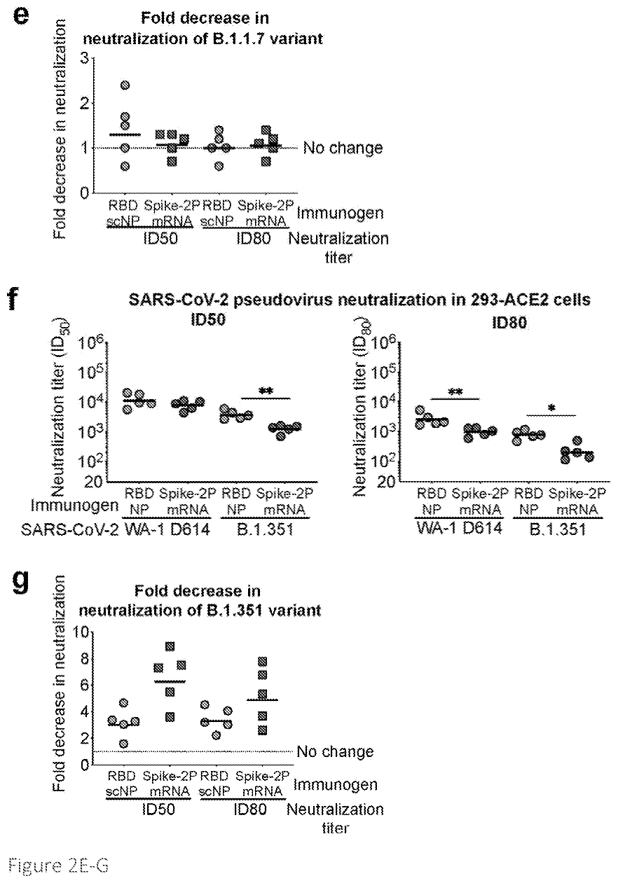

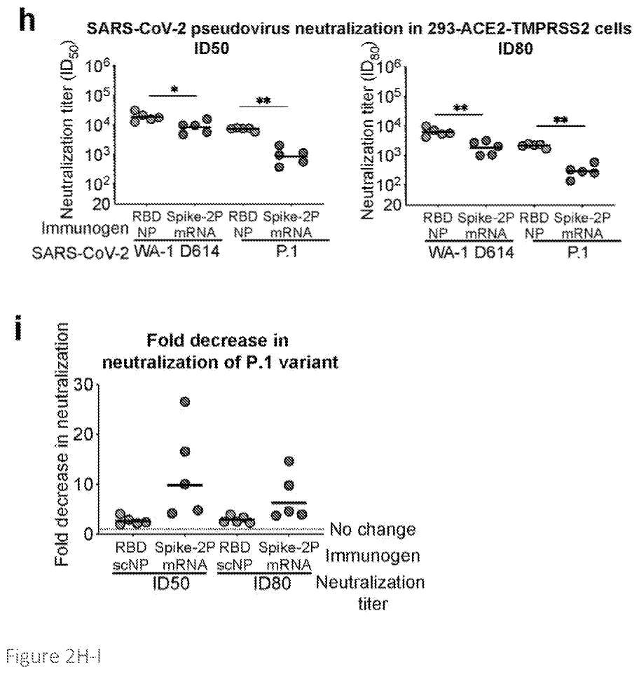

An RBDscNP immunogen generated from HV1302118 is shown in FIG. 18C-1.

Throughout some sequences represented in figures, various elements are annotated, e.g. signal peptide, linkers, and so forth. These annotated sequences are representative and are used as a guide to identify such elements in sequences which are not annotated.

Sortase conjugated nanoparticles (scNP) are made by conjugating sortase tagged RBDs, spike proteins or other spike protein portions thereof with the multimeric complex, e.g. a ferritin nanoparticle, in a reaction mediated by the enzyme sortase A. Sortase enzyme conjugation reaction conditions are known and could be further optimized. See M. W. Popp, J. M. Antos, H. L. Ploegh, Site-specific protein labeling via sortase-mediated transpeptidation. Curr Protoc Protein Sci Chapter 15, Unit 15.13 (2009); J. M. Antos et al., Site-Specific Protein Labeling via Sortase-Mediated Transpeptidation. Curr Protoc Protein Sci 89, 15.13.11-15.13.19 (2017). Sortase conjugated mosaic nanoparticles are made by mixing equimolar concentrations of the different sortase tagged RBDs, spike proteins or other spike protein portions thereof with the multimeric complex, e.g. a ferritin nanoparticle, in a single reaction mediated by the enzyme sortase A. For example, sortase conjugated mosaic nanoparticles comprising two different RBDs are made by mixing equimolar concentrations, for example without limitation 50 micromolar of each of the two RBDs, of different sortase tagged RBDs with ferritin nanoparticles in a single sortase A reaction. The conjugate nanoparticle is purified away from unconjugated nanoparticle, and/or the unconjugated proteins, e.g. RBDs. The presence of the two RBDs can be determined by sandwich ELISA or equivalent binding assays using antibodies specific for only one of the RBDs. In some embodiments of the scNP approach, the equal representation of the two RBDs on each nanoparticle is not controlled but instead the equivalent amount of each RBD is assessed for the entire population of nanoparticles. In other embodiments, reaction is carried out under conditions suitable for relatively equal incorporation of each RBD in a nanoparticle.

A multivalent nanoparticle can be formed by two multimerizing components that interact together to form a nanoparticle. In embodiments where the multivalent RBD nanoparticle is be formed by two components that form the nanoparticle, this allows for each of the two RBDs to be genetically fused to a different component of the nanoparticle. For single component nanoparticles, the two RBDs are genetically fused to the same subunit sequence. Upon expression of a two component nanoparticle such as T. ni ferritin the two chains spontaneously form into a nanoparticle with 12 RBD-ferritin heavy chain subunits assembling with 12 RBD-ferritin light chain subunits. When the nanoparticle is composed of a single subunit, the nanoparticle forms with a mixture of subunits fused to each RBD. The presence of the two RBDs can be determined by sandwich ELISA or equivalent binding assays using antibodies specific for only one of the RBDs. In the single component nanoparticle approach, the equal representation of the two RBDs on each nanoparticle is not controlled but instead the equivalent amount of each RBD is assessed for the entire population of nanoparticles. Mass spectrometry is used to verify the presence of all intended RBDs. Relative abundance of each RBD is determined in western blots using an antibody specific for each RBD and in vitro transfections with mRNA-LNPs.

To make lipid nanoparticle encapsulated-mRNA encoding multispecific RBD nanoparticles-nanoparticles displaying more than one receptor binding domain—an mRNA encoding each RBD fused to a ferritin sequences which forms a nanoparticle is made. The mRNA that encode each RBD ferritin subunit is mixed together and encapsulated in lipid nanoparticles. Since a single cell can take up many mRNA-LNPs it is not necessary to equally load each LNP with equal amounts of the two mRNAs.

Making mosaic NP versus two or more different specific nanoparticles mixed in the vaccine formulation may provide advantages. Mosaic nanoparticles are reported to give higher neutralizing antibodies than mixing two nanoparticles together. In theory, mosaic nanoparticles bind to the B cell receptor of cross-reactive B cells since such B cells can engage all of the antigens presented on the nanoparticle surface. The crosslinking of more B cell receptors leads to a stronger activation signal for the B cell. The effect was recently reported by Walls et al for coronavirus RBD (Cell. 2021 Oct. 14; 184(21):5432-5447). When the RBDs are not expected to bind to single BCR it is unknown whether mosaic nanoparticles will be better than mixing two NPs together. We will determine the difference in preclinical mouse experiments.

The following terms have the meanings listed unless otherwise specified. Any undefined terms have their art recognized meanings.

“Alkyl” can refer to a straight chain or branched, noncyclic or cyclic, unsaturated or saturated aliphatic hydrocarbon containing from 1 to 30 carbon atoms, and in certain embodiments containing from 1 to 25, or 1 to 20, or 1 to 15, or 1 to 12, or 1 to 10, or 1 to 8, or 1 to 6, carbon atoms, unless specified elsewhere herein. Representative saturated straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while saturated branched alkyls include isopropyl, sec-butyl, isobutyl, tert-butyl, isopentyl, and the like, Representative saturated cyclic alkyls include cyclopropyl, cyclobutyl cyclopentyl, cyclohexyl and the like; while unsaturated cyclic alkyls include cyclopentenyl and cyclohexenyl, and the like. Cyclic alkyls are also referred to herein as “homocycles” or “homocyclic rings” Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like: while representative straight chain and branched alkynyls include acetylenyl, propynyl, 1-butynyl, 2-butynyl, 1-pentynyl, 2-pentynyl, 3-methyl-1-butynyl, and the like.

“Halo” or “halogen” refers to fluoro, chloro, bromo, and iodo.

“Hydroxy” or “hydroxyl” refers to the group —OH.

“Alkoxy” refers to the group —O-alkyl, wherein alkyl is as defined herein.

Alkoxy includes, by way of example, methoxy, ethoxy, n-propoxy, isopropoxy, n-butoxy, t-butoxy, sec-butoxy, n-pentoxy, and the like.

“Acylamino” refers to the groups —NR²⁰C(O)R²¹, wherein R²⁰ and R²¹ are independently selected from hydrogen, alkyl, and aryl.

The term “aryl” refers to a monovalent group that is aromatic and, optionally, carbocyclic. The aryl has at least one aromatic ring. Any additional rings can be unsaturated, partially saturated, saturated, or aromatic. Optionally, the aromatic ring can have one or more additional carbocyclic rings that are fused to the aromatic ring. Unless otherwise indicated, the aryl groups can contain from 6 to 30 carbon atoms. In some embodiments, the aryl groups contain 6 to 20, 6 to 18, 6 to 16, 6 to 12, or 6 to 10, carbon atoms. Examples of an aryl group include phenyl, naphthyl, biphenyl, phenanthryl, and anthracyl.

The term “comprises” and variations thereof do not have a limiting meaning where these terms appear in the description and claims. Such terms comprise the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other claims may also be preferred, under the same or other circumstances. Furthermore, the recitation of one or more claims does not imply that other claims are not useful, and is not intended to exclude other claims from the scope of the disclosure.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

The term “or” can be employed in its usual sense including “and/or” unless the content dictates otherwise.

The term “and/or” can refer to one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

The term “room temperature” refers to a temperature of 20° C. to 25° C. or 22° C. to 25° C.

The term “in the range” or “within a range” (and similar statements) includes the endpoints of the stated range.

Groupings of alternative elements or embodiments disclosed herein are not to be construed as limitations. Each group member may be referred to and claimed individually or in any combination with other members of the group or other elements found therein. Without wishing to be bound by theory, one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

When a group is present more than once in a formula described herein, each group is “independently” selected, whether specifically stated or not. For example, when more than one Y group is present in a formula, each Y group is independently selected.

Furthermore, subgroups contained within these groups are also independently selected. For example, when each Y group contains an R, then each R is also independently selected.

Reference throughout this specification to “one embodiment,” “an embodiment,” “certain embodiments,” or “some embodiments,” etc., can refer to a particular feature, configuration, composition, or characteristic described in connection with the embodiment is included in at least one embodiment of the invention. Thus, the appearances of such phrases in various places throughout this specification are not necessarily referring to the same embodiment of the invention. Furthermore, the particular features, configurations, compositions, or characteristics may be combined in any suitable manner in one or more embodiments.

The summary of the t disclosure provided herein is not intended to describe each disclosed embodiment or every implementation of the invention. The description more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and mechanisms may have been discussed herein, in no event should such discussions serve to limit the claimable subject matter.

In non-limiting embodiments, compositions can be formulated with appropriate carriers and adjuvants using techniques to yield compositions suitable for immunization. The compositions can include an adjuvant, such as, for example but not limited to alum, 3M052, poly IC, MF-59 or another squalene-based oil-in-water emulsion adjuvant, AS01B, or other liposomal based adjuvant suitable for protein or nucleic acid immunization. In non-limiting embodiments, LNPs are used as adjuvants for immunogenic formulations comprising proteins, including multimeric protein complexes and nanoparticles. In certain embodiments, the adjuvant is GSK AS01E adjuvant containing MPL and QS21. This adjuvant has been shown by GSK to be as potent as the similar adjuvant AS01B but to be less reactogenic. In certain embodiments, AS04, a combination of alum and 3-O-desacyl-4′-monophosphoryl lipid A (MPL) developed by GSK. In certain embodiments the adjuvant is AS03, an oil-in-water emulsion combination adjuvant developed by GSK. Non-limiting embodiments of adjuvants are described in the NIH 2018 Strategic Plan for Research on Vaccine Adjuvants. https://www.niaid.nih.gov/sites/default/files/NIAIDStrategicPlanVaccineAdjuvants2018.pdf

In certain embodiments, the composition and methods comprise an adjuvant. Non-limiting examples of adjuvants include GLA/SE, alum, Poly I poly C (poly IC), polyIC/long chain (LC) TLR agonists, TLR7/8 and/or 9 agonists (e.g. CpG-oligodeoxynucleotide (oCpG)), or a combination of TLR7/8 and TLR9 agonists (see Moody et al. (2014) J. Virol. March 2014 vol. 88 no. 6 3329-3339), or any other suitable adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and CpG-oligodeoxynucleotide (oCpG) in STS (see Moody et al. (2014) J. Virol. March 2014 vol. 88 no. 6 3329-3339).

In certain embodiments, the adjuvant can be Alum (aluminum hydroxide) or variants of Alum such as pSer Alum (Moyer et al. Nat Med 2020 March; 26(3):430-440; doi: 10.1038/s41591-020-0753-3. Epub 2020 Feb. 17.).

In certain embodiments, TLR agonists are used as adjuvants. In certain embodiments, TLR agonists are TLR7/8 agonist. Non-limiting examples of TLR 7/8 are described in Evans et al. ACS Omega 2019, 4, 13, 15665-15677; Patinote et al. Eur J Med Chem. 2020 May 1; 193: 112238, Miller et al. in Front. Immunol., 10 Mar. 2020| https://doi.org/10.3389/fimmu.2020.00406. In certain embodiments, adjuvants are TLR 4 agonists. Is some embodiments, the adjuvants are saponins. See e.g. WO2019079160A1 and references cited therein. In some embodiments, saponins are natural, synthetic, or semi-synthetic saponins.

Non-limiting embodiments of adjuvants include without limitation inulin based adjuvants, see e.g. Advax in Petrovsky and Cooper, Vaccine 2015 Nov. 4; 33(44): 5920-5926, Advax+TLR agonists and Advax+CpG, see e.g. Counoupas et al. Sci Rep. 2017; 7: 8582), saponins, Alhydroxiquim (TLR7/8), IMDQ-Dendrimer (TLR7), Polymeric TLR7/8 adjuvants, Mastoparn-7 (M7), adjuvant LT(R192G/L211A) also referred to as dmLT (see e.g. Celemens and Norton, mSphere. 2018 July-August; 3(4): e00215-18).

In certain embodiment, adjuvants which break immune tolerance are included in the immunogenic compositions (e.g. Verkoczy et al. J Immunol. 2013 Sep. 1; 191(5):2538-50; doi: 10.4049/jimmunol.1300971. Epub 2013 Aug. 5.).

In non-limiting embodiments, different adjuvants could be combined.

Other non-limiting embodiments of adjuvants that may be used include without limitation Matrix M (e.g. Gorman et al. in bioRxiv. 2021 Feb. 5:2021.02.05.429759. doi: 10.1101/2021.02.05.429759. Preprint. PMID: 33564763), ALFQ from the Military HIV Research Program (e.g. “Army Liposome Formulation (ALF) family of vaccine adjuvants” Alving C R, Peachman K K, Matyas G R, Rao M, Beck Z. Expert Rev Vaccines. 2020 March; 19(3):279-292. doi: 10.1080/14760584.2020.1745636. Epub 2020 Mar. 31.PMID: 32228108), MF-59 (e.g. Safety and effectiveness of MF-59 adjuvanted influenza vaccines in children and adults. Black S. Vaccine. 2015 Jun. 8; 33 Suppl 2:B3-5. doi: 10.1016/j.vaccine.2014.11.062.PMID: 26022564), GLA-SE or aqueous formulation (e.g. Felber et al. Cell Rep. 2020 May 12; 31(6):107624. doi: 10.1016/j.celrep.2020.107624.PMID: 32402293), CpG 1018 (e.g “Development of CpG-adjuvanted stable prefusion SARS-CoV-2 spike antigen as a subunit vaccine against COVID-19” Kuo T Y, Lin M Y, Coffman R L, Campbell J D, Traquina P, Lin Y J, Liu L T, Cheng J, Wu Y C, Wu C C, Tang W H, Huang C G, Tsao K C, Chen C. Sci Rep. 2020 Nov. 18; 10(1):20085. doi: 10.1038/s41598-020-77077-z.PMID: 33208827), Adjuplex (e.g. “Carbomer-based adjuvant elicits CD8 T-cell immunity by inducing a distinct metabolic state in cross-presenting dendritic cells.” Lee W, Kingstad-Bakke B, Paulson B, Larsen A, Overmyer K, Marinaik C B, Dulli K, Toy R, Vogel G, Mueller K P, Tweed K, Walsh A J, Russell J, Saha K, Reyes L, Skala M C, Sauer J D, Shayakhmetov D M, Coon J, Roy K, Suresh M. PLoS Pathog. 2021 Jan. 14; 17(1):e1009168. doi: 10.1371/joumal.ppat.1009168. eCollection 2021 January PMID: 33444400) and inulin based adjuvants (e.g. “Research Note: The immune enhancement ability of inulin on ptfA gene DNA vaccine of avian Pasteurella multocida.” Gong Q, Peng Y G, Niu M F, Qin C L. Poult Sci. 2020 June; 99(6):3015-3019. doi: 10.1016/j.psj.2020.03.006. Epub 2020 Mar. 24.PMID: 32475437).

The content of each and every citation is incorporated by reference in its entirety.

### TLR7/8 Agonists

Provided herein are TLR7/8 agonists that can be used in the compositions described herein. As used herein, a “TLR7/8 agonist” refers to an agonist that affects its biological activities through its interaction with TLR7, TLR8, or both. Such biological activities include, but are not limited to, the induction of TLR7 and/or TLR8 mediated signal transduction to potentiate immune responses via the innate immune system. In some embodiments, the TLR is an imidazoquinoline amine derivative (see. e.g., U.S. Pat. No. 4,689,338 (Gerster)), but other compound classes are known as well (see, e.g., U.S. Pat. No. 5,446,153 (Lindstrom et al.); U.S. Pat. No. 6,194,425 (Gerster et al.); and U.S. Pat. No. 6,110,929 (Gerster et al.); and International Publication Number WO2005/079195 (Hays et al)).

Such TLR7/8 agonists are hydrophobic or relatively hydrophobic, and in the absence of a helper lipid as described herein. do not substantially form stable aqueous nanosuspensions when mixed with water, such as in the presence or absence of an input from a high energy source. In some embodiments, the TLR7/8 agonists of the disclosure contain nonpolar moieties such as hydrocarbon chains. In some embodiments, the TLR7/8 agonists are soluble in the organic solvents but have low solubility in water, or are insoluble in water. and have a tendency to array into large aggregates in aqueous solutions in the absence of a helper lipid, as described herein. In this context, “insoluble in water” refers to a compound that does not dissolve when the compound is mixed with water, for example, when mixed with water at a temperature of 25° C. to 50° C.; and “low solubility in water” refers to a compound that has a solubility in water of less than 30 mg/mL, for example, when mixed with water at a temperature of 25° C. to 50° C. As used herein. “poorly soluble in water” can be used to refer to compounds, for example, non-polar compounds. that are water insoluble or have low water solubility

In certain embodiments, the TLR7/8 agonist is a compound of the following structure of Formula (I):

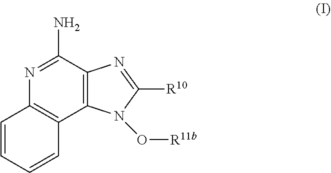

or a pharmaceutically acceptable salt thereof, wherein:
R¹⁰ is selected from the group consisting of hydrogen and C₁₋₆alkyl; and
R^{11b} is C₁₋₆alkyl optionally substituted with one or more groups selected from the group consisting of halo, hydroxyl, C₁₋₆alkoxy. and acylamino.

In some embodiments of Formula (I). R¹⁰ is hydrogen. In some embodiments of Formula (I), R¹⁰ is C₁₋₆alkyl. In some embodiments. R¹⁰ is methyl, ethyl, n-propyl, or n-butyl. In some embodiments. R¹⁰ is n-butyl.

In some embodiments of Formula (I), R^{11b} is C₂₋₄alkyl, which is substituted with acylamino. In some embodiments. R^{11b} is —(CH₂)₄-acylamino. In some embodiments, R^{11b} is —(CH₂)₄—NH—C(O)—C₁₋₂₅alkyl. In some embodiments. R^{11b} is —(CH₂)₄—NH—C(O)—C₁₅₋₂₅alkyl. In some embodiments, R^{11b} is —(CH₂)₄—NH—C(O)—C₁₅₋₂₀alkyl. In some embodiments, R^{11b} is —(CH₂)₄—NH—C(O—C₁₇alkyl.

In certain embodiments. the TLR7/8 agonist is a compound of the following structure or pharmaceutically acceptable salts thereof:

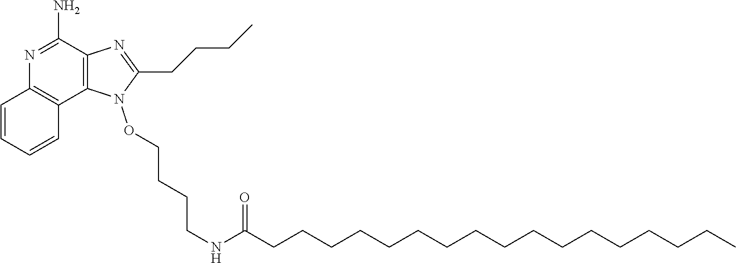 [SMILES: CCCCCCCCCCCCCCCCCC(=O)NCCCCOn1c(CCCC)nc2c(N)nc3ccccc3c21]

In certain embodiments. a TLR7/8 agonist used in the compositions herein comprises a N-(4-[4-amino-2-butyl-1H-imidazo[4,5-c]quinolin-1-yl]oxy}butyl)octadecanamide), 3M-052 as described in U.S. Pat. No. 9,242,980 (Wightman).

### Preparation of N-(4-{[4-amino-2-butyl-1H-imidazo[4,5-c]quinolin-1-yl]oxy}butyl)octadecanamide

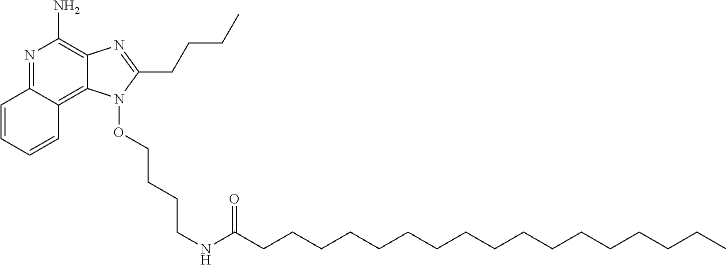 [SMILES: CCCCCCCCCCCCCCCCCC(=O)NCCCCOn1c(CCCC)nc2c(N)nc3ccccc3c21]

Unless otherwise noted, all parts, percentages, ratios, etc. in the examples and the rest of the specification are by weight, and all reagents used in the examples were obtained, or are available, from general chemical suppliers such as, for example, Sigma-Aldrich Company, Saint Louis, Missouri, or may be synthesized by conventional methods.

These abbreviations are used in the following examples: ppm=parts per million; phr=parts per hundred rubber; mL=milliliter; L=liter; g=grams, min=minutes, h=hour, C=degrees Celsius, MPa=megapascals, and N-m=Newton-meter.

### Part A

A solution of valeric anhydride (6.03 g) and pyridine hydrochloride (0.198 g) in pyridine (8.28 g) was added to a solution of 3-amino-4-chloroquinoline (2.94 g) in pyridine (5.0 g) and the reaction was stirred at room temperature for 16 hours followed by heating at 60° C. for 3 hours. The reaction was concentrated under reduced pressure and sodium carbonate (15 mL of a 10% aqueous solution) was added. The reaction was stirred for 30 minutes and then filtered. The resulting solid was washed with water (60 mL) and dried under vacuum for 4 hours to provide 4.59 g of crude N-(4-chloroquinolin-3-yl)valeramide as brown flakes. The crude product was recrystallized from heptane (10 mL) and the recovered product was further purified by soxhlet extraction using refluxing heptane for 16 hours. The collection flask from the soxhlet extraction apparatus was cooled in a freezer for 2 hours. The resulting solid was collected by filtration and dried under vacuum to yield 2.00 g of N-(4-chloroquinolin-3-yl)valeramide as a white solid.

### Part B

A solution of 4-amino-1-butanol (7.68 g) and pyridine (7.00 g) in dichloromethane (100 mL) was chilled in an ice bath and a solution of benzylchloroformate (14.37 g) in dichloromethane (100 mL) was slowly added with stirring over a period of thirty minutes. The ice bath was removed and the reaction was stirred for an additional 16 hours. Hydrochloric acid (1.2 M, 200 mL) was added and phases were separated. The organic phase was dried (MgSO₄), filtered and concentrated under reduced pressure. The resulting residue was recrystallized from toluene and dried under vacuum to provide 5.15 g of benzyl (4-hydroxybutyl)carbamate.

A solution of N-hydroxyphthalimide (3.36 g), benzyl (4-hydroxybutyl)carbamate (4.18 g) and triphenylphosphine (7.41 g) in dichloromethane (100 mL) was chilled in an ice bath and approximately two-thirds of a solution of diisopropylazodicarboxylate (DIAD, 5.68 g) in dichloromethane (50 mL) was slowly added with stirring. The internal temperature of the reaction was monitored and the addition of the DIAD solution was stopped when an exotherm could no longer be detected. The ice bath was removed and the reaction was allowed to warm to room temperature. The reaction was concentrated under reduced pressure and the resulting residue was dissolved in ethanol (200 proof, 100 mL). Hydrazine (1.98 g, 35% in water) was added and the reaction was stirred for 6 hours. The reaction was cooled in the freezer and the resulting solid was removed by filtration. The solid was washed with ethanol (50 mL). The combined filtrate was concentrated under reduced pressure and diethyl ether (100 mL) was added. Insoluble impurities were removed by filtration and 2.0 M HCl in ether (10 mL) was added to the solution. A precipitate formed immediately. The crude product was added to toluene (100 mL) and heated at reflux temperature for one hour. After cooling to room temperature, the solid product was recovered by filtration, washed with toluene, and dried under vacuum to yield 3.76 g of benzyl (4-aminooxybutyl)carbamate.

### Part C

N-(4-chloroquinolin-3-yl)valeramide (1.97 g), benzyl (4-aminooxybutyl)carbamate (2.99 g), triethylamine (0.89 g) and 2-propanol (40.69 g) were combined and heated at 80° C. for 3.5 hours. The reaction was cooled to room temperature, filtered, and the filtrate concentrated under reduced pressure. Dichloromethane (20 mL) was added to the resulting solid and the mixture was stirred for twenty minutes. Undissolved solid was removed by filtration and the filtrate was washed with two 10 mL portions of water that had been made slightly acidic by the addition of 20 drops of hydrochloric acid (1.2 M).

The organic fraction was dried and concentrated under reduced pressure. The crude solid was recrystallized from tetrahydrofuran to provide 2.56 g of benzyl 4-{[2-butyl-1H-imidazo[4,5-c]quinolin-1-yl]oxy}butylcarbamate.

### Part D

Benzyl 4-{[2-butyl-1H-imidazo[4,5-c]quinolin-1-yl]oxy}butylcarbamate hydrochloride (10.05 g) was dissolved in dichloromethane (80 mL) and extracted with a solution of sodium carbonate (2.02 g) in 30 ml H₂O. The organic layer was cooled in an ice bath and a solution of m-chloroperbenzoic acid (5.93 g, 1.24 eq) dissolved in dichloromethane (30 mL) was slowly added. After 6 hr, ammonium hydroxide (10 mL of a 28-30% aqueous solution) was added to the reaction. A solution of benzenesulfonyl chloride (6.96 g) dissolved in 10 ml dichloromethane was slowly added with vigorous stirring. The cooling bath was removed and the reaction was stirred for an additional 12 hours. The reaction was diluted with water (100 mL) and the organic and aqueous fractions were separated. The aqueous fraction was extracted with dichloromethane (30 mL). The combined organic fractions were washed with two 90 ml portions of 5% sodium carbonate.

The dichloromethane solution was transferred to a distillation apparatus and 1-pentanol (50 mL) was added. This was warmed to 40° C. and the dichloromethane was removed under reduced pressure. Concentrated hydrochloric acid (50 ml) was then added and the reaction was stirred and heated to 80°. After 11 hours, the solution was cooled to room temperature and diluted with water (100 mL). The aqueous fraction was separated from the 1-pentanol and the 1-pentanol was extracted with water (25 mL). The aqueous fractions were combined. 1-Pentanol (50 mL) was added to the combined aqueous fraction and this was cooled in an ice-bath. With vigorous stirring, solid sodium carbonate was added to bring the pH to 9-10. The mixture was transferred to a separatory funnel and the fractions were separated. The aqueous fraction was extracted with two 25 ml portions of 1-pentanol. The combined 1-pentanol fractions were dried over sodium sulfate and filtered to provide 1-(4-aminobutoxy)-2-butyl-1H-imidazo[4,5-c]quinolin-4-amine dissolved in 1-pentanol.

The maleate salt of 1-(4-aminobutoxy)-2-butyl-1H-imidazo[4,5-c]quinolin-4-amine was prepared by dissolving maleic acid (4.83 g) in 1-pentanol (50 mL) and adding it with stirring to the solution of 1-(4-aminobutoxy)-2-butyl-1H-imidazo[4,5-c]quinolin-4-amine in 1-pentanol. The resulting precipitate was collected by filtration and dried to yield 7.69 g of 1-(4-aminobutoxy)-2-butyl-1H-imidazo[4,5-c]quinolin-4-amine bis maleate salt. ¹H-NMR (DMSO-d6): δ 0.96 (t, 3H), 1.44 (m, 2H), 1.7-1.95 (m, 4H), 2.02 (m, 2H), 2.8-3.1 (m, 4H), δ 4.43 (t, 2H), 6.07 (s, 4H), 7.57 (t, 1H), 7.73 (t, 1H), 7.80 (d, 1H), 8.16 (d, 1H). Broad peaks for the ammonium protons are seen at approximately δ 7.8 and δ 8.7.

As an alternative the fumarate salt of 1-(4-aminobutoxy)-2-butyl-1H-imidazo[4,5-c]quinolin-4-amine was prepared by the following procedure. 1-(4-aminobutoxy)-2-butyl-1H-imidazo[4,5-c]quinolin-4-amine (6.53 g) was dissolved in 2-propanol (75 mL) and decolorizing carbon was added. The reaction was heated to reflux, filtered while hot, and cooled to room temperature. A solution of fumaric acid (2.5 g) in 2-propanol was added and the reaction was heated at reflux temperature for 5 minutes. Upon cooling to room temperature a precipitate formed. Filtration followed by drying the product under vacuum yielded 6.6 g of 1-(4-aminobutoxy)-2-butyl-1H-imidazo[4,5-c]quinolin-4-amine fumarate.

¹H-NMR (DMSO-d6): δ 0.95 (t, 3H), 1.42 (m, 2H), 1.70-1.92 (m, 4H), 1.92-2.10 (m, 2H), 2.85-3.05 (m, 4H), 4.34 (t, 3H), δ 6.46 (s, 2H), 7.30 (t, 1H), 7.47 (t, 1H), 7.60 (d, 1H), 8.02 (d, 1H). A broad ammonium peak appears at δ 6.77.

### Part E

1-(4-Aminobutoxy)-2-butyl-1H-imidazo[4,5-c]quinolin-4-amine fumarate (1.30 g) was dissolved in dichloromethane (25 mL) and the solution washed with 3×15 ml portions of saturated sodium carbonate. The organic fraction was then washed with 15 ml saturated sodium chloride and dried over MgSO₄. The solution was filtered, the solvent removed under reduced pressure and the product was dried under vacuum to give 0.79 g of 1-(4-aminobutoxy)-2-butyl-1H-imidazo[4,5-c]quinolin-4-amine as the free base.

The 1-(4-aminobutoxy)-2-butyl-1H-imidazo[4,5-c]quinolin-4-amine was dissolved in dichloromethane (20 mL) and methanol (5 mL). Stearic acid (0.71 g) was added and the reaction was stirred to dissolve the stearic acid. 1-Ethyl-3-(3-dimethylaminopropyl) carbodiimide HCl (EDC, 0.45 g) was added and the reaction was stirred at ambient temperature for 16 hours. An additional portion of EDC was added (0.23 g) and the reaction was stirred for an additional 24 hours. Final portions of stearic acid (0.22 g) and EDC (0.37 g) were added to drive the reaction to completion and the reaction was stirred at ambient temperature for another 24 hours. The reaction was concentrated under reduced pressure and the resulting residue was purified by flash column chromatography using a Biotage chromatography system (Si40+M2358-1 SiGel column, 85:15 dichloromethane/methanol isocratic elution). The semi-pure product was purified by flash column chromatography two more times using a 90:10 dichloromethane/methanol isocratic elution, followed by a 95:5 dichloromethane/methanol isocratic elution The fractions containing product were concentrated to yield 1.12 g of N-(4-{[4-amino-2-butyl-1H-imidazo[4,5-c]quinolin-1-yl]oxy}butyl)octadecanamide as an off white waxy solid.

¹H-NMR (CDCl₃): δ 0.89 (t, 3H), 1.01 (t, 3H), 1.14-1.42 (m, 28H), 1.50 (m, 2H), 1.65 (m, 2H), 1.74-1.94 (m, 4H), 2.02 (m, 2H), 2.20 (t, 2H), 2.95 (t, 2H), 3.40 (q, 2H), 4.33 (t, 2H), 5.59 (t, 1H), 6.10 (broad s, 2H), 7.39 (m, 1H), δ 7.57 (m, 1H), 7.83 (d, 1H), 8.07 (m, 1H).

### Aqueous Formulation of TLR7/8 Agonist and Helper Lipid

In certain embodiments, a TLR718 agonist and a helper lipid are combined and subjected to a high energy source to produce an aqueous formulation, for example, a nanosuspension composition. In the certain embodiments. the aqueous formulation includes nanosuspension particles of TLR7/8 agonist and helper lipid having a particle size of at least 1 nm, at least 10 nm, at least 20 nm, at least 30 nm, at least 40 nm. or at least 50 nm. In the certain embodiments, the aqueous formulation includes nanosuspension particles of TLR7/8 agonist and helper lipid having a particle size of up to 450 nm, up to 400 nm, up to 350 nm, up to 300 nm, up to 250 nm, up to 200 nm, up to 150 nm, up to 100 nm, or up to 75 nm

In the certain embodiments. the aqueous formulation includes nanosuspension particles of TLR7/8 agonist and helper lipid that range in size from 1 nm to 450 nm. such as 1 to 400 nm, 1 to 200 nm, 50 to 200 nm, 50 to 150 nm, 50 to 100 nm, 50 nm to 75 nm. In some embodiments, the size of the nanosuspension particles range from 20 to 100 nm or 20 to 50 nm. In some embodiments, the size of the nanosuspension particles range from 10 to 200 nm, 10 to 100 nm, or 10 to 50 nm.

In some embodiments, the nanosuspension particles can be filtered through at least a 0 45 micron filter. In some embodiments, the nanosuspension particles can be filtered through a 0.45 micron or smaller pore size filter. In some embodiments, the nanosuspension particles can be filtered through a 0.45 micron filter. In some embodiments, the nanosuspension particles can be filtered through a 0 20 micron filter. In some embodiments, the nanosuspension particles can be filtered through a 0.22 micron filter.

In some embodiments provided herein, the 1-450 nm size of the aqueous nanosuspension particles that include a TLR7/8 agonist and a helper lipid is stable. In this context. “stable” can refer to a nanosuspension particle's size of less than 450 nm is maintained, and the particles exhibit reduced aggregation, or no aggregation, when compared to a TLR7/8 agonist in the absence of a helper lipid of the disclosure.

In some embodiments, “stable” refers to a formulation or composition comprised of nanosuspension particles which display little to no aggregation, or reduced aggregation, or demonstrate little to no overall increase in average particle size or polydispersity of the formulation over time compared to the initial particle size.

The stability of the nanosuspension particles can be measured by techniques familiar to those of skill in the art. In some embodiments, the stability is observed visually.

Visual inspection can include inspection for particulates, flocculence, or aggregates. In some embodiments, the stability is determined by the size of the nanosuspension particles. For example, the size can be assessed by known techniques in the art, including but not limited to, x-ray and laser diffraction, dynamic light scattering (DLS), CryoEM, or Malvern Zetasize. In some embodiments, the size of the nanosuspension particles refers to the Z-average diameter. In some embodiments, the stability is assessed by the ability of the nanosuspension particles to pass through a filter of a particular size. for example through a 0.20, 0.22 or 0.45 micron filter. In some embodiments. stability is determined by pH. In some embodiments, stability is determined by measurement of the polydispersity index (PdI), for example, with the use of the dynamic light scattering (DLS) technique.

In some embodiments, the Z-average diameter of the nanosuspension particles increase by less than 50%, less than 40%, less than 30%, less than 25%, less than 20%, less than 15%, less than 12%, less than 10%, less than 7%, less than 5%, less than 3%, or less than 1%. over the time period assayed.

In some embodiments, the nanosuspension particles are stable at 0-8° C. such as 2-8° C. In some embodiments, the nanosuspension particles are stable at 0° C., 1° C. 2° C., 3° C., 4° C., 5° C., 6° C., 7° C., or 8° C., for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours. for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months. for at least 7 months. for at least 8 months, for at least 9 months, for at least 10 months. for at least 11 months. for at least 1 year, for at least 2 years. or for at least 5 years.

In some embodiments, the nanosuspension particles are stable at 20-30° C. In some embodiments. the nanosuspension particle is stable at 25° C. for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours. for at least 18 hours, for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years.

In some embodiments, the nanosuspension particles are stable at 35-40° C. In some embodiments, the nanosuspension particles are stable at 35° C. 36° C., 37° C., 38° C., 39° C., or 40° C. for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours. for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years.

In some embodiments, the nanosuspension particle is stable at 57-62° C. In some embodiments, the nanosuspension particles are stable at 57° C., 58° C. 59° C., 60° C., 61° C., or 62° C. for at least 1 minute. for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours. for at least 24 hours, for at least 48 hours, for at least 72 hours. for at least 1 week, for at least 2 weeks, for at least 3 weeks, for at least 1 month.

In some embodiments, the nanosuspension particles are stable after 1-4 freeze thaws. In some embodiments, the nanosuspension particles are stable after 1, after 2, after 3, or after 4 freeze thaws.

### Helper Lipids

The helper lipids form stable aqueous compositions, such as nanosuspensions, that include a TLR7/8 agonist described herein when mixed with water, for example in the presence or absence of an input from a high energy source. In some embodiments, the TLR7/8 agonists of the disclosure contain nonpolar moieties such as hydrocarbon chains. The helper lipid aids in the adsorption of the TLR7/8 agonist to, for example, an aluminum salt.

In certain embodiments, the helper lipid is a phospholipid or a quaternary ammonium salt lipid. In certain embodiments, the helper lipid is a phospholipid that is a phosphatidylcholine or a phosphoglyceride. In certain embodiments. the helper lipid includes any of the following moieties.

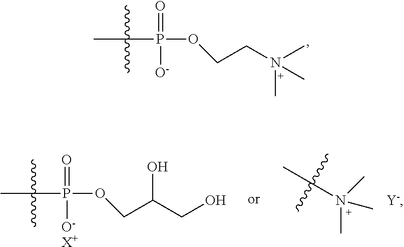

wherein X⁻ is an alkali metal counterion and Y⁺ is a halide counterion.

In certain embodiments. the helper lipid includes a C₁₀₋₂₀alkyl chain. In certain embodiments, the helper lipid includes a C₁₂₋₁₈alkyl chain.

In certain embodiments, the helper lipid is anionic. In certain embodiments. the helper lipid is cationic. In certain embodiments, the helper lipid is overall neutrally charged. In certain embodiments, the helper lipid is a zwitterion.

In certain embodiments, suitable helper lipids are shown herein.

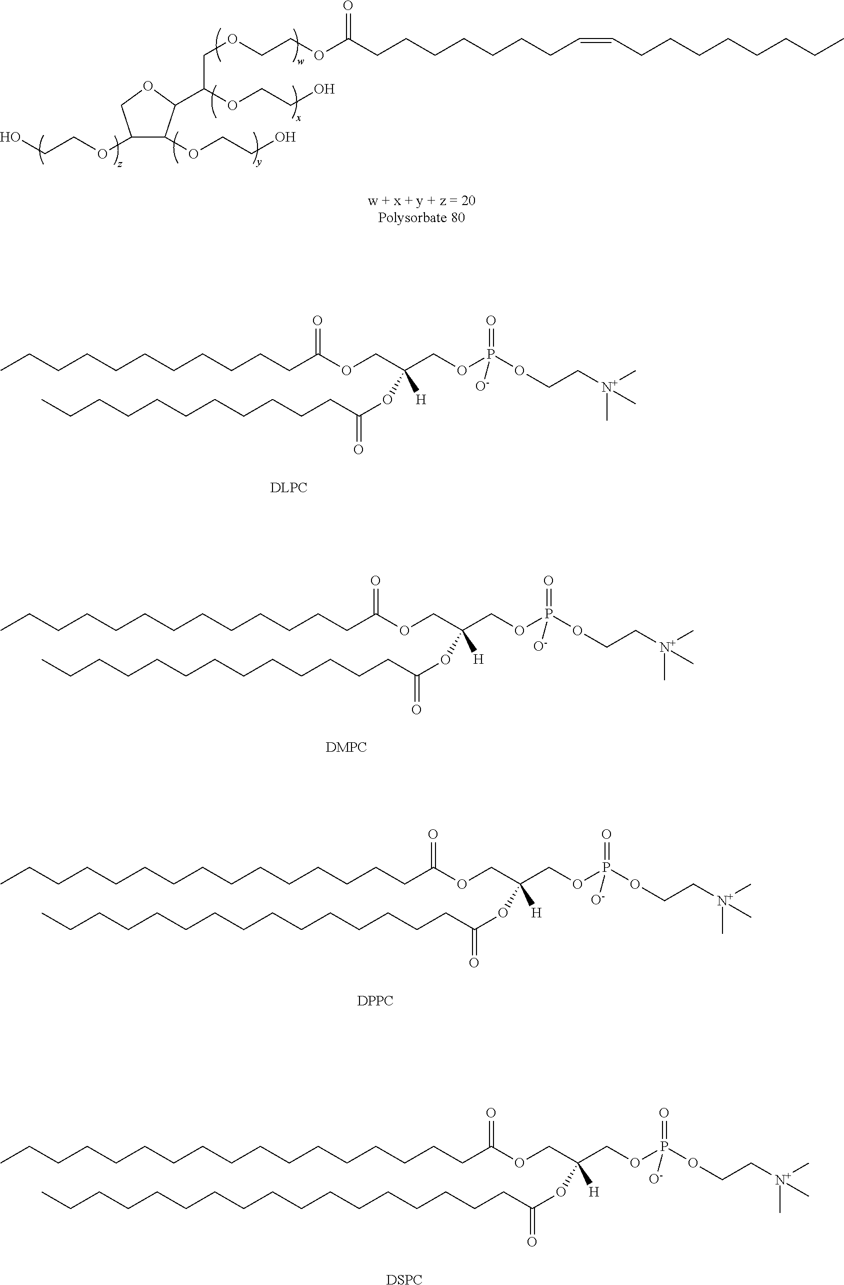

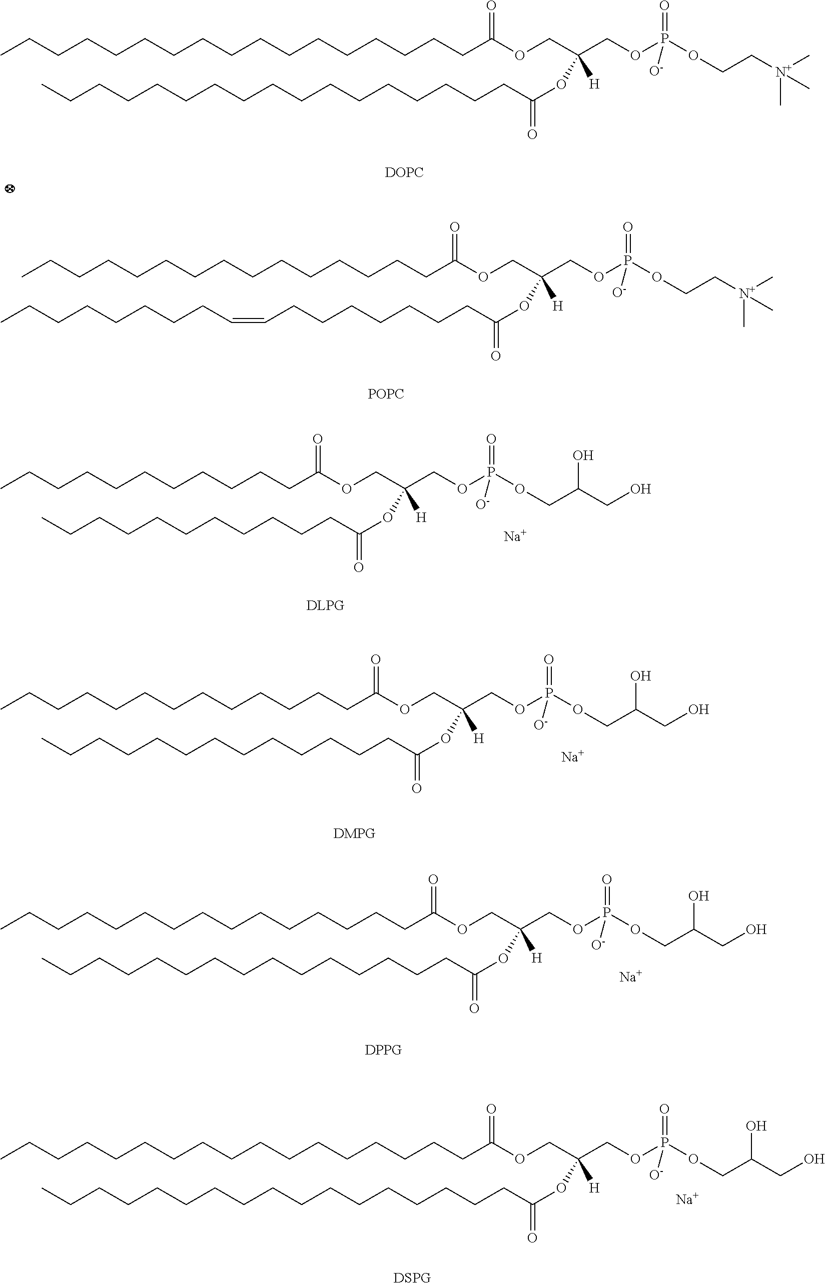

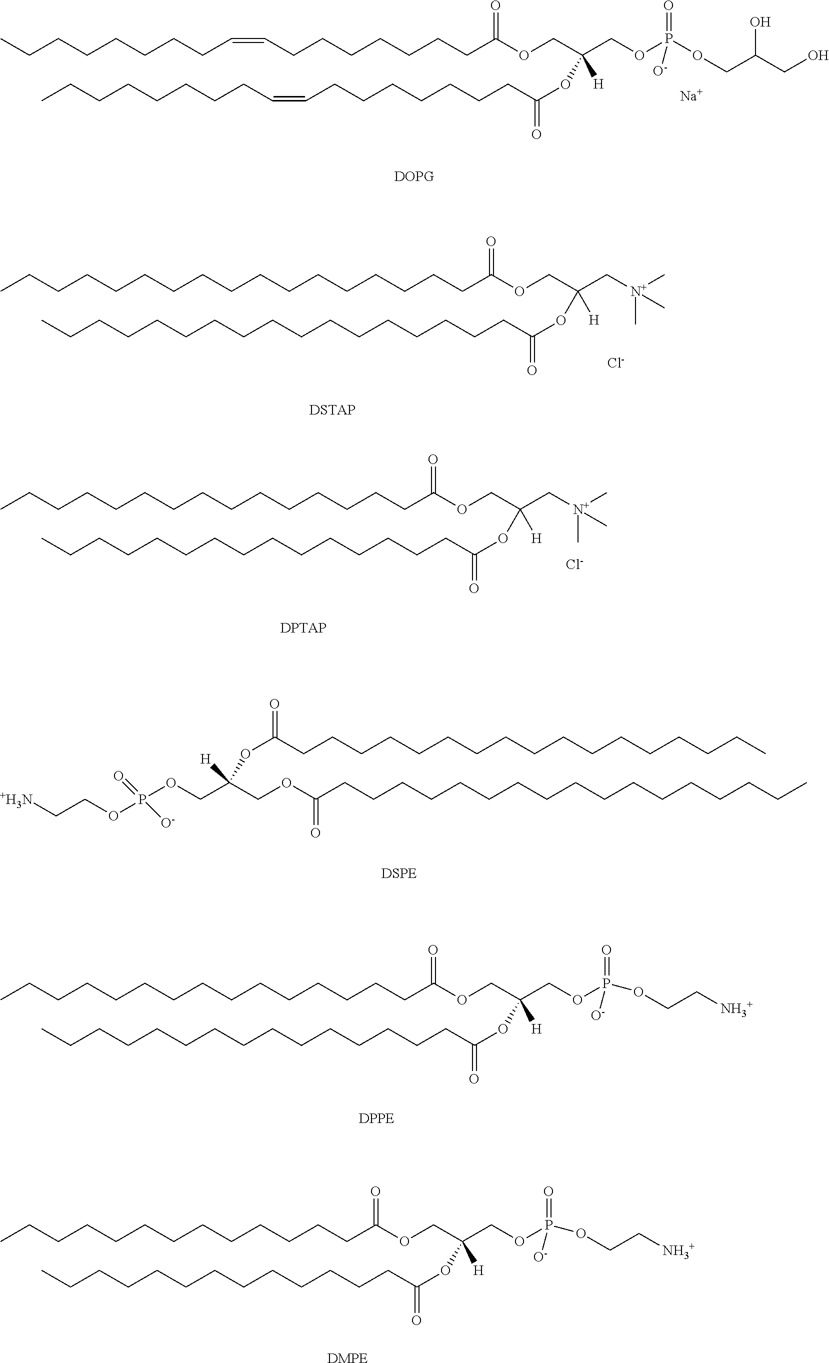

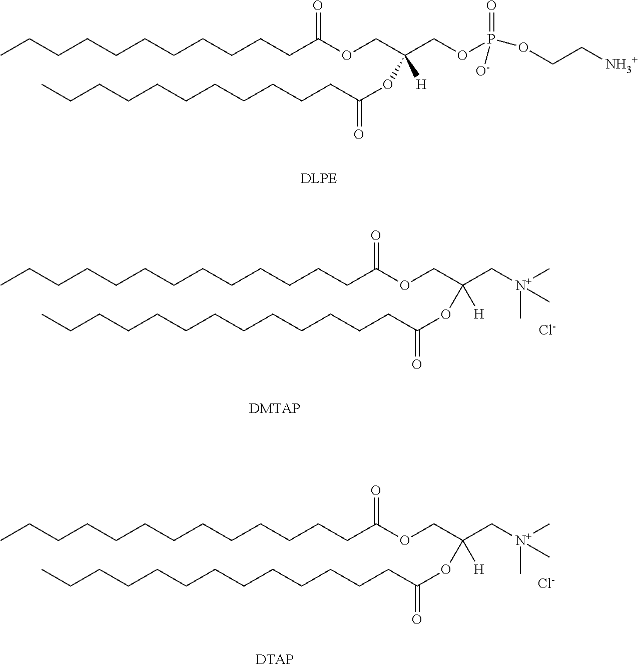

In certain embodiments, the helper lipid is selected from DLPG, DMPG, DPPG, DSPG, DOPG, DSTAP, and DVTAP In certain embodiments. the helper lipid is selected from DLPG, DMPG, DPPG, DSPG, DOPG, and DSTAP In certain embodiments. the helper lipid is selected from DLPG, DMPG, DPPG, DSPG, and DOPG.

In certain embodiments, the helper lipid is selected from DOPC, DSPG, DSTAP, and Polysorbate 80. In certain embodiments. the helper lipid is selected from DSPG and DSTAP. In certain embodiments, the helper lipid is DSPG. In certain embodiments, the helper lipid is DSTAP.

### Combination of TLR7/8 Agonist and Aluminum Salt

The compositions described herein may also include an aluminum salt, which can be referred to herein as alum. Suitable aluminum salts include aluminum hydroxide, aluminum trihydrate, aluminum oxyhydroxide, aluminum phosphate, aluminum hydroxyphosphate. aluminum hydroxyphosphate sulfate, and potassium aluminum sulfate. Aluminum salts can also be referred to by the formulae: Al(OH)₃, AlH₃O₃, AlH₆O₃, AlO(OH), Al(OH)(PO₄), and KAl(SO₄)₂. Aluminum salts used as co-adjuvants are advantageous because they have a good safety record, augment antibody responses, stabilize antigens, and are relatively simple for large-scale production. (Edelman 2002 Mol. Biotechnol. 21:129-148; Edelman, R. 1980 Rev. Infect. Dis. 2:370-383.)

In certain embodiments, a stable aqueous formulation of adjuvant including a TLR7/8 agonist with a helper lipid that are adsorbed to an aluminum salt is provided. In certain embodiments, the helper lipid is selected from DOPC, DSPG, DSTAP, and Polysorbate 80, and in certain embodiments, the helper lipid is selected from DSPG and DSTAP.

The TLR7/8 agonist is associated with, for example, adsorbed onto, the aluminum salt with the aid of the helper lipid. The factors that relate to the selection of each of the components include, but are not limited to, the charges of the components and presence of exchangeable ligands With proper selection of the components, the TLR7/8 agonist and helper lipid can be associated with, such as adsorbed to, the aluminum salt. In certain embodiments, the association, such as adsorption, occurs with in vitro conditions.

Association or adsorption refers to an interaction between molecules or portions thereof that exhibit mutual affinity or binding capacity, for example due to specific or non-specific binding or interaction, including, but not limited to, biochemical. physiological, and/or chemical interactions. In certain embodiments, such association. for example adsorption, can be determined by UV spectroscopy, SDS-PAGE, or centrifugation studies.

In some embodiments, at least 25%, at least 40%, at least 50%, at least 65%, at least 70%, at least 75%, at least 80%. at least 85%, at least 90%, at least 95%. at least 97%, at least 99%, of the TLR7/8 agonist with helper lipid present in the composition is associated with aluminum salt particles.

A skilled artisan can readily determine the dose and number of immunizations needed to induce immune response. Various assays are known and used in the art to measure to level, breadth and durability of the induced immune response. In non-limiting embodiments the methods comprise two immunizations. The interval between immunizations could be readily determined by a skilled artisan. In non-limiting embodiments, the first and second immunization are about 2, 3, 4, 5, 6, 7, 8, 9, 10, 11, 12, 13, 14, 15, 16 weeks apart.

In certain embodiments the protein dose is in the range of 1-1000 micrograms.

In certain embodiments the protein dose is in the range of 10-1000 micrograms. In certain embodiments the protein dose is in the range of 100-1000 micrograms. In certain embodiments the protein dose is in the range of 100-200 micrograms. In certain embodiments the protein dose is in the range of 100-300 micrograms. In certain embodiments the protein dose is in the range of 100-400 micrograms. In certain embodiments the protein dose is in the range of 100-500 micrograms. In certain embodiments the protein dose is in the range of 100-600 micrograms. In certain embodiments the protein dose is in the range of 50-100 micrograms. In certain embodiments the protein dose is in the range of 50-150 micrograms. In certain embodiments the protein dose is in the range of 50-200 micrograms. In certain embodiments the protein dose is in the range of 50-250 micrograms. In certain embodiments the protein dose is in the range of 50-300 micrograms. In certain embodiments the protein dose is in the range of 50-350 micrograms. In certain embodiments the protein dose is in the range of 50-400 micrograms. In certain embodiments the protein dose is in the range of 50-450 micrograms. In certain embodiments the protein dose is in the range of 50-500 micrograms. In certain embodiments the protein dose is in the range of 50-550 micrograms. In certain embodiments the protein dose is in the range of 50-600 micrograms. In certain embodiments the protein dose is in the range of 75-100 micrograms. In certain embodiments the protein dose is in the range of 75-125 micrograms. In certain embodiments the protein dose is in the range of 75-150 micrograms. In certain embodiments the protein dose is in the range of 75-175 micrograms. In certain embodiments the protein dose is in the range of 75-200 micrograms. In certain embodiments the protein dose is in the range of 75-225 micrograms. In certain embodiments the protein dose is in the range of 75-250 micrograms. In certain embodiments the protein dose is 10, 25, 50, 75, 100, 125, 150, 175, 200, 225, 250, 275, 300, 325, 350, 375, 400, 425, 450, 475, 500, 525 550, 575, 600, 625, 650, 700, 750, 800, 850, 900, 950 or 1000 micrograms.

In certain embodiments adjuvant dose is in the range of 1-200 micrograms. In certain embodiments adjuvant dose is in the range of 1-100 micrograms. In certain embodiments the adjuvant dose is 1-50 micrograms. In certain embodiments the adjuvant dose is 1-25 micrograms. In certain embodiments the adjuvant dose is 1-50 micrograms. In certain embodiments the adjuvant dose is 1-20 micrograms. In certain embodiments the adjuvant dose is 1-50 micrograms. In certain embodiments the adjuvant dose is 1-15 micrograms. In certain embodiments the adjuvant dose is 1-50 micrograms. In certain embodiments the adjuvant dose is 1-10 micrograms. In certain embodiments the adjuvant dose is 1-5 micrograms. In certain embodiments the adjuvant dose is 5-10 micrograms. In certain embodiments the adjuvant dose is 5-15 micrograms. In certain embodiments the adjuvant dose is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 45-50 micrograms.

### EXAMPLES

### Example 1A: SARS-CoV-2 Vaccination Induces Neutralizing Antibodies Against Pandemic and Pre-Emergent SARS-Related Coronaviruses in Monkeys

Betacoronaviruses (betaCoVs) caused the severe acute respiratory syndrome (SARS) and Middle East Respiratory Syndrome (MERS) outbreaks, and now the SARS-CoV-2 pandemic¹⁻⁴. Vaccines that elicit protective immune responses against SARS-CoV-2 and betaCoVs circulating in animals have the potential to prevent future betaCoV pandemics. Here, we show that immunization of macaques with a multimeric SARS-CoV-2 receptor binding domain (RBD) nanoparticle adjuvanted with 3M-052-Alum elicited cross-neutralizing antibody responses against batCoVs, SARS-CoV-1, SARS-CoV-2, and SARS-CoV-2 variants B.1.1.7, P.1, and B.1.351. Nanoparticle vaccination resulted in a SARS-CoV-2 reciprocal geometric mean neutralization IC50 titer of 47,216, and protection against SARS-CoV-2 in macaque upper and lower respiratory tracts. Importantly, nucleoside-modified mRNA encoding a stabilized transmembrane spike or monomeric RBD also induced SARS-CoV-1 and batCoV cross-neutralizing antibodies, albeit at lower titers. These results demonstrate current mRNA vaccines may provide some protection from future zoonotic betaCoV outbreaks, and provide a platform for further development of pan-betaCoV vaccines.

The emergence of three betacoronavirus (BetaCoV) outbreaks over the last 18 years necessitates the development of countermeasures that can prevent future pandemics¹⁻⁴ SARS-CoV-1 and SARS-CoV-2 (betaCoV group 2b) and MERS (betaCoV group 2c) emerged from cross-species transmission events where humans were infected with bat or camel CoVs⁵⁻⁹. For example, betaCoVs that are genetically similar to SARS-CoV-1 and SARS-CoV-2 and bind to the human ACE2 receptor for virus entry circulate in civets, bats, and Malayan pangolins^{5,6,10-12}. Thus, SARS-related animal coronaviruses represent betaCoVs that can be transmitted to humans. Neutralizing antibodies can prevent or treat betaCoV infection and represent potential countermeasures against current human betaCoVs and pre-emergent viruses¹³⁻²². Cross-neutralizing antibodies that can neutralize multiple betaCoVs, have been isolated from SARS-CoV-1 infected humans^{13,23,24} validating the development of betaCoV vaccines against group 2b Sarbecoviruses²⁵. A critical target of cross-neutralizing antibodies is the receptor binding domain (RBD)^{22,23,25}. RBD immunogenicity can be augmented by arraying multiple copies on nanoparticles, mimicking virus-like particles²⁶⁻³⁰. Vaccine induction of cross-neutralizing antibodies has been reported in in vitro neutralization assays against CoV pseudoviruses in mice^{29,30}. However, it is unknown whether spike vaccination of primates can elicit cross-neutralizing betaCoV antibodies against SARS-CoV-1, bat betaCoVs, or against SARS-CoV-2 escape viruses. Here, we demonstrate the ability of a SARS-CoV-2 RBD 24-mer subunit nanoparticle vaccine-produced with an easily modifiable sortase-ferritin platform³¹—to elicit in monkeys potent cross-neutralizing antibodies against SARS-CoV-1, SARS-CoV-2, SARS-CoV-2 variants B.1.1.7, P.1, and B.1.351, as well as SARS-related bat betaCoVs. RBD nanoparticle vaccination protected monkeys against SARS-CoV-2 after respiratory challenge. Lastly, we show lipid nanoparticle-encapsulated Spike mRNA vaccines similar to those in clinical use elicit cross-neutralizing CoV antibodies, albeit at lower titers.

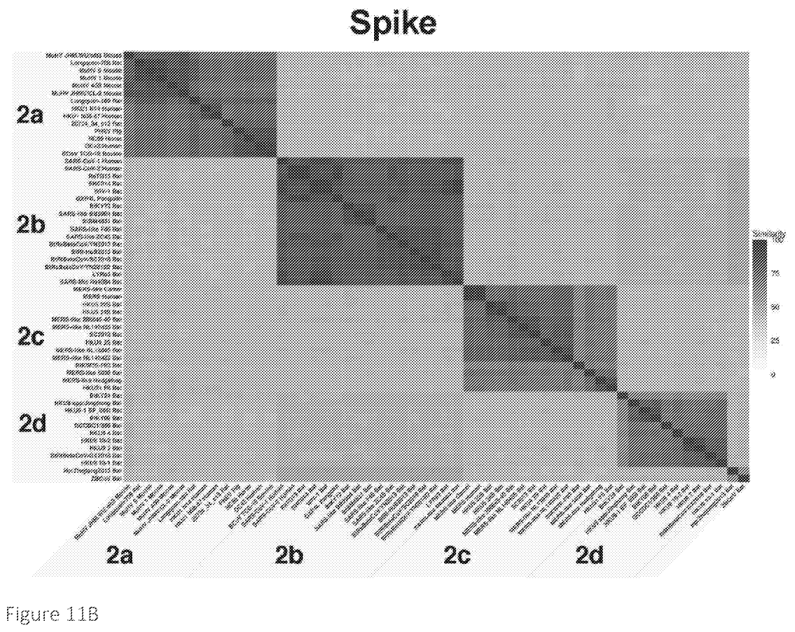

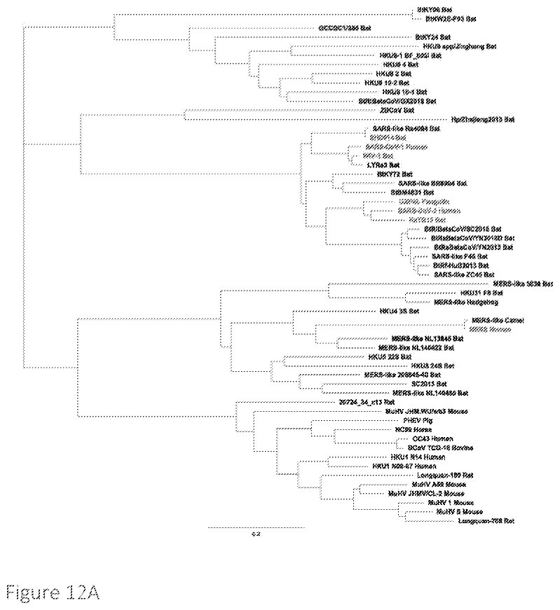

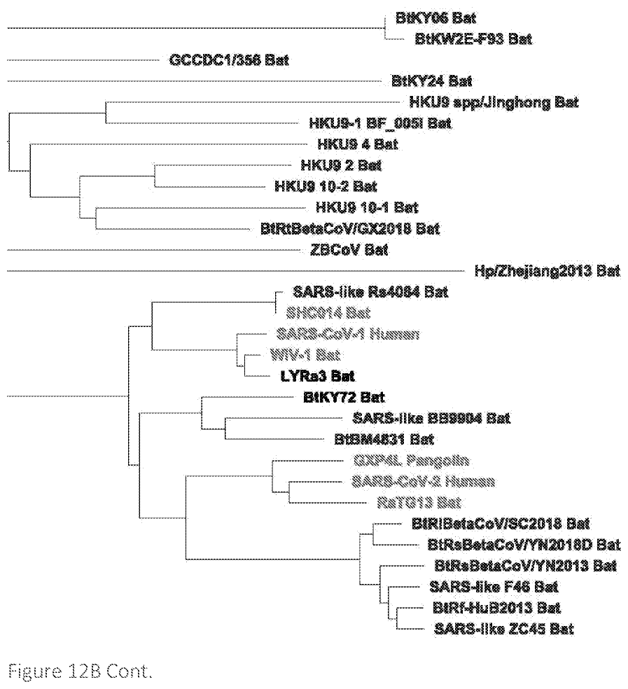

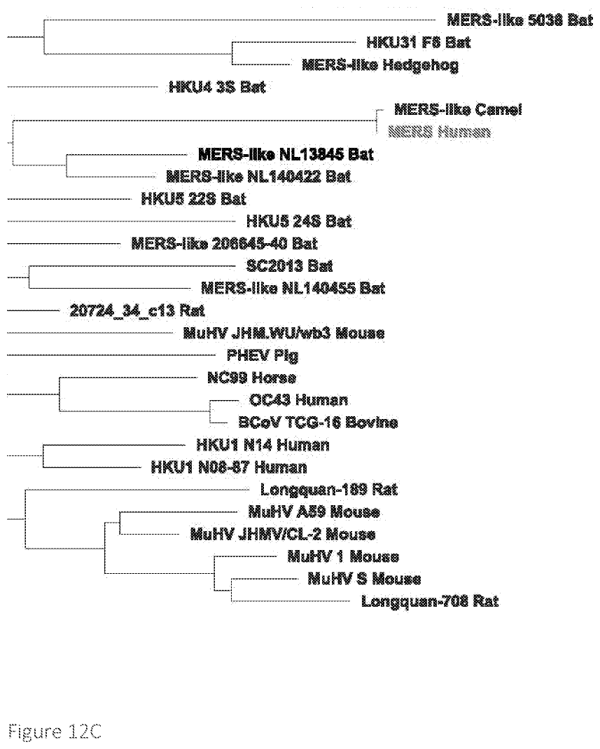

We and others have demonstrated the SARS-CoV-2 RBD has an epitope to which broadly cross-reactive neutralizing antibodies can bind^{13,24,32}. The DH1047 RBD antibody cross-neutralizes SARS-CoV-1, CoV-2 and bat CoVs²⁴. To focus the immune response on cross-reactive neutralizing epitopes on betaCoVs, we designed a 24-mer SARS-CoV-2 RBD-ferritin nanoparticle vaccine. The RBD nanoparticle was constructed by first expressing recombinant SARS-CoV-2 RBD with a C-terminal sortase A donor sequence. Next, we expressed the 24-subunit, self-assembling protein nanoparticle Helicobacter pylori ferritin with an N-terminal sortase A acceptor sequence³¹. The RBD and H. pylori ferritin nanoparticle were conjugated together by a sortase A reaction (FIG. 1a, FIG. 5)³¹. Analytical size exclusion chromatography and negative stain electron microscopy confirmed that RBD was conjugated to the surface of the ferritin nanoparticle (FIG. 1a, FIG. 5b,c). The RBD sortase A conjugated nanoparticle (RBD-scNP) bound to human ACE2, the receptor for SARS-CoV-2, and to potently neutralizing RBD antibodies DH1041, DH1042, DH1043, DH1044, and DH1045²⁴ (FIG. 1b). The epitopes of these antibodies are focused on the receptor binding motif within the RBD²⁴. Cross-neutralizing antibody DH1047 also bound to the RBD-scNP (FIG. 1b). The RBD scNP lacked binding to SARS-CoV-2 spike antibodies that bound outside of the RBD (FIG. 1b).

To assess immunogenicity of the RBD-scNP, we immunized five cynomolgus macaques three times intramuscularly four weeks apart with 100 μg of RBD-scNP (FIG. 1c and FIG. 6a). Immunogenicity of the RBD-scNP was adjuvanted with 5 μg of the TLR7/8 agonist 3M-052 absorbed to 500 μg of alum³³. Immunizations were well-tolerated in macaques (FIG. 7). After a single immunization, all five macaques generated binding IgG antibodies against SARS-CoV-2 RBD and stabilized Spike ectodomain (S-2P) (FIG. 1d). 3M-052-alum enhanced RBD-scNP immunogenicity, but did not elicit SARS-CoV-2 RBD antibodies on its own (FIG. 8a,b). Boosting once maximally increased plasma SARS-CoV-2 RBD and S-2P-specific IgG antibody titers (FIG. 1d). ACE2 competitive binding assays demonstrated the presence of antibodies against the ACE2 binding site within the receptor binding domain. Plasma antibodies blocked the ACE2 binding site on SARS-CoV-2 S-2P by 52% after one immunization and blocked 100% after two immunizations (FIG. 1e). Similarly, plasma antibodies blocked the binding of ACE2-binding site-focused, RBD neutralizing antibody DH1041, although to a lesser degree than ACE2 (FIG. 1e). Vaccine induction of neutralizing antibodies was assessed against a SARS-CoV-2 pseudovirus with an aspartic acid to glycine substitution at position 614 (D614G)³⁴. Two RBD scNP immunizations induced potent serum neutralizing antibodies (FIG. 1f-h). The fifty percent inhibitory reciprocal serum dilution (ID50) neutralization titers ranged from 21,292 to 162,603 (FIG. 1g). We compared these neutralizing titers to cynomolgus macaques immunized twice with 50 μg of a lipid-encapsulated nucleoside-modified mRNA (mRNA-LNP) encoding stabilized transmembrane (TM) Spike (S-2P) that is analogous to the immunogen and vaccine platform in the COVID-19 vaccines authorized for emergency use (FIG. 6b). In cynomolgus macaques, serum neutralization titers against SARS-CoV-2 D614G pseudovirus elicited by RBD-scNP immunization were significantly higher than titers elicited by two S-2P mRNA-LNP immunizations (FIG. 1i, P<0.01 Exact Wilcoxon test, n=5)^{35,36}. The group geometric mean ID50, measured as reciprocal serum dilution, for the macaques immunized with RBD-ScNP was 47,216 compared to 6,469 for mRNA-LNP immunized macaques. When compared to natural infection, RBD-scNP vaccination elicited ID50 neutralization titers higher than those elicited in humans with SARS-CoV-2 symptomatic infection, asymptomatic infection, or infection requiring hospitalization (FIG. 1j). Thus, RBD-scNP adjuvanted with 3M-052-Alum elicits significantly higher neutralizing titers in macaques compared to current vaccine platforms or to natural infection in humans.

The SARS-CoV-2 variant B.1.1.7 is widespread in the United Kingdom (UK) and is spreading globally^{37,38}. Moreover, the B.1.1.7 variant can have higher infectivity and has mutations in the receptor binding domain that may limit neutralization efficacy of RBD-specific antibodies^{37,38}. Additionally, SARS-CoV-2 variants B1.351 and P.1 share the N501Y mutation with B.1.1.7, and B1.351 lineage viruses have become the dominant strain in the Republic of South Africa³⁸⁻⁴⁰. P.1 and B.1.351 are of concern due to their neutralization-resistant phenotype and mutations in the RBD at K417N, E484K, and N501Y⁴¹. Examination of the cryo-electron microscopy structures of DH1041 or DH1047 bound to S trimer showed that the E484K mutation was present in the binding surface of RBD nAb DH1041, but was distal to the cross-neutralizing nAb DH1047 binding site (FIG. 2a,b). None of the three RBD mutations present in B.1.351, were within the DH1047 binding surface because the DH1047 uses a long 24 amino acid HCDR3 to reach down and contact the RBD without the rest of the antibody contacting N501 or K417 (FIG. 2b). Consistent with these structures, DH1041 binding to wild-type SARS-CoV-2 RBD was knocked out by the E484K mutation (FIG. 2c and FIG. 8), however, DH1047 binding to RBD was unaffected by K417N, E484K, or N501Y (FIG. 2d).

To determine whether RBD-scNP or mRNA-LNP immunization elicited antibodies can neutralize SARS-CoV-2 variants B.1.1.7, B.1.351, or P.1, we performed pseudovirus neutralization assays with serum from the RBD-scNP or S-2P mRNA-LNP-vaccinated macaques. RBD-scNP macaque serum potently neutralized a pseudovirus bearing the Spike from the B.1.1.7 variant of SARS-CoV-2 (FIG. 2d,e). Similarly, neutralizing antibodies elicited by mRNA-LNP encoding the stabilized TM spike neutralized the B.1.1.7 variant of SARS-CoV-2 equally as well as the D614G variant of SARS-CoV-2, albeit at titers below those observed with RBD-scNP immunization (FIG. 2d,e). Thus, the potentially more transmissible B.1.1.7 variant of SARS-CoV-2 was equally as susceptible to vaccine-induced neutralizing antibodies as the SARS-CoV-2 D614G variant. Next, we determined the neutralizing activity of immune sera against SARS-CoV-2 WA-1, B.1.351, and P.1 pseudoviruses. Macaque serum from RBD-scNP or mRNA-LNP immunization neutralized all three strains of SARS-CoV-2 with ID80 titers more potent for the RBD-scNP group (FIG. 2f-i). Neutralization titers for both groups of immunized macaques decreased against the B.1.351 and P.1 strains of SARS-CoV-2 compared to the WA-1 strain (FIG. 2g,i). On average, the RBD-scNP group neutralization titers decreased by 3-fold against B.1.351 or P.1, whereas the mRNA-LNP group decreased by 6-fold for B.1.351 and 10-fold for P.1 based on ID50 titer (FIG. 2g,i). Additionally, we determined RBD-scNP and S-2P mRNA-LNP immune plasma IgG binding to S ectodomain proteins containing mutations observed in strains circulating in Danish minks, as well as B1.351, P.1, and B.1.1.7 SARS-CoV-2 strains^{38,39,42,43}. Similar to DH1047, the presence of mutations from mink strains of SARS-CoV-2, the South African B1.351, Brazilian P.1 strain, and UK B.1.1.7 strains did not change the binding magnitude for RBD-scNP or mRNA-LNP macaque plasma IgG (FIG. 8). In summary, both vaccines tested here elicited neutralizing antibodies that were unaffected by the mutations in the B.1.1.7 strain. However, neutralizing antibodies elicited by RBD-scNP were more potent for the difficult-to-neutralize B.1.351 and P.1 virus strains than antibodies elicited with transmembrane Spike-2P mRNA-LNP immunization.

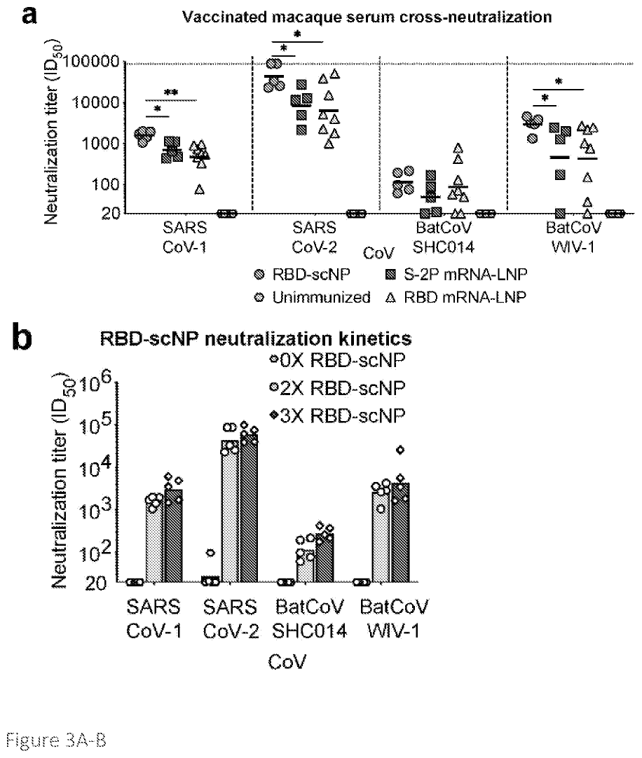

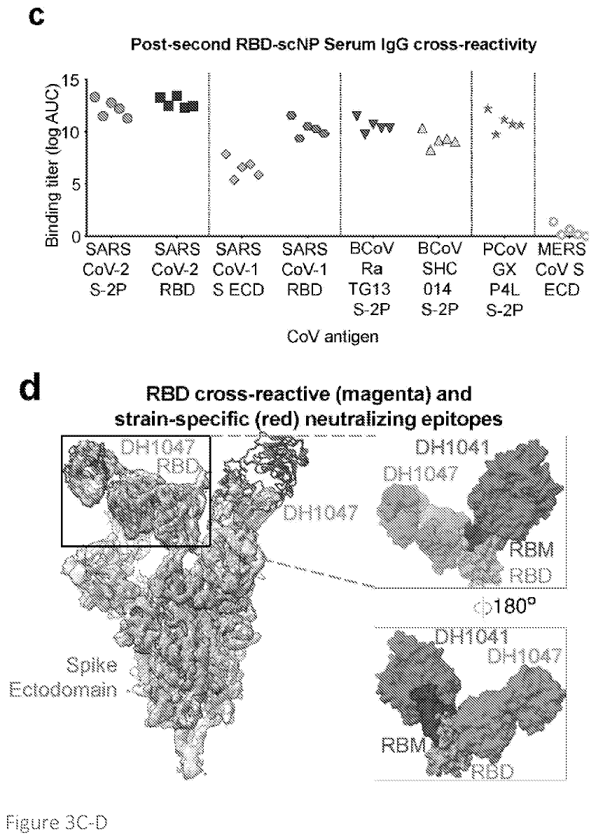

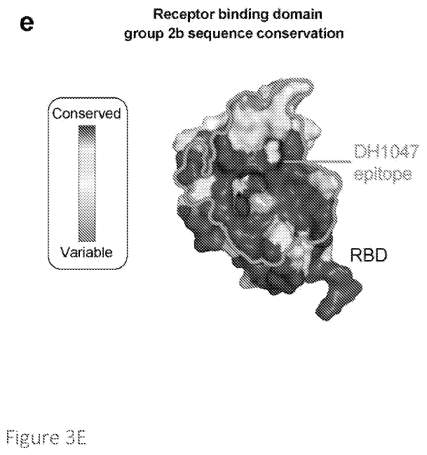

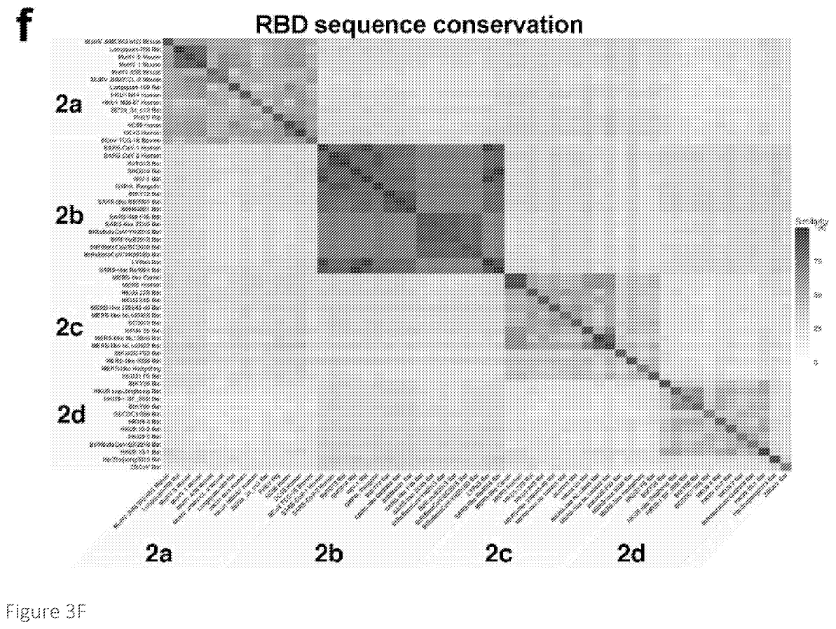

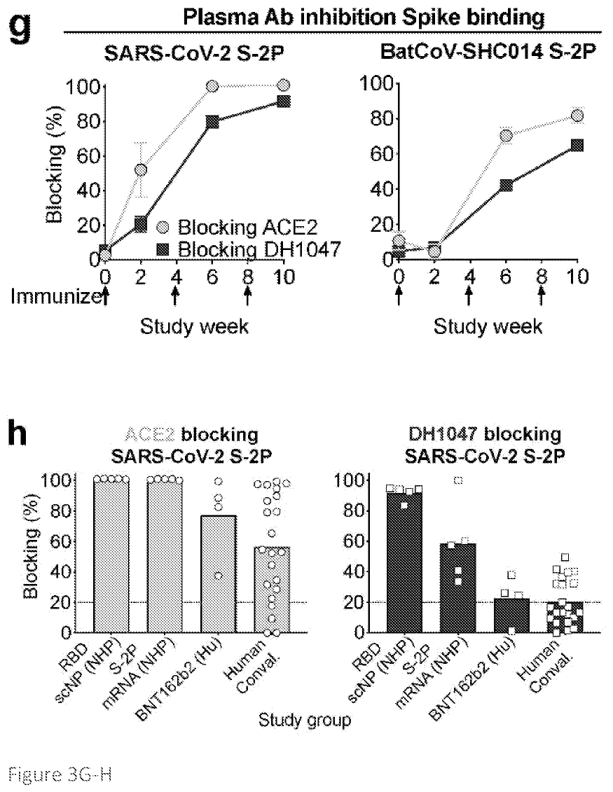

While SARS-CoV-2 wild-type and variant neutralization remain a priority for halting the current pandemic, additional SARS-related CoVs that circulate in humans and animals remain a threat for future outbreaks⁴⁴⁻⁴⁶. Therefore, we examined neutralization of SARS-CoV-1 and SARS-related group 2b batCoV-WIV-1 and SARS-related batCoV-SHC014 viruses by immune sera from macaques vaccinated with the RBD-scNP, mRNA-LNP encoding monomeric RBD or S-2P (Extended Data FIG. 2a-c)^{6,10,454,6}. After two immunizations, RBD-scNP, S-2P mRNA-LNP, and the RBD monomer mRNA-LNP elicited neutralizing antibodies against SARS-CoV-1, batCoV-WIV-1, and batCoV-SHC014 (FIG. 3a, FIG. 9). Neutralization was more potent for replication-competent SARS-CoV-2 virus compared to the other three SARS-related viruses (FIG. 3a, FIG. 9), with neutralization titers varying up to 4-fold within the RBD-scNP group (FIG. 9). Among the three immunogens, RBD-scNP elicited the highest neutralization titers and mRNA-LNP expressing monomer RBD elicited the lowest neutralization titers (FIG. 3a, FIG. 9). Small increases in neutralization potency were gained by boosting a third time with the RBD-scNP (FIG. 3b). Also, RBD-scNP immunization elicited cross-reactive IgG binding against SARS-CoV-2, SARS-CoV-1, batCoV-RaTG13, batCoV-SHCO14, pangolin CoV-GXP4L Spike proteins (FIG. 3c and FIGS. 10a,c). Binding antibody titers were high for these spikes, even in instances where neutralization titers were low indicating non-neutralizing antibodies contributed to binding titers. RBD-scNP immune plasma IgG did not bind the S ectodomain of four endemic human CoVs, nor did it bind MERS-CoV S ectodomain (FIGS. 10a,c). The lack of binding by plasma IgG to these latter five S ectodomains was consistent with RBD sequence divergence among groups 1, 2a, 2b and 2c coronaviruses (FIG. 3f and FIGS. 11-13). Nonetheless, the SARS-CoV-2 spike induced cross-reactive antibodies against multiple group 2b SARS-related betaCoVs, with the highest titers induced by RBD-scNP.

Immune sera from RBD-scNP-immunized macaques exhibited a similar cross-neutralizing profile as the cross-neutralizing antibody DH1047. DH1047 bound with <0.02 nM affinity to monomeric SARS-CoV-2 RBD (Extended data FIG. 10b), and bound the RBD-scNP (FIG. 1b). DH1047 protects against SARS-CoV-2 infection in macaques and batCoV-WIV-1 infection in mice²⁴; thus the DH1047 epitope is a principal betacoronavirus (CoV) cross-neutralizing antibody target for vaccination. The cross-reactive DH1047 epitope is adjacent to the N-terminus of the receptor binding motif (RBM) distinguishing it from dominant ACE2 binding site-focused neutralizing antibodies such as DH1041²⁴ (FIG. 3d). Antibodies targeting near the DH1047 epitope would be predicted to be cross-reactive with group 2b betaCoVs given the high sequence conservation present in and immediately proximal to the DH1047 epitope (FIG. 3e). Comparison of RBD sequences showed them to be relatively conserved within betaCoV group 2b, but minimally conserved between groups 2b and 2c (FIG. 3f and data FIGS. 11-13). To examine whether RBD-scNP-induced antibodies bound near the DH1047 epitope, we assessed plasma antibody blocking of DH1047 binding to SARS-CoV-2 S-2P ectodomain. Plasma from all RBD-scNP immunized macaques blocked the binding of ACE2 and DH1047 to SARS-CoV-2 S-2P ectodomain (FIG. 3g and FIG. 10d). Since DH1047-blocking plasma antibodies could be SARS-CoV-2-specific, we also examined plasma IgG blocking of DH1047 binding to batCoV-SHCO14. Similar to SARS-CoV-2 S binding, RBD-scNP plasma also potently blocked DH1047 binding to batCoV-SHCO14 (FIG. 3g).

To determine if RBD-scNP immunization elicited higher frequencies of DH1047 blocking antibodies than other vaccine modalities or SARS-CoV-2 infection, we compared serum DH1047 blocking activity elicited by S-2P mRNA-LNP immunization of macaques (N=5), elicited by Pfizer BNT162b2 mRNA-LNP immunization of humans (N=4), or elicited by SARS-CoV-2 infection in convalescent individuals (N=22). S-2P mRNA-LNP and RBD-scNP immunization of macaques elicited high ACE2 blocking activity in all macaques, but S-2P mRNA-LNP blocking of DH1047 was ˜40% lower compared to RBD-scNP immunization (FIG. 3h and Fi10. d). Vaccination with the Pfizer/BioNTech COVID-19 vaccine BNT162b2 (n=4) elicited ACE2 blocking activity in each individual, and 78% of SARS-CoV-2 convalescent individuals tested had serum AC2 blocking activity (FIG. 3h and FIG. 10d). While 5/5 RBD-scNP vaccinated macaques had 1005 DH1047 serum blocking activity, only 3 of 4 SARS-CoV-2 convalescent and 31% of COVID-19 convalescent individuals had low levels of serum DH1047 blocking activity (FIG. 3h). Thus, in human and macaque S-2P mRNA vaccination and in COVID-19 convalescent individuals, the serum cross-reactive DH1047 antibody response was subdominant, while DH1047-targeted antibodies was a dominant response following RBD-scNP vaccination.

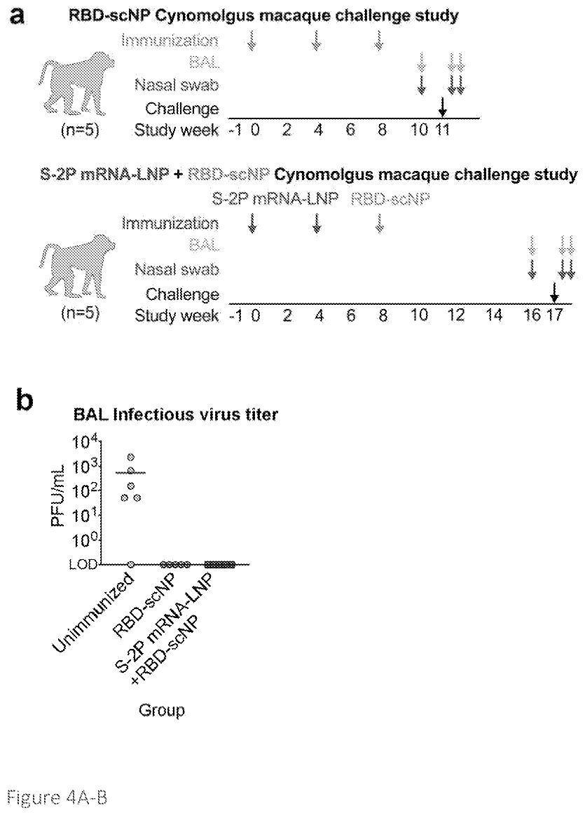

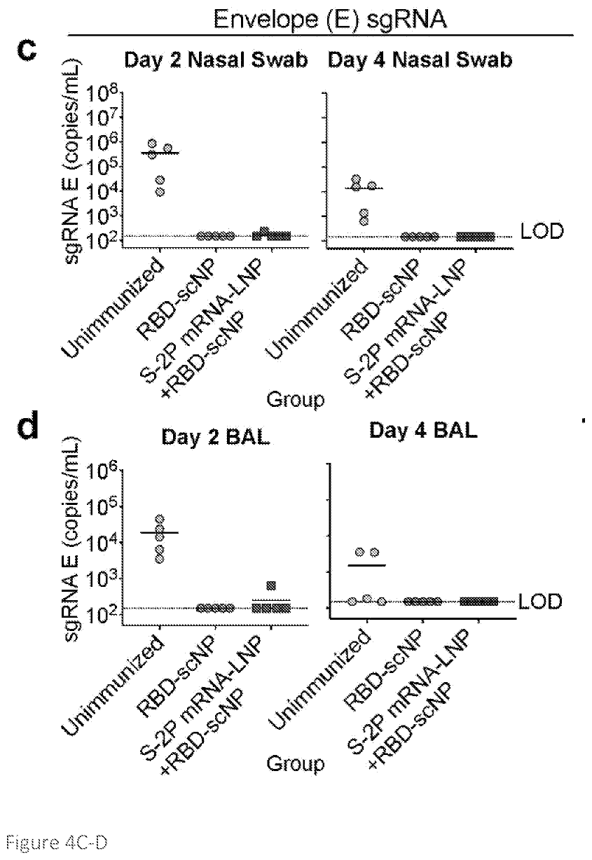

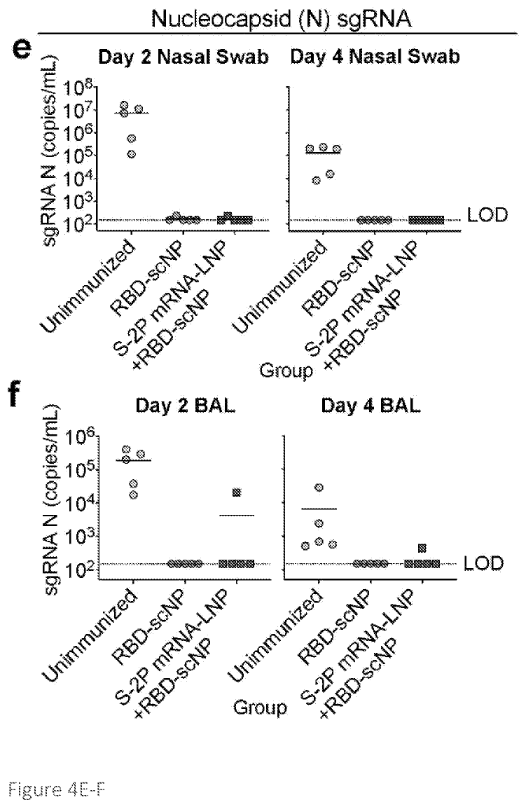

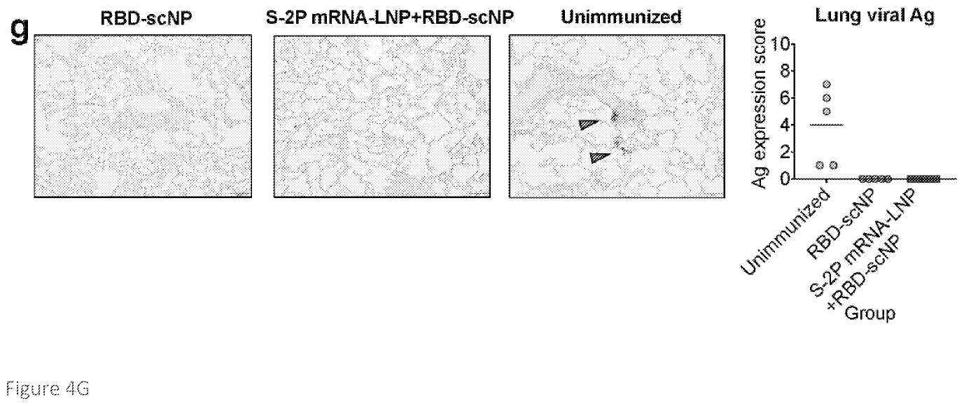

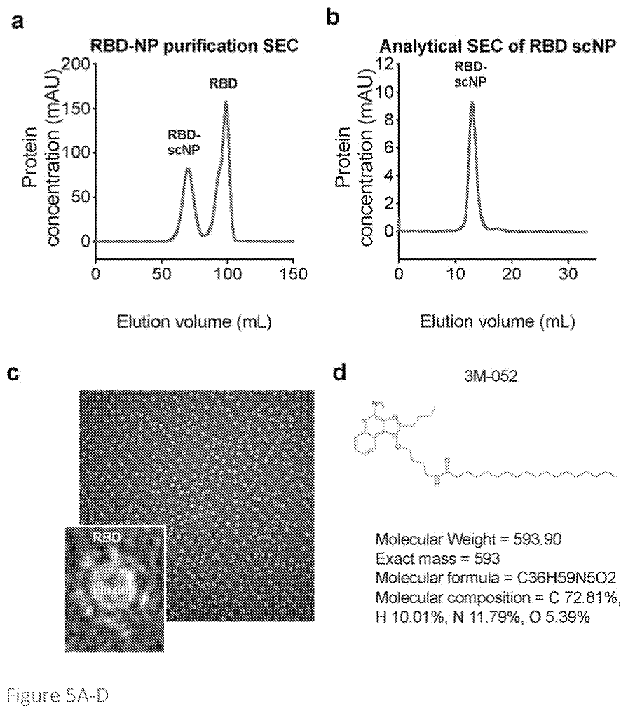

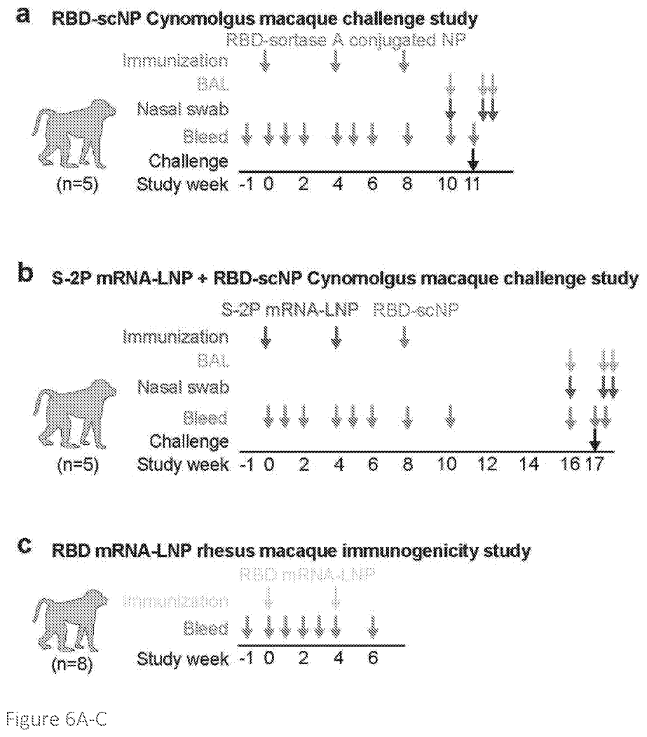

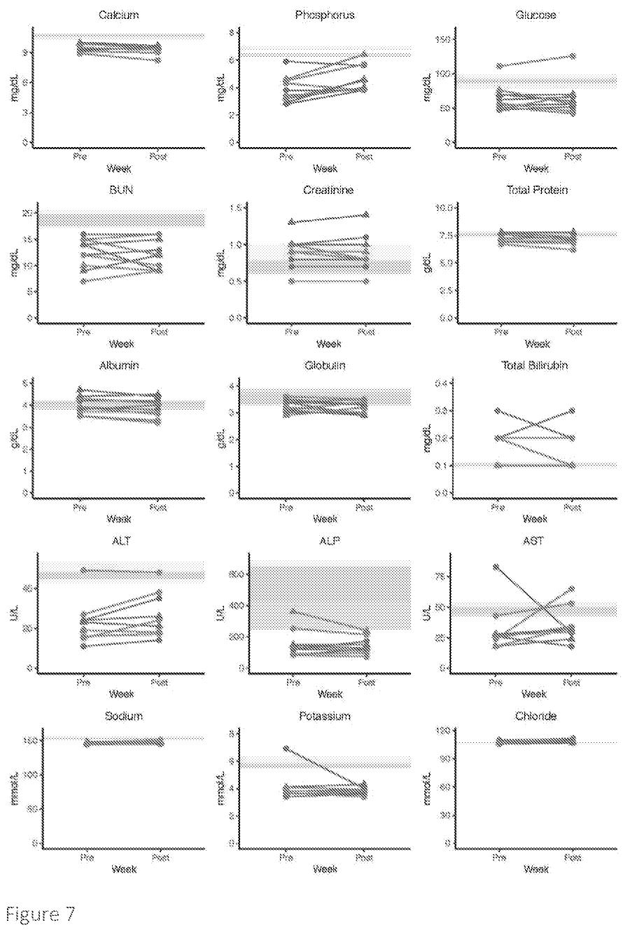

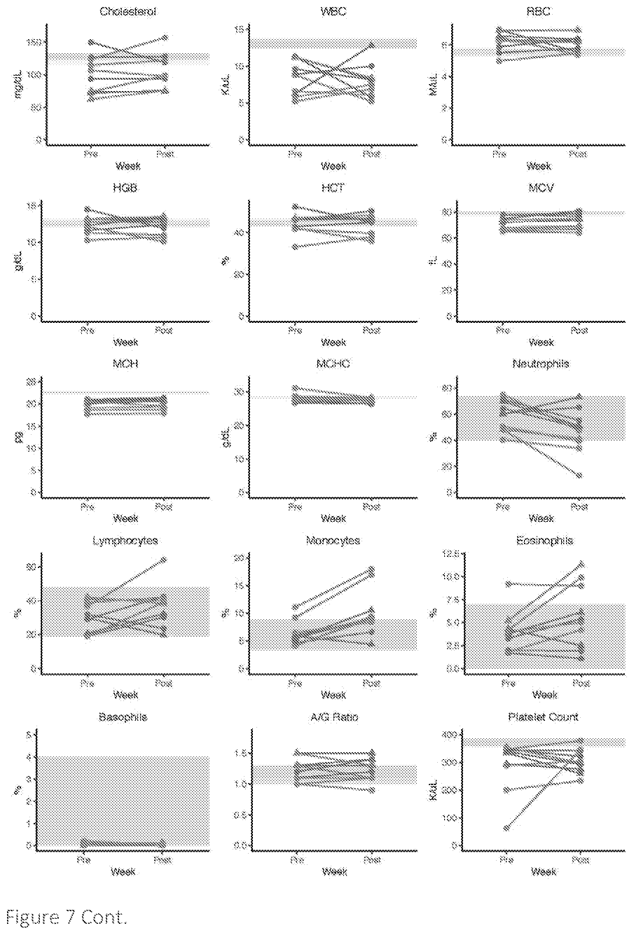

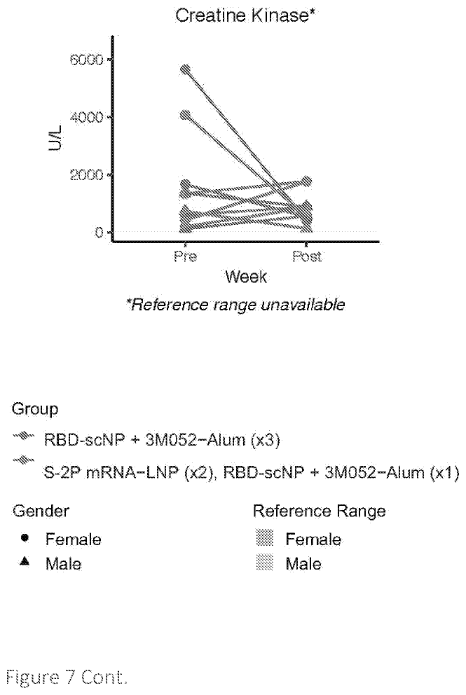

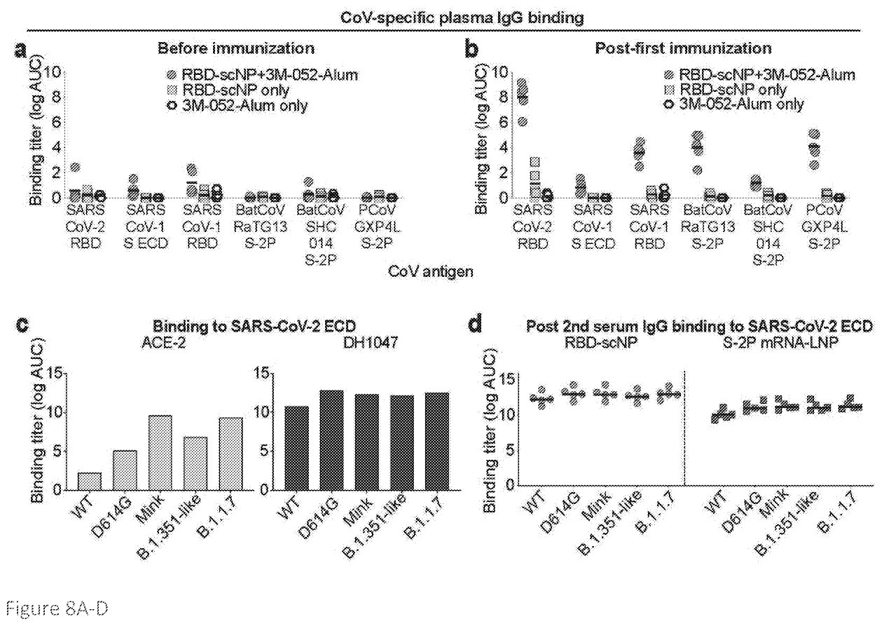

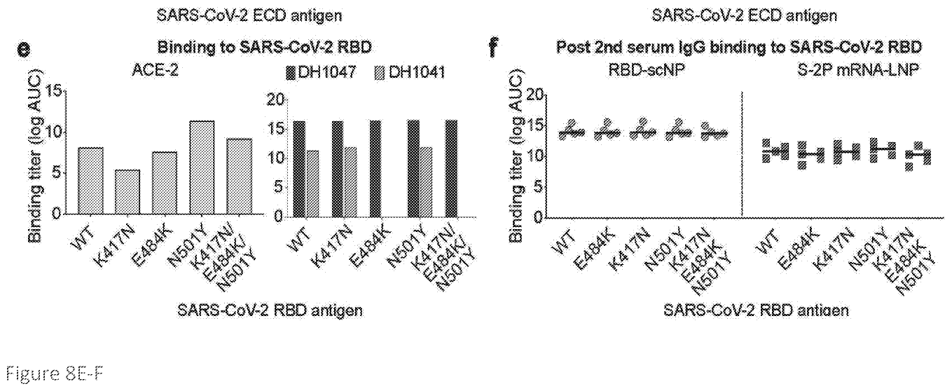

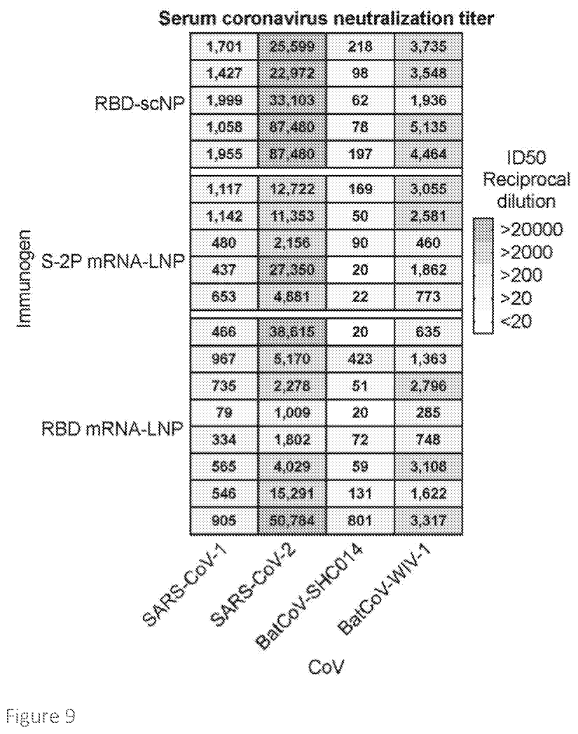

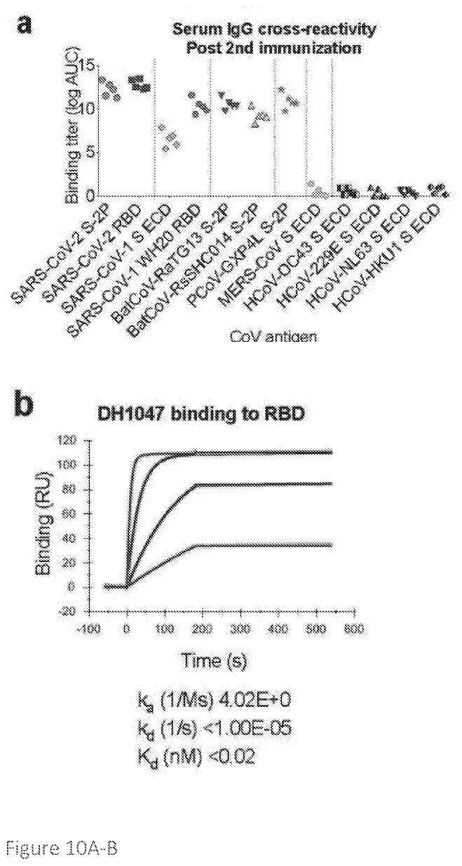

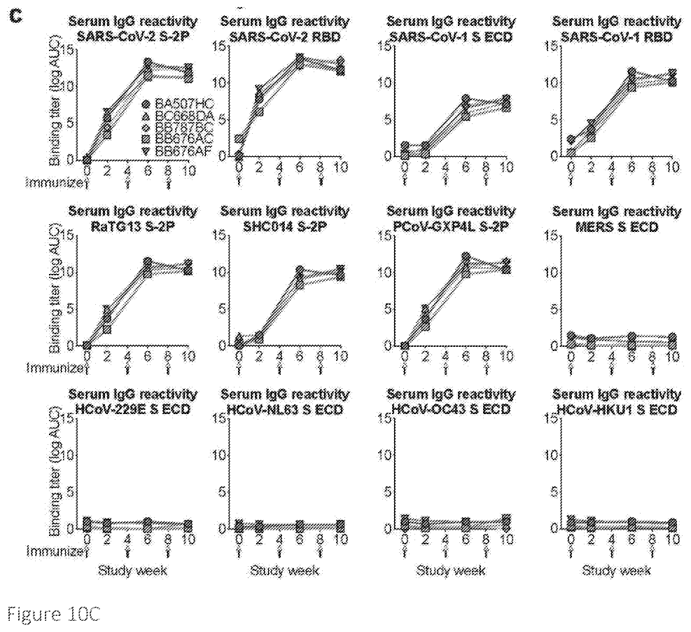

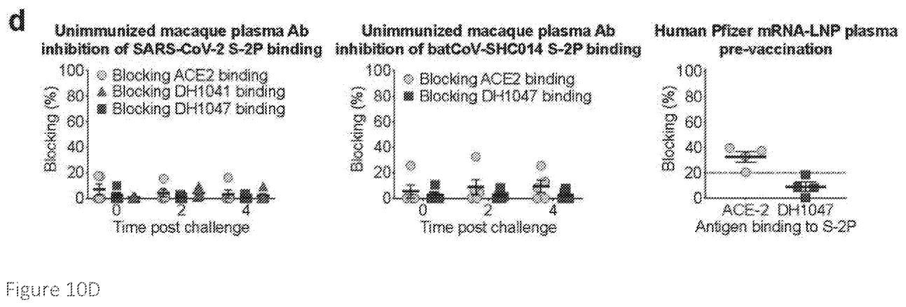

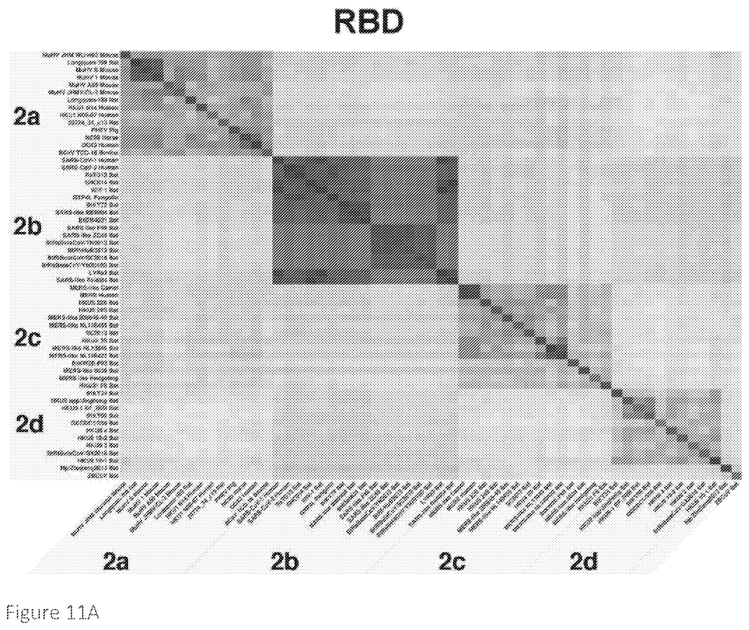

To determine protection against mucosal coronavirus infection, we challenged RBD scNP-vaccinated and S-2P mRNA-LNP primed/RBD-scNP boosted monkeys with 10⁵ plaque forming units of SARS-CoV-2 virus via intratracheal and intranasal routes (FIG. 4a). RBD scNP-vaccinated or mRNA-LNP primed/RBD-scNP boosted monkeys were challenged after their last boost. Neutralizing antibodies were detectable in all macaques 2 weeks after the final immunization (FIG. 3b and FIG. 14). Bronchoalveolar lavage (BAL) fluid was collected 2 days post challenge (FIG. 4a), and the presence of infectious SARS-CoV-2 in plaque forming units (PFUs) in the lower respiratory tract was determined. Infectious SARS-CoV-2 was detectable in BAL fluid from 5 of 6 unimmunized macaques, but was undetectable in all RBD-scNP and S-2P mRNA-LNP/RBD-scNP-immunized macaques two days after challenge (FIG. 4b). SARS-CoV-2 replication was quantified in the upper and lower respiratory tract as envelope (E) and nucleocapsid (N) subgenomic RNA. Subgenomic (sg) RNA was quantified in fluid from nasal swabs and bronchoalveolar lavage (BAL) two and four days after challenge (FIG. 4a). On day 2 after challenge, in control, unimmunized macaques, there was an average of 1.3×10⁵ and 1.2×10⁴ copies/mL of E gene sgRNA in the nasal swab and BAL fluids, respectively (FIGS. 4c,d). In contrast, RBD-scNP-vaccinated monkeys and 4 of 5 S-2P mRNA-LNP monkeys had undetectable levels of subgenomic envelope E gene RNA in the upper and lower respiratory tract (FIGS. 4c,d). We sampled monkeys again 2 days later to determine if detectable virus replication was present, but found no detectable E gene sgRNA in any vaccinated monkey BAL or nasal swab samples (FIGS. 4b,c). Similarly, all RBD-scNP-vaccinated macaque had undetectable N gene sgRNA in BAL and the nasal swab fluid (FIGS. 4e,f), except one macaque that had 234 copies/mL of N gene sgRNA detected on day 2 in nasal swab fluid (FIG. 4e). Virus replication was undetectable in this macaque by the fourth day after challenge (FIG. 4e). Additionally, all but one macaque administered mRNA-LNP prime/RBD-scNP boost had undetectable N gene sgRNA in BAL or nasal swab samples (FIGS. 4e,f). Lung tissue on from post-challenge day 7, demonstrated SARS-CoV nucleocapsid antigen was undetectable in the lung tissue of all vaccinated macaques, but was detected in all control macaques (FIG. 4g and FIG. 15). Hematoxylin and eosin staining of lung tissue showed reduction in inflammation in immunized compared to control macaques (FIG. 15 and FIG. 17).

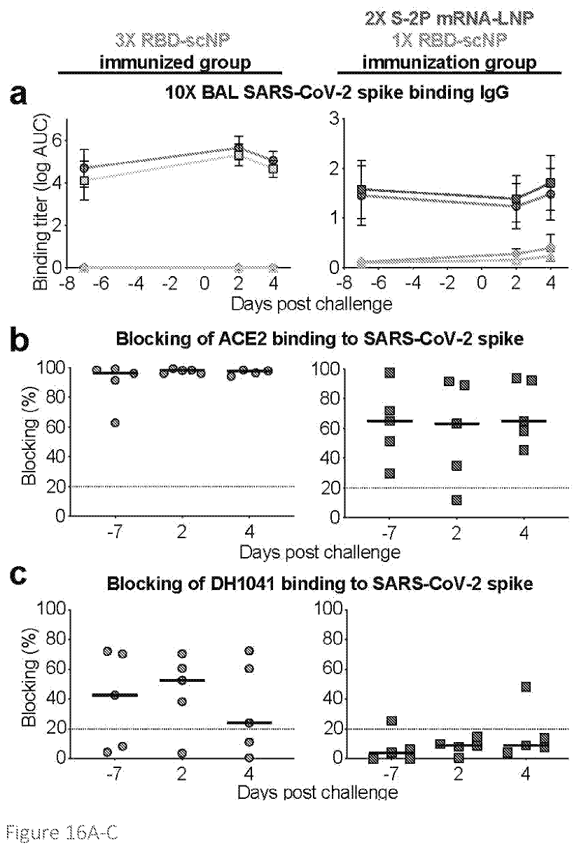

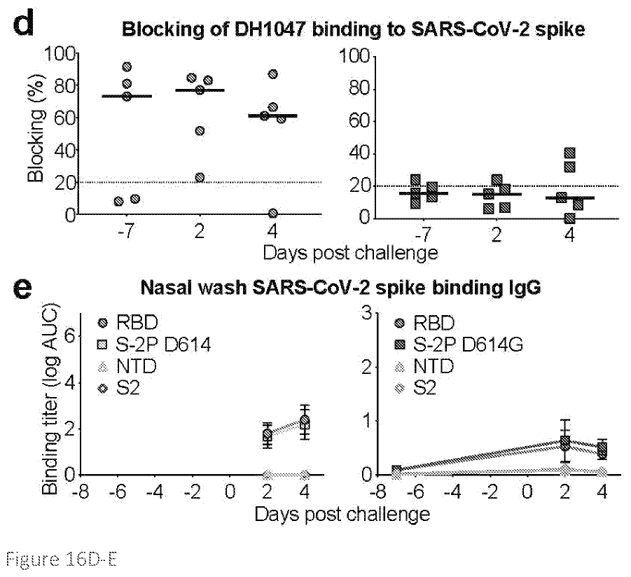

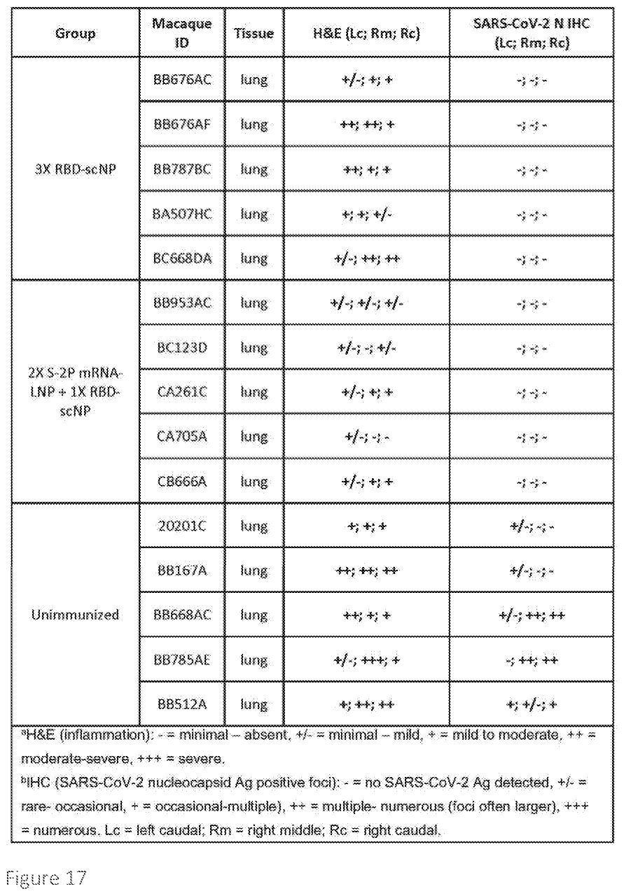

Mucosal responses to SARS-CoV-2 were examined when possible both before and after SARS-CoV-2 challenge (FIG. 16). Concentrated bronchoalveolar lavage fluid (BAL) from both groups of macaques was examined for IgG binding to spike (FIG. 16a), and blocking of ACE-2, DH1041, and DH1047 binding to spike (FIG. 16b-d). Each response was higher in the BAL from monkeys immunized three times with RBD-scNP compared to monkeys immunized two times with S-2P mRNA-LNP and boosted once with RBD-scNP (FIG. 16a-d). However, it should be noted that the BAL was collected six weeks later in macaques immunized twice with S-2P mRNA-LNP and boosted with RBD-scNP compared to macaques immunized with RBD-scNP alone. Unconcentrated nasal wash samples from monkeys immunized with RBD-scNPs or S-2P mRNA-LNP prime/RBD-scNP boost showed similar low levels of spike-binding IgG post challenge (FIG. 16e). Nonetheless, RBD-scNP immunization elicited RBD-specific mucosal antibody responses.

This study demonstrates that immunization with SARS-CoV-2 Spike as a protein RBD-scNP or as an mRNA-LNP elicits a cross-reactive antibody response can neutralize multiple SARS-related human and bat betaCoVs. These results demonstrate that SARS-CoV-2 vaccination with the RBD-scNP or the stabilized transmembrane spike mRNA-LNP vaccines currently approved for use in humans, can elicit cross-neutralizing antibodies with the potential to prevent future group 2b betaCoV spillover events to humans^{30,44,47}.

The identification of betaCoV cross-neutralizing antibodies such as DH1047 have shown that the SARS-CoV-2 RBD contains a conserved betaCoV group 2b cross-neutralizing epitope^{13,23,24,48,49}. Thus, the epitope of DH1047 and other cross-neutralizing antibodies are major targets for pancoronavirus vaccines and as well, vaccines aiming to neutralize SARS-CoV-2 variants. Vaccination of macaques in this study showed that cross-neutralizing epitopes on RBD can be potently targeted by primates, with RBD-scNP optimally inducing cross-neutralizing responses. A recent study reported mosaic RBD nanoparticles arrayed with RBDs from various CoVs induced cross-neutralizing antibodies in mice³⁰. Here, we show that cross-neutralizing antibodies are not only elicited by RBD nanoparticles, but also by mRNA-LNPs expressing SARS-CoV-2 stabilized spike of the design currently in mRNA COVID-19 vaccines. The 24-mer RBD-scNP nanoparticle protein adjuvanted with a toll-like receptor agonist 3M-052 adsorbed to alum elicited the highest group 2b cross-neutralizing antibody titers. 3M-052 formulated with alum or in an emulsion has induced long-lasting memory B cells and protection against SHIV challenge in non-human primates^{50,51}. In our HIV-1 studies, we have found that the addition of 3M-052 as an adjuvant boosts neutralizing antibody titers versus no adjuvant. A Phase I clinical study using 3M-052/Alum to induce neutralizing antibody responses to an HIV vaccine candidate is underway (NCT04177355). Thus, this vaccine modality represents a promising first-generation pan-group 2b betaCoV vaccine with the potential to durably inhibit future zoonotic transmission²⁵.

The emergence of SARS-CoV-2 neutralization-resistant and highly infectious variants continues to be a concern for vaccine efficacy. We found here that RBD protein nanoparticle or mRNA-LNP SARS-CoV-2 spike immunization elicited SARS-CoV-2 neutralizing antibodies can neutralize the predominant SARS-CoV-2 variant D614G as well as the newly-emerged B.1.1.7 UK variant. Thus, while the B.1.1.7 variant may be more transmissible, it was equally as sensitive to vaccine-induced serum neutralization as the predominant circulating SARS-CoV-2 D614G strain. Importantly, cross-reactive RBD-scNP immune sera elicited more potent neutralization of the P.1 and B.1.351 strains of SARS-CoV-2 than two immunizations with transmembrane S-2P mRNA-LNP. The neutralizing antibodies elicited by RBD-scNP and transmembrane S-2P mRNA-LNP were of different specificities since RBD-scNP-induced neutralizing antibodies showed a smaller reduction in neutralization when compared to transmembrane S-2P mRNA-LNP immune sera. Overall, the reduction in neutralization, but not complete knockout of neutralization by each vaccine-induced sera is compatible with the recent demonstration that current COVID-19 vaccines have efficacy, albeit reduced, against the UK B.1.1.7 and South African B1.351 SARS-CoV-2 variants⁵²⁻⁵⁷.

Protection against asymptomatic infection and the durability of protection remain concerns for current vaccines. The RBD-scNP vaccine induced robust protective immunity for SARS-CoV-2 replication in the upper and lower respiratory tract. This degree of virus suppression in the upper respiratory tract has not been routinely achieved with SARS-CoV-2 challenge in macaques^{58,59} and indicates the goal of inducing SARS-CoV-2 sterilizing immunity with vaccination could be attainable. Additionally, the extraordinarily high neutralization titers achieved by RBD-scNP vaccination bode well for an extended duration of protection. Critically, as we have had 3 coronavirus epidemics in the past 20 years, there is a need to develop effective pancoronavirus vaccines prior to the next pandemic. The RBD-scNP vaccine induces neutralizing antibodies to SARS-CoV-1, SARS-CoV-2, batCoV-WIV-1 and batCoV-SCH014 and represents a platform for producing pancoronavirus vaccines that could prevent, rapidly temper, or extinguish the next spillover of a coronavirus into humans.

### REFERENCES

1 Wang, C., Horby, P. W., Hayden, F. G. & Gao, G. F. A novel coronavirus outbreak of global health concern. Lancet 395, 470-473, doi:10.1016/s0140-6736(20)30185-9 (2020).
2 Zaki, A. M., van Boheemen, S., Bestebroer, T. M., Osterhaus, A. D. & Fouchier, R. A. Isolation of a novel coronavirus from a man with pneumonia in Saudi Arabia. N Engl J Med 367, 1814-1820, doi:10.1056/NEJMoa1211721 (2012).
3 Zhong, N. S. et al. Epidemiology and cause of severe acute respiratory syndrome (SARS) in Guangdong, People's Republic of China, in February, 2003. Lancet 362, 1353-1358, doi:10.1016/s0140-6736(03)14630-2 (2003).
4 Zhu, N. et al. A Novel Coronavirus from Patients with Pneumonia in China, 2019. N Engl J Med 382, 727-733, doi:10.1056/NEJMoa2001017 (2020). Zhou, P. et al. A pneumonia outbreak associated with a new coronavirus of probable bat origin. Nature 579, 270-273, doi:10.1038/s41586-020-2012-7 (2020).
6 Li, W. et al. Bats are natural reservoirs of SARS-like coronaviruses. Science 310, 676-679, doi:10.1126/science.1118391 (2005).
7 Olival, K. J. et al. Host and viral traits predict zoonotic spillover from mammals. Nature 546, 646-650, doi:10.1038/nature22975 (2017).
8 Memish, Z. A. et al. Human infection with MERS coronavirus after exposure to infected camels, Saudi Arabia, 2013. Emerg Infect Dis 20, 1012-1015, doi:10.3201/eid2006.140402 (2014).
9 Sabir, J. S. et al. Co-circulation of three camel coronavirus species and recombination of MERS-CoVs in Saudi Arabia. Science 351, 81-84, doi:10.1126/science.aac8608 (2016).
10 Ge, X. Y. et al. Isolation and characterization of a bat SARS-like coronavirus that uses the ACE2 receptor. Nature 503, 535-538, doi:10.1038/nature12711 (2013).
11 Guan, Y. et al. Isolation and characterization of viruses related to the SARS coronavirus from animals in southern China. Science 302, 276-278, doi:10.1126/science.1087139 (2003).
12 Xiao, K. et al. Isolation of SARS-CoV-2-related coronavirus from Malayan pangolins. Nature 583, 286-289, doi:10.1038/s41586-020-2313-x (2020).
13 Pinto, D. et al. Cross-neutralization of SARS-CoV-2 by a human monoclonal SARS-CoV antibody. Nature 583, 290-295, doi:10.1038/s41586-020-2349-y (2020).
14 Shi, R. et al. A human neutralizing antibody targets the receptor-binding site of SARS-CoV-2. Nature 584, 120-124, doi:10.1038/s41586-020-2381-y (2020).
15 Ju, B. et al. Human neutralizing antibodies elicited by SARS-CoV-2 infection. Nature 584, 115-119, doi:10.1038/s41586-020-2380-z (2020).
16 Robbiani, D. F. et al. Convergent antibody responses to SARS-CoV-2 in convalescent individuals. Nature 584, 437-442, doi:10.1038/s41586-020-2456-9 (2020).
17 Zost, S. J. et al. Potently neutralizing and protective human antibodies against SARS-CoV-2. Nature 584, 443-449, doi:10.1038/s41586-020-2548-6 (2020).
18 Liu, L. et al. Potent neutralizing antibodies against multiple epitopes on SARS-CoV-2 spike. Nature 584, 450-456, doi:10.1038/s41586-020-2571-7 (2020).
19 Hansen, J. et al. Studies in humanized mice and convalescent humans yield a SARS-CoV-2 antibody cocktail. Science 369, 1010-1014, doi:10.1126/science.abd0827 (2020).
20 Brouwer, P. J. M. et al. Potent neutralizing antibodies from COVID-19 patients define multiple targets of vulnerability. Science 369, 643-650, doi:10.1126/science.abc5902 (2020).
21 Rogers, T. F. et al. Isolation of potent SARS-CoV-2 neutralizing antibodies and protection from disease in a small animal model. Science 369, 956-963, doi:10.1126/science.abc7520 (2020).
22 Piccoli, L. et al. Mapping Neutralizing and Immunodominant Sites on the SARS-CoV-2 Spike Receptor-Binding Domain by Structure-Guided High-Resolution Serology. Cell 183, 1024-1042.e1021, doi:10.1016/j.cell.2020.09.037 (2020).
23 Wec, A. Z. et al. Broad neutralization of SARS-related viruses by human monoclonal antibodies. Science 369, 731-736, doi:10.1126/science.abc7424 (2020).
24 Li, D. et al. The functions of SARS-CoV-2 neutralizing and infection-enhancing antibodies in vitro and in mice and nonhuman primates. bioRxiv, doi:10.1101/2020.12.31.424729 (2021).
25 Burton, D. R. & Walker, L. M. Rational Vaccine Design in the Time of COVID-19. Cell Host Microbe 27, 695-698, doi:10.1016/j.chom.2020.04.022 (2020).
26 López-Sagaseta, J., Malito, E., Rappuoli, R. & Bottomley, M. J. Self-assembling protein nanoparticles in the design of vaccines. Comput Struct Biotechnol J 14, 58-68, doi:10.1016/j.csbj.2015.11.001 (2016).
27 Bangaru, S. et al. Structural analysis of full-length SARS-CoV-2 spike protein from an advanced vaccine candidate. Science 370, 1089-1094, doi:10.1126/science.abe1502 (2020).
28 Walls, A. C. et al. Elicitation of Potent Neutralizing Antibody Responses by Designed Protein Nanoparticle Vaccines for SARS-CoV-2. Cell 183, 1367-1382 e1317, doi:10.1016/j.cell.2020.10.043 (2020).
29 Ma, X. et al. Nanoparticle Vaccines Based on the Receptor Binding Domain (RBD) and Heptad Repeat (HR) of SARS-CoV-2 Elicit Robust Protective Immune Responses. Immunity 53, 1315-1330 e1319, doi:10.1016/j.immuni.2020.11.015 (2020).
30 Cohen, A. A. et al. Mosaic nanoparticles elicit cross-reactive immune responses to zoonotic coronaviruses in mice. Science, doi:10.1126/science.abf6840 (2021).
31 Saunders, K. O. et al. Targeted selection of HIV-specific antibody mutations by engineering B cell maturation. Science 366, doi:10.1126/science.aay7199 (2019).
32 Rappazzo, C. G. et al. Broad and potent activity against SARS-like viruses by an engineered human monoclonal antibody. Science, doi:10.1126/science.abf4830 (2021).
33 Fox, C. B. et al. Adsorption of a synthetic TLR7/8 ligand to aluminum oxyhydroxide for enhanced vaccine adjuvant activity: A formulation approach. J Control Release 244, 98-107, doi:10.1016/j.jconrel.2016.11.011 (2016).
34 Korber, B. et al. Tracking Changes in SARS-CoV-2 Spike: Evidence that D614G Increases Infectivity of the COVID-19 Virus. Cell 182, 812-827.e819, doi:10.1016/j.cell.2020.06.043 (2020).
35 Baden, L. R. et al. Efficacy and Safety of the mRNA-1273 SARS-CoV-2 Vaccine. N Engl J Med, doi:10.1056/NEJMoa2035389 (2020).
36 Polack, F. P. et al. Safety and Efficacy of the BNT162b2 mRNA Covid-19 Vaccine. N Engl J Med 383, 2603-2615, doi:10.1056/NEJMoa2034577 (2020).
37 Galloway, S. E. et al. Emergence of SARS-CoV-2 B.1.1.7 Lineage—United States, Dec. 29, 2020-Jan. 12, 2021. MMDWR Morb Mortal Wkly Rep 70, 95-99, doi:10.15585/mmwr.mm7003e2 (2021).
38 Leung, K., Shum, M. H., Leung, G. M., Lam, T. T. & Wu, J. T. Early transmissibility assessment of the N501Y mutant strains of SARS-CoV-2 in the United Kingdom, October to November 2020. Euro Surveill 26, doi:10.2807/1560-7917.ES.2020.26.1.2002106 (2021).
39 Faria, N. R. et al. Genomic characterisation of an emergent SARS-CoV-2 lineage in Manaus: preliminary findings. Virological (2021).
40 Tegally, H. et al. Sixteen novel lineages of SARS-CoV-2 in South Africa. Nat Med, doi:10.1038/s41591-021-01255-3 (2021).
41 Wibmer, C. K. et al. SARS-CoV-2 501Y.V2 escapes neutralization by South African COVID-19 donor plasma. bioRxiv, doi:10.1101/2021.01.18.427166 (2021).
42 Lassaunibre, R. et al. SARS-CoV-2 spike mutations arising in Danish mink and their spread to humans, <https://files.ssi.dk/Mink-cluster-5-short-report_AFO2> (2021).
43 Basile, K. et al. Cell-based culture of SARS-CoV-2 informs infectivity and safe de-isolation assessments during COVID-19. Clin Infect Dis, doi:10.1093/cid/ciaal579 (2020).
44 Menachery, V. D., Graham, R. L. & Baric, R. S. Jumping species—a mechanism for coronavirus persistence and survival. Curr Opin Virol 23, 1-7, doi:10.1016/j.coviro.2017.01.002 (2017).
45 Menachery, V. D. et al. A SARS-like cluster of circulating bat coronaviruses shows potential for human emergence. Nat Med 21, 1508-1513, doi:10.1038/nm.3985 (2015).
46 Menachery, V. D. et al. SARS-like WIV1-CoV poised for human emergence. Proc Natl Acad Sci USA 113, 3048-3053, doi:10.1073/pnas.1517719113 (2016).
47 Cui, J., Li, F. & Shi, Z. L. Origin and evolution of pathogenic coronaviruses. Nat Rev Microbiol 17, 181-192, doi:10.1038/s41579-018-0118-9 (2019).
48 Liu, H. et al. Cross-Neutralization of a SARS-CoV-2 Antibody to a Functionally Conserved Site Is Mediated by Avidity. Immunity 53, 1272-1280 e1275, doi:10.1016/j.immuni.2020.10.023 (2020).
49 Wang, C. et al. A human monoclonal antibody blocking SARS-CoV-2 infection. Nat Commun 11, 2251, doi:10.1038/s41467-020-16256-y (2020).
50 Arunachalam, P. S. et al. T cell-inducing vaccine durably prevents mucosal SHIV infection even with lower neutralizing antibody titers. Nat Med 26, 932-940, doi:10.1038/s41591-020-0858-8 (2020).
51 Petitdemange, C. et al. Vaccine induction of antibodies and tissue-resident CD8+ T cells enhances protection against mucosal SHIV-infection in young macaques. JCI Insight 4, doi:10.1172/jci.insight.126047 (2019).
52 Muik, A. et al. Neutralization of SARS-CoV-2 lineage B.1.1.7 pseudovirus by BNT162b2 vaccine-elicited human sera. Science, doi:10.1126/science.abg6105 (2021).
53 Wu, K. et al. mRNA-1273 vaccine induces neutralizing antibodies against spike mutants from global SARS-CoV-2 variants. bioRxiv, doi:10.1101/2021.01.25.427948 (2021).
54 Cele, S. et al. Escape of SARS-CoV-2 501Y.V2 variants from neutralization by convalescent plasma. medRxiv, 2021.2001.2026.21250224, doi:10.1101/2021.01.26.21250224 (2021).
55 Collier, D. et al. Impact of SARS-CoV-2 B.1.1.7 Spike variant on neutralisation potency of sera from individuals vaccinated with Pfizer vaccine BNT162b2. medRxiv, 2021.2001.2019.21249840, doi:10.1101/2021.01.19.21249840 (2021).
56 Wang, Z. et al. mRNA vaccine-elicited antibodies to SARS-CoV-2 and circulating variants. bioRxiv, 2021.2001.2015.426911, doi:10.1101/2021.01.15.426911 (2021).
57 Wibmer, C. K. et al. SARS-CoV-2 501Y.V2 escapes neutralization by South African COVID-19 donor plasma. bioRxiv, 2021.2001.2018.427166, doi:10.1101/2021.01.18.427166 (2021).
58 Yu, J. et al. DNA vaccine protection against SARS-CoV-2 in rhesus macaques. Science 369, 806-811, doi:10.1126/science.abc6284 (2020).
59 Mercado, N. B. et al. Single-shot Ad26 vaccine protects against SARS-CoV-2 in rhesus macaques. Nature 586, 583-588, doi:10.1038/s41586-020-2607-z (2020).
60 Laczko, D. et al. A Single Immunization with Nucleoside-Modified mRNA Vaccines Elicits Strong Cellular and Humoral Immune Responses against SARS-CoV-2 in Mice. Immunity 53, 724-732 e727, doi:10.1016/j.immuni.2020.07.019 (2020).
61 Pardi, N. et al. Zika virus protection by a single low-dose nucleoside-modified mRNA vaccination. Nature 543, 248-251, doi:10.1038/nature21428 (2017).
62 Wrapp, D. et al. Cryo-EM structure of the 2019-nCoV spike in the prefusion conformation. Science 367, 1260-1263, doi:10.1126/science.abb2507 (2020).
63 Hsieh, C. L. et al. Structure-based Design of Prefusion-stabilized SARS-CoV-2 Spikes. bioRxiv, doi:10.1101/2020.05.30.125484 (2020).
64 Zhou, T. et al. Structure-Based Design with Tag-Based Purification and In-Process Biotinylation Enable Streamlined Development of SARS-CoV-2 Spike Molecular Probes. Cell Rep 33, 108322, doi:10.1016/j.celrep.2020.108322 (2020).
65 Berry, J. D. et al. Development and characterisation of neutralising monoclonal antibody to the SARS-coronavirus. J Virol Methods 120, 87-96, doi:10.1016/j.jviromet.2004.04.009 (2004).
66 Coleman, C. M. & Frieman, M. B. Growth and Quantification of MERS-CoV Infection. Curr Protoc Microbiol 37, 15E 12 11-19, doi:10.1002/9780471729259.mc15e02s37 (2015).
67 Kint, J., Maier, H. J. & Jagt, E. Quantification of infectious bronchitis coronavirus by titration in vitro and in ovo. Methods Mol Biol 1282, 89-98, doi:10.1007/978-1-4939-2438-7_9 (2015).
68 Naldini, L., Blomer, U., Gage, F. H., Trono, D. & Verma, I. M. Efficient transfer, integration, and sustained long-term expression of the transgene in adult rat brains injected with a lentiviral vector. Proc Natl Acad Sci USA 93, 11382-11388, doi:10.1073/pnas.93.21.11382 (1996).
69 Yount, B. et al. Reverse genetics with a full-length infectious cDNA of severe acute respiratory syndrome coronavirus. Proc Natl Acad Sci USA 100, 12995-13000, doi:10.1073/pnas.1735582100 (2003).
70 Scobey, T. et al. Reverse genetics with a full-length infectious cDNA of the Middle East respiratory syndrome coronavirus. Proc Natl Acad Sci USA 110, 16157-16162, doi:10.1073/pnas.1311542110 (2013).
71 Hou, Y. J. et al. SARS-CoV-2 Reverse Genetics Reveals a Variable Infection Gradient in the Respiratory Tract. Cell 182, 429-446 e414, doi:10.1016/j.cell.2020.05.042 (2020).
72 Edgar, R. C. Search and clustering orders of magnitude faster than BLAST. Bioinformatics 26, 2460-2461, doi:10.1093/bioinformatics/btq461 (2010).
73 Nakamura, T., Yamada, K. D., Tomii, K. & Katoh, K. Parallelization of MAFFT for large-scale multiple sequence alignments. Bioinformatics 34, 2490-2492, doi:10.1093/bioinformatics/bty121 (2018).
74 Valdar, W. S. Scoring residue conservation. Proteins 48, 227-241, doi:10.1002/prot.10146 (2002).

### Methods

Animals and immunizations. Rhesus and cynomolgus macaques were housed and treated in AAALAC-accredited institutions. The study protocol and all veterinarian procedures were approved by the Bioqual IACUC per a memorandum of understanding with the Duke IACUC, and were performed based on standard operating procedures. Nucleoside-modified messenger RNA encapsulated in lipid nanoparticles (mRNA-LNP) was prepared as previously stated^{60,61} Rhesus macaques (n=8) were immunized intramuscularly with 50 μg of mRNA-LNP encoding the receptor binding domain monomer. Cynomolgus macaques (n=5) were immunized twice with 50 μg of mRNA-LNP encoding the transmembrane Spike protein stabilized with K986P and V987P mutations and boosted once with 100 μg of RBD-scNP adjuvanted with 5 μg of 3M-052 aqueous formulation (AF) admixed with 500 μg of Alum in PBS. An additional group of cynomolgus macaques (n=5) were immunized in the right and left quadriceps with 100 μg of RBD-scNP adjuvanted with 5 μg of 3M-052 aqueous formulation (AF) admixed with 500 μg of Alum in PBS 33. The mixture for immunization consisted of 250 μL of RBD-scNP mixed with 250 μL of 0.02 mg/ml 3M-052, 2 mg/ml Alum. Animals were evaluated by Bioqual veterinary staff before during and after immunizations. In the animals studied, CBCs and chemistries were obtained throughout the immunization regimen and no significant abnormalities were noted. Of the 10 cynomolgus macaques, there were no adverse events reported at injection sites. Over the course of the study, 2 cynomolgus macaques experienced slight weight loss. Two cynomolgus macaques showed a single incidence of poor appetite, with one additional cynomolgus macaque showing poor appetite intermittently throughout the study. Additionally, one macaque presented with an infected lymph node biopsy site that responded to appropriate veterinary treatment. Biospecimens before challenge, 2 days post-challenge, and 4 days post challenge were collected as described previously²⁴.

SARS-CoV-2 intranasal and intratracheal challenge. All animals were challenged at week 11 (3 weeks after last vaccination) through combined intratracheal (IT, 3.0 mL) and intranasal (IN, 0.5 mL per nostril) inoculation with an infectious dose of 105 PFU of SARS-CoV-2 (2019-nCoV/USA-WA1/2020). The stock was generated at BIOQUAL (lot #030120-1030, 3.31×10⁵ PFU/mL) from a p4 seed stock obtained from BEI Resources (NR-52281). The stock underwent deep sequencing to confirm homology with the WA1/2020 isolate. Virus was stored at −80° C. prior to use, thawed by hand and placed immediately on wet ice. Stock was diluted to 2.5×10⁴ PFU/mL in PBS and vortexed gently for 5 seconds prior to inoculation. Nasal swabs, bronchoalveolar lavage (BAL), plasma, and serum samples were collected seven days before, two days after, and four days after challenge. Unimmunized macaques (n=50 were used as a negative control group. Protection from SARS-CoV-2 infection was determined by quantitative PCR of SARS-CoV-2 subgenomic envelope (E) and the more sensitive nucleocapsid (N) RNA (E or N sgRNA)³⁹ as stated herein.

SARS-CoV-2 protein production. The CoV ectodomain constructs were produced and purified as described previously⁶². The Spike (S) ectodomain was stabilized by the introduction of 2 prolines at amino acid positions 986 and 987 and referred to as S-2P. Plasmids encoding Spike-2P and HexaPro⁶³ were transiently transfected in FreeStyle 293 cells (Thermo Fisher) using Turbo293 (SpeedBiosystems) or 293Fectin (ThermoFisher). The constructs contained an HRV 3C-cleavable C-terminal twinStrepTagII-8×His tag. On day 6, cell-free culture supernatant was generated by centrifugation of the culture and filtering through a 0.8 um filter. Protein was purified from filtered cell culture supernatants by StrepTactin resin (IBA) and by size exclusion chromatography using Superose 6 column (GE Healthcare) in 10 mM Tris pH8,150 mM NaCl or 2 mM Tris pH 8, 200 mM NaCl, 0.02% NaN₃. ACE-2-Fc was expressed by transient transfection of Freestyle 293-F cells⁶². ACE2-Fc was purified from cell culture supernatant by HiTrap protein A column chromatography and Superdex200 size exclusion chromatography in 10 mM Tris pH8,150 mM NaCl. SARS-CoV-2 NTD was produced as previously described⁶⁴. SARS-CoV-2 fusion peptide was synthesized (GenScript).

Sortase A conjugation of SARS-CoV-2 RBD to H. pylori ferritin nanoparticles. Wuhan strain SARS-CoV-2 RBD was expressed with sortase A donor sequence LPETGG (SEQ ID NO: 16) encoded at its c-terminus. C-terminal to the sortase A donor sequence was an HRV-3C cleavage site, 8×his tag (SEQ ID NO: 17), and a twin StrepTagII (IBA). The SARS-CoV-2 RBD was expressed in Freestyle293 cells and purified by StrepTactin affinity chromatography (IBA) and superdex200 size exclusion chromatography as stated herein. H. pylori ferritin particles were expressed with a pentaglycine sortase A acceptor sequence encoded at its N-terminus of each subunit. For affinity purification of ferritin particles, 6×His tags (SEQ ID NO: 48) were appended C-terminal to a HRV3C cleavage site. Ferritin particles with a sortase A N-terminal tag were buffer exchanged into 50 mM Tris, 150 mM NaCl, 5 mM CaCI2, pH7.5. 180 μM SARS-CoV-2 RBD was mixed with 120 μM of ferritin subunits and incubated with 100 μM of sortase A overnight at room temperature. Following incubation conjugated particles were isolated from free ferritin or free RBD by size exclusion chromatography using a Superose6 16/60 column.

Biolayer interferometry binding assays. Binding was measured using an OctetRed 96 (ForteBio). Anti-human IgG capture (AHC) sensor tips (Forte Bio) were hydrated for at least 10 minutes in PBS. ACE2 and monoclonal antibodies were diluted to 20 μg/mL in PBS and placed in black 96-well assay plate. The influenza antibody CH65 was used as the background reference antibody. The RBD nanoparticle was diluted to 50 μg/mL in PBS and added to the assay plate. Sensor tips were loaded with antibody for 120 s. Subsequently, the sensor tips were washed for 60 s in PBS to removed unbound antibody. The sensor tips were incubated in a fresh well of PBS to establish baseline reading before being dipped into RBD-scNP to allow association for 400 s. To measure dissociation of the antibody-RBD-scNP complex, the tip was incubated in PBS for 600 s. At the end of dissociation, the tip was ejected and a new tip was attached to load another antibody. The data was analyzed with Data Analysis HT v12 (ForteBio). Background binding observed with CH65 was subtracted from all values. All binding curves were aligned to the start of association. The binding response at the end of the 400 s association phase was plotted in GraphPad Prism v9.0.

Surface plasmon resonance (SPR) assays. SPR measurements of DH1047 antigen binding fragment (Fab) binding to monomeric SARS-CoV-2 receptor binding domain (RBD) proteins were performed in HBS-EP+ running buffer using a Biacore S200 instrument (Cytiva). Assays were performed in the DHVI BIA Core Facility. The RBD was first captured via its twin-StrepTagII onto a Series S Streptavidin chip to a level of 300-400 resonance units (RU). The antibody Fabs were injected at 0.5 to 500 nM over the captured S proteins using the single cycle kinetics injection mode at a flow rate of 50 μL/min. Fab association occurred for 180 s followed by a dissociation of 360 seconds after the end of the association phase. At the end of the dissociation phase the RBD was regenerated with a 30 s injection of glycine pH1.5. Binding values were analyzed with Biacore S200 Evaluation software (Cytiva). References included blank streptavidin surface along with blank buffer binding and was subtracted from DH1047 values to account for signal drift and non-specific protein binding. A 1:1 Langmuir model with a local Rmax was used for curve fitting. Binding rates and constants were derived from the curve. Representative results from two independent experiments are shown.

BAL plaque assay. SARS-CoV-2 Plaque assays were performed in the Duke Regional Biocontainment BSL3 Laboratory (Durham, NC) as previously described⁶⁵. Serial dilutions of BAL fluid-were incubated with Vero E6 cells in a standard plaque assay^{66,67}. BAL and cells were incubated at 37° C., 5% C02 for 1 hour. At the end of the incubation, 1 mL of a viscous overlay (1:1 2×DMEM and 1.2% methylcellulose) was added to each well. Plates are incubated for 4 days. After fixation, staining and washing, plates were dried and plaques from each dilution of BAL sample were counted. Data are reported as plaque forming units per milliliter of BAL fluid.

SARS-CoV-2 pseudovirus neutralization. For SARS-CoV-2 D614G and SARS-CoV-2 B.1.1.7 pseudovirus neutralization assays, neutralization of SARS-CoV-2 Spike-pseudotyped virus was performed by adapting an infection assay described previously with lentiviral vectors and infection in 293T/ACE2.MF (the cell line was kindly provided by Drs. Mike Farzan and Huihui Mu at Scripps). Cells were maintained in DMEM containing 10% FBS and 50 μg/ml gentamicin. An expression plasmid encoding codon-optimized full-length spike of the Wuhan-1 strain (VRC7480), was provided by Drs. Barney Graham and Kizzmekia Corbett at the Vaccine Research Center, National Institutes of Health (USA). The D614G mutation was introduced into VRC7480 by site-directed mutagenesis using the QuikChange Lightning Site-Directed Mutagenesis Kit from Agilent Technologies (Catalog #210518). The mutation was confirmed by full-length spike gene sequencing. Pseudovirions were produced in HEK 293T/17 cells (ATCC cat. no. CRL-11268) by transfection using Fugene 6 (Promega, Catalog #E2692). Pseudovirions for 293T/ACE2 infection were produced by co-transfection with a lentiviral backbone (pCMV ΔR8.2) and firefly luciferase reporter gene (pHR′ CMV Luc)⁶⁸. Culture supernatants from transfections were clarified of cells by low-speed centrifugation and filtration (0.45 μm filter) and stored in 1 ml aliquots at −80° C.

For 293T/ACE2 neutralization assays, a pre-titrated dose of virus was incubated with 8 serial 3-fold or 5-fold dilutions of mAbs in duplicate in a total volume of 150 μL for 1 h at 37° C. in 96-well flat-bottom poly-L-lysine-coated culture plates (Corning Biocoat). Cells were suspended using TrypLE express enzyme solution (Thermo Fisher Scientific) and immediately added to all wells (10,000 cells in 100 μL of growth medium per well). One set of 8 control wells received cells+virus (virus control) and another set of 8 wells received cells only (background control). After 66-72 h of incubation, medium was removed by gentle aspiration and 30 μL of Promega 1× lysis buffer was added to all wells. After a 10-minute incubation at room temperature, 100 μl of Bright-Glo luciferase reagent was added to all wells. After 1-2 minutes, 110 μl of the cell lysate was transferred to a black/white plate (Perkin-Elmer). Luminescence was measured using a PerkinElmer Life Sciences, Model Victor2 luminometer.

To make WA-1, P.1, and B.1.351 SARS-CoV-2 pseudoviruses, human codon-optimized cDNA encoding SARS-CoV-2 S glycoproteins of various strains were synthesized by GenScript and cloned into eukaryotic cell expression vector pcDNA 3.1 between the BamHI and XhoI sites. Pseudovirions were produced by co-transfection of Lenti-X 293T cells with psPAX2(gag/pol), pTrip-luc lentiviral vector and pcDNA 3.1 SARS-CoV-2-spike-deltaC19, using Lipofectamine 3000. The supernatants were harvested at 48h post transfection and filtered through 0.45-μm membranes and titrated using 293-ACE2-TMPRSS2 cells (HEK293T cells that express ACE2 protein).

For the neutralization assay, 50 μL of SARS-CoV-2 S pseudovirions were pre-incubated with an equal volume of medium containing serum at varying dilutions at room temperature for 1 h, then virus-antibody mixtures were added to 293T-ACE2 (WA-1 and B.1.351 assays) or 293-ACE2-TMPRSS2 (WA-1 and P.1 assays) cells in a 96-well plate. After a 3 h incubation, the inoculum was replaced with fresh medium. Cells were lysed 24 h later, and luciferase activity was measured using luciferin. Controls included cell only control, virus without any antibody control and positive control sera. Neutralization titers are the serum dilution (ID50/ID80) at which relative luminescence units (RLU) were reduced by 50% and 80% compared to virus control wells after subtraction of background RLUs.

Live virus neutralization assays. Full-length SARS-CoV-2, SARS-CoV, WIV-1, and RsSHC014 viruses were designed to express nanoluciferase (nLuc) and were recovered via reverse genetics as described previously 6971 Virus titers were measured in Vero E6 USAMRIID cells, as defined by plaque forming units (PFU) per ml, in a 6-well plate format in quadruplicate biological replicates for accuracy. For the 96-well neutralization assay, Vero E6 USAMRID cells were plated at 20,000 cells per well the day prior in clear bottom black walled plates. Cells were inspected to ensure confluency on the day of assay. Serum samples were tested at a starting dilution of 1:20 and were serially diluted 3-fold up to nine dilution spots. Serially diluted serum samples were mixed in equal volume with diluted virus. Antibody-virus and virus only mixtures were then incubated at 37° C. with 5% C02 for one hour. Following incubation, serially diluted sera and virus only controls were added in duplicate to the cells at 75 PFU at 37° C. with 5% C02. After 24 hours, cells were lysed, and luciferase activity was measured via Nano-Glo Luciferase Assay System (Promega) according to the manufacturer specifications. Luminescence was measured by a Spectramax M3 plate reader (Molecular Devices, San Jose, CA). Virus neutralization titers were defined as the sample dilution at which a 50% reduction in RLU was observed relative to the average of the virus control wells. Prebleed or unimmunized control macaque values were subtracted from WIV-1 neutralization titers, but all other viruses were not background subtracted.

Biocontainment and biosafety. All work described here was performed with approved standard operating procedures for SARS-CoV-2 in a biosafety level 3 (BSL-3) facility conforming to requirements recommended in the Microbiological and Biomedical Laboratories, by the U.S. Department of Health and Human Service, the U.S. Public Health Service, and the U.S. Center for Disease Control and Prevention (CDC), and the National Institutes of Health (NIH).

Plasma and mucosal IgG blocking of ACE2 binding. For ACE2 blocking assays, plates were coated with 2 μg/mL recombinant ACE2 protein, then washed and blocked with 3% BSA in 1×PBS. While assay plates blocked, purified antibodies were diluted as stated herein, only in 1% BSA with 0.05% Tween-20. In a separate dilution plate Spike-2P protein was mixed with the antibodies at a final concentration equal to the EC50 at which spike binds to ACE2 protein. The mixture was allowed to incubate at room temperature for 1 hour. Blocked assay plates were then washed and the antibody-spike mixture was added to the assay plates for a period of 1 hour at room temperature. Plates were washed and a polyclonal rabbit serum against the same spike protein (nCoV-1 nCoV-2P.293F) was added for 1 hour, washed and detected with goat anti rabbit-HRP (Abcam cat #ab97080) followed by TMB substrate. The extent to which antibodies were able to block the binding spike protein to ACE2 was determined by comparing the OD of antibody samples at 450 nm to the OD of samples containing spike protein only with no antibody. The following formula was used to calculate percent blocking: blocking %=(100−(OD sample/OD of spike only)*100).

Plasma and mucosal IgG blocking of RBD monoclonal antibody binding. Blocking assays for DH1041 and DH1047 were performed as stated herein for ACE2, except plates were coated with DH1041 or DH1047 instead of ACE2.

Plasma and mucosal IgG ELISA binding assays. For ELISA binding assays of Coronavirus Spike antibodies, the antigen panel included SARS-CoV-2 Spike S1+S2 ectodomain (ECD) (SINO, Catalog #40589—V08B1), SARS-CoV-2 Spike-2P⁶², SARS-CoV-2 Spike S2 ECD (SINO, Catalog #40590—V08B), SARS-CoV-2 Spike RBD from insect cell sf9 (SINO, Catalog #40592—V08B), SARS-CoV-2 Spike RBD from mammalian cell 293 (SINO, Catalog #40592—V08H), SARS-CoV-2 Spike NTD-Biotin, SARS-CoV Spike Protein Delta™ (BEI, Catalog #NR-722), SARS-CoV WH20 Spike RBD (SINO, Catalog #40150—V08B2), SARS-CoV WH20 Spike S1 (SINO, Catalog #40150—V08B1), SARS-CoV-1 RBD, MERS-CoV Spike S1+S2 (SINO, Catalog #40069—V08B), MERS-CoV Spike S1 (SINO, Catalog #40069—V08B1), MERS-CoV Spike S2 (SINO, Catalog #40070—V08B), MERS-CoV Spike RBD (SINO, Catalog #40071—V08B1), MERS-CoV Spike RBD.

For binding ELISA, 384-well ELISA plates were coated with 2 μg/mL of antigens in 0.1 M sodium bicarbonate overnight at 4° C. Plates were washed with PBS+0.05% Tween 20 and blocked with assay diluent (PBS containing 4% (w/v) whey protein, 15% Normal Goat Serum, 0.5% Tween-20, and 0.05% Sodium Azide) at room temperature for 1 hour. Plasma or mucosal fluid were serially diluted three-fold in superblock starting at a 1:30 dilution. Nasal was fluid started from neat and diluted 1:30, whereas BAL fluid was concentrated 10-fold. To concentrate BAL, individual BAL aliquots from the same animal and same time point were pooled in 3 KDa MWCO ultrafiltration tubes (Sartorious #VS2091). Pooled BAL was concentrated by centrifugation at 3500 rpm for 30 minutes or until volume was reduced by a factor of 10. Pool was then aliquoted and frozen at −80° C. until its use in an assay. Purified mAb samples were diluted to 100 μg/mL and then serially diluted 3-fold in assay diluent. Samples were added to the antigen-coated plates, and incubated for 1 h, followed by washes with PBS-0.1% Tween 20. HRP-conjugated goat anti-human IgG secondary Ab or mouse anti-rhesus IgG secondary antibody (SouthernBiotech, catalog #2040-05) was diluted to 1:10,000 and incubated at room temperature for 1 hour. These plates were washed four times and developed with tetramethylbenzidine substrate (SureBlue Reserve—KPL). The reaction was stopped with 1 M HCl, and optical density at 450 nm (OD₄₅₀) was determined.

Subgenomic RNA real time PCR quantification. The assay for SARS-CoV-2 quantitative Polymerase Chain Reaction (qPCR) detects total RNA using the WHO primer/probe set E_Sarbeco (Charité/Berlin). A QIAsymphony SP (Qiagen, Hilden, Germany) automated sample preparation platform along with a virus/pathogen DSP midi kit and the complex800 protocol were used to extract viral RNA from 800 μL of pooled samples. A reverse primer specific to the envelope gene of SARS-CoV-2 (5′-ATA TTG CAG CAG TAC GCA CAC A-3′ (SEQ ID NO: 49)) was annealed to the extracted RNA and then reverse transcribed into cDNA using SuperScript™ III Reverse Transcriptase (Thermo Fisher Scientific, Waltham, MA) along with RNAse Out (Thermo Fisher Scientific, Waltham, MA). The resulting cDNA was treated with RNase H (Thermo Fisher Scientific, Waltham, MA) and then added to a custom 4× TaqMan™ Gene Expression Master Mix (Thermo Fisher Scientific, Waltham, MA) containing primers and a fluorescently labeled hydrolysis probe specific for the envelope gene of SARS-CoV-2 (forward primer 5′-ACA GGT ACG TTA ATA GTT AAT AGC GT-3′ (SEQ ID NO: 50), reverse primer 5′-ATA TTG CAG CAG TAC GCA CAC A-3′ (SEQ ID NO: 49), probe 5′-6FAM/AC ACT AGC C/ZEN/A TCC TTA CTG CGC TTC G/IABkFQ-3′ (SEQ ID NO: 51)). The qPCR was carried out on a QuantStudio 3 Real-Time PCR System (Thermo Fisher Scientific, Waltham, MA) using the following thermal cycler parameters: heat to 50° C., hold for 2 min, heat to 95° C., hold for 10 min, then the following parameters are repeated for 50 cycles: heat to 95° C., hold for 15 seconds, cool to 60° C. and hold for 1 minute. SARS-CoV-2 RNA copies per reaction were interpolated using quantification cycle data and a serial dilution of a highly characterized custom DNA plasmid containing the SARS-CoV-2 envelope gene sequence. Mean RNA copies per milliliter were then calculated by applying the assay dilution factor (DF=11.7). The limit of detection (LOD) for this assay is approximately 62 RNA copies per mL of sample.

Recombinant IgG production. Expi293-F cells were diluted to 2.5E6 cells/mL on the day of transfection. Cells were co-transfected with Expifectamine and heavy and light chain expression plasmids. Enhancers were added 16h after transfection. On day 5, the cell culture was cleared of cells by centrifugation, filtered, and incubated with protein A beads overnight. The next day the protein A resin was washed with Tris buffered saline and then added to a 25 mL column. The resin was washed again and then glacial acetic acid was used to elute antibody off of the protein A resin. The pH of the solution was neutralized with 1M Tris pH8. The antibody was buffer exchanged into 25 mM sodium citrate pH6 supplemented with 150 mM NaCl, 0.2 μm filtered, and frozen at −80° C.

Negative stain electron microscopy. The RBD nanoparticle protein at ˜1-5 mg/ml concentration that had been flash frozen and stored at −80° C. was thawed in an aluminum block at 37° C. for 5 minutes; then 1-4 μL of RBD nanoparticle was diluted to a final concentration of 0.1 mg/ml into room-temperature buffer containing 150 mM NaCl, 20 mM HEPES pH 7.4, 5% glycerol, and 7.5 mM glutaraldehyde. After 5 minutes cross-linking, excess glutaraldehyde was quenched by adding sufficient 1 M Tris pH 7.4 stock to give a final concentration of 75 mM Tris and incubated for 5 minutes. For negative stain, carbon-coated grids (EMS, CF300-cu-UL) were glow-discharged for 20s at 15 mA, after which a 5-μl drop of quenched sample was incubated on the grid for 10-15 s, blotted, and then stained with 2% uranyl formate. After air drying grids were imaged with a Philips EM420 electron microscope operated at 120 kV, at 82,000× magnification and images captured with a 2k×2k CCD camera at a pixel size of 4.02 Å.

Processing of negative stain images. The RELION 3.0 program was used for all negative stain image processing. Images were imported, CTF-corrected with CTFFIND, and particles were picked using a spike template from previous 2D class averages of spike alone. Extracted particle stacks were subjected to 2-3 rounds of 2D class averaging and selection to discard junk particles and background picks. Cleaned particle stacks were then subjected to 3D classification using a starting model created from a bare spike model, PDB 6vsb, low-pass filtered to 30 Å. Classes that showed clearly-defined Fabs were selected for final refinements followed by automatic filtering and B-factor sharpening with the default Relion post-processing parameters.

Betacoronavirus sequence analysis. Heatmaps of amino acid sequence similarity were computed for a representative set of betacoronaviruses using the ComplexHeatmap package in R. Briefly, 1408 betacoronavirus sequences were retrieved from NCBI Genbank, aligned to the Wuhan-1 spike protein sequence, and trimmed to the aligned region. The 1408 spike sequences were then clustered using USEARCH⁷² with a sequence identity threshold of 0.90 resulting in 52 clusters. We sampled one sequence from each cluster to generate a representative set of sequences. Five betacoronavirus sequences of interest not originally included in the clustered set were added: SARS-CoV-2, GXP4L, batCoV-RaTG13, batCoV-SHC014, batCoV-WIV-1. This resulted in a set of 57 representative spike sequences. Pairs of spike amino acid sequences were aligned using a global alignment and the BLOSUM62 scoring matrix. For RBD and NTD domain alignments, spike sequences were aligned to the Wuhan 1 spike protein RBD region (residues 330-521) and NTD region (residues 27-292), respectively, and trimmed to the aligned region. Phylogenetic tree construction of RBD sequences was performed with Geneious Prime 2020.1.2 using the Neighbor Joining method and default parameters. To map group 2b betaCoV sequence conservation onto the RBD structure, group 2b spike sequences were retrieved from Genbank and clustered using USEARCH⁷² with a sequence identity threshold of 0.99 resulting in 39 clusters. For clusters of size >5, 5 spike sequences were randomly downsampled from each cluster. The resulting set of 73 sequences was aligned using MAFFT⁷³. Conservation scores for each position in the multiple sequence alignment were calculated using the trident scoring method⁷⁴ and computed using the MstatX program (https://github.com/gcollet/MstatX). The conservation scores were then mapped to the RBD domain coordinates (PDB: 7LD1) and images rendered with PyMol version 2.3.5.

Histopathology. Lung specimen from macaques were fixed in 10% neutral-buffered formalin, processed, and blocked in paraffin for histology analyses. All tissues were sectioned at 5 μm and stained with hematoxylin-eosin (H&E) for to assess histopathology. Stained sections were evaluated by a board-certified veterinary pathologist in a blinded manner. Sections were examined under light microscopy using an Olympus BX51 microscope and photographs were taken using an Olympus DP73 camera.

Immunohistochemistry (IHC). Staining for SARS-CoV-2 nucleocapsid antigen was performed by the Bond RX automated system with the Polymer Define Detection System (Leica) following the manufacturer's protocol. Tissue sections were dewaxed with Bond Dewaxing Solution (Leica) at 72° C. for 30 min, then subsequently rehydrated with graded alcohol washes and 1× Immuno Wash (StatLab). Heat-induced epitope retrieval (HIER) was performed using Epitope Retrieval Solution 1 (Leica) and by heating the tissue section to 100° C. for 20 min. A peroxide block (Leica) was applied for 5 min to quench endogenous peroxidase activity prior to applying the SARS-CoV-2 nucleocapsid antibody (1:2000, GeneTex, GTX135357). Antibodies were diluted in Background Reducing Antibody Diluent (Agilent). The tissue was subsequently incubated with an anti-rabbit HRP polymer (Leica) and colorized with 3,3′-Diaminobenzidine (DAB) chromogen for 10 min. Slides were counterstained with hematoxylin.

Statistics Analysis. Data were plotted using Prism GraphPad 9.0. Wilcoxon rank sum exact test was performed to compare differences between groups with p-value<0.05 considered significant using SAS 9.4 (SAS Institute, Cary, NC). No adjustments were made to the p-values for multiple comparisons. IC50 and IC80 values were calculated using R statistical software (version 4.0.0; R Foundation for Statistical Computing, Vienna, Austria). The R package ‘nplr’ was used to fit 4-Parameter Logistic (4-PL) regression curves to the average values from duplicate experiments, and these fits were used to estimate the concentrations corresponding to 50% and 80% neutralization.

### Example 1B

FIG. 18, FIG. 20, FIG. 21, FIG. 22, FIG. 23 and FIG. 43, and Tables 1, 2, 3, and 5 show non-limiting embodiments of amino acid sequences of immunogens of the invention.

In some embodiments these sequences are sequence optimized as nucleic acids sequences for expression as mRNA or DNA. Immunogen designs from Table 1, Table 2 and Table 3 will be expressed, characterized and tested for antigenicity and immunogenicity. Immunogenicity studies include animal challenge studies.

In non-limiting embodiments, the goal is to identify an immunogen design that can be expressed as a protein nanoparticles displaying coronavirus, including SARS-CoV-2, viral antigens.

In non-limiting embodiments, immunogenic composition comprise viral subunits derived from the Spike (S) protein of coronaviruses arrayed on self-assembling protein nanoparticles. The S subunit will include different versions of the receptor binding domain or other neutralizing determinants on the coronavirus spike protein.

Nucleic acid sequences encoding the amino acid sequences depicted in FIG. 18, FIG. 20, FIG. 21, FIG. 22, FIG. 23 and FIG. 43 will be codon-optimized for mammalian cell expression, including without limitation for expression as modified mRNAs.

Codon-optimized DNA encoding the amino acid sequences depicted in FIG. 18, FIG. 20, FIG. 21, FIG. 22, FIG. 23 and FIG. 43 will be made for production of recombinant protein encoding the coronavirus subunit nanoparticle vaccines.

Recombinant proteins will be evaluated for expression, stability, antigenicity, and immunogenicity in any suitable assay.

The expression of subunit nanoparticles will be quantified by ELISA. Also, antigenicity of each virus subunit nanoparticles expressed by RNA or DNA will be determined by biolayer interferometry binding to ACE-2 as a positive control, NTD and S2 antibodies as negative controls, and RBD antibodies as test antibodies. Constructs with binding to RBD antibodies and ACE-2 will be ranked based on magnitude of binding. Constructs that are antigenic for RBD antibodies and ACE-2 will then be confirmed to be nanoparticles by negative stain electron microscopy. The nanoparticles that bind to RBD antibodies and form nanoparticles will be subjected to immunogenicity testing in wildtype BALB/c mice. Mouse sera will be tested for reactivity with recombinant RBD to determine whether delivery of the mRNA expressing the viral subunit nanoparticle was immunogenic. Additionally, mouse sera will be tested for the ability to inhibit ACE-2 binding to SARS-CoV-2 spike, including different spike variants circulating worldwide (e.g. UK and South African variants). Sera will be tested for various betacoronavirus neutralization in pseudovirus and live virus assays. For each assay, the different designs will be ranked and the viral subunit nanoparticle with the lowest sum of their rankings for expression, blocking of ACE-2, and neutralization potency and breadth will be selected for additional animal studies, and further product development.

### Example 2

Additional animal studies, including mouse models, rabbits, ferrets, or non-human primates (NHPs) will be conducted with any of the immunogens. Immune response is evaluated and animals are challenged with coronavirus strain, including any suitable variant.

Analyses of the animal study will include immunogenicity, levels of antibodies, types of antibodies-neutralizing or not, serum neutralization of pseudo-virus, diversity of epitopes targeted by the induced antibodies, protection after challenge with virus, and any other suitable assay. Non-limiting embodiments of assays that could be used to characterize the immunogens of the invention are used and described in Examples 1 and 3.

Any suitable mouse model could be used, including without limitation humanized mouse models to determine the types of immune responses and antibodies induced by the immunogens of the invention.

### Example 3: Breadth of SARS-CoV-2 Neutralization and Protection Induced by a Nanoparticle Vaccine

Coronavirus vaccines that are highly effective against SARS-CoV-2 variants are needed to control the current pandemic. We previously reported a receptor-binding domain (RBD) sortase A-conjugated ferritin nanoparticle (RBD-scNP) vaccine that induced neutralizing antibodies against SARS-CoV-2 and pre-emergent sarbecoviruses and protected monkeys from SARS-CoV-2 WA-1 infection. Here, we demonstrate SARS-CoV-2 RBD-scNP immunization induces potent neutralizing antibodies against eight SARS-CoV-2 variants tested including the Beta, Delta, and Omicron variants in non-human primates (NHPs). The Omicron variant was neutralized by RBD-scNP-induced serum antibodies with a 3 to 9-fold reduction of ID80 titers compared to SARS-CoV-2 D614G. Immunization with RBD-scNPs protected NHPs from SARS-CoV-2 WA-1, Beta and Delta variants challenge, and protected mice from challenges of SARS-CoV-2 Beta variant and two other heterologous Sarbecoviruses. These results demonstrate the ability of RBD-scNPs to induce broad neutralization of SARS-CoV-2 variants and to protect NHPs or mice from multiple different SARS-related viruses. Such a vaccine could provide the needed immunity to slow the spread of and reduce disease caused by SARS-CoV-2 variants such as Delta and Omicron.

Despite the remarkable success of the approved COVID-19 vaccines, additional broadly protective vaccines may be needed to combat breakthrough infections caused by emerging SARS-CoV-2 variants and waning immunity. Moreover, pan-Sarbecovirus vaccines are needed for the prevention of new animal SARS-like viruses that may jump to humans in the future¹. Modified mRNA vaccines encapsulated in lipid nanoparticles (LNPs) have proved transformative for COVID-19 vaccine development and for vaccine development in general²⁻⁴. Developed in 11 months and providing >90% efficacy from transmission, the mRNA-1273 and the BNT162b2 COVID-1 vaccines, while showing the most reduction in efficacy from SARS-CoV-2 Beta and Omicron variants, continue to provide significant protection from serious COVID-19 disease, hospitalization, and death⁵⁷. The Omicron variant, however, has proved to be more transmissible than previous variants, now accounting for the majority of global isolates⁸. Arising from immunocompromised individuals in South Africa, the Omicron variant spike protein contains 30 mutations compared to the WA-1 strain, and continues to evolve⁹. While less pathogenic than Delta and other SARS-CoV-2 variants, the enhanced transmissibility of Omicron, coupled with the sheer number of resulting cases, has resulted in a higher absolute number of COVID patients compared to previous variant infections, thus providing a continued burden on global health care systems.

We have previously reported an RBD-based, sortase A-conjugated nanoparticle (RBD-scNP) vaccine formulated in the TLR7/8 agonist 3M-052-aqueous formulation (AF) (hereafter 3M-052-AF) plus Alum, that elicited cross-neutralizing antibody responses against SARS-CoV-2 and other sarbecoviruses, and protected against the WA-1 SARS-CoV-2 strain in non-human primates (NHPs)¹⁰. Here, we found RBD-scNPs induced antibodies that neutralized all variants tested including Beta and Omicron, and protected against Beta and Delta variant challenges in macaques. Moreover, RBD-scNP immunization protected in highly susceptible aged mouse models against challenges of SARS-CoV-2 Beta variant and other betacoronaviruses. In addition, while Alum, 3M-052-AF, or 3M-052-AF+Alum as adjuvant for RBD-scNP, each protected animals from WA-1 challenge, the 3M-052-AF/RBD-scNP formulation was optimal for induction of neutralization titers to variants and protection from lung inflammation. Finally, we found that RBD-, N-terminal domain (NTD)- and spike-2P (S2P)-scNPs each protected comparably in the upper and lower airways from WA-1, but boosting with the NTD-scNP protected less well than RBD- or S2P-scNP.

RBD-scNPs induce neutralizing antibodies against SARS-CoV-2 B.1.1.529 (Omicron) and other variants. RBD-scNPs were used to immunize macaques X3 four weeks apart (FIG. 28a). To test whether RBD-scNP-induced antibodies can neutralize SARS-CoV-2 variants, we collected macaque plasma samples two weeks after the 3^{rd} RBD-scNP immunization¹⁰ and assessed its ability to neutralize pseudovirus infection of 293T-ACE2-TMPRSS2 cells by SARS-CoV-2 WA-1 and 8 variants (FIG. 28a). RBD-scNP induced potent plasma neutralizing antibodies against the WA-1 strain with average ID50 titer of 12,266.7, while reduced ID50 titers were observed to different extents for the variants (FIG. 28b-c). For example, the highest reduction of neutralizing activities was observed in the B.1.351 (Beta) variant, ranged from 4.1- to 10.2-fold (FIG. 28c). We then asked if the B.1.1.529 (Omicron) variant could escape RBD-scNP-induced neutralizing antibodies. In a pseudovirus assay in 293T-ACE2 cells, monkey serum antibodies induced by three doses of RBD-scNP immunization¹⁰ neutralized both the D614G (ID₅₀=16,531, ID80=5,484) and Omicron (ID₅₀=3,858, ID₈₀=980) pseudoviruses. A 4.3-fold drop in the average ID₅₀ titer and 5.5-fold drop in the average ID₈₀ titer were observed (FIG. 28d). Thus, high titers of neutralizing antibodies against SARS-CoV-2 variants, including Omicron, were elicited by the RBD-scNP immunization in macaques.

RBD-scNPs induced higher titers of neutralizing antibodies than soluble RBD. Next, we asked if two doses of RBD-scNP vaccination could protect NHPs from challenge by SARS-CoV-2 WA-1, Beta or Delta variants. We immunized cynomolgus macaques with two doses of RBD-scNP vaccine, PBS, or adjuvant alone (FIG. 29a). One group of macaques received soluble RBD for comparison to RBD-scNP immunization. RBD-scNP and soluble RBD monomer immunization elicited similar titers of antibodies binding to SARS-CoV-2 and other CoV spike antigens (FIG. 34a), which also similarly blocked ACE2-binding on SARS-CoV-2 spike and bat CoV RsSHC014 spike (FIG. 34b-c). RBD-scNPs and soluble RBD induced similar levels of antibodies targeting the Sarbecovirus cross-neutralizing DH0147-epitope¹¹⁻¹³ on SARS-CoV-2 spike as well as on RsSHC014 spike (FIG. 34b-c). In pseudovirus neutralization assays, the RBD-scNP group exhibited higher titers of neutralizing antibodies than the soluble RBD group against the WA-1, Alpha, Epsilon, Iota, and Delta viruses, with comparable neutralizing titers against Beta, Gamma, and Kappa variants (FIG. 34d). In both groups, reduced neutralizing titers compared to WA-1 were seen for the Beta, Gamma, and Iota variants (FIG. 34e). Thus, RBD-scNP induced higher neutralizing antibodies than soluble RBD monomer for 5 out of 8 SARS-CoV-2 variant pseudoviruses we tested.

RBD-scNPs protected macaques from SARS-CoV-2 WA-1, Beta and Delta challenge. Two weeks after the second vaccination, macaques were challenged with SARS-CoV-2 WA-1 (n=5 per group), SARS-CoV-2 B.1.351 (Beta) variant (n=5 per group), or SARS-CoV-2 B.1.617.2 (Delta) variant (n=5 per group) (FIG. 29a). In the PBS or adjuvant alone group, high copies of envelope (E) and nucleocapsid (N) gene subgenomic RNA (sgRNA) were detected in both BAL and nasal swab samples collected on day 2 and 4 post-challenge (FIG. 29b-d). By contrast, 5 of 5 animals in the RBD-scNP group, and 4 of 5 animals in the soluble RBD group, were completely protected from the WA-1 strain infection, as indicated by no detectable sgRNA in BAL or nasal swab (FIG. 29b). After the SARS-CoV-2 Beta variant challenge, nasal N gene sgRNA was detected in only ⅕ of the RBD-scNP immunized monkeys but in 4 of 5 of the soluble RBD immunized monkeys (FIG. 29c). In addition, after the SARS-CoV-2 Delta variant challenge, all animals that received two doses of RBD-scNP immunization showed no detectable sgRNA in BAL or nasal swab samples (FIG. 29d).

Animals were necropsied 4 days after challenge for histopathologic analysis to determine SARS-CoV-2-associated lung inflammation. After WA-1 and Beta variant challenge, lung tissue Haematoxylin and eosin (H&E) staining revealed no difference between groups (FIG. 29e-f). However, immunohistochemistry (IHC) staining showed the presence of SARS-CoV-2 nucleocapsid antigen in the lungs of macaques administered PBS or adjuvant alone, but not in the lungs of RBD-scNP or soluble RBD immunized monkeys (FIG. 29e-f). Thus, while lung inflammation was observed in immunized macaques, two doses of RBD-scNP immunization protected against viral replication of WA-1, the Beta variant, or Delta variant in both lower and upper airways. In addition, RBD-scNP was superior to soluble RBD in terms of protecting from the hard-to-neutralize Beta variant infection in the upper respiratory tract.

RBD-scNPs induce protective responses in mice against SARS-CoV-2 Beta variant and other betacoronaviruses. To define the protective efficacy of the RBD-scNP vaccination against different betacoronaviruses, we immunized aged mice with two doses of RBD-scNPs, challenged the mice with mouse-adapted SARS-CoV-2 WA-1, SARS-CoV-2 Beta variant, SARS-CoV-1 or a bat CoV RsSHC014. In the SARS-CoV-2 WA-1 challenge study, RBD-scNP protected mice from weight loss through 4 dpi (FIG. 30a) and protected from viral replication in lungs (FIG. 30b). Similar protection from weight loss and lung viral replication were observed in the SARS-CoV-2 Beta variant challenged mice (FIG. 30c-d). Mice immunized with RBD-scNP were also protected against weight loss induced by SARS-CoV-1 (FIG. 30e) and showed ˜3-log lower average PFU titer in lungs compared to adjuvant alone and unimmunized groups (FIG. 30f). Lastly, RBD-scNP immunization conferred protection against RSHC014 challenge-induced weight loss (FIG. 3g) and resulted in ˜2-log lower average PFU titer than unimmunized animals (FIG. 30h). Thus, two doses of RBD-scNP immunization elicited protective immune responses against SARS-CoV-2 Beta variant and other betacoronaviruses in aged mouse models.

Adjuvant is required for RBD-scNP induction of potent plasma and mucosal antibody responses. To optimize adjuvant formulations for the RBD-scNP vaccine, we next formulated the RBD-scNP immunogen with the TLR7/8 agonist 3M-052-AF alone, with Aluminum hydroxide (Alum) alone, or with the original formulation of 3M-052-AF adsorbed to Alum (3M-052-AF+Alum). Control groups included NHPs immunized with immunogen alone (RBD-scNP without adjuvant), adjuvant alone (3M-052-AF, Alum, or 3M-052-AF+Alum without immunogen), or PBS alone (FIG. 31a). After three immunizations, RBD-scNP alone without adjuvant induced minimal binding antibodies to SARS-CoV-2 and other CoV spike antigens, whereas higher titers of binding antibodies were induced by RBD-scNP formulated with each adjuvant formulation (FIG. 35a). While all three adjuvant formulations were highly immunogenic, RBD-scNP adjuvanted with 3M-052-AF induced the highest DH1047-blocking plasma antibodies (p<0.05; Wilcoxon rank sum exact test; FIG. 35b-c). Mucosal antibody levels tended to be comparable for macaques who received RBD-scNP formulated with 3M-052-AF or 3M-052+Alum, with only low titers being seen when Alum was used to adjuvant the RBD-scNP (FIG. 35d-e).

Robust neutralizing antibodies and in vivo protection induced by adjuvanted RBD-scNP. In SARS-CoV-2 pseudovirus assay, while the RBD-scNP alone group showed minimal neutralizing antibody titers, the RBD-scNP+3M-052-AF group induced antibodies showed highest neutralizing antibody titers against SARS-CoV-2 WA-1 strain, with average neutralization titers (ID₅₀) of 59,497. The average neutralization titers of RBD-scNP+3M-052-Alum and RBD-scNP+Alum groups against WA-1 were 12,267 and 12,610, respectively (FIG. 31b). Similarly, RBD-scNP+3M-052-AF immunized animals exhibited the highest magnitudes of neutralizing antibodies against each variant we tested (FIG. 31b). The neutralization titers of all three RBD-scNP+adjuvant groups decreased by 2.3- to 10.2-fold against the Beta variant and decreased by 1.9- to 8.6-fold against the Gamma variant (FIG. 31c). RBD-scNP immunization elicited predominately T helper 1 (TH1)-biased cellular immune responses¹⁴ (FIG. 37f). NHPs that received RBD-scNP also showed IFN-γ-, IL-2- and TNF-α-secreting CD8+ T cell responses (FIG. 37g).

To compare in vivo protection of RBD-scNP with different adjuvant formulations, cynomolgus macaques were challenged with the WA-1 strain of SARS-CoV-2 three weeks after the third immunization (FIG. 31a). Compared to unimmunized monkeys, the adjuvant alone groups exhibited similar or higher levels of E and N sgRNA, and the RBD-scNP immunogen alone reduced sgRNA copies by only ˜1-2 logs (FIG. 31d-e), demonstrating that adjuvant was required for eliciting potent protection from SARS-CoV-2 challenge. Immunization with RBD-scNP adjuvanted with 3M-052-AF+Alum or 3M-052-AF conferred robust protection against SARS-CoV-2 infection, showing under-detection-limit or near-baseline sgRNA N and E in both lower and upper respiratory tracts, whereas 1 of 5 of the RBD-scNP+Alum immunized animals showed positive E gene sgRNA and 2/5 animals showed positive N gene sgRNA in BAL samples collected on day 2 post-challenge. All RBD-scNP adjuvanted groups showed no detectable sgRNA by day 4 post-challenge (FIG. 31d-e).

Histologic analysis of lung tissue showed that the RBD-scNP+3M-052-AF group had significantly lower inflammation scores than the 3M-052-AF alone group (p=0.0079, exact Wilcoxon test), whereas no significant difference was observed across other groups (FIG. 31f). IHC staining of the lung tissues exhibited high SARS-CoV-2 nucleocapsid antigen expression in the unimmunized and adjuvant alone groups. By contrast, 1 of 5 of the RBD-scNP+Alum immunized animals and 2 of 5 of the immunogen alone immunized animals had low level nucleocapsid antigen expression, and no viral antigen was detected in the RBD-scNP plus 3M-052-AF+Alum or RBD-scNP plus 3M-052-AF immunized animals (FIG. 31f). Therefore, the three adjuvants conferred comparable protection against viral replication by day 4 post-challenge, but 3M-052-AF-adjuvanted RBD-scNP protected animals better against SARS-CoV-2-associated lung inflammation.

RBD-scNP and S2P-scNP induced both ADCC-mediating and neutralizing antibodies. While 90% of neutralizing antibodies target the RBD, neutralizing antibodies can target other sites on Spike. Thus, we generated scNPs with NTD and S-2P and compared the antibody response elicited by these ferritin nanoparticles to RBD-scNPs (FIG. 32a,b). Cynomolgus macaques were immunized three times with one of the scNPs formulated with 3M-052-AF+Alum. After three immunizations, binding and blocking antibodies were observed in all three groups (FIG. 36a-b). In the RBD-scNP and S2P-scNP immunized animals, neutralizing antibodies against SARS-CoV-2 D614G pseudovirus were detected after the first dose and were boosted after the second dose at week 6 (FIG. 32c). However, NTD-scNP failed to elicit neutralizing antibodies against pseudovirus or live SARS-CoV-2 virus (FIG. 32c-d). Importantly, S2P-scNP induced comparable plasma neutralizing antibody titers with RBD-scNP against SARS-CoV-2 WA-1 strain and all eight variants we tested (p>0.05; Wilcoxon rank sum exact test; FIG. 32e) indicating the RBD was the main domain eliciting neutralizing antibodies. Among the different variants, the Beta variant showed the largest reduction in neutralization ID₅₀ titer (5.0- to 10.9-fold) (FIG. 32f). To examine other antibody functions, we examined plasma antibody binding to cell surface-expressed SARS-CoV-2 spike and antibody-dependent cellular cytotoxicity (ADCC). Plasma antibodies induced by three doses of RBD-scNP, NTD-scNP and S2P-scNP vaccination bound to SARS-CoV-2 spike on the surface of transfected cells (FIG. 36d). In a CD107a degranulation ADCC assay (FIG. 36e), plasma antibodies from all three scNP groups mediated CD107a degranulation of human NK cells in the presence of both SARS-CoV-2 spike-transfected cells and SARS-CoV-2-infected cells (FIG. 36f). Thus, all three groups had ADCC antibodies, only RBD-scNP- and S2P-scNP-immunized groups also generated neutralizing antibodies.

RBD-scNP, NTD-scNP, and S2P-scNP vaccines protected macaques against SARS-CoV-2 WA-1 challenge. To determine whether NTD-scNP- and S2P-scNP immunization conferred protection against SARS-CoV-2, we challenged the macaques with SARS-CoV-2 WA-1 strain via the intratracheal and intranasal routes after the 3^{rd} vaccination. Remarkably, all macaques received RBD-scNP, NTD-scNP or S2P-scNP were fully protected, showing undetectable or near-detection-limit E or N gene sgRNA (FIG. 32g-h). IHC staining of the lung tissues demonstrated high SARS-CoV-2 nucleocapsid protein expression in the control animals, whereas no viral NC antigen was detected in any of the scNP-immunized animals (FIG. 32i). The sgRNA and histopathology data demonstrated that three doses of NTD-scNP or S2P-scNP immunization provided the same in vivo protection as RBD-scNP immunization, preventing SARS-CoV-2 infection in both lower and upper respiratory tracts. That the NTD-scNP group had no or minimal serum neutralizing activity but did have ADCC activity, indicated that non-neutralizing Fc receptor-mediated antibody activities or T cells could have been involved in protection.

RBD-scNP, NTD-scNP and S2P-scNP as boosts for mRNA-LNP vaccine elicited various neutralizing antibody responses. We next accessed the efficacy of the RBD-scNP, NTD-scNP and S2P-scNP as boosts in animals that received two doses of mRNA vaccine. Cynomolgus macaques (n=5) were immunized twice with 50 μg of S-2P-encoding, nucleoside-modified mRNA encapsulated in lipid nanoparticles (S-2P mRNA-LNP), which phenocopies the Pfizer/BioNTech and the Modema COVID-19 vaccines. Animals were then injected with a heterologous boost with RBD-, NTD- or S2P-scNPs (FIG. 33a). Plasma antibody binding patterns were similar among the three groups until animals received the scNP boosting (FIG. 37a). Plasma antibodies targeting to ACE2-binding site and neutralizing epitopes were detected after the scNP boosting with cross-reactive antibodies in the DH1047 blocking assay highest after RBD-scNP_and S2P-NP boosting (FIG. 37b-c). BAL and nasal wash mucosal ACE2-blocking and DH1047-blocking activities were highest in RBD-scNP×3 and S2P-scNP×3 groups with trends of higher values in the RBD-scNP group (FIG. 37d-e).

Serum neutralizing titers against the WA-1 strain pseudovirus were similar in the RBD-scNP-boosted group (average ID₅₀=10,912.1) and S2P-scNP-boosted group (average ID₅₀=7799.9) (FIG. 33b), while the NTD-scNP-boosted group showed significant lower titers (average ID₅₀=3229.8; p=0.027, exact Wilcoxon test). The same differences were also observed in other major variants (FIG. 33b). In addition, in the RBD-scNP- and S2P-scNP-boosted groups, reduced ID₅₀ titers were mostly seen for the Beta and Gamma variants, whereas in the NTD-scNP-boosted group, Alpha, Beta, Gamma, Delta, Iota and Kappa variants all showed >5-fold reduction of ID₅₀ titers (FIG. 33c).

Protection of mRNA-LNP-primed and scNP-boosted macaques from SARS-CoV-2 challenge. Macaques that received mRNA-LNP prime and scNP boost were challenged with SARS-CoV-2 WA-1 strain after the RBD-scNP, NTD-scNP or S2P-scNP boosting. Four of five RBD-scNP-boosted monkeys and Four of five of the S2P-scNP-boosted monkeys were completely protected from SARS-CoV-2 infection, showing no detectable E or N gene sgRNA in BAL or nasal swab samples (FIG. 33d-e). However, NTD-scNP-boosted animals were not as well protected; in the NTD-scNP boost group, N gene sgRNA was detected in BAL from three of five animals and in nasal swab samples from two of five animals (FIG. 33d-e). Macaques that received mRNA-LNP prime and RBD-scNP boost had the lowest extent of lung inflammation, although it was not significantly lower (FIG. 33f). In addition, no viral antigen was observed in lung tissues from the immunized groups as indicated by IHC staining for SARS-CoV-2 N protein (FIG. 33f). These data demonstrated that animals received mRNA-LNP-prime and scNP-boost regimens were not as well protected as those received three doses of RBD-scNP, NTD-scNP or S2P-scNP vaccination.

In this Example, we demonstrated that the RBD-scNP induced antibodies that neutralize SARS-CoV-2 variants including Beta, Delta and Omicron, and as well, protected against Beta and Delta variants in NHPs. When the individual components of 3M-052/Alum adjuvant were studied for optimal formulation of RBD-scNPs, we found 3M-052-AF alone plus RBD-scNP was optimal. Finally, NTD- and S2P-scNPs protected NHPs from WA-1 challenge as well as RBD-NPs, but NTD-scNPs were less effective as a boosting immunogen in protecting NHPs compared to RBD- or S2P-scNPs.

Several protein-based SARS-CoV-2 vaccines have been designed as VLPs¹⁵, RBD monomers¹⁶, dimers¹⁷ or trimers¹⁸ as well as multimeric nanoparticles^{19,20}. In our previous study, we demonstrated that RBD multimerized on sortase A-conjugated ferritin nanoparticles is a promising vaccine platform for SARS-CoV-2 and other Sarbecoviruses¹⁰. Although the currently approved mRNA and viral vectored vaccines showed high efficacy against hospitalization and death of current SARS-CoV-2 variants^{5,7,21-35}, it is important to develop new vaccines to combat any new variants that might emerge. Ferritin nanoparticle-based Influenza vaccines have been demonstrated to be safe and immunogenic in animal models³⁶ and have been tested in clinical trials (NCT03186781, NCT03814720). We and others have shown that RBD-ferritin nanoparticle vaccines elicited high-titer neutralizing antibodies and conferred potent in vivo protection against SARS-CoV-2 challenge^{10,37-40}. Here, we engineered RBD, NTD and S2P on scNP and compared their capacities to elicit neutralizing antibodies and protect against SARS-CoV-2 WA-1 strain challenge in cynomolgus macaques, given as homologous immunization for 3 doses, or as a 3^{rd} dose heterologous boost for the S-2P mRNA-LNP vaccine. We also demonstrated the success of two-doses of RBD-scNP vaccine in the setting of protecting macaques from SARS-CoV-2 WA-1, Beta variant and Delta variant challenge.

SARS-CoV-2 vaccine-induced neutralizing antibody activity is associated with vaccine protection against COVID-19 in non-human primates and humans⁴¹⁻⁴⁷. The Novavax NVX-CoV2373 adjuvanted virus-like particle vaccine that contains a full-length spike and transmembrane domain, has demonstrated an efficacy of 89.7% against SARS-CoV-2 infection in clinical trials⁴⁸⁻⁵⁰. While the RBD subunit has been shown to protect against SARS-CoV-2 challenge in animal models^{10,16,17,51,52}, the NTD is also an immunodominant region for neutralizing antibodies^{11,32,53-55} However, NTD is the site of multiple mutation and NTD antibody neutralization is less potent than RBD antibodies. Here, no or minimal neutralizing antibody was detected in NTD-scNP vaccinated monkeys, yet they were fully protected from WA-1 challenge. Without wishing to be bound by theory, the non-neutralizing antibodies induced by NTD-scNP played essential roles in the protection against SARS-CoV-2. Recent studies in SARS-CoV-2 mouse models demonstrated that Fc effector functions contribute to the protective activity of SARS-CoV-2 neutralizing antibodies⁵⁶⁻⁵⁸. In this regard, we previously found that a non-neutralizing NTD antibody DH1052 provided partial protection in mice and non-human primates¹¹. Moreover, we found that whereas RBD-scNP and S2P-scNP after boosting S2P mRNA-LNPs both protected completely monkeys after WA-1 challenge, NTD-scNP boosting of S2P mRNA-LNPs led to incomplete protection. The mechanism of this finding is currently under investigation.

Adjuvants play essential roles in vaccine formulation to elicit strong protective immune responses⁵⁹ and Alum is used in many currently approved vaccines⁶⁰. Thus, it was encouraging to see that the RBD-NP vaccine was protective in NHPs when adsorbed to Alum. Compared to Alum, 3M-052-AF+Alum demonstrated superior capacities to elicit neutralizing antibodies against SARS-CoV-2 WA-1 live virus when formulated with SARS-CoV-2 RBD trimer in mice but not in rhesus macaques¹⁸. In addition, 3M-052-adjuvanted gp140 Env vaccine augmented neutralizing antibodies against tier 1A HIV-1 pseudovirus in rhesus macaques⁶¹. 3M-052-AF and 3M-052-AF+Alum are both in clinical testing for HIV-1 vaccines (NCT04915768 and NCT04177355). Here we found that 3M-052-AF-adjuvanted vaccine induced not only superior systemic and mucosal antibody responses, but also higher titers of neutralizing antibodies than 3M-052-AF+Alum-adjuvanted vaccine, demonstrating that 3M-052-AF in the absence of Alum is an optimal adjuvant for scNP. One explanation for this difference could be while 3M-052 provokes a strong Th1 response⁶², Alum is a Th2-response stimulator⁶³. Coronavirus vaccines formulated with Alum have been reported to be associated with enhanced lung inflammation, for example with killed vaccines^{64,65}. However, it is important to note that no enhancement of lung inflammation or virus replication was seen with RBD-scNP/Alum formulations. The RBD-scNP+3M-052-AF group exhibited the highest neutralizing antibody titers and was the only group showing reduced severity of lung inflammation.

This study did not evaluate the durability of vaccine-induced immune responses and protection against SARS-CoV-2 variants. Second, we did not set up longer time intervals between the second and the third booster vaccination, to mimic 4-6 month boosting interval in humans. Lastly, we challenged the animals with WA-1 strain, the Beta variant and the Delta variant; future in vivo protection studies will be required upon availability of viral stocks of other SARS-CoV-2 variants such as the Omicron variant.

Thus, our study showed that scNP vaccines with SARS-CoV-2 spike or spike subunits conferred potent protection for WA-1, Beta and Delta variants in NHPs and induced neutralizing antibodies to all SARS-CoV-2 variants tested in vitro. These findings have important implications for development of the next generation of COVID-19 vaccines.

### Materials and Methods

### Animals and Immunizations

The study protocol and all veterinarian procedures were approved by the Bioqual IACUC per a memorandum of understanding with the Duke IACUC, and were performed based on standard operating procedures. Macaques studied were housed and maintained in an Association for Assessment and Accreditation of Laboratory Animal Care-accredited institution in accordance with the principles of the National Institutes of Health. All studies were carried out in strict accordance with the recommendations in the Guide for the Care and Use of Laboratory Animals of the National Institutes of Health in BIOQUAL (Rockville, MD). BIOQUAL is fully accredited by AAALAC and through OLAW, Assurance Number A-3086. All physical procedures associated with this work were done under anesthesia to minimize pain and distress in accordance with the recommendations of the Weatherall report, “The use of non-human primates in research.” Teklad 5038 Primate Diet was provided once daily by animal size and weight. The diet was supplemented with fresh fruit and vegetables. Fresh water was given ad libitum. All monkeys were maintained in accordance with the Guide for the Care and Use of Laboratory Animals.

Cynomolgus macaques were on average 8-9 years old and ranged from 2.75 to 8 kg in body weight. Male and female macaques per group were balanced when availability permitted. Studies were performed unblinded. The RBD-scNP, NTD-scNP and S2P-scNP immunogens were formulated with adjuvants as previously described⁶⁶ and given intramuscularly in the right and left quadriceps. In the first study, cynomolgus macaques (n=5) were immunized for three times with 100 μg of RBD-scNP, NTD-scNP and S2P-scNP adjuvanted with 5 μg of 3M-052 aqueous formulation admixed with 500 μg of alum in PBS. In the second study, cynomolgus macaques (n=5) were immunized twice with 50 μg of S-2P mRNA-LNP (encoding the transmembrane spike protein stabilized with K986P and V987P mutations) and boosted once with 100 μg of RBD-scNP, NTD-scNP and S2P-scNP adjuvanted with 5 μg of 3M-052 aqueous formulation admixed with 500 μg of alum in PBS. In the third study, cynomolgus macaques were immunized for twice with 100 μg of RBD-scNP or recombinant soluble RBD with 5 μg of 3M-052 aqueous formulation admixed with 500 μg of alum in PBS. In the fourth study, macaques were divided into 8 groups (n=5 per group) as following: 1) control group: no immunization; 2) immunogen alone group: 100 μg of RBD-scNP; 3) RBD-scNP+3M-052-Alum group: 100 μg of RBD-scNP+5 μg of 3M-052 in aqueous formulation+500 μg of Alum (i.e. aluminum ion); 4) 3M-052-Alum alone group: 5 μg of 3M-052 in aqueous formulation+500 μg of Alum; 5) RBD-scNP+Alum group: 100 μg of RBD-scNP+500 μg of Alum; 6) Alum alone group: 500 μg of Alum; 7) RBD-scNP+3M-052-AF group: 100 μg of RBD-scNP+5 μg of 3M-052 in aqueous formulation; 8) 3M-052-AF alone group: 5 μg of 3M-052 in aqueous formulation.

### SARS-CoV-2 Viral Challenge

For SARS-CoV-2 challenge, 10⁵ plaque-forming units (PFU) of SARS-CoV-2 virus Isolate USA-WA1/2020 (˜10⁶ TCID50) were diluted in 4 mL and were given by 1 mL intranasally and 3 mL intratracheally on Day 0. Biospecimens, including nasal swabs, BAL, plasma, and serum samples, were collected before immunization, after every immunization, before challenge, 2 days post-challenge and 4 days post-challenge. Animals were necropsied on Day 4 post-challenge, and lungs were collected for histopathology and immunohistochemistry (IHC) analysis.

### Recombinant Protein Production

The coronavirus ectodomain proteins were produced and purified as previously described^{10,11,67,68}. S-2P was stabilized by the introduction of 2 prolines at amino acid positions 986 and 987. Plasmids encoding SARS-CoV-2 and other coronavirus S-2P (Genscript) were transiently transfected in FreeStyle 293 cells (Thermo Fisher) using Turbo293 (SpeedBiosystems) or 293Fectin (ThermoFisher). All cells were tested monthly for mycoplasma. The constructs contained an HRV 3C-cleavable C-terminal twinStrepTagII-8×His tag (SEQ ID NO: 17). On day 6, cell-free culture supernatant was generated by centrifugation of the culture and filtering through a 0.8-μm filter. Protein was purified from filtered cell culture supernatants by StrepTactin resin (IBA) and by size-exclusion chromatography using Superose 6 column (GE Healthcare) in 10 mM Tris pH8,150 mM NaCl or 2 mM Tris pH 8, 200 mM NaCl, 0.02% NaN₃. ACE2-Fc was expressed by transient transfection of Freestyle 293-F cells. ACE2-Fc was purified from cell culture supernatant by HiTrap protein A column chromatography and Superdex200 size-exclusion chromatography in 10 mM Tris pH8,150 mM NaCl. SARS-CoV-2 RBD and NTD were produced as previously described^{10,68}.

RBD-scNP, NTD-scNP, and S2P-scNP were produced by conjugating SARS-CoV-2 RBD to H. pylori ferritin nanoparticles using Sortase A as previously described¹⁰. Briefly, SARS-CoV-2 Wuhan strain RBD, NTD or S-2P (with a C-terminal foldon trimerization motif) was expressed with a sortase A donor sequence LPETGG (SEQ ID NO: 16) encoded at its C terminus. C-terminal to the sortase A donor sequence was an HRV-3C cleavage site, 8×His tag (SEQ ID NO: 17) and a twin StrepTagII (IBA). The proteins were expressed in Freestyle 293 cells and purified by StrepTactin affinity chromatography and Superdex 200 size-exclusion chromatography. Helicobacter pylori ferritin particles were expressed with a pentaglycine sortase A acceptor sequence encoded at its N terminus of each subunit. For affinity purification of ferritin particles, 6×His tags (SEQ ID NO: 48) were appended C-terminal to a HRV3C cleavage site. Ferritin particles with a sortase A N-terminal tag were buffer exchanged into 50 mM Tris, 150 mM NaCl, 5 mM CaCl₂), pH 7.5. Then 180 μM SARS-CoV-2 RBD was mixed with 120 μM of ferritin subunits and incubated with 100 μM of sortase A overnight at room temperature. Following incubation, conjugated particles were isolated from free ferritin or free RBD/NTD/S-2P by size-exclusion chromatography using a Superose 6 16/60 column.

### Antibody Binding ELISA

For binding ELISA, 384-well ELISA plates were coated with 2 μg/mL of antigens in 0.1 M sodium bicarbonate overnight at 4° C. Plates were washed with PBS+0.05% Tween 20 and blocked with blocked with assay diluent (PBS containing 4% (w/v) whey protein, 15% Normal Goat Serum, 0.5% Tween-20, and 0.05% Sodium Azide) at room temperature for 1 hour. Plasma or mucosal fluid were serially diluted threefold in superblock starting at a 1:30 dilution. Nasal fluid was started from neat, whereas BAL fluid was concentrated ten-fold. To concentrate BAL, individual BAL aliquots from the same macaque and same time point were pooled in 3-kDa MWCO ultrafiltration tubes (Sartorious, catalog #VS2091). Pooled BAL was concentrated by centrifugation at 3,500 rpm for 30 min or until volume was reduced by a factor of 10. The pool was then aliquoted and frozen at −80° C. until its use in an assay. Serially diluted samples were added and incubated for 1 hour, followed by washing with PBS-0.1% Tween 20. HRP-conjugated goat anti-human IgG secondary Ab (SouthernBiotech, catalog #2040-05) was diluted to 1:10,000 and incubated at room temperature for 1 hour. These plates were washed four times and developed with tetramethylbenzidine substrate (SureBlue Reserve—KPL). The reaction was stopped with 1 M HCl, and optical density at 450 nm (OD₄₅₀) was determined.

### ACE2 and Neutralizing Antibody Blocking Assay

ELISA plates were coated as stated herein with 2 μg/mL recombinant ACE-2 protein or neutralizing antibodies, then washed and blocked with 3% BSA in 1×PBS. While assay plates blocked, plasma or mucosal samples were diluted as stated herein, only in 1% BSA with 0.05% Tween-20. In a separate dilution plate spike-2P protein was mixed with the antibodies at a final concentration equal to the EC50 at which spike binds to ACE-2 protein. The mixture was incubated at room temperature for 1 hour. Blocked assay plates were then washed and the antibody-spike mixture was added to the assay plates for a period of 1 hour at room temperature. Plates were washed and a polyclonal rabbit serum against the same spike protein (nCoV-1 nCoV-2P.293F) was added for 1 hour, washed and detected with goat anti rabbit-HRP (Abcam catalog #ab97080) followed by TMB substrate. The extent to which antibodies were able to block the binding spike protein to ACE-2 or neutralizing antibodies was determined by comparing the OD of antibody samples at 450 nm to the OD of samples containing spike protein only with no antibody. The following formula was used to calculate percent blocking: blocking %=(100−(OD sample/OD of spike only)*100).

### Pseudo-Typed SARS-CoV-2 Neutralization Assay

Neutralization of SARS-CoV-2 Spike-pseudotyped virus was performed by adopting an infection assay described previously⁶⁹ with lentiviral vectors and infection in 293T/ACE2.MF (the cell line was kindly provided by Drs. Mike Farzan and Huihui Mu at Scripps). Cells were maintained in DMEM containing 10% FBS and 50 μg/ml gentamicin. An expression plasmid encoding codon-optimized full-length spike of the Wuhan-1 strain (VRC7480), was provided by Drs. Barney Graham and Kizzmekia Corbett at the Vaccine Research Center, National Institutes of Health (USA). The D614G mutation was introduced into VRC7480 by site-directed mutagenesis using the QuikChange Lightning Site-Directed Mutagenesis Kit from Agilent Technologies (Catalog #210518). The mutation was confirmed by full-length spike gene sequencing. Pseudovirions were produced in HEK 293T/17 cells (ATCC cat. no. CRL-11268) by transfection using Fugene 6 (Promega, Catalog #E2692). Pseudovirions for 293T/ACE2 infection were produced by co-transfection with a lentiviral backbone (pCMV ΔR8.2) and firefly luciferase reporter gene (pHR′ CMV Luc)⁷⁰. Culture supernatants from transfections were clarified of cells by low-speed centrifugation and filtration (0.45 μm filter) and stored in 1 ml aliquots at −80° C. A pre-titrated dose of virus was incubated with 8 serial 3-fold or 5-fold dilutions of mAbs in duplicate in a total volume of 150 μl for 1 hr at 37° C. in 96-well flat-bottom poly-L-lysine-coated culture plates (Corning Biocoat). Cells were suspended using TrypLE express enzyme solution (Thermo Fisher Scientific) and immediately added to all wells (10,000 cells in 100 μL of growth medium per well). One set of 8 control wells received cells+virus (virus control) and another set of 8 wells received cells only (background control). After 66-72 hrs of incubation, medium was removed by gentle aspiration and 30 μL of Promega 1× lysis buffer was added to all wells. After a 10-minute incubation at room temperature, 100 μl of Bright-Glo luciferase reagent was added to all wells. After 1-2 minutes, 110 μl of the cell lysate was transferred to a black/white plate (Perkin-Elmer). Luminescence was measured using a PerkinElmer Life Sciences, Model Victor2 luminometer. Neutralization titers are the mAb concentration (IC50/IC80) at which relative luminescence units (RLU) were reduced by 50% and 80% compared to virus control wells after subtraction of background RLUs. Negative neutralization values are indicative of infection-enhancement. Maximum percent inhibition (MPI) is the reduction in RLU at the highest mAb concentration tested.

Another protocol was used to test plasma neutralization against pseudoviruses of SARS-CoV-2 WA-1 strain and variants. Human codon-optimized cDNA encoding SARS-CoV-2 spike glycoproteins of various strains were synthesized by GenScript and cloned into eukaryotic cell expression vector pcDNA 3.1 between the BamHI and XhoI sites. Pseudovirions were produced by co-transfection of Lenti-X 293T cells with psPAX2(gag/pol), pTrip-luc lentiviral vector and pcDNA 3.1 SARS-CoV-2-spike-deltaC19, using Lipofectamine 3000. The supernatants were collected at 48 h after transfection and filtered through 0.45-μm membranes and titrated using HEK293T cells that express ACE2 and TMPRSS2 protein (293T-ACE2-TMPRSS2 cells). For the neutralization assay, 50 μl of SARS-CoV-2 spike pseudovirions were pre-incubated with an equal volume of medium containing serum at varying dilutions at room temperature for 1 h, then virus-antibody mixtures were added to 293T-ACE2-TMPRSS2 cells in a 96-well plate. After a 3-h incubation, the inoculum was replaced with fresh medium. Cells were lysed 24 h later, and luciferase activity was measured using luciferin. Controls included cell-only control, virus without any antibody control and positive control sera. Neutralization titres are the serum dilution (ID50 or ID80) at which relative luminescence units (RLU) were reduced by 50% or 80%, respectively, compared to virus control wells after subtraction of background RLUs.

### Live SARS-CoV-2 Neutralization Assays

The SARS-CoV-2 virus (Isolate USA-WA1/2020, NR-52281) was deposited by the Centers for Disease Control and Prevention and obtained through BEI Resources, NIAID, NIH. SARS-CoV-2 Plaque Reduction Neutralization Test (PRNT) were performed in the Duke Regional Biocontaiment Laboratory BSL3 (Durham, NC) as previously described with virus-specific modifications⁷¹. Briefly, two-fold dilutions of plasma samples were incubated with 50 PFU SARS-CoV-2 virus (Isolate USA-WA1/2020, NR-52281) for 1 hour. The antibody/virus mixture is used to inoculate Vero E6 cells in a standard plaque assay^{72,73}. Briefly, infected cultures are incubated at 37° C., 5% C02 for 1 hour. At the end of the incubation, 1 mL of a viscous overlay (1:1 2×DMEM and 1.2% methylcellulose) is added to each well. Plates are incubated for 4 days. After fixation, staining and washing, plates are dried and plaques from each dilution of each sample are counted. Data are reported as the concentration at which 50% of input virus is neutralized. A known neutralizing control antibody is included in each batch run (Clone D001; SINO, CAT #40150-D001). GraphPad Prism was used to determine IC/EC₅₀ values.

### Spike Protein-Expressing Cell Antibody Binding Assay

The cell antibody binding assay was performed as previously described (Pino et al., 2021). Briefly, target cells were derived by transfection with plasmids designed to express the SARS-CoV-2 D614 Spike protein with a c-terminus flag tag (kindly provided by Dr. Farzan, Addgene plasmid no. 156420 (Zhang et al., 2020)). Cells not transfected with any plasmid (mock transfected) were used as a negative control condition. After resuspension, washing and counting, 1×10⁵ Spike-transfected target cells were dispensed into 96-well V-bottom plates and incubated with six serial dilutions of human plasma from infected participants starting at 1:50 dilution. Mock transfected cells were used as a negative infection control. After 30 minutes incubation at 37° C., cells are washed twice with 250 μL/well of PBS, stained with vital dye (Live/Dead Far Red Dead Cell Stain, Invitrogen) to exclude nonviable cells from subsequent analysis, washed with Wash Buffer (1% FBS-PBS; WB), permeabilized with CytoFix/CytoPerm (BD Biosciences), and stained with 1.25 μg/mL anti-human IgG Fc-PE/Cy7 (Clone HP6017; Biolegend) and 5 μg/mL anti-flag-FITC (clone M2; Sigma Aldrich) in the dark for 20 minutes at room temperature. After three washes with Perm Wash (BD Biosciences), the cells were resuspended in 125 μL PBS-1% paraformaldehyde. Samples were acquired within 24 h using a BD Fortessa cytometer and a High Throughput Sampler (HTS, BD Biosciences). Data analysis was performed using FlowJo 10 software (BD Biosciences). A minimum of 50,000 total events were acquired for each analysis. Gates were set to include singlet, live, flag+ and IgG+ events. All final data represent specific binding, determined by subtraction of non-specific binding observed in assays performed with mock-transfected cells.

### Antibody-Dependent NK Cell Degranulation Assay

Cell-surface expression of CD107a was used as a marker for NK cell degranulation, a prerequisite process for ADCC (Ferrari et al., 2011), was performed as previously described (Pino et al., 2021). Briefly, target cells were Vero E6 cells after a 2 day-infection with SARS-CoV-2 USA-WA1/2020 or 293T cells 2-days post transfection with a SARS-CoV-2 S protein (D614) expression plasmid. NK cells were purified from peripheral blood of a healthy human volunteer by negative selection (Miltenyi Biotech), and were incubated with target cells at a 1:1 ratio in the presence of diluted plasma or monoclonal antibodies, Brefeldin A (GolgiPlug, 1 μl/ml, BD Biosciences), monensin (GolgiStop, 4 μl/6 mL, BD Biosciences), and anti-CD107a-FITC (BD Biosciences, clone H4A3) in 96-well flat bottom plates for 6 hours at 37° C. in a humidified 5% C02 incubator. NK cells were then recovered and stained for viability prior to staining with CD56-PECy7 (BD Biosciences, clone NCAM16.2), CD16-PacBlue (BD Biosciences, clone 3G8), and CD69-BV785 (Biolegend, Clone FN50). Flow cytometry data analysis was performed using FlowJo software (v10.8.0). Data is reported as the % of CD107A+ live NK cells (gates included singlets, lymphocytes, aqua blue-, CD56+ and/or CD16+, CD107A+). All final data represent specific activity, determined by subtraction of non-specific activity observed in assays performed with mock-infected cells and in absence of antibodies.

### Intracellular Cytokine Staining (ICS) Assay

Cryopreserved PBMC were thawed and rested 4 hours at 37° C. in a 5% CO₂ environment. PBMC were then incubated for 6 hours in the presence of RPMI containing 10% fetal bovine serum (unstimulated), Staphylococcus enterotoxin B (SEB) as positive control, or pool peptide spanning the entire SARS-CoV-2 spike protein. All cultures contained a protein transport inhibitor, monensin (Golgi Plug; Becton, Dickinson and Company), and 1 μg/ml of anti-CD49d (Becton, Dickinson and Company, Cat #340976). Cultured cells were then stained with a cell viability marker and pre-titered quantities of antibodies against CD3/CD4/CD8/CD45RA/ICOS/CCR7/CXCR3/PD-1/CXCR5/CD69/CD154/IL-2/IFN-g/TNF-α/IL-4/IL-21/IL-13/IL-17A. Samples were analyzed on a LSR II instrument (Becton, Dickinson and Company, Franklin Lakes, NJ) using FlowJo software.

Viral RNA Extraction and Subgenomic mRNA Quantification

SARS-CoV-2 E gene and N gene subgenomic mRNA (sgRNA) was measured by a one-step RT-qPCR adapted from previously described methods^{45,74}. To generate standard curves, a SARS-CoV-2 E gene sgRNA sequence, including the 5′UTR leader sequence, transcriptional regulatory sequence (TRS), and the first 228 bp of E gene, was cloned into a pcDNA3.1 plasmid. For generating SARS-CoV-2 N gene sgRNA, the E gene was replaced with the first 227 bp of N gene. The recombinant pcDNA3.1 plasmid was linearized, transcribed using MEGAscript T7 Transcription Kit (ThermoFisher, catalog #AM1334), and purified with MEGAclear Transcription Clean-Up Kit (ThermoFisher, catalog #AM1908). The purified RNA products were quantified on Nanodrop, serial diluted, and aliquoted as E sgRNA or N sgRNA standards.

A QIAsymphony SP (Qiagen, Hilden, Germany) automated sample preparation platform along with a virus/pathogen DSP midi kit. RNA extracted from animal samples or standards were then measured in Tagman custom gene expression assays (ThermoFisher). For these assays we used TaqMan Fast Virus 1-Step Master Mix (ThermoFisher, catalog #4444432) and custom primers/probes targeting the E gene sgRNA (forward primer: 5′ CGA TCT CTT GTA GAT CTG TTC TCE 3′ (SEQ ID NO: 52); reverse primer: 5′ ATA TTG CAG CAG TAC GCA CAC A 3′ (SEQ ID NO: 49); probe: 5′ FAM-ACA CTA GCC ATC CTT ACT GCG CTT CG-BHQ1 3′ (SEQ ID NO: 53)) or the N gene sgRNA (forward primer: 5′ CGA TCT CTT GTA GAT CTG TTC TC 3′ (SEQ ID NO: 52); reverse primer: 5′ GGT GAA CCA AGA CGC AGT AT 3′ (SEQ ID NO: 54); probe: 5′ FAM-TAA CCA GAA TGG AGA ACG CAG TGG G-BHQ1 3′ (SEQ ID NO: 55)). RT-qPCR reactions were carried out on CFX384 Touch Real-Time PCR System (Bio-Rad) using a program below: reverse transcription at 50° C. for 5 minutes, initial denaturation at 95° C. for 20 seconds, then 40 cycles of denaturation-annealing-extension at 95° C. for 15 seconds and 60° C. for 30 seconds. Standard curves were used to calculate E or N sgRNA in copies per ml; the limit of detections (LOD) for both E and N sgRNA assays were 12.5 copies per reaction or 150 copies per mL of BAL/nasal swab.

### Histopathology

Lung specimen from nonhuman primates were fixed in 10% neutral buffered formalin, processed, and blocked in paraffin for histological analysis. All samples were sectioned at 5 μm and stained with hematoxylin-eosin (H&E) for routine histopathology. Sections were examined under light microscopy using an Olympus BX51 microscope and photographs were taken using an Olympus DP73 camera. Samples were scored by a board-certified veterinary pathologist in a blinded manner. The representative images are to characterize the types and arrangement of inflammatory cells, while the scores show the relative severity of the tissue section.

### Immunohistochemistry (IHC)

Staining for SARS-CoV-2 antigen was achieved on the Bond RX automated system with the Polymer Define Detection System (Leica) used per manufacturer's protocol. Tissue sections were dewaxed with Bond Dewaxing Solution (Leica) at 72° C. for 30 min then subsequently rehydrated with graded alcohol washes and 1× Immuno Wash (StatLab). Heat-induced epitope retrieval (HIER) was performed using Epitope Retrieval Solution 1 (Leica), heated to 100° C. for 20 minutes. A peroxide block (Leica) was applied for 5 min to quench endogenous peroxidase activity prior to applying the SARS-CoV-2 antibody (1:2000, GeneTex, GTX135357). Antibodies were diluted in Background Reducing Antibody Diluent (Agilent). The tissue was subsequently incubated with an anti-rabbit HRP polymer (Leica) and colorized with 3,3′-Diaminobenzidine (DAB) chromogen for 10 min. Slides were counterstained with hematoxylin.

### Mouse Immunization and Challenge

Eleven-month-old female BALB/c mice were purchased from Envigo (#047) and were used for the SARS-CoV, SARS-CoV-2 WA-1, SARS-CoV-2 B.1.351, and RsSHC014-CoV protection experiments. The study was carried out in accordance with the recommendations for care and use of animals by the Office of Laboratory Animal Welfare (OLAW), National Institutes of Health and the Institutional Animal Care and Use Committee (IACUC) of University of North Carolina (UNC permit no. A-3410-01). Animals were housed in groups of five and fed standard chow diets. Virus inoculations were performed under anesthesia and all efforts were made to minimize animal suffering. Mice were intramuscularly immunized with RBD-scNP formulated with 3M-052-AF+Alum or GLA-SE. For the SARS-CoV-2 WA-1 and RsSHC014 study, mice were immunized on week 0 and 2, and challenged on week 7. For the SARS-CoV-2 B.1.351 and SARS-CoV-1 study, mice were immunized on week 0 and 4, and challenged on week 6. All mice were anesthetized and infected intranasally with 1×10⁴ PFU/ml of SARS-CoV MA15, 1×10⁴ PFU/ml of SARS-CoV-2 WA1-MA10 or B.1.351-MA10, 1×10⁴ PFU/ml RsSHC014, which have been described previously⁷⁵⁻⁷⁷. Mice were weighted daily and monitored for signs of clinical disease, and selected groups were subjected to daily whole-body plethysmography. For all mouse studies, groups of n=10 mice were included per arm of the study. Lung viral titers and weight loss were measured from individual mice per group.

### Biocontainment and Biosafety

Studies were approved by the UNC Institutional Biosafety Committee approved by animal and experimental protocols in the Baric laboratory. All work described here was performed with approved standard operating procedures for SARS-CoV-2 in a biosafety level 3 (BSL-3) facility conforming to requirements recommended in the Microbiological and Biomedical Laboratories, by the U.S. Department of Health and Human Service, the U.S. Public Health Service, and the U.S. Center for Disease Control and Prevention (CDC), and the National Institutes of Health (NIH).

### Statistics Analysis

Data were plotted using Prism GraphPad 8.0. Wilcoxon rank sum exact test was performed to compare differences between groups with p-value<0.05 considered significant using SAS 9.4 (SAS Institute, Cary, NC). No adjustments were made to the p-values for multiple comparisons.

### REFERENCES

1 Levin, E. G. et al. Waning Immune Humoral Response to BNT162b2 Covid-19 Vaccine over 6 Months. N Engl J Med, doi:10.1056/NEJMoa2114583 (2021).
2 Chaudhary, N., Weissman, D. & Whitehead, K. A. mRNA vaccines for infectious diseases: principles, delivery and clinical translation. Nat Rev Drug Discov 20, 817-838, doi:10.1038/s41573-021-00283-5 (2021).
3 Pardi, N., Hogan, M. J., Porter, F. W. & Weissman, D. mRNA vaccines—a new era in vaccinology. Nat Rev Drug Discov 17, 261-279, doi:10.1038/nrd.2017.243 (2018).
4 Pardi, N., Hogan, M. J. & Weissman, D. Recent advances in mRNA vaccine technology. Curr Opin Immunol 65, 14-20, doi:10.1016/j.coi.2020.01.008 (2020). Baden, L. R. et al. Efficacy and Safety of the mRNA-1273 SARS-CoV-2 Vaccine. N Engl J Med 384, 403-416, doi:10.1056/NEJMoa2035389 (2021).
6 Haynes, B. F. A New Vaccine to Battle Covid-19. N Engl J Med 384, 470-471, doi:10.1056/NEJMe2035557 (2021).
7 Polack, F. P. et al. Safety and Efficacy of the BNT162b2 mRNA Covid-19 Vaccine. N Engl J Med 383, 2603-2615, doi:10.1056/NEJMoa2034577 (2020).
8<https://www.gisaid.org/hcov19-variants/>(
9 Wang, L. & Cheng, G. Sequence analysis of the emerging SARS-CoV-2 variant Omicron in South Africa. J Med Virol, doi:10.1002/jmv.27516 (2021).
10 Saunders, K. O. et al. Neutralizing antibody vaccine for pandemic and pre-emergent coronaviruses. Nature 594, 553-559, doi:10.1038/s41586-021-03594-0 (2021).
11 Li, D. et al. In vitro and in vivo functions of SARS-CoV-2 infection-enhancing and neutralizing antibodies. Cell 184, 4203-4219 e4232, doi:10.1016/j.cell.2021.06.021 (2021).
12 Li, D., Sempowski, G. D., Saunders, K. O., Acharya, P. & Haynes, B. F. SARS-CoV-2 Neutralizing Antibodies for COVID-19 Prevention and Treatment. Annu Rev Med, doi:10.1146/annurev-med-042420-113838 (2021).
13 Hastie, K. M. et al. Defining variant-resistant epitopes targeted by SARS-CoV-2 antibodies: A global consortium study. Science 374, 472-478, doi:10.1126/science.abh2315 (2021).
14 Corbett, K. S. et al. Evaluation of the mRNA-1273 Vaccine against SARS-CoV-2 in Nonhuman Primates. N Engl J Med 383, 1544-1555, doi:10.1056/NEJMoa2024671 (2020).
15 Dunkle, L. M. et al. Efficacy and Safety of NVX-CoV2373 in Adults in the United States and Mexico. N Engl J Med, doi:10.1056/NEJMoa2116185 (2021).
16 Pino, M. et al. A yeast expressed RBD-based SARS-CoV-2 vaccine formulated with 3M-052-alum adjuvant promotes protective efficacy in non-human primates. Sci Immunol 6, doi:10.1126/sciimmunol.abh3634 (2021).
17 Dai, L. et al. A Universal Design of Betacoronavirus Vaccines against COVID-19, MERS, and SARS. Cell 182, 722-733 e711, doi:10.1016/j.cell.2020.06.035 (2020).
18 Routhu, N. K. et al. SARS-CoV-2 RBD trimer protein adjuvanted with Alum-3M-052 protects from SARS-CoV-2 infection and immune pathology in the lung. Nat Commun 12, 3587, doi:10.1038/s41467-021-23942-y (2021).
19 Joyce, M. G. et al. A SARS-CoV-2 ferritin nanoparticle vaccine elicits protective immune responses in nonhuman primates. Sci Transl Med, eabi5735 (2021).
20 Walls, A. C. et al. Elicitation of broadly protective sarbecovirus immunity by receptor-binding domain nanoparticle vaccines. Cell 184, 5432-5447 e5416, doi:10.1016/j.cell.2021.09.015 (2021).
21 Benenson, S., Oster, Y., Cohen, M. J. & Nir-Paz, R. BNT162b2 mRNA Covid-19 Vaccine Effectiveness among Health Care Workers. N Engl J Med, doi:10.1056/NEJMc2101951 (2021).
22 Dagan, N. et al. BNT162b2 mRNA Covid-19 Vaccine in a Nationwide Mass Vaccination Setting. N Engl J Med, doi:10.1056/NEJMoa2101765 (2021).
23 Bar-On, Y. M. et al. Protection of BNT162b2 Vaccine Booster against Covid-19 in Israel. N Engl J Med, doi:10.1056/NEJMoa2114255 (2021).
24 Doria-Rose, N. et al. Antibody Persistence through 6 Months after the Second Dose of mRNA-1273 Vaccine for Covid-19. N Engl J Med 384, 2259-2261, doi:10.1056/NEJMc2103916 (2021).
25 Roest, S., Hoek, R. A. S. & Manintveld, O. C. BNT162b2 mRNA Covid-19 Vaccine in a Nationwide Mass Vaccination Setting. N Engl J Med 384, 1968-1970, doi:10.1056/NEJMc2104281 (2021).
26 Thomas, S. J. et al. Safety and Efficacy of the BNT162b2 mRNA Covid-19 Vaccine through 6 Months. N Engl J Med, doi:10.1056/NEJMoa2110345 (2021).
27 Thompson, M. G. et al. Prevention and Attenuation of Covid-19 with the BNT162b2 and mRNA-1273 Vaccines. N Engl J Med 385, 320-329, doi:10.1056/NEJMoa2107058 (2021).
28 Widge, A. T. et al. Durability of Responses after SARS-CoV-2 mRNA-1273 Vaccination. N Engl J Med 384, 80-82, doi:10.1056/NEJMc2032195 (2021).
29 Manaf, A. et al. Morbidity and mortality from COVID-19 post-vaccination breakthrough infections in association with vaccines and the emergence of variants in Bahrain. Research Square, doi:10.21203/rs.3.rs-828021/v1 (2021).
30 Emary, K. R. W. et al. Efficacy of ChAdOx1 nCoV-19 (AZD1222) vaccine against SARS-CoV-2 variant of concern 202012/01 (B.1.1.7): an exploratory analysis of a randomised controlled trial. Lancet 397, 1351-1362, doi:10.1016/S0140-6736(21)00628-0 (2021).
31 Jones, I. & Roy, P. Sputnik V COVID-19 vaccine candidate appears safe and effective. Lancet 397, 642-643, doi:10.1016/S0140-6736(21)00191-4 (2021).
32 Voysey, M. et al. Safety and efficacy of the ChAdOx1 nCoV-19 vaccine (AZD1222) against SARS-CoV-2: an interim analysis of four randomised controlled trials in Brazil, South Africa, and the UK. Lancet 397, 99-111, doi:10.1016/S0140-6736(20)32661-1(2021).
33 Voysey, M. et al. Single-dose administration and the influence of the timing of the booster dose on immunogenicity and efficacy of ChAdOx1 nCoV-19 (AZD1222) vaccine: a pooled analysis of four randomised trials. Lancet 397, 881-891, doi:10.1016/S0140-6736(21)00432-3 (2021).
34 Tregoning, J. S., Flight, K. E., Higham, S. L., Wang, Z. & Pierce, B. F. Progress of the COVID-19 vaccine effort: viruses, vaccines and variants versus efficacy, effectiveness and escape. Nat Rev Immunol 21, 626-636, doi:10.1038/s41577-021-00592-1 (2021).
35 Collie, S., Champion, J., Moultrie, H., Bekker, L. G. & Gray, G. Effectiveness of BNT162b2 Vaccine against Omicron Variant in South Africa. N Engl J Med, doi:10.1056/NEJMc2119270 (2021).
36 Kanekiyo, M. et al. Self-assembling influenza nanoparticle vaccines elicit broadly neutralizing H1N1 antibodies. Nature 499, 102-106, doi:10.1038/naturel2202 (2013).
37 King, H. A. D. et al. Efficacy and breadth of adjuvanted SARS-CoV-2 receptor-binding domain nanoparticle vaccine in macaques. Proc Natl Acad Sci USA 118, doi:10.1073/pnas.2106433118 (2021).
38 Ma, X. et al. Nanoparticle Vaccines Based on the Receptor Binding Domain (RBD) and Heptad Repeat (HR) of SARS-CoV-2 Elicit Robust Protective Immune Responses. Immunity 53, 1315-1330 e1319, doi:10.1016/j.immuni.2020.11.015 (2020).
39 Wang, W., Huang, B., Zhu, Y., Tan, W. & Zhu, M. Ferritin nanoparticle-based SARS-CoV-2 RBD vaccine induces a persistent antibody response and long-term memory in mice. Cell Mol Immunol 18, 749-751, doi:10.1038/s41423-021-00643-6 (2021).
40 Wuertz, K. M. et al. A SARS-CoV-2 spike ferritin nanoparticle vaccine protects against heterologous challenge with B.1.1.7 and B.1.351 virus variants in Syrian golden hamsters. bioRxiv, doi:10.1101/2021.06.16.448525 (2021).
41 Earle, K. A. et al. Evidence for antibody as a protective correlate for COVID-19 vaccines. Vaccine 39, 4423-4428, doi:10.1016/j.vaccine.2021.05.063 (2021).
42 Khoury, D. S. et al. Neutralizing antibody levels are highly predictive of immune protection from symptomatic SARS-CoV-2 infection. Nat Med 27, 1205-1211, doi:10.1038/s41591-021-01377-8 (2021).
43 He, X. et al. Low-dose Ad26.COV2.S protection against SARS-CoV-2 challenge in rhesus macaques. Cell 184, 3467-3473 e3411, doi:10.1016/j.cell.2021.05.040 (2021).
44 Mercado, N. B. et al. Single-shot Ad26 vaccine protects against SARS-CoV-2 in rhesus macaques. Nature 586, 583-588, doi:10.1038/s41586-020-2607-z (2020).
45 Yu, J. et al. DNA vaccine protection against SARS-CoV-2 in rhesus macaques. Science 369, 806-811, doi:10.1126/science.abc6284 (2020).
46 Corbett, K. S. et al. Immune correlates of protection by mRNA-1273 vaccine against SARS-CoV-2 in nonhuman primates. Science 373, eabj0299, doi:10.1126/science.abj0299 (2021).
47 Gilbert, P. B. et al. Immune Correlates Analysis of the mRNA-1273 COVID-19 Vaccine Efficacy Trial. medRxiv, doi:10.1101/2021.08.09.21261290 (2021).
48 Heath, P. T. et al. Safety and Efficacy of NVX-CoV2373 Covid-19 Vaccine. N Engl J Med 385, 1172-1183, doi:10.1056/NEJMoa2107659 (2021).
49 Mahase, E. Covid-19: Novavax vaccine efficacy is 86% against UK variant and 60% against South African variant. BMJ 372, n296, doi:10.1136/bmj.n296 (2021).
50 Shinde, V. et al. Efficacy of NVX-CoV2373 Covid-19 Vaccine against the B.1.351 Variant. N Engl J Med 384, 1899-1909, doi:10.1056/NEJMoa2103055 (2021).
51 Yang, J. et al. A vaccine targeting the RBD of the S protein of SARS-CoV-2 induces protective immunity. Nature 586, 572-577, doi:10.1038/s41586-020-2599-8 (2020).
52 Francica, J. R. et al. Protective antibodies elicited by SARS-CoV-2 spike protein vaccination are boosted in the lung after challenge in nonhuman primates. Sci Transl Med 13, doi:10.1126/scitranslmed.abi4547 (2021).
53 Chi, X. et al. A potent neutralizing human antibody reveals the N-terminal domain of the Spike protein of SARS-CoV-2 as a site of vulnerability. doi:10.1101/2020.05.08.083964 (2020).
54 Cerutti, G. et al. Potent SARS-CoV-2 neutralizing antibodies directed against spike N-terminal domain target a single supersite. Cell Host Microbe, doi:10.1016/j.chom.2021.03.005 (2021).
55 McCallum, M. et al. N-terminal domain antigenic mapping reveals a site of vulnerability for SARS-CoV-2. Cell, doi:10.1016/j.cell.2021.03.028 (2021).
56 Schafer, A. et al. Antibody potency, effector function, and combinations in protection and therapy for SARS-CoV-2 infection in vivo. J Exp Med 218, doi:10.1084/jem.20201993 (2021).
57 Winkler, E. S. et al. Human neutralizing antibodies against SARS-CoV-2 require intact Fc effector functions for optimal therapeutic protection. Cell 184, 1804-1820 e1816, doi:10.1016/j.cell.2021.02.026 (2021).
58 Yamin, R. et al. Fc-engineered antibody therapeutics with improved anti-SARS-CoV-2 efficacy. Nature, doi:10.1038/s41586-021-04017-w (2021).
59 Coffman, R. L., Sher, A. & Seder, R. A. Vaccine adjuvants: putting innate immunity to work. Immunity 33, 492-503, doi:10.1016/j.immuni.2010.10.002 (2010).
60 HogenEsch, H., O'Hagan, D. T. & Fox, C. B. Optimizing the utilization of aluminum adjuvants in vaccines: you might just get what you want. NPJ Vaccines 3, 51, doi:10.1038/s41541-018-0089-x (2018).
61 Kasturi, S. P. et al. 3M-052, a synthetic TLR-7/8 agonist, induces durable HIV-1 envelope-specific plasma cells and humoral immunity in nonhuman primates. Sci Immunol 5, doi:10.1126/sciimmunol.abb1025 (2020).
62 Smimov, D., Schmidt, J. J., Capecchi, J. T. & Wightman, P. D. Vaccine adjuvant activity of 3M-052: an imidazoquinoline designed for local activity without systemic cytokine induction. Vaccine 29, 5434-5442, doi:10.1016/j.vaccine.2011.05.061 (2011).
63 Marrack, P., McKee, A. S. & Munks, M. W. Towards an understanding of the adjuvant action of aluminium. Nat Rev Immunol 9, 287-293, doi:10.1038/nri2510 (2009).
64 Arvin, A. M. et al. A perspective on potential antibody-dependent enhancement of SARS-CoV-2. Nature 584, 353-363, doi:10.1038/s41586-020-2538-8 (2020).
65 Haynes, B. F. et al. Prospects for a safe COVID-19 vaccine. Sci Transl Med 12, doi:10.1126/scitranslmed.abe0948 (2020).
66 Fox, C. B. et al. Adsorption of a synthetic TLR7/8 ligand to aluminum oxyhydroxide for enhanced vaccine adjuvant activity: A formulation approach. J Control Release 244, 98-107, doi:10.1016/j.jconrel.2016.11.011 (2016).
67 Wrapp, D. et al. Cryo-EM structure of the 2019-nCoV spike in the prefusion conformation. Science 367, 1260-1263, doi:10.1126/science.abb2507 (2020).
68 Zhou, T. et al. Structure-Based Design with Tag-Based Purification and In-Process Biotinylation Enable Streamlined Development of SARS-CoV-2 Spike Molecular Probes. Cell Rep 33, 108322, doi:10.1016/j.celrep.2020.108322 (2020).
69 Korber, B. et al. Tracking Changes in SARS-CoV-2 Spike: Evidence that D614G Increases Infectivity of the COVID-19 Virus. Cell 182, 812-827 e819, doi:10.1016/j.cell.2020.06.043 (2020).
70 Naldini, L., Blomer, U., Gage, F. H., Trono, D. & Verma, I. M. Efficient transfer, integration, and sustained long-term expression of the transgene in adult rat brains injected with a lentiviral vector. Proc Natl Acad Sci USA 93, 11382-11388, doi:10.1073/pnas.93.21.11382 (1996).
71 Berry, J. D. et al. Development and characterisation of neutralising monoclonal antibody to the SARS-coronavirus. J Virol Methods 120, 87-96, doi:10.1016/j.jviromet.2004.04.009 (2004).
72 Coleman, C. M. & Frieman, M. B. Growth and Quantification of MERS-CoV Infection. Curr Protoc Microbiol 37, 15E 12 11-19, doi:10.1002/9780471729259.mc15e02s37 (2015).
73 Kint, J., Maier, H. J. & Jagt, E. Quantification of infectious bronchitis coronavirus by titration in vitro and in ovo. Methods Mol Biol 1282, 89-98, doi:10.1007/978-1-4939-2438-7_9 (2015).
74 Wolfel, R. et al. Virological assessment of hospitalized patients with COVID-2019. Nature 581, 465-469, doi:10.1038/s41586-020-2196-x (2020).
75 Menachery, V. D. et al. A SARS-like cluster of circulating bat coronaviruses shows potential for human emergence. Nat Med 21, 1508-1513, doi:10.1038/nm.3985 (2015).
76 Leist, S. R. et al. A Mouse-Adapted SARS-CoV-2 Induces Acute Lung Injury and Mortality in Standard Laboratory Mice. Cell 183, 1070-1085.e1012, doi:10.1016/j.cell.2020.09.050 (2020).
77 Roberts, A. et al. A mouse-adapted SARS-coronavirus causes disease and mortality in BALB/c mice. PLoS Pathog 3, e5, doi:10.1371/journal.ppat.0030005 (2007).

This example summarizes data from non-human primate studies (NHP) #177, #183 and #188.

In this Example 3 and in Example 1 the RBD vaccine is HV1302118.

In this Example 3 and in Example 1 the NTD vaccine is HV1302125 SARS-COV-2_NTD_C-SORTA_c3C8HTWSTII

In the Example 3 and in Example, 1 the Ectodomain vaccines is HV1302119 SARS-CoV-2 S-2P-foldon SortA_3C8HtS2.

Data in this Example 3 and FIGS. 38-42 show as follows.

All adjuvants protected macaques from WA-1 SARS-CoV-2.

RBD-scNP, or spike S2P-scNP induced both neutralizing and antibody-dependent cellular cytotoxicity (ADCC) antibodies in macaques, and protected all immunized monkeys against SARS-CoV-2 WA-1 strain.

NTD-scNP induce only ADCC but not neutralizing antibodies, yet it protected equally well with RBD-scNP or S2P-scNP from SARS-CoV-2 WA-1 challenge.

Whereas three doses of NTD-scNP protected all immunized macaques from SARS-CoV-2 challenge, it did not protect as well when boosting after two primes with mRNA-LNPs encoding S2P.

Two doses of RBD-scNP were sufficient to protect against the difficult-to-neutralize SARS-CoV-2 Beta variant.

FIGS. 38-42 summarize analyses of immunization studies from Example 1 and Example 3 with respect to omicron coronavirus variant.

### Example 4: Gene Fusion Nanoparticles

Three major coronavirus (CoV) outbreaks have occurred since 2003 (1-3). Two of these outbreaks were caused by group 2b coronaviruses with the remaining outbreak caused by a group 2C CoV (4, 5). Therefore, a universal CoV vaccine should protect against at least both group 2b and 2c betaCoVs. Neutralizing antibodies elicited by CoV infection or vaccination have exhibited broad neutralization of group 2b viruses (6-11), but cross-neutralization of group 2b and 2c viruses by a single antibody has yet to be reported (12). To expand the neutralization breadth of current vaccines, we will immunize with group 2b and 2c CoV immunogens as a bivalent nanoparticle (NP). The rationale for this design is supported by mosaic NPs displaying up to 8 different CoV receptor-binding domains (RBDs) have generated group 2B and 2C reactive antibodies in mice (13). The antigens expressed on the NPs will be the receptor-binding domain of the spike protein (RBD) of MERS-CoV and SARS-CoV-1. The RBD was chosen as the immunogen because: it is responsible for receptor recognition by the viral Spike (S) protein (14, 15), blocking the interaction between host receptor and the RBD is a principal mechanism of neutralizing Abs, and when humans make serum neutralizing antibodies they can target the RBD (16). Similarly, we have found after vaccination, RBD focused immunogens elicit comparable or higher neutralizing antibody titers than spike ectodomain immunogens (12). A nanoparticle format was chosen since RBD immunogenicity can be augmented by arraying multiple copies of it on NPs, mimicking virus-like particles (12, 13, 17-21). The bivalent NP vaccine can be encoded by nucleic acid such as nucleoside modified mRNA encapsulated in lipid nanoparticles (mRNA-LNPs) or plasmid DNA. In other embodiments of the vaccine the nanoparticle can be recombinant protein formulated with or without an adjuvant.

In some embodiments the NP is a 24-subunit self-assembling NP derived from H. pylori or T. ni ferritin (FIG. 24A). H. pylori and T. ni ferritin are distinct from human ferritin in sequence. H. pylori ferritin is composed a single subunit. In contrast, T. ni ferritin is composed of a heavy and light chain that each can display a single viral antigen. Thus, when assembled it displays 12 copies of each antigen on the same NP. We have successfully used this approach for protein HIV-1 subunit vaccines displaying the HIV-1 fusion peptide or the HIV-1 envelope trimer (FIG. 24B), and this approach is similar to the protein RBD NP we recently published (12). Ferritin NPs have a known safety profile since H. pylori ferritin NPs have been administered in clinical trials for SARS-CoV-2 spike ectodomain and influenza HA vaccines (NCT03186781; NCT04784767).

Recombinant protein NP immunogens have elicited neutralizing antibodies against multiple CoVs and increased neutralizing antibody titers compared to Spike ectodomain mRNA-LNP immunization (12). However, nucleoside-modified mRNA is a favorable vaccine platform because of its rapid manufacturability and distribution capability. Also, mRNA-LNP SARS-CoV-2 immunization has been shown to be 95% effective against SARS-CoV-2 infection and is well-tolerated in the general population (22, 23). Thus, the goal of this immunogen design is to combine the enhanced immunogenicity of Spike RBD NPs and high efficacy and manufacturability of nucleoside-modified mRNA-LNP vaccination. To use mRNAs the RBD and ferritin need to be expressed from a single gene encoding a fusion protein. To encode two different RBDs for bivalent immunogens, two mRNAs encoding each ferritin chain fused to a different RBD will be encapsulated in one lipid nanoparticle (LNP).

Antibodies, such as ADG-2, DH1047, and S309, can cross-neutralize SARS-CoV-1 and SARS-CoV-2 and other SARS-related viruses have been isolated from individuals infected with SARS-CoV-1 (6, 24, 25). The presence of these cross-neutralizing antibodies in SARS-CoV-1 infected individuals indicated that antibodies could target shared epitopes among multiple SARS-related viruses. Indeed, SARS-CoV-2 BNT162b6 immunization of SARS-CoV-1 convalescent individuals resulted in high titers of sarbecovirus neutralizing antibodies (26). Without wishing to be bound by theory, cross-reactive antibodies can be induced to high levels by boosting SARS-CoV-2 reactive B cells with a SARS-related CoV spike such as SARS-CoV-1 Spike. By administering a bivalent SARS-CoV-1 and MERS-CoV RBD NPs to individuals who have been vaccinated previously against SARS-CoV-2, pan group 2b neutralizing antibodies and broad group 2C-neutralizing antibodies will be elicited. Thus one use of this immunogen would be used to boost SARS-CoV-2 vaccine recipients with two immunizations of SARS-CoV-1 and MERS-CoV RBD bivalent NPs.

To display the receptor binding domain on the surface of ferritin nanoparticles, the two proteins have to be fused together to create a single fusion protein. Long linkers (>15 amino acids (aa)) are one way to separate the RBD in space from the ferritin subunit so that both proteins can fold without interfering with each other. Additionally, long linkers allow the protein of interest to be distanced away from the nanoparticle subunits that are trying to oligomerize into a nanoparticle. Our designs with 25 aa linkers were analyzed by AlphaFold and predicted to place the SARS-CoV-2 RBD inside the nanoparticle (FIG. 25). Due to the flexible glycine serine linker the RBD was able to turn and position itself below the ferritin subunit (FIG. 25D). We redesigned the linker for the ferritin and RBD to keep the rigid alpha helical linker and to shorten the glycine-serine linker to 3 aa. Alpha fold predicted this linker would not allow the RBD to fold under the ferritin subunit (FIG. 26). When modelled on an intact H. pylori ferritin nanoparticle the RBD was displayed on the surface. Moreover, the RBDs on the surface were distanced sufficiently not to clash with each other (FIG. 3). This linker was applied for designs using H. pylori ferritin. Here we show SARS-CoV-1, SARS-CoV-2, and MERS-CoV RBDs fused to the linker and ferritin subunit, but this design could be applied to any coronavirus receptor binding domain.

For T. ni ferritin we considered the same phenomenon could occur where the RBD was folding inside the ferritin. Since the N-terminus of the heavy and light chains are flexible loops this concern seemed reasonable. We introduced the 14 aa linker between MERS-CoV RBD and T. ni ferritin heavy or light chain and predicted the structure with AlphaFold. Interestingly AlphaFold predicted that for the light chain the RBD would be on the outside of the nanoparticle, but for the heavy chain the RBD was still predicted to fold inside the lumen of the nanoparticle. Thus, for T. ni ferritin where the N-termini of the protein was very flexible we removed the rigid linker and kept the GGS linker. The rationale for the design was that a shorter linker may not be long enough to stick the RBD inside the T. ni nanoparticle.

Non-limiting embodiments of sequences of gene fusion designs are described in Table 3 and FIG. 23.

Expression data are shown in FIG. 27A-B. These proteins will be further characterized in any suitable assay and/or animal study.

Various approached can be used to optimize protein and/or nucleic acid expression. We will change the signal peptide sequence which can boost expression in some cases. The linker will be optimized—the linker GGSEKAAKAEEAARPP (SEQ ID NO: 14) in the fusion RBD-GGSEKAAKAEEAARPP-ferritin (“GGSEKAAKAEEAARPP” disclosed as SEQ ID NO: 14) and the linker GGSEKAAKAEEAARP (SEQ ID NO: 13) in the fusion RBD-GGSEKAAKAEEAARP-ferritin (“GGSEKAAKAEEAARP” disclosed as SEQ ID NO: 13). This linker would have the rigidity needed to distance the RBD from the ferritin but it would also turn the rigid linker to orient more vertically rather than horizontally. The design goal would be to have the RBD be more perpendicular to the ferritin subunit.

The RBD sequence could be changed of the disulfide bond-stabilized designs are helpful or for the elimination of hydrophobic patches. Dalvie et al. PNAS Sep. 21, 2021 118 (38) e21068451 18; https://doi.org/10.1073/pnas.2106845118 have reported that hydrophobic patches on the RBD limit its expression and cause aggregation when it is expressed as a monomer in yeast. Some embodiments eliminate hydrophobic patches by introducing L452K and F490W mutations. MERS and SARS1 sequences with a similar design-having comparable mutations—will be generated.

### REFERENCES FOR EXAMPLE 4

1. Guarner J. Three Emerging Coronaviruses in Two Decades. Am J Clin Pathol. 2020; 153(4):420-1. Epub 2020/02/14. doi: 10.1093/ajcp/aqaa029. PubMed PMID: 32053148; PMCID: PMC7109697.
2. Peeri N C, Shrestha N, Rahman M S, Zaki R, Tan Z, Bibi S, Baghbanzadeh M, Aghamohammadi N, Zhang W, Haque U. The SARS, MERS and novel coronavirus (COVID-19) epidemics, the newest and biggest global health threats: what lessons have we learned? Int J Epidemiol. 2020; 49(3):717-26. Epub 2020/02/23. doi: 10.1093/ije/dyaa033. PubMed PMID: 32086938; PMCID: PMC7197734.
3. Sempowski G D, Saunders K O, Acharya P, Wiehe K J, Haynes B F. Pandemic Preparedness: Developing Vaccines and Therapeutic Antibodies For COVID-19. Cell. 2020; 181(7):1458-63. Epub 2020/06/04. doi: 10.1016/j.cell.2020.05.041. PubMed PMID: 32492407; PMCID: PMC7250787.
4. Gralinski L E, Menachery V D. Return of the Coronavirus: 2019-nCoV. Viruses. 2020; 12(2). Epub 2020/01/30. doi: 10.3390/v12020135. PubMed PMID: 31991541; PMCID: PMC7077245.
5. Menachery V D, Graham R L, Baric R S. Jumping species—a mechanism for coronavirus persistence and survival. Curr Opin Virol. 2017; 23:1-7. Epub 2017/02/20. doi: 10.1016/j.coviro.2017.01.002. PubMed PMID: 28214731; PMCID: PMC5474123.
6. Pinto D, Park Y J, Beltramello M, Walls A C, Tortorici M A, Bianchi S, Jaconi S, Culap K, Zatta F, De Marco A, Peter A, Guarino B, Spreafico R, Cameroni E, Case J B, Chen R E, Havenar-Daughton C, Snell G, Telenti A, Virgin H W, Lanzavecchia A, Diamond M S, Fink K, Veesler D, Corti D. Cross-neutralization of SARS-CoV-2 by a human monoclonal SARS-CoV antibody. Nature. 2020; 583(7815):290-5. Epub 2020/05/19. doi: 10.1038/s41586-020-2349-y. PubMed PMID: 32422645.
7. Wec A Z, Wrapp D, Herbert A S, Maurer D P, Haslwanter D, Sakharkar M, Jangra R K, Dieterle M E, Lilov A, Huang D, Tse L V, Johnson N V, Hsieh C L, Wang N, Nett J H, Champney E, Burnina I, Brown M, Lin S, Sinclair M, Johnson C, Pudi S, Bortz R, 3rd, Wirchnianski A S, Laudermilch E, Florez C, Fels J M, O'Brien C M, Graham B S, Nemazee D, Burton D R, Baric R S, Voss J E, Chandran K, Dye J M, McLellan J S, Walker L M. Broad neutralization of SARS-related viruses by human monoclonal antibodies. Science. 2020; 369(6504):731-6. Epub 2020/06/17. doi: 10.1126/science.abc7424. PubMed PMID: 32540900; PMCID: PMC7299279.
8. Rappazzo C G, Tse L V, Kaku C I, Wrapp D, Sakharkar M, Huang D, Deveau L M, Yockachonis T J, Herbert A S, Battles M B, O'Brien C M, Brown M E, Geoghegan J C, Belk J, Peng L, Yang L, Hou Y, Scobey T D, Burton D R, Nemazee D, Dye J M, Voss J E, Gunn B M, McLellan J S, Baric R S, Gralinski L E, Walker L M. Broad and potent activity against SARS-like viruses by an engineered human monoclonal antibody. Science. 2021; 371(6531):823-9. Epub 2021/01/27. doi: 10.1126/science.abf4830. PubMed PMID: 33495307; PMCID: PMC7963221.
9. Martinez D R, Schaefer A, Gobeil S, Li D, De la Cruz G, Parks R, Lu X, Barr M, Manne K, Mansouri K, Edwards R J, Yount B, Anasti K, Montgomery S A, Shen S, Zhou T, Kwong P D, Graham B S, Mascola J R, Montefiori D C, Alam M, Sempowski G D, Wiehe K, Saunders K O, Acharya P, Haynes B F, Baric R S. A broadly neutralizing antibody protects against SARS-CoV, pre-emergent bat CoVs, and SARS-CoV-2 variants in mice. bioRxiv. 2021. Epub 2021/05/06. doi: 10.1101/2021.04.27.441655. PubMed PMID: 33948590; PMCID: PMC8095197.
10. Li D, Edwards R J, Manne K, Martinez D R, Schafer A, Alam S M, Wiehe K, Lu X, Parks R, Sutherland L L, Oguin T H, 3rd, McDanal C, Perez L G, Mansouri K, Gobeil S M C, Janowska K, Stalls V, Kopp M, Cai F, Lee E, Foulger A, Hernandez G E, Sanzone A, Tilahun K, Jiang C, Tse L V, Bock K W, Minai M, Nagata B M, Cronin K, Gee-Lai V, Deyton M, Barr M, Von Holle T, Macintyre A N, Stover E, Feldman J, Hauser B M, Caradonna™, Scobey T D, Rountree W, Wang Y, Moody M A, Cain D W, DeMarco C T, Denny T N, Woods C W, Petzold E W, Schmidt A G, Teng I T, Zhou T, Kwong P D, Mascola J R, Graham B S, Moore I N, Seder R, Andersen H, Lewis M G, Montefiori D C, Sempowski G D, Baric R S, Acharya P, Haynes B F, Saunders K O. In vitro and in vivo functions of SARS-CoV-2 infection-enhancing and neutralizing antibodies. Cell. 2021; 184(16):4203-19 e32. Epub 2021/07/10. doi: 10.1016/j.cell.2021.06.021. PubMed PMID: 34242577; PMCID: PMC8232969.
11. Li D, Sempowski G D, Saunders K O, Acharya P, Haynes B F. SARS-CoV-2 Neutralizing Antibodies for COVID-19 Prevention and Treatment. Annu Rev Med. 2021. Epub 2021/08/25. doi: 10.1146/annurev-med-042420-113838. PubMed PMID: 34428080.
12. Saunders K O, Lee E, Parks R, Martinez D R, Li D, Chen H, Edwards R J, Gobeil S, Barr M, Mansouri K, Alam S M, Sutherland L L, Cai F, Sanzone A M, Berry M, Manne K, Bock K W, Minai M, Nagata B M, Kapingidza A B, Azoitei M, Tse L V, Scobey T D, Spreng R L, Rountree R W, DeMarco C T, Denny T N, Woods C W, Petzold E W, Tang J, Oguin T H, 3rd, Sempowski G D, Gagne M, Douek D C, Tomai M A, Fox C B, Seder R, Wiehe K, Weissman D, Pardi N, Golding H, Khurana S, Acharya P, Andersen H, Lewis M G, Moore I N, Montefiori D C, Baric R S, Haynes B F. Neutralizing antibody vaccine for pandemic and pre-emergent coronaviruses. Nature. 2021; 594(7864):553-9. Epub 2021/05/11. doi: 10.1038/s41586-021-03594-0. PubMed PMID: 33971664.
13. Cohen A A, Gnanapragasam P N P, Lee Y E, Hoffman P R, Ou S, Kakutani L M, Keeffe J R, Wu H J, Howarth M, West A P, Barnes C O, Nussenzweig M C, Bjorkman P J. Mosaic nanoparticles elicit cross-reactive immune responses to zoonotic coronaviruses in mice. Science. 2021. Epub 2021/01/14. doi: 10.1126/science.abf6840. PubMed PMID: 33436524.
14. Cai Y, Zhang J, Xiao T, Peng H, Sterling S M, Walsh R M, Rawson S, Rits-Volloch S, Chen B. Distinct conformational states of SARS-CoV-2 spike protein. bioRxiv. 2020. Epub 2020/06/09. doi: 10.1101/2020.05.16.099317. PubMed PMID: 32511405; PMCID: PMC7263552.
15. Shang J, Wan Y, Luo C, Ye G, Geng Q, Auerbach A, Li F. Cell entry mechanisms of SARS-CoV-2. Proc Natl Acad Sci USA. 2020; 117(21):11727-34. Epub 2020/05/08. doi: 10.1073/pnas.2003138117. PubMed PMID: 32376634; PMCID: PMC7260975.
16. Piccoli L, Park Y J, Tortorici M A, Czudnochowski N, Walls A C, Beltramello M, Silacci-Fregni C, Pinto D, Rosen L E, Bowen J E, Acton O J, Jaconi S, Guarino B, Minola A, Zatta F, Sprugasci N, Bassi J, Peter A, De Marco A, Nix J C, Mele F, Jovic S, Rodriguez B F, Gupta S V, Jin F, Piumatti G, Lo Presti G, Pellanda A F, Biggiogero M, Tarkowski M, Pizzuto M S, Cameroni E, Havenar-Daughton C, Smithey M, Hong D, Lepori V, Albanese E, Ceschi A, Bernasconi E, Elzi L, Ferrari P, Garzoni C, Riva A, Snell G, Sallusto F, Fink K, Virgin H W, Lanzavecchia A, Corti D, Veesler D. Mapping Neutralizing and Immunodominant Sites on the SARS-CoV-2 Spike Receptor-Binding Domain by Structure-Guided High-Resolution Serology. Cell. 2020; 183(4):1024-42 e21. Epub 2020/09/30. doi: 10.1016/j.cell.2020.09.037. PubMed PMID: 32991844; PMCID: PMC7494283.
17. King H A D, Joyce M G, Lakhal-Naouar I, Ahmed A, Cincotta C M, Subra C, Peachman K K, Hack H R, Chen R E, Thomas P V, Chen W H, Sankhala R S, Hajduczki A, Martinez E J, Peterson C E, Chang W C, Choe M, Smith C, Headley J A, Elyard H A, Cook A, Anderson A, Wuertz K M, Dong M, Swafford I, Case J B, Currier J R, Lal K G, Amare M F, Dussupt V, Molnar S, Daye S P, Zeng X, Barkei E K, Alfson K, Staples H M, Carrion R, Krebs S J, Paquin-Proulx D, Karasavvas N, Polonis V R, Jagodzinski L L, Vasan S, Scott P T, Huang Y, Nair M S, Ho D D, de Val N, Diamond M S, Lewis M G, Rao M, Matyas G R, Gromowski G D, Peel S A, Michael N L, Modjarrad K, Bolton D L. Efficacy and breadth of adjuvanted SARS-CoV-2 receptor-binding domain nanoparticle vaccine in macaques. Proc Natl Acad Sci USA. 2021; 118(38). Epub 2021/09/03. doi: 10.1073/pnas.2106433118. PubMed PMID: 34470866.
18. López-Sagaseta J, Malito E, Rappuoli R, Bottomley M J. Self-assembling protein nanoparticles in the design of vaccines. Comput Struct Biotechnol J. 2016; 14:58-68. Epub 2016/02/11. doi: 10.1016/j.csbj.2015.11.001. PubMed PMID: 26862374; PMCID: PMC4706605.
19. Bangaru S, Ozorowski G, Turner H L, Antanasijevic A, Huang D, Wang X, Torres J L, Diedrich J K, Tian J H, Portnoff A D, Patel N, Massare M J, Yates J R, 3rd, Nemazee D, Paulson J C, Glenn G, Smith G, Ward A B. Structural analysis of full-length SARS-CoV-2 spike protein from an advanced vaccine candidate. Science. 2020; 370(6520):1089-94. Epub 2020/10/22. doi: 10.1126/science.abel502. PubMed PMID: 33082295.
20. Walls A C, Fiala B, Schafer A, Wrenn S, Pham M N, Murphy M, Tse L V, Shehata L, O'Connor M A, Chen C, Navarro M J, Miranda M C, Pettie D, Ravichandran R, Kraft J C, Ogohara C, Palser A, Chalk S, Lee E C, Guerriero K, Kepl E, Chow C M, Sydeman C, Hodge E A, Brown B, Fuller J T, Dinnon K H, 3rd, Gralinski L E, Leist S R, Gully K L, Lewis T B, Guttman M, Chu H Y, Lee K K, Fuller D H, Baric R S, Kellam P, Carter L, Pepper M, Sheahan T P, Veesler D, King N P. Elicitation of Potent Neutralizing Antibody Responses by Designed Protein Nanoparticle Vaccines for SARS-CoV-2. Cell. 2020; 183(5):1367-82 e17. Epub 2020/11/09. doi: 10.1016/j.cell.2020.10.043. PubMed PMID: 33160446; PMCID: PMC7604136.
21. Ma X, Zou F, Yu F, Li R, Yuan Y, Zhang Y, Zhang X, Deng J, Chen T, Song Z, Qiao Y, Zhan Y, Liu J, Zhang J, Zhang X, Peng Z, Li Y, Lin Y, Liang L, Wang G, Chen Y, Chen Q, Pan T, He X, Zhang H. Nanoparticle Vaccines Based on the Receptor Binding Domain (RBD) and Heptad Repeat (HR) of SARS-CoV-2 Elicit Robust Protective Immune Responses. Immunity. 2020; 53(6):1315-30 e9. Epub 2020/12/05. doi: 10.1016/j.immuni.2020.11.015. PubMed PMID: 33275896; PMCID: PMC7687490.
22. Polack F P, Thomas S J, Kitchin N, Absalon J, Gurtman A, Lockhart S, Perez J L, Perez Marc G, Moreira E D, Zerbini C, Bailey R, Swanson K A, Roychoudhury S, Koury K, Li P, Kalina W V, Cooper D, Frenck R W, Jr., Hammitt L L, Tureci 0, Nell H, Schaefer A, Unal S, Tresnan D B, Mather S, Dormitzer P R, Sahin U, Jansen K U, Gruber W C, Group CCT. Safety and Efficacy of the BNT162b2 mRNA Covid-19 Vaccine. N Engl J Med. 2020; 383(27):2603-15. Epub 2020/12/11. doi: 10.1056/NEJMoa2034577. PubMed PMID: 33301246; PMCID: PMC7745181.
23. Baden L R, El Sahly H M, Essink B, Kotloff K, Frey S, Novak R, Diemert D, Spector S A, Rouphael N, Creech C B, McGettigan J, Khetan S, Segall N, Solis J, Brosz A, Fierro C, Schwartz H, Neuzil K, Corey L, Gilbert P, Janes H, Follmann D, Marovich M, Mascola J, Polakowski L, Ledgerwood J, Graham B S, Bennett H, Pajon R, Knightly C, Leav B, Deng W, Zhou H, Han S, Ivarsson M, Miller J, Zaks T, Group C S. Efficacy and Safety of the mRNA-1273 SARS-CoV-2 Vaccine. N Engl J Med. 2021; 384(5):403-16. Epub 2020/12/31. doi: 10.1056/NEJMoa2035389. PubMed PMID: 33378609; PMCID: PMC7787219.
24. Li D, Edwards R J, Manne K, Martinez D R, Schafer A, Alam S M, Wiehe K, Lu X, Parks R, Sutherland L L, Oguin T H, McDanal C, Perez L G, Mansouri K, Gobeil S M C, Janowska K, Stalls V, Kopp M, Cai F, Lee E, Foulger A, Hernandez G E, Sanzone A, Tilahun K, Jiang C, Tse L V, Bock K W, Minai M, Nagata B M, Cronin K, Gee-Lai V, Deyton M, Barr M, Von Holle T, Macintyre A N, Stover E, Feldman J, Hauser B M, Caradonna™, Scobey T D, Moody M A, Cain D W, DeMarco C T, Denny T N, Woods C W, Petzold E W, Schmidt A G, Teng I T, Zhou T, Kwong P D, Mascola J R, Graham B S, Moore I N, Seder R, Andersen H, Lewis M G, Montefiori D C, Sempowski G D, Baric R S, Acharya P, Haynes B F, Saunders K O. The functions of SARS-CoV-2 neutralizing and infection-enhancing antibodies in vitro and in mice and nonhuman primates. bioRxiv. 2021. Epub 2021/01/15. doi: 10.1101/2020.12.31.424729. PubMed PMID: 33442694; PMCID: PMC7805451.
25. Rappazzo C G, Tse L V, Kaku C I, Wrapp D, Sakharkar M, Huang D, Deveau L M, Yockachonis T J, Herbert A S, Battles M B, O'Brien C M, Brown M E, Geoghegan J C, Belk J, Peng L, Yang L, Hou Y, Scobey T D, Burton D R, Nemazee D, Dye J M, Voss J E, Gunn B M, McLellan J S, Baric R S, Gralinski L E, Walker L M. Broad and potent activity against SARS-like viruses by an engineered human monoclonal antibody. Science. 2021. Epub 2021/01/27. doi: 10.1126/science.abf4830. PubMed PMID: 33495307.
26. Tan C W, Chia W N, Young B E, Zhu F, Lim B L, Sia W R, Them T L, Chen M I, Leo Y S, Lye D C, Wang L F. Pan-Sarbecovirus Neutralizing Antibodies in BNT162b2-Immunized SARS-CoV-1 Survivors. N Engl J Med. 2021. Epub 2021/08/19. doi: 10.1056/NEJMoa2108453. PubMed PMID: 34407341; PMCID: PMC8422514.

### Example 5: Disulfide Stabilized RBD

To stabilize the receptor binding domain of various coronaviruses, we introduced cysteine residues that make new disulfide bonds within each RBD molecule. These new disulfide bonds hold individual strands of receptor binding domain together such that the RBD is constrained from changing conformation. The cysteine locations were determined by loading the structures of SARS-CoV-2, MERS-CoV, or SARS-CoV S proteins (PDB: 6VXX, 5W9H, 7AKJ) into Disulfide by Design (http://cptweb.cpt.wayne.edu/DbD2/index.php). The sites for introduction of cysteines were noted for the amino acids that correspond to the receptor binding domain of each protein.

Two wildtype amino acids were replaced with pairs of cysteines predicted based on the structure to be correctly spaced apart and orientated correctly for a disulfide bond to form. Each pair of cysteines were evaluated individually for improvement in thermostability and overall protein yield. Mutations that improved these protein features were combined into a single protein in various combinations to get a more stable, higher-yield expressing molecule.

TABLE 5
Listing of SARS-CoV2 RBD disulfide designs. Non-limiting
embodiments of sequences are shown in FIG. 43.

| Protein ID | Protein name |
|---|---|
| HV1302813 | SARS2RBDcore_ext_v2_3C-twST2_W353C_F400C |
| HV1302814 | SARS2RBDcore_ext_v2_3C-twST2_I358C_V395C |
| HV1302815 | SARS2RBDcore_ext_v2_3C-twST2_A435C_V510C |
| HV1302816 | SARS2RBDcore_ext_v2_3C-twST2_P412C_F429C |
| HV1302817 | SARS2RBDcore_ext_v2_3C-twST2_D405C_G504C |
| HV1302818 | SARS2RBDcore_ext_v2_3C-twST2_S438C_D442C |
| HV1302819 | SARS2RBDcore_ext_v2_3C-twST2_N501C_Y505C |
| HV1302820 | SARS2RBDcore_ext_v2_3C-twST2_G447C_G496C |
| HV1302821 | SARS2RBDcore_ext_v2_3C-twST2_Y365C_L387C |
| HV1302822 | SARS2RBDcore_ext_v2_3C-twST2_F497C_P507C |
| HV1302823 | SARS2RBDcore_ext_v2_3C-twST2_Q409C_A419C |

Sites where cysteines will be introduced in MERS-CoV Spike protein for stabilizing the RBD.

TABLE 6
MERS-CoV S sites for Cys residues to form new disulfide bonds.
MERS-CoV S sites for Cys residues to form new disulfide bonds

| Mutation | Amino acid | Amino acid |
|---|---|---|
| pair number | position 1 | position 2 |
| 1 | 383 | 409 |
| 2 | 390 | 490 |
| 3 | 393 | 447 |
| 4 | 394 | 398 |
| 5 | 399 | 446 |
| 6 | 408 | 587 |
| 7 | 410 | 413 |
| 8 | 418 | 482 |
| 9 | 422 | 481 |
| 10 | 428 | 476 |
| 11 | 429 | 432 |
| 12 | 439 | 581 |
| 13 | 449 | 561 |
| 14 | 455 | 464 |
| 15 | 460 | 465 |
| 16 | 463 | 501 |
| 17 | 471 | 477 |
| 18 | 484 | 567 |
| 19 | 498 | 534 |
| 20 | 502 | 557 |
| 21 | 506 | 553 |
| 22 | 507 | 512 |
| 23 | 526 | 556 |
| 24 | 534 | 558 |
| 25 | 540 | 557 |
| 26 | 545 | 549 |
| 27 | 578 | 582 |
| 28 | 588 | 591 |

Sites where cysteines will be introduced in SARS-CoV Spike protein for stabilizing the RBD.

TABLE 7
SARS-CoV S sites for Cys residues to form new disulfide bonds.
SARS-CoV S sites for Cys residues to form new disulfide bonds

| Mutation | Amino acid | Amino acid |
|---|---|---|
| pair number | position 1 | position 2 |
| 1 | 323 | 348 |
| 2 | 323 | 350 |
| 3 | 340 | 387 |
| 4 | 346 | 509 |
| 5 | 352 | 374 |
| 6 | 366 | 419 |
| 7 | 368 | 417 |
| 8 | 378 | 508 |
| 9 | 378 | 511 |
| 10 | 380 | 504 |
| 11 | 401 | 406 |
| 12 | 413 | 450 |
| 13 | 425 | 429 |
| 14 | 429 | 493 |
| 15 | 434 | 482 |
| 16 | 436 | 482 |
| 17 | 441 | 478 |
| 18 | 443 | 477 |
| 19 | 448 | 452 |
| 20 | 469 | 474 |
| 21 | 487 | 491 |
| 22 | 524 | 576 |

Each pair of cysteines modifications in SARS1 and MERS1 sequences will evaluated individually for improvement in thermostability and overall protein yield. Mutations that improve these protein features will be combined into a single protein in various combinations to get a more stable, higher-yield expressing molecule.

1. A recombinant fusion protein comprising in order a full-length coronavirus spike protein or a portion thereof, a peptide linker and a self-assembling protein, wherein the fusion protein is comprised in a multimeric protein complex, wherein the recombinant fusion protein is shown in FIG. 18, FIG. 20, FIG. 21, FIG. 22, FIG. 23, and FIG. 45 and Table 1, Table 2, Table 3 and Table 5.

2. The recombinant fusion protein of claim 1, wherein the coronavirus spike proteins or portions thereof is RBD—amino acids 319-541; RBDcore—amino acids 333-524 see FIG. 21B, RBDcoreExt—amino acids 333-527, RBDcoreExtKK—amino acids 333-529 see FIG. 22, or N-terminal domain (NTD)—amino acids 27-292, wherein amino acid positions are with respect to CoV2 sequence.

3. The recombinant fusion protein of claim 1 wherein the linker is described in Table 4,
or is GGS(n)EKAAKAEEAAR(PP) (SEQ ID NO: 4) or EKAAKAEEAAR(PP)GGS(n), wherein n=1, 2, or 3 (SEQ ID NO: 5).

4. (canceled)

5. (canceled)

6. (canceled)

7. (canceled)

8. The recombinant fusion protein of claim 1 wherein the self-assembling protein is ferritin and the multimeric protein complex is a ferritin nanoparticle.

9. A nucleic acid encoding the coronavirus spike protein or fragment thereof in the recombinant fusion protein of claim 1.

10. A multimeric protein complex comprising the recombinant fusion protein sequence of claim 1.

11. (canceled)

12. (canceled)

13. (canceled)

14. (canceled)

15. (canceled)

16. (canceled)

17. (canceled)

18. The multimeric protein complex of claim 10, wherein the multimeric protein complex is multispecific comprising:
(a) two, three, four or more different recombinant fusion proteins comprising in order a full-length coronavirus spike protein or a portion thereof, wherein the two, three, four or more full-length coronavirus spike protein or a portion thereof have different sequences, a peptide linker and a self-assembling protein,
(b) two or three different recombinant fusion proteins comprising in order a full-length coronavirus spike protein or a portion thereof, a peptide linker and a self-assembling protein, wherein the recombinant fusion proteins represent different coronaviruses from subgroups 2a, 2b, 2c, 2d or combinations thereof,
(c) two or three different recombinant fusion proteins comprising in order a full-length coronavirus spike protein or a portion thereof, a peptide linker and a self-assembling protein, wherein the recombinant fusion proteins comprise beta coronavirus sequences, delta coronavirus sequences or combinations thereof, or
(d) one or more recombinant fusion proteins comprising in order a full-length coronavirus spike protein or a portion thereof, a peptide linker and a self-assembling protein, wherein the recombinant fusion proteins comprise recombinant fusion proteins from group 2b and group 2c betacoronaviruses.

19. (canceled)

20. (canceled)

21. (canceled)

22. (canceled)

23. A composition comprising the recombinantly fusion protein or a portion thereof of claim 1 and a pharmaceutically acceptable carrier.

24. A composition comprising a plurality of multimeric protein complexes of claim 10.

25. The composition of claim 24 wherein the plurality of multimeric protein complexes comprises:
(a) complexes each one having an identical recombinant fusion protein sequence comprising in order a full-length coronavirus spike protein or a portion thereof, a peptide linker and a self-assembling protein, or
(b) two, three, or four multimeric complexes each one having a different recombinant fusion protein sequence comprising in order a full-length coronavirus spike protein or a portion thereof, a peptide linker and a self-assembling protein.

26. (canceled)

27. A composition comprising one or more nucleic acids encoding a recombinant fusion protein of claim 1 and a pharmaceutically acceptable carrier.

28. (canceled)

29. The composition of claim 27 wherein the nucleic acid is an mRNA.

30. A virus-like particle comprising the recombinant fusion protein of claim 1.

31. A host cell comprising a nucleic acid molecule encoding a recombinant fusion protein of claim 1.

32. A method of producing the multimeric protein complex of claim 10 comprising:
expressing a nucleic acid molecule or vector encoding a recombinant fusion protein in a host cell under conditions suitable to produce the recombinant fusion protein;
purifying the recombinant fusion protein;
conjugating the recombinant fusion protein to a self-assembling ferritin protein in the presence of a sortase enzyme, under suitable conjugation conditions to form a multimeric ferritin protein complex comprising the recombinant fusion protein; and
isolating the multimeric ferritin protein complex comprising the recombinant fusion protein from the conjugation reaction.

33. An immunogenic composition comprising the recombinant fusion protein of claim 1, a nucleic acid encoding the recombinant fusion protein, a multimeric protein complex or a VLP comprising the recombinant fusion protein and a pharmaceutically acceptable carrier and an adjuvant.

34. A method for inducing an immune response to a coronavirus in a subject, comprising administering to the subject an effective amount of the recombinant fusion protein of claim 1, a nucleic acid encoding the recombinant fusion protein, or a multimeric protein complex or a VLP in an amount sufficient to induce an immune response.

35. The method of claim 34, wherein the effective amount is administered as a boost.
